SEQUENCE LISTING (1 sequence)

SEQ ID NO: 1 - protein, 584 aa, Hepatitis C virus
MASSMSYTWTGALITPCAAEESKLPINPLSNSLLRHHNMVYATTSRSASLRQKKVTFDRLQVLDDHYRDVLKEMKAKASTVKAKLLSIEEACKLTPPHSAKSKFGYGAKDVRNLSSRAVNHIRSVWEDLLEDTETPIDTTIMAKSEVFCVQPEKGGRKPARLIVFPDLGVRVCEKMALYDVVSTLPQAVMGSSYGFQYSPKQRVEFLVNTWKSKKCPMGFSYDTRCFDSTVTESDIRVEESIYQCCDLAPEARQAIRSLTERLYIGGPLTNSKGQNCGYRRCRASGVLTTSCGNTLTCYLKATAACRAAKLQDCTMLVNGDDLVVICESAGTQEDAAALRAFTEAMTRYSAPPGDPPQPEYDLELITSCSSNVSVAHDASGKRVYYLTRDPTTPLARAAWETARHTPINSWLGNIIMYAPTLWARMILMTHFFSILLAQEQLEKALDCQIYGACYSIEPLDLPQIIERLHGLSAFTLHSYSPGEINRVASCLRKLGVPPLRTWRHRARSVRAKLLSQGGRAATCGRYLFNWAVRTKLKLTPIPAASQLDLSGWFVAGYSGGDIYHSLSRARPRAAALEHHHHHH

(19) United States
(12) Patent Application Publication
(10) Pub. No.: US 2009/0221559 A1
(43) Pub. Date: Sep. 3, 2009

(54) BENZODIAZEPINES AS HCV INHIBITORS

(76) Inventors: Jean-Francois Bonfanti, Ande (FR); Frederic Marc Maurice Doublet, Isneauville (FR); Origene Nyanguile, Brussels (BE); Pierre Jean-Marie Bernard Raboisson, Rosieres (BE); Anne-Sophie Helene Marie Rebstock, Volmerange-les-mines (FR); Carlo Willy Maurice Boutton, Wielsbeke (BE)

Correspondence Address: PHILIP S. JOHNSON;JOHNSON & JOHNSON, ONE JOHNSON & JOHNSON PLAZA, NEW BRUNSWICK, NJ, 08933-7003, US

(21) Appl. No.: 12/065,345

(22) PCT Filed: Sep. 1, 2006
(86) PCT No.: PCT/EP06/65938

§ 371 Date: Feb. 29, 2008

(30) Foreign Application Priority Data
Sep. 2, 2005 (EP) 05108058.8
Nov. 10, 2005 (EP) 05110606.0

(51) Int. Cl. A61K 31/5513; C07D 243/14; A61P 31/12

(52) U.S. Cl. 514/220; 540/557

(57) ABSTRACT

The present invention relates to the use of benzodiazepines as inhibitors of HCV replication as well as their use in pharmaceutical compositions aimed to treat or combat HCV infections. In addition, the present invention relates to benzodiazepine compounds per se and their use as medicines. The present invention also concerns processes for the preparation of such compounds, pharmaceutical compositions comprising them, and combinations of said compounds with other anti-HCV agents.

This application claims priority of the benefits of the filing of PCT/EP2006/065938 filed Sep. 1, 2006; EP 05108058.8 filed Sep. 2, 2005; and EP 05110606 filed Nov. 10, 2005. The complete disclosures of the aforementioned related patent applications are hereby incorporated herein by reference for all purposes.

The present invention relates to the use of benzodiazepines as inhibitors of HCV replication as well as their use in pharmaceutical compositions aimed to treat or combat HCV infections. In addition, the present invention relates to compounds per se. The present invention also concerns processes for the preparation of such compounds, pharmaceutical compositions comprising them, and combinations of said compounds with other anti-HCV agents.

Following its discovery in 1989 as the agent implicated in the majority of viral non-A, non-B hepatitis (Choo et al., Science 244, 359-362, 1989), hepatitis C virus (HCV) has become a focus of considerable medical research (Lauer, G. M and Walker, B. D., New Eng. J. Med. 345, 41-52, 2001). HCV is a member of the Flaviviridae family of viruses in the hepacivirus genus, and is closely related to the flavivirus genus, which includes a number of viruses implicated in human disease, such as dengue virus and yellow fever virus, and to the animal pestivirus family, which includes bovine viral diarrhea virus (BVDV). HCV is a positive-sense, single-stranded RNA virus, with a genome of around 9,600 bases. The genome comprises both 5′ and 3′ untranslated regions which adopt RNA secondary structures, and a central open reading frame that encodes a single polyprotein of around 3,010-3,030 amino acids. The polyprotein encodes ten gene products which are generated from the precursor polyprotein by an orchestrated series of co- and posttranslational endoproteolytic cleavages mediated by both host and viral proteases. The viral structural proteins include the core nucleocapsid protein, and two envelope glycoproteins E1 and E2. The non-structural (NS) proteins encode some essential viral enzymatic functions (helicase, polymerase, protease), as well as proteins of unknown function. Replication of the viral genome is mediated by an RNA-dependent RNA polymerase, encoded by non-structural protein 5b (NS5b). In addition to the polymerase, the viral helicase and protease functions, both encoded in the bifunctional NS3 protein, have been shown to be essential for replication of HCV RNA in chimpanzee models of infection (Kolykhalov, A. A., Mihalik, K., Feinstone, S. M., and Rice, C. M. J. Virol. 74, 2046-2051, 2000). In addition to the NS3 serine protease, HCV also encodes a metalloproteinase in the NS2 region.

HCV replicates preferentially in hepatocytes but is not directly cytopathic, leading to persistent infection. In particular, the lack of a vigorous T-lymphocyte response and the high propensity of the virus to mutate appear to promote a high rate of chronic infection. There are 6 major HCV genotypes and more than 50 subtypes, which are differently distributed geographically. HCV type 1 is the predominant genotype in the US and Europe. For instance, HCV type 1 accounts for 70 to 75 percent of all HCV infections in the United States. The extensive genetic heterogeneity of HCV has important diagnostic and clinical implications, perhaps explaining difficulties in vaccine development and the lack of response to therapy. An estimated 170 million persons worldwide are infected with hepatitis C virus (HCV). Following the initial acute infection, a majority of infected individuals develop chronic hepatitis, which can progress to liver fibrosis leading to cirrhosis, end-stage liver disease, and HCC (hepatocellular carcinoma) (National Institutes of Health Consensus Development Conference Statement: Management of Hepatitis C. Hepatology, 36, 5 Suppl. S3-S20, 2002). Liver cirrhosis due to HCV infection is responsible for about 10,000 deaths per year in the U.S.A. alone, and is the leading cause for liver transplantations. Transmission of HCV can occur through contact with contaminated blood or blood products, for example following blood transfusion or intravenous drug use. The introduction of diagnostic tests used in blood screening has led to a downward trend in post-transfusion HCV incidence. However, given the slow progression to the end-stage liver disease, the existing infections will continue to present a serious medical and economic burden for decades (Kim, W. R. Hepatology, 36, 5 Suppl. S30-S34, 2002).

The treatment of this chronic disease is an unmet clinical need, since current therapy is only partially effective and limited by undesirable side effects.

Current HCV therapies are based on (pegylated) interferon-alpha (IFN-α) in combination with ribavirin. This combination therapy yields a sustained virologic response in more than 40% of patients infected by genotype 1 viruses and about 80% of those infected by genotypes 2 and 3. Beside the limited efficacy on HCV type 1, combination therapy has significant side effects and is poorly tolerated in many patients. For instance, in registration trials of pegylated interferon and ribavirin, significant side effects resulted in discontinuation of treatment in approximately 10 to 14 percent of patients. Major side effects of combination therapy include influenza-like symptoms, hematologic abnormalities, and neuropsychiatric symptoms. The development of more effective, convenient and tolerated treatments is a major public health objective.

One area of particular focus has been the search for inhibitors of the NS5b RNA-dependent RNA polymerase referred to above as close structural homologs of this polymerase do not exist within the uninfected host cell and such inhibitors will provide a more specific mode of action. Inhibitors which are currently under investigation can be classified as either nucleoside inhibitors (NIs) or non-nucleoside inhibitors (NNIs). NIs directly compete with nucleotide substrates for binding to highly conserved active sites. Greater specificity may be achieved by NNIs, which may interact outside of the highly conserved active site at a unique allosteric site common only to structurally related polymerases. Preliminary clinical trials have resulted in a high failure rate, thereby highlighting the need to pursue the search for novel NS5b inhibitors.

Thus, there is a high medical need for low molecular weight compounds that lead to an inhibition of HCV replication.

It has been surprisingly found that certain benzodiazepine derivatives exhibit antiviral activity in mammals infected with HCV. These compounds are therefore useful in treating or combating HCV infections.

WO00/66106 discloses 1,4-benzodiazepine-2-one and 1,4-benzodiazepine-2,5-dione compounds, enantiomers, pharmaceutically acceptable salts, prodrugs or derivatives of the benzodiazepine compounds. These benzodiazepine compounds can be used to treat a variety of dysregulatory disorders related to cellular death, such as autoimmune disorders, inflammatory conditions, hyperproliferative conditions, viral infections, and atherosclerosis.

WO99/58117 relates to the use of compounds for reducing apoptosis. Said compounds are ligands of benzodiazepine peripheral receptor.

WO00/12547 relates to 1,4-benzodiazepines or 1,4-benzothiazepines derivatized with a peptide that can inhibit the interaction between annexin and annexin binding proteins, in particular, the interaction between annexin and viral proteins that bind annexins such as the HBsAg protein of HBV, glycoprotein B of the cytomegalovirus or any annexin binding protein from the influenza virus. These 1,4-benzodiazepines or 1,4-benzo-thiazepines derivatives can be used to prevent or treat diseases in which interactions between annexin family members and annexin binding proteins are involved such as HBV and/or HDV infections, cytomegalovirus infections or influenza virus infections.

EP0574781 discloses 2-amino-5-heterocyclic-substituted-1,4-benzodiazepines and their use in the treatment of AIDS and AIDS-related diseases.

Cortés E C et al.: “Efficient synthesis and spectral determination of 11-[(o-; m-; and p-substituted)-phenyl]-8-chloro-3,3-dimethyl-2,3,4,5,10,11-hexahydro-1H-dibenzo[b,e][1,4]diazepin-1-ones”. Journal of Heterocyclic Chemistry 2004, 41(2), 277-280. This publication discloses the synthesis of 11-aryl-8-chloro-3,3-dimethyl-2,3,4,5,10,11-hexahydro-1H-dibenzo[b,e][1,4]diazepin-1-ones, with possible pharmacological activity in the central nervous system.

Cortés Cortés E et al.: “Synthesis and spectral properties of 11-[(o-; and p-substituted)-phenyl]-8-[(o-; m-; p-methoxy)phenylthio]-3,3-dimethyl-2,3,4,5,10,11-hexahydro-1H-dibenzo[b,e][1,4]diazepin-1-ones”. Journal of Heterocyclic Chemistry 2002, 39(1), 55-59. This publication discloses the preparation of twelve 2,3,4,5,10,11-hexahydro-1H-dibenzo[b,e][1,4]diazepin-1-ones which have potentially useful pharmacological properties; by condensation and cyclization between 3-{[4-(o-; m-; p-methoxy)-phenylthio]-1,2-phenylenediamine}-5,5-dimethyl-2-cyclohexenone with (o-; and p-substituted)benzaldehyde.

Matsuo K et al.: “Synthesis and reactions of 11-substituted 3,3-dimethyl-2,3,4,5-tetra-hydro-1H-dibenzo[b,e][1,4]diazepin-1-ones”. Chemical & Pharmaceutical Bulletin 1985, 33(9), 4057-62. This publication discloses 11-substituted 3,3-dimethyl-2,3,4,5-tetrahydro-1H-dibenzo[b,e][1,4]diazepin-1-ones which are prepared by dehydrative cyclization of 3-(2-acylaminoanilino)-5,5-dimethyl-2-cyclohexen-1-ones with polyphosphoric acid, showing moderate analgesic activity in mice at 50 mg/kg.

WO 04/001058 describes certain 2,3,4,5,10,11-hexahydro-3,3-dimethyl-1H-dibenzo-[b,e][1,4]diazepin-1-one derivatives as transcription modulating agents useful as anti-infective agents.

US 2005/123906 describes certain 2,3,4,5,10,11-hexahydro-1H-dibenzo[b,e][1,4]-diazepin-1-one derivatives as protein modulating agents.

WO 05/007141 describes certain 2,3,4,5,10,11-hexahydro-3,3-dimethyl-1H-dibenzo-[b,e][1,4]diazepin-1-one derivatives as inhibitors of RING domain ubiquitin ligases.

US 2003/229065 describes certain 2,3,4,5,10,11-hexahydro-3,3-dimethyl-1H-dibenzo-[b,e][1,4]diazepin-1-one derivatives as transcription modulating agents useful as anti-infective agents.

Other 2,3,4,5,10,11-hexahydro-3,3-dimethyl-1H-dibenzo[b,e][1,4]diazepin-1-one derivatives are described in the following references, generally without reference to any specific pharmaceutical utility:
Chemistry of Heterocyclic Compounds (New York, N.Y., United States)(Translation of Khimiya Geterotsiklicheskikh Soedinenii) (2004), 40(7), 949-955;
Journal of Heterocyclic Chemistry (2004), 41(2), 277-280; Rigas Tehniskas Universitates Zinatniskie Raksti, Serija 1:
Materialzinatne un Lietiska Kimija (2002), 4, 84-88; Rigas Tehniskas Universitates Zinatniskie Raksti, Serija 1:
Materialzinatne un Lietiska Kimija (2001), (3), 24-27;
Journal of Heterocyclic Chemistry (2002), 39(1), 55-59;
THEOCHEM (1999), 489(1), 7-17;
Heterocyclic Communications (1996), 2(1), 47-50;
Alexandria Journal of Pharmaceutical Sciences (1993), 7(2), 137-9;
Journal of the Chinese Chemical Society (Taipei, Taiwan) (1993), 40(2), 189-94
Journal of the Indian Chemical Society (1992), 69(9), 596-8;
Bulletin des Societes Chimiques Belges (1992), 101(9), 801-6;
Chemistry Express (1992), 7(2), 133-6;
Journal of the Indian Chemical Society (1990), 67(7), 609-10;
Acta Crystallographica, Section C: Crystal Structure Communications (1987), C43(6), 1161-3;
Chemical & Pharmaceutical Bulletin (1985), 33(9), 4057-62;
Journal of Heterocyclic Chemistry (1982), 19(2), 321-6;
JP 47029385; and
Chemical & Pharmaceutical Bulletin (1972), 20(7), 1588-9.

The present invention thus relates to the use of the compounds of the formula (I) for the manufacture of a medicament useful for inhibiting HCV activity in a mammal infected with HCV, said compounds being benzodiazepines of the formula (I):

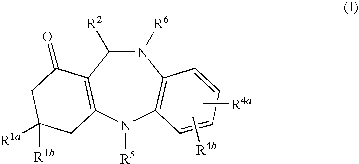

and the salts, stereoisomeric forms, and racemic mixtures thereof in which
R^{1a} and R^{1b} are independently, hydrogen; C₃₋₇cycloalkyl; aryl; Het; or C₁₋₆alkyl optionally substituted independently with one, two or three substituents selected from halo, C₁₋₆alkoxy, aryl and Het; or with a cyano, polyhaloC₁₋₆alkoxy or C₃₋₇cycloalkyl;
R² is hydrogen;
   C₁₋₆alkyl optionally substituted independently with one, two or three substituents selected from halo, C₁₋₆alkoxy, aryl and Het; or with a cyano, polyhaloC₁₋₆alkoxy or C₃₋₇cycloalkyl;
   C₃₋₇cycloalkyl optionally substituted independently with one, two or three substituents selected from halo, C₁₋₆alkoxy, aryl and Het; or with a cyano, polyhaloC₁₋₆alkoxy or C₃₋₇cycloalkyl,
   C₃₋₇cycloalkylC₁₋₆alkyl optionally substituted independently with one, two or three substituents selected from halo, C₁₋₆alkoxy, aryl and Het; or with a cyano, polyhaloC₁₋₆alkoxy or C₃₋₇cycloalkyl;
   C₂₋₆alkenyl optionally substituted independently with one, two or three substituents selected from halo, C₁₋₆alkoxy, aryl and Het; or with a cyano, polyhaloC₁₋₆alkoxy or C₃₋₇cycloalkyl;
   C₄₋₇cycloalkenyl optionally substituted independently with one, two or three substituents selected from halo, C₁₋₆alkoxy, aryl and Het; or with a cyano, polyhaloC₁₋₆alkoxy or C₃₋₇cycloalkyl;
   C₄₋₈cycloalkenylC₁₋₆alkyl optionally substituted independently with one, two or three substituents selected from halo, C₁₋₆alkoxy, aryl and Het; or with a cyano, polyhaloC₁₋₆alkoxy or C₃₋₇cycloalkyl;
   aryl²; or
   Het²;
R⁶ is hydrogen;
   C₁₋₆alkyl optionally substituted with carboxyl, C₁₋₆alkylcarbonyl, C₁₋₆alkoxy-carbonyl, Het-C₁₋₆alkylaminocarbonyl;
   —C(═O)—C₁₋₇alkyl, the C₁₋₇alkyl being optionally substituted independently with one, two or three substituents selected from halo, C₁₋₆alkoxy, aryl, Het, cyano, polyhaloC₁₋₆alkoxy, C₃₋₇cycloalkyl, and carboxyl;
   —C(═O)—C₂₋₆alkenyl;
   —C(═O)—C₃₋₇cycloalkyl, the C₃₋₇cycloalkyl being optionally substituted independently with one, two or three substituents selected from halo, C₁₋₆alkoxy, aryl, Het, cyano, polyhaloC₁₋₆alkoxy, and C₃₋₇cycloalkyl;
   —C(═O)-aryl;
   —C(═O)-Het;
   —C(═O)—NR^{12a}R^{12b},
   in which each R^{12a} and R^{12b} is, independently, hydrogen, C₃₋₇cycloalkyl, aryl, Het, or C₁₋₆alkyl optionally substituted independently with one, two or three substituents selected from halo, C₁₋₆alkoxy, aryl, Het, cyano, polyhaloC₁₋₆alkoxy, and C₃₋₇cycloalkyl;
   —C(═O)—OR^{13a},
   in which R¹³ is hydrogen, C₂₋₆alkenyl, C₃₋₇cycloalkyl, Het, or C₁₋₆alkyl optionally substituted with a C₃₋₇cycloalkyl or Het;
   —C(═O)—C₁₋₆alkyloxycarbonylC₁₋₆alkyl;
   —C(═O)—Het-thioC₁₋₆alkyl; or
   —C(═O)-Het-oxyC₁₋₆alkyl; or
R² and R⁶, together with the intervening grouping in formula (I) of sub-formula:

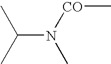

form a ring of formula:

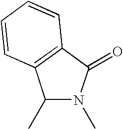

R^{4a} and R^{4b} are independently hydrogen; halo; cyano; C₁₋₆alkyl optionally substituted with halo, hydroxy, Het, —OR^{14a}, or —NR^{14a}R^{14b}; C₁₋₆alkoxy optionally substituted with amino, hydroxy, C₁₋₆alkoxy, hydroxycarbonyl, aryl, or Het; aryloxy; Het-oxy; carboxyl; C₁₋₆alkylcarbonyloxy; C₁₋₆alkoxycarbonyl; arylcarbonyl; —NR^{14a}R^{14b}; or —C(═O)—NR^{14a}R^{14b};
   in which each R^{14a} and R^{14b} is, independently, hydrogen; C₃₋₇cycloalkyl; aryl; Het; or C₁₋₆alkyl optionally substituted independently with one, two or three substituents selected from halo, C₁₋₆alkoxy, mono- or diC₁₋₆alkylamino, aryl, Het, cyano, polyhaloC₁₋₆alkoxy, and C₃₋₇cycloalkyl;
R⁵ is hydrogen; C₃₋₇cycloalkyl; or C₁₋₆alkyl optionally substituted with a C₃₋₇cyclo-alkyl, aryl, Het, —C(═O)NR^{15a}R^{15b}, —NR^{15a}R^{15b}, —C(═O)R¹⁷, —NR^{15a}C(═O)R¹⁷, —NR^{15a}SOₚR¹⁸, —SOₚR¹⁸, —SOₚNR^{15a}R^{15b}, —C(═O)OR¹⁶, or —NR^{15a}C(═O)OR^{16a} in which
   p is 0, 1 or 2;
   each R^{15a} and R^{15b} is, independently, hydrogen; C₃₋₇cycloalkyl; aryl; Het; or C₁₋₆alkyl optionally substituted independently with one, two or three substituents selected from halo, C₁₋₆alkoxy, aryl and Het; or with a cyano, polyhaloC₁₋₆alkoxy or C₃₋₇cycloalkyl;
   R¹⁶ is hydrogen; C₂₋₆alkenyl; C₃₋₇cycloalkyl; Het; or C₁₋₆alkyl optionally substituted with a C₃₋₇cycloalkyl or Het;
   R^{16a} is C₂₋₆alkenyl; C₃₋₇cycloalkyl; Het; or C₁₋₆alkyl optionally substituted with a C₃₋₇cycloalkyl or Het;
   R¹⁷ is hydrogen, C₁₋₆alkyl, C₃₋₇cycloalkyl or aryl;
   R¹⁸ is hydrogen; polyhaloC₁₋₆alkyl; C₃₋₇cycloalkyl; aryl; Het; or C₁₋₆alkyl optionally substituted with a C₃₋₇cycloalkyl, aryl or Het;
      aryl as a group or part of a group is phenyl, naphthyl, indanyl, or 1,2,3,4-tetrahydro-naphthyl, each of which may be optionally independently substituted with
(a) one, two or three substituents selected from halo, C₁₋₆alkyl, polyhaloC₁₋₆alkyl, hydroxy, trifluoromethyl, alkylenedioxy, C₁₋₆alkoxy, C₁₋₆alkylthio, polyhalo-C₁₋₆alkoxy, C₁₋₆alkoxyC₁₋₆alkyl, carboxyl, C₁₋₆alkylcarbonyl, cyano, cyanoC₁₋₆alkyl, nitro, amino, mono- or diC₁₋₆alkylamino, azido, mercapto, C₃₋₇cycloalkyl, pyrrolidinyl, piperidinyl, piperazinyl, 4-C₁₋₆alkylpiperazinyl, 4-C₁₋₆alkylcarbonyl-piperazinyl, and morpholinyl; or
(b) phenyl- or naphthyl-alkoxy optionally substituted with one, two or three substituents defined for (a) above; or
(c) phenyl- or naphthyl-carbonyloxy optionally substituted with one, two or three substituents defined for (a) above; and
   Het as a group or part of a group is a 5 or 6 membered saturated, partially unsaturated or completely unsaturated heterocyclic ring containing 1 to 4 heteroatoms each independently selected from nitrogen, oxygen and sulfur, being optionally condensed with one or two benzene rings, and wherein the group Het as a whole may be optionally substituted with one, two or three substituents each independently selected from the group consisting of halo, C₁₋₆alkyl, polyhaloC₁₋₆alkyl, hydroxy, aryl, C₁₋₆alkoxy, polyhaloC₁₋₆alkoxy, C₁₋₆alkoxyC₁₋₆alkyl, carboxyl, C₁₋₆alkylcarbonyl, cyano, nitro, amino, mono- or diC₁₋₆alkylamino, aminocarbonyl, C₃₋₇cycloalkyl, pyrrolidinyl, piperidinyl, piperazinyl, 4-C₁₋₆alkylpiperazinyl, 4-C₁₋₆alkylcarbonyl-piperazinyl, and morpholinyl;
   aryl² as a group or part of a group is phenyl, naphthyl, indanyl, or 1,2,3,4-tetrahydro-naphthyl, each of which may be optionally independently substituted with one, two or three substituents selected from
   (a) halo, C₁₋₆alkyl, polyhaloC₁₋₆alkyl, hydroxy, trifluoromethyl, alkylenedioxy, C₁₋₆alkoxy, C₁₋₆alkylthio, polyhalo-C₁₋₆alkoxy, C₁₋₆alkylcarbonyloxy, C₁₋₆alkoxyC₁₋₆alkyl, carboxyl, C₁₋₆alkylcarbonyl, cyano, nitro, amino, mono- or diC₁₋₆alkylamino, azido, mercapto, C₃₋₇cycloalkyl, pyrrolidinyl, piperidinyl, piperazinyl, 4-C₁₋₆alkylpiperazinyl, 4-C₁₋₆alkyl-carbonyl-piperazinyl, morpholinyl; phenyl- or napthyl-alkoxy optionally substituted with halogen; phenyl- or naphthyl-carbonyloxy optionally substituted with halogen, polyhaloC₁₋₆alkoxy, C₁₋₆alkoxyC₁₋₆alkyl, carboxyl, C₁₋₆alkylcarbonyl, cyano, nitro, amino, mono- or diC₁₋₆alkylamino, azido, mercapto, C₃₋₇cycloalkyl, pyrrolidinyl, piperidinyl, piperazinyl, 4-C₁₋₆alkylpiperazinyl, 4-C₁₋₆alkyl-carbonyl-piperazinyl, morpholinyl; or
   (b) a radical of formula —(X)ₙ-aryl or —(X)ₙ-Het in which n is 0 or 1 and X is —C₁₋₆alkanediyl-, C₁₋₆alkenediyl-, —NR²⁰—, —NR²⁰—C₁₋₆alkanediyl-, —NR²⁰—CO—C₁₋₆alkanediyl-, —CO—NR²⁰—C₁₋₆alkanediyl-, —O—, —CO—NR²⁰—, —NR²⁰—CO—, —NR²⁰—SO₂—, —SO₂—NR²⁰—, —O—C₁₋₆alkanediyl-, —O—CO—, —CO—, —O—CO—C₁₋₆alkanediyl-, —S— or —S—C₁₋₆alkanediyl-
      in which R²⁰ is hydrogen, C₃₋₇cycloalkyl, aryl, Het, C₁₋₆alkyl optionally substituted independently with one, two or three substituents selected from halo, C₁₋₆alkoxy, aryl, Het, cyano, polyhaloC₁₋₆alkoxy, and C₃₋₇cycloalkyl;
      Het² as a group or part of a group is a 5 or 6 membered saturated, partially unsaturated or completely unsaturated heterocyclic ring containing 1 to 4 heteroatoms each independently selected from nitrogen, oxygen and sulfur, being optionally condensed with one or two benzene rings, and wherein the group Het as a whole may be optionally substituted with one, two or three substituents each independently selected from the group consisting of halo, C₁₋₆alkyl, polyhaloC₁₋₆alkyl, hydroxy, oxo, aryl, C₁₋₆alkoxy, polyhaloC₁₋₆alkoxy, C₁₋₆alkoxyC₁₋₆alkyl, carboxyl, C₁₋₆alkylcarbonyl, cyano, nitro, amino, mono- or diC₁₋₆alkylamino, cycloalkyl, pyrrolidinyl, piperidinyl, piperazinyl, 4-C₁₋₆alkylpiperazinyl, 4-C₁₋₆alkylcarbonyl-piperazinyl, morpholinyl; or Het² is substituted with a radical of formula —(X)ₙ-aryl or —(X)ₙ-Het in which n is 0 or 1 and X is —C₁₋₆alkanediyl-, C₁₋₆alkenediyl-, —NR²¹—, —NR²¹—C₁₋₆alkanediyl-, —NR²¹—CO—C₁₋₆alkanediyl-, —CO—NR²¹—C₁₋₆alkanediyl-, —O—, —O—C₁₋₆alkanediyl-, —O—CO—, —O—CO—C₁₋₆alkanediyl-, —S—, or —S—C₁₋₆alkanediyl-
   in which R²¹ is hydrogen, C₃₋₇cycloalkyl, aryl, Het, C₁₋₆alkyl optionally substituted independently with one, two or three substituents selected from halo, C₁₋₆alkoxyaryl and Het; or with a cyano, polyhaloC₁₋₆alkoxy or C₃₋₇cycloalkyl.

In one embodiment, the invention relates to the use of the compounds of formula (I) for the manufacture of a medicament useful for inhibiting HCV activity in a mammal infected with HCV, said compounds being benzodiazepines of the formula (I):

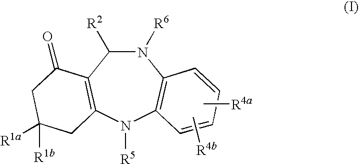

and the salts, stereoisomeric forms, and racemic mixtures thereof in which
R^{1a} and R^{1b} are independently, hydrogen; C₃₋₇cycloalkyl; aryl; Het; or C₁₋₆alkyl optionally substituted independently with one, two or three substituents selected from halo, C₁₋₆alkoxy, aryl and Het; or with a cyano, polyhaloC₁₋₆alkoxy or C₃₋₇cycloalkyl;
R² is hydrogen;
   C₁₋₆alkyl optionally substituted independently with one, two or three substituents selected from halo, C₁₋₆alkoxy, aryl and Het; or with a cyano, polyhaloC₁₋₆alkoxy or C₃₋₇cycloalkyl;
   C₃₋₇cycloalkyl optionally substituted independently with one, two or three substituents selected from halo, C₁₋₆alkoxy, aryl and Het; or with a cyano, polyhaloC₁₋₆alkoxy or C₃₋₇cycloalkyl,
   C₃₋₇cycloalkylC₁₋₆alkyl optionally substituted independently with one, two or three substituents selected from halo, C₁₋₆alkoxy, aryl and Het; or with a cyano, polyhaloC₁₋₆alkoxy or C₃₋₇cycloalkyl;
   C₂₋₆alkenyl optionally substituted independently with one, two or three substituents selected from halo, C₁₋₆alkoxy, aryl and Het; or with a cyano, polyhaloC₁₋₆alkoxy or C₃₋₇cycloalkyl;
   C₄₋₇cycloalkenyl optionally substituted independently with one, two or three substituents selected from halo, C₁₋₆alkoxy, aryl and Het; or with a cyano, polyhaloC₁₋₆alkoxy or C₃₋₇cycloalkyl;
   C₄₋₈cycloalkenylC₁₋₆alkyl optionally substituted independently with one, two or three substituents selected from halo, C₁₋₆alkoxy, aryl and Het; or with a cyano, polyhaloC₁₋₆alkoxy or C₃₋₇cycloalkyl;
   aryl²; or
   Het²;
R⁶ is hydrogen;
   C₁₋₆alkyl;
   —C(═O)—C₁₋₇alkyl, the C₁₋₇alkyl being optionally substituted independently with one, two or three substituents selected from halo, C₁₋₆alkoxy, aryl, Het, cyano, polyhaloC₁₋₆alkoxy, C₃₋₇cycloalkyl, and carboxyl;
   —C(═O)—C₃₋₇cycloalkyl, the C₃₋₇cycloalkyl being optionally substituted independently with one, two or three substituents selected from halo, C₁₋₆alkoxy, aryl, Het, cyano, polyhaloC₁₋₆alkoxy, and C₃₋₇cycloalkyl;
   —C(═O)-aryl;
   —C(═O)—Het;
   —C(═O)—NR^{12a}R^{12b},
      in which each R^{12a} and R^{12b} is, independently, hydrogen, C₃₋₇cycloalkyl, aryl, Het, or C₁₋₆alkyl optionally substituted independently with one, two or three substituents selected from halo, C₁₋₆alkoxy, aryl and Het; or with a cyano, polyhaloC₁₋₆alkoxy or C₃₋₇cycloalkyl;
   —C(═O)—OR^{13a},
      in which R¹³ is hydrogen, C₂₋₆alkenyl, C₃₋₇cycloalkyl, Het, or C₁₋₆alkyl optionally substituted with a C₃₋₇cycloalkyl or Het;
   —C(═O)—C₁₋₆alkyloxycarbonylC₁₋₆alkyl;
   —C(═O)—Het-thioC₁₋₆alkyl; or
   —C(═O)—Het-oxyC₁₋₆alkyl; or
R² and R⁶, together with the intervening grouping in formula (I) of sub-formula:

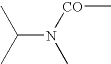

form a ring of formula:

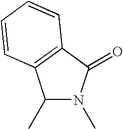

R^{4a} and R^{4b} are independently hydrogen, halo, cyano, C₁₋₆alkyl, C₁₋₆alkoxy, arylcarbonyl or —NR^{14a}R^{4b};
   in which each R^{14a} and R^{14b} is, independently, hydrogen; C₃₋₇cycloalkyl; aryl; Het; or C₁₋₆alkyl optionally substituted independently with one, two or three substituents selected from halo, C₁₋₆alkoxy, aryl, Het, cyano, polyhalo-C₁₋₆alkoxy, and C₃₋₇cycloalkyl;
R⁵ is hydrogen; C₃₋₇cycloalkyl; or C₁₋₆alkyl optionally substituted with a C₃₋₇cyclo-alkyl, aryl, Het, —C(═O)NR^{15a}R^{15b}, —NR^{15a}R^{15b}, —C(═O)R¹⁷, —NR^{15a}C(═O)R¹⁷, —NR^{15a}SOₚR¹⁸, is —SOₚR¹⁸, —SOₚNR^{15a}R^{15b}, —C(═O)OR¹⁶, or —NR^{15a}C(═O)OR^{16a} in which
   p is 0, 1 or 2;
   each R^{15a} and R^{15b} is, independently, hydrogen; C₃₋₇cycloalkyl; aryl; Het; or C₁₋₆alkyl optionally substituted independently with one, two or three substituents selected from halo, C₁₋₆alkoxy, aryl and Het; or with a cyano, polyhaloC₁₋₆alkoxy or C₃₋₇cycloalkyl;
   R¹⁶ is hydrogen; C₂₋₆alkenyl; C₃₋₇cycloalkyl; Het; or C₁₋₆alkyl optionally substituted with a C₃₋₇cycloalkyl or Het;
   R^{16a} is C₂₋₆alkenyl; C₃₋₇cycloalkyl; Het; or C₁₋₆alkyl optionally substituted with a C₃₋₇cycloalkyl or Het;
   R¹⁷ is hydrogen, C₁₋₆alkyl, C₃₋₇cycloalkyl or aryl;
   R¹⁸ is hydrogen; polyhaloC₁₋₆alkyl; C₃₋₇cycloalkyl; aryl; Het; or C₁₋₆alkyl optionally substituted with a C₃₋₇cycloalkyl, aryl or Het;
      aryl as a group or part of a group is phenyl, naphthyl, indanyl, or 1,2,3,4-tetrahydro-naphthyl, each of which may be optionally independently substituted with
(a) one, two or three substituents selected from halo, C₁₋₆alkyl, polyhaloC₁₋₆alkyl, hydroxy, trifluoromethyl, alkylenedioxy, C₁₋₆alkoxy, C₁₋₆alkylthio, polyhalo-C₁₋₆alkoxy, C₁₋₆alkoxyC₁₋₆alkyl, carboxyl, C₁₋₆alkylcarbonyl, cyano, nitro, amino, mono- or diC₁₋₆alkylamino, azido, mercapto, C₃₋₇cycloalkyl, pyrrolidinyl, piperidinyl, piperazinyl, 4-C₁₋₆alkylpiperazinyl, 4-C₁₋₆alkylcarbonyl-piperazinyl, and morpholinyl; or
(b) phenyl- or naphthyl-alkoxy optionally substituted with one, two or three substituents defined for (a) above; or
(c) phenyl- or naphthyl-carbonyloxy optionally substituted with one, two or three substituents defined for (a) above; and
   Het as a group or part of a group is a 5 or 6 membered saturated, partially unsaturated or completely unsaturated heterocyclic ring containing 1 to 4 heteroatoms each independently selected from nitrogen, oxygen and sulfur, being optionally condensed with one or two benzene rings, and wherein the group Het as a whole may be optionally substituted with one, two or three substituents each independently selected from the group consisting of halo, C₁₋₆alkyl, polyhaloC₁₋₆alkyl, hydroxy, aryl, C₁₋₆alkoxy, polyhaloC₁₋₆alkoxy, C₁₋₆alkoxyC₁₋₆alkyl, carboxyl, C₁₋₆alkylcarbonyl, cyano, nitro, amino, mono- or diC₁₋₆alkylamino, C₃₋₇cycloalkyl, pyrrolidinyl, piperidinyl, piperazinyl, 4-C₁₋₆alkylpiperazinyl, 4-C₁₋₆alkylcarbonyl-piperazinyl, and morpholinyl;
   aryl² as a group or part of a group is phenyl, naphthyl, indanyl, or 1,2,3,4-tetrahydro-naphthyl, each of which may be optionally independently substituted with
   (c) one, two or three substituents selected from halo, C₁₋₆alkyl, polyhaloC₁₋₆alkyl, hydroxy, trifluoromethyl, alkylenedioxy, C₁₋₆alkoxy, C₁₋₆alkylthio, polyhalo-C₁₋₆alkoxy, C₁₋₆alkoxyC₁₋₆alkyl, carboxyl, C₁₋₆alkylcarbonyl, cyano, nitro, amino, mono- or diC₁₋₆alkylamino, azido, mercapto, C₃₋₇cycloalkyl, pyrrolidinyl, piperidinyl, piperazinyl, 4-C₁₋₆alkylpiperazinyl, 4-C₁₋₆alkyl-carbonyl-piperazinyl, morpholinyl; phenyl- or napthyl-alkoxy optionally substituted with halogen; phenyl- or naphthyl-carbonyloxy optionally substituted with halogen, polyhaloC₁₋₆alkoxy, C₁₋₆alkoxyC₁₋₆alkyl, carboxyl, C₁₋₆alkylcarbonyl, cyano, nitro, amino, mono- or diC₁₋₆alkylamino, azido, mercapto, C₃₋₇cycloalkyl, pyrrolidinyl, piperidinyl, piperazinyl, 4-C₁₋₆alkylpiperazinyl, 4-C₁₋₆alkylcarbonyl-piperazinyl, morpholinyl; or
   (d) a radical of formula —(X)ₙ-aryl or —(X)ₙ-Het in which n is 0 or 1 and X is —C₁₋₆alkanediyl-, C₁₋₆alkenediyl-, —NR²⁰—, —NR²⁰—C₁₋₆alkanediyl-, —NR²⁰—CO—C₁₋₆alkanediyl-, —CO—NR²⁰—C₁₋₆alkanediyl-, —O—, —O—C₁₋₆alkanediyl-, —O—CO—, —O—CO—C₁₋₆alkanediyl-, —S— or —S—C₁₋₆alkanediyl-
      in which R²⁰ is hydrogen, C₃₋₇cycloalkyl, aryl, Het, C₁₋₆alkyl optionally substituted independently with one, two or three substituents selected from halo, C₁₋₆alkoxy, aryl, Het, cyano, polyhaloC₁₋₆alkoxy, and C₃₋₇cycloalkyl;
      Het² as a group or part of a group is a 5 or 6 membered saturated, partially unsaturated or completely unsaturated heterocyclic ring containing 1 to 4 heteroatoms each independently selected from nitrogen, oxygen and sulfur, being optionally condensed with one or two benzene rings, and wherein the group Het as a whole may be optionally substituted with one, two or three substituents each independently selected from the group consisting of halo, C₁₋₆alkyl, polyhaloC₁₋₆alkyl, hydroxy, oxo, aryl, C₁₋₆alkoxy, polyhaloC₁₋₆alkoxy, C₁₋₆alkoxyC₁₋₆alkyl, carboxyl, C₁₋₆alkylcarbonyl, cyano, nitro, amino, mono- or diC₁₋₆alkylamino, cycloalkyl, pyrrolidinyl, piperidinyl, piperazinyl, 4-C₁₋₆alkylpiperazinyl, 4-C₁₋₆alkylcarbonyl-piperazinyl, morpholinyl; or Het² is substituted with a radical of formula —(X)ₙ-aryl or —(X)ₙ-Het in which n is 0 or 1 and X is —C₁₋₆alkanediyl-, C₁₋₆alkenediyl-, —NR²¹—, —NR²¹—C₁₋₆alkanediyl-, —NR²¹—CO—C₁₋₆alkanediyl-, —CO—NR²¹—C₁₋₆alkanediyl-, —O—, —O—C₁₋₆alkanediyl-, —O—CO—, —O—CO—C₁₋₆alkanediyl-, —S—, or —S—C₁₋₆alkanediyl-
   in which R²¹ is hydrogen, C₃₋₇cycloalkyl, aryl, Het, C₁₋₆alkyl optionally substituted independently with one, two or three substituents selected from halo, C₁₋₆alkoxyaryl and Het; or with a cyano, polyhaloC₁₋₆alkoxy or C₃₋₇cycloalkyl.

In a further embodiment, the present invention relates to the following novel compounds of formula (I) per se,

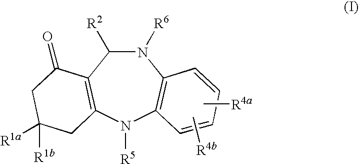

and the salts, stereoisomeric forms, and racemic mixtures thereof in which
R^{1a} and R^{1b} are independently, hydrogen, aryl, Het, or C₁₋₆alkyl;
R² is C₂₋₆alkenyl optionally substituted independently with one or two substituents selected from halo, and aryl;
   aryl²; or
   Het²;
R⁶ is hydrogen;
   C₁₋₆alkyl optionally substituted with carboxyl, C₁₋₆alkylcarbonyl, C₁₋₆alkoxy-carbonyl, Het-C₁₋₆alkylaminocarbonyl;
   —C(═O)—C₁₋₇alkyl, the C₁₋₇alkyl being optionally substituted independently with one, two or three substituents selected from halo, aryl, and cyano;
   —C(═O)—C₂₋₆alkenyl;
   —C(═O)-aryl;
   —C(═O)-Het;
   —C(═O)—NR^{12a}R^{12b};
   in which each R^{12a} and R^{12b} is, independently, hydrogen, aryl, or C₁₋₆alkyl optionally substituted independently with one or two substituents selected from aryl and Het;
R^{4a} and R^{4b} are independently hydrogen; halo; cyano; C₁₋₆alkyl optionally substituted with halo, hydroxy, Het, —OR^{14a}, or —NR^{14a}R^{14b}; C₁₋₆alkoxy optionally substituted with amino, hydroxy, C₁₋₆alkoxy, hydroxycarbonyl, aryl, or Het; aryloxy; Het-oxy; carboxyl; C₁₋₆alkylcarbonyloxy; C₁₋₆alkoxycarbonyl; —NR^{14a}R^{14b}; or
   —C(═O)—NR^{14a}R^{14b};
      in which each R^{14a} and R^{14b} is, independently, hydrogen; or C₁₋₆alkyl optionally substituted independently with one or two substituents selected from mono- or diC₁₋₆alkylamino, and Het;
R⁵ is hydrogen; or C₁₋₆alkyl optionally substituted with aryl;
   aryl as a group or part of a group is phenyl or naphthyl, each of which may be optionally independently substituted with
(a) one, two or three substituents selected from halo, C₁₋₆alkyl, trifluoromethyl, C₁₋₆alkoxy, carboxyl, C₁₋₆alkylcarbonyl, cyano, cyanoC₁₋₆alkyl, nitro, mono- or diC₁₋₆alkylamino; or
(b) phenyl-alkoxy optionally substituted with one, two or three substituents defined for (a) above;
   Het as a group or part of a group is a 5 or 6 membered saturated, partially unsaturated or completely unsaturated heterocyclic ring containing 1 to 4 heteroatoms each independently selected from nitrogen, oxygen and sulfur, being optionally condensed with one or two benzene rings, and wherein the group Het as a whole may be optionally substituted with one, two or three substituents each independently selected from the group consisting of C₁₋₆alkyl, and aminocarbonyl;
   aryl² as a group or part of a group is phenyl or naphthyl, each of which may be optionally independently substituted with one, two or three substituents selected from
(a) halo, C₁₋₆alkyl, hydroxy, trifluoromethyl, C₁₋₆alkoxy, polyhaloC₁₋₆alkoxy, C₁₋₆alkylcarbonyloxy, carboxyl, nitro, mono- or diC₁₋₆alkylamino; or
(b) a radical of formula —(X)ₙ-aryl or —(X)ₙ-Het in which n is 1 and X is —O—, —CO—NH—, —NH—CO—, —NH—SO₂—, —SO₂—NH—, —O—C₁₋₆alkanediyl-, —O—CO—, —CO—;
   Het² as a group or part of a group is a 5 or 6 membered saturated, partially unsaturated or completely unsaturated heterocyclic ring containing 1 to 4 heteroatoms each independently selected from nitrogen, oxygen and sulfur, being optionally condensed with one or two benzene rings, and wherein the group Het as a whole may be optionally substituted with one, two or three substituents each independently selected from the group consisting of halo, C₁₋₆alkyl, aryl, and nitro.

In one embodiment, the invention relates to the use of the compounds of formula (Ia) for the manufacture of a medicament useful for inhibiting HCV activity in a mammal infected with HCV, said compounds being acylated benzodiazepines of the formula (Ia):

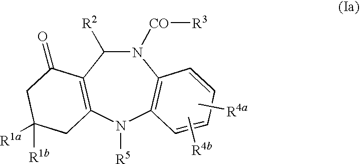

and the salts, stereoisomeric forms, and racemic mixtures thereof in which
R^{1a} and R^{1b} are independently, hydrogen; C₃₋₇cycloalkyl; aryl; Het; or C₁₋₆alkyl optionally substituted independently with one, two or three substituents selected from halo, C₁₋₆alkoxy, aryl and Het; or with a cyano, polyhaloC₁₋₆alkoxy or C₃₋₇cycloalkyl;
R² is hydrogen;
   C₁₋₆alkyl optionally substituted independently with one, two or three substituents selected from halo, C₁₋₆alkoxy, aryl and Het; or with a cyano, polyhaloC₁₋₆alkoxy or C₃₋₇cycloalkyl;
   C₃₋₇cycloalkyl optionally substituted independently with one, two or three substituents selected from halo, C₁₋₆alkoxy, aryl and Het; or with a cyano, polyhaloC₁₋₆alkoxy or C₃₋₇cycloalkyl,
   C₃₋₇cycloalkylC₁₋₆alkyl optionally substituted independently with one, two or three substituents selected from halo, C₁₋₆alkoxy, aryl and Het; or with a cyano, polyhaloC₁₋₆alkoxy or C₃₋₇cycloalkyl;
   C₂₋₆alkenyl optionally substituted independently with one, two or three substituents selected from halo, C₁₋₆alkoxy, aryl and Het; or with a cyano, polyhaloC₁₋₆alkoxy or C₃₋₇cycloalkyl;
   C₄₋₇cycloalkenyl optionally substituted independently with one, two or three substituents selected from halo, C₁₋₆alkoxy, aryl and Het; or with a cyano, polyhaloC₁₋₆alkoxy or C₃₋₇cycloalkyl;
   C₄₋₈cycloalkenylC₁₋₆alkyl optionally substituted independently with one, two or three substituents selected from halo, C₁₋₆alkoxy, aryl and Het; or with a cyano, polyhaloC₁₋₆alkoxy or C₃₋₇cycloalkyl;
   aryl²; or
   Het²;
R³ is C₁₋₇alkyl optionally substituted independently with one, two or three substituents selected from halo, C₁₋₆alkoxy, aryl, and Het; or with a cyano, polyhaloC₁₋₆alkoxy, C₃₋₇cycloalkyl or carboxyl;
   —C(═O)—C₂₋₆alkenyl;
   C₃₋₇cycloalkyl optionally substituted independently with one, two or three substituents selected from halo, C₁₋₆alkoxy, aryl and Het; or with a cyano, polyhaloC₁₋₆alkoxy, C₃₋₇cycloalkyl,
   aryl;
   Het;
   —NR^{12a}R^{12b}, OR^{13a};
      in which each R^{12a} and R^{12b} is, independently, hydrogen, C₃₋₇cycloalkyl, aryl, Het, or C₁₋₆alkyl optionally substituted independently with one, two or three substituents selected from halo, C₁₋₆alkoxy, aryl and Het; or with a cyano, polyhaloC₁₋₆alkoxy or C₃₋₇cycloalkyl;
      R¹³ is hydrogen, C₂₋₆alkenyl, C₃₋₇cycloalkyl, Het, or C₁₋₆alkyl optionally substituted with a C₃₋₇cycloalkyl or Het;
   C₁₋₆alkyloxycarbonylC₁₋₆alkyl;
   Het-thioC₁₋₆alkyl; or
   Het-oxyC₁₋₆alkyl; or
R² and R³, together with the intervening grouping in formula (I) of sub-formula:

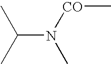

form a ring of formula:

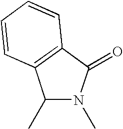

R^{4a} and R^{4b} are independently hydrogen; halo; cyano; C₁₋₆alkyl optionally substituted with halo, hydroxy, Het, —OR^{14a}, or —NR^{14a}R^{14b}; C₁₋₆alkoxy optionally substituted with amino, hydroxy, C₁₋₆alkoxy, hydroxycarbonyl, aryl, or Het; aryloxy; Het-oxy; carboxyl; C₁₋₆alkylcarbonyloxy; C₁₋₆alkoxycarbonyl; arylcarbonyl;
   —NR^{14a}R^{14b}; or —C(═O)—NR^{14a}R^{14b};
      in which each R^{14a} and R^{14b} is, independently, hydrogen; C₃₋₇cycloalkyl; aryl; Het; or C₁₋₆alkyl optionally substituted independently with one, two or three substituents selected from halo, C₁₋₆alkoxy, mono- or diC₁₋₆alkylamino, aryl, Het, cyano, polyhaloC₁₋₆alkoxy, and C₃₋₇cycloalkyl;
R⁵ is hydrogen; C₃₋₇cycloalkyl; or C₁₋₆alkyl optionally substituted with a C₃₋₇cyclo-alkyl, aryl, Het, —C(═O)NR^{15a}R^{15b}, —NR^{15a}R^{15b}, —C(═O)R¹⁷, —NR^{15a}C(═O)R¹⁷, —NR^{15a}SOₚR¹⁸, —SOₚR¹⁸, —SOₚNR^{15a}R^{15b}, —C(═O)R¹⁶, or —NR^{15a}C(═O)OR^{16a} in which
   p is 0, 1 or 2;
   each R^{15a} and R^{15b} is, independently, hydrogen; C₃₋₇cycloalkyl; aryl; Het; or C₁₋₆alkyl optionally substituted independently with one, two or three substituents selected from halo, C₁₋₆alkoxy, aryl and Het; or with a cyano, polyhaloC₁₋₆alkoxy or C₃₋₇cycloalkyl;
   R¹⁶ is hydrogen; C₂₋₆alkenyl; C₃₋₇cycloalkyl; Het; or C₁₋₆alkyl optionally substituted with a C₃₋₇cycloalkyl or Het;
   R^{16a} is C₂₋₆alkenyl; C₃₋₇cycloalkyl; Het; or C₁₋₆alkyl optionally substituted with a C₃₋₇cycloalkyl or Het;
   R¹⁷ is hydrogen, C₁₋₆alkyl, C₃₋₇cycloalkyl or aryl;
   R¹⁸ is hydrogen; polyhaloC₁₋₆alkyl; C₃₋₇cycloalkyl; aryl; Het; or C₁₋₆alkyl optionally substituted with a C₃₋₇cycloalkyl, aryl or Het;
      aryl as a group or part of a group is phenyl, naphthyl, indanyl, or 1,2,3,4-tetrahydro-naphthyl, each of which may be optionally independently substituted with
(a) one, two or three substituents selected from halo, C₁₋₆alkyl, polyhaloC₁₋₆alkyl, hydroxy, trifluoromethyl, alkylenedioxy, C₁₋₆alkoxy, polyhaloC₁₋₆alkoxy, C₁₋₆alkoxyC₁₋₆alkyl, carboxyl, C₁₋₆alkylcarbonyl, cyano, cyanoC₁₋₆alkyl, nitro, amino, mono- or diC₁₋₆alkylamino, azido, mercapto, C₃₋₇cycloalkyl, pyrrolidinyl, piperidinyl, piperazinyl, 4-C₁₋₆alkylpiperazinyl, 4-C₁₋₆alkylcarbonyl-piperazinyl, and morpholinyl; or
(b) phenyl- or naphthyl-alkoxy optionally substituted with one, two or three substituents defined for (a) above; or
(c) phenyl- or naphthyl-carbonyloxy optionally substituted with one, two or three substituents defined for (a) above; and
   Het as a group or part of a group is a 5 or 6 membered saturated, partially unsaturated or completely unsaturated heterocyclic ring containing 1 to 4 heteroatoms each independently selected from nitrogen, oxygen and sulfur, being optionally condensed with a benzene ring, and wherein the group Het as a whole may be optionally substituted with one, two or three substituents each independently selected from the group consisting of halo, C₁₋₆alkyl, polyhaloC₁₋₆alkyl, hydroxy, aryl, C₁₋₆alkoxy, polyhaloC₁₋₆alkoxy, C₁₋₆alkoxyC₁₋₆alkyl, carboxyl, C₁₋₆alkylcarbonyl, cyano, nitro, amino, mono- or diC₁₋₆alkylamino, aminocarbonyl, C₃₋₇cycloalkyl, pyrrolidinyl, piperidinyl, piperazinyl, 4-C₁₋₆alkylpiperazinyl, 4-C₁₋₆alkylcarbonyl-piperazinyl, and morpholinyl;
   aryl² as a group or part of a group is phenyl, naphthyl, indanyl, or 1,2,3,4-tetrahydro-naphthyl, each of which may be optionally substituted with one, two or three substituents selected from halo, C₁₋₆alkyl, polyhaloC₁₋₆alkyl, hydroxy, trifluoromethyl, alkylenedioxy, C₁₋₆alkoxy, phenyl- or napthyl-alkoxy optionally substituted with halogen; mono- or di-alkylamino; C₁₋₆alkylcarbonyloxy; nitro; polyhaloC₁₋₆alkoxy; phenyl- or naphthyl-carbonyloxy optionally substituted with halogen, polyhalo-C₁₋₆alkoxy, C₁₋₆alkoxyC₁₋₆alkyl, carboxyl, C₁₋₆alkylcarbonyl, mono or dialkylamino, cyano, nitro, amino, mono- or diC₁₋₆alkylamino, azido, mercapto, C₃₋₇cycloalkyl, pyrrolidinyl, piperidinyl, piperazinyl, 4-C₁₋₆alkylpiperazinyl, 4-C₁₋₆alkylcarbonyl-piperazinyl, morpholinyl; or aryl² is substituted with a radical of formula —(X)ₙ-aryl or —(X)ₙ-Het in which n is 0 or 1 and
   X is —C₁₋₆alkanediyl-, C₁₋₆alkenediyl-, —NR²⁰—, —NR²⁰—C₁₋₆alkanediyl-, —NR²⁰—CO—C₁₋₆alkanediyl-, —CO—NR²⁰—C₁₋₆alkanediyl-, —O—, —CO—NR²⁰—, —NR²⁰—CO—, —NR²⁰—SO₂—, —SO₂—NR²⁰—, —O—C₁₋₆alkanediyl-, —O—CO—, —CO—, —O—CO—C₁₋₆alkanediyl-, —S— or —S—C₁₋₆alkanediyl-
   in which R²⁰ is hydrogen, C₃₋₇cycloalkyl, aryl, Het, C₁₋₆alkyl optionally substituted independently with one, two or three substituents selected from halo, C₁₋₆alkoxy, aryl, Het, cyano, polyhaloC₁₋₆alkoxy, and C₃₋₇cycloalkyl;
      Het² as a group or part of a group is a 5 or 6 membered saturated, partially unsaturated or completely unsaturated heterocyclic ring containing 1 to 4 heteroatoms each independently selected from nitrogen, oxygen and sulfur, being optionally condensed with a benzene ring, and wherein the group Het as a whole may be optionally substituted with one, two or three substituents each independently selected from the group consisting of halo, C₁₋₆alkyl, polyhaloC₁₋₆alkyl, hydroxy, aryl, C₁₋₆alkoxy, polyhaloC₁₋₆alkoxy, C₁₋₆alkoxyC₁₋₆alkyl, carboxyl, C₁₋₆alkylcarbonyl, cyano, nitro, amino, mono- or diC₁₋₆alkylamino, cycloalkyl, pyrrolidinyl, piperidinyl, piperazinyl, 4-C₁₋₆alkylpiperazinyl, 4-C₁₋₆alkylcarbonyl-piperazinyl, morpholinyl; or Het² is substituted with a radical of formula —(X)ₙ-aryl or —(X)ₙ-Het in which n is 0 or 1 and X is —C₁₋₆alkanediyl-, C₁₋₆alkenediyl-, —NR²¹—, —NR²¹—C₁₋₆alkanediyl-, —NR²¹—CO—C₁₋₆alkanediyl-, —CO—NR²¹—C₁₋₆alkanediyl-, —O—, —O—C₁₋₆alkanediyl-, —O—CO—, —O—CO—C₁₋₆alkanediyl-, —S—, —S—C₁₋₆alkanediyl-
   in which R²¹ is hydrogen, C₃₋₇cycloalkyl, aryl, Het, C₁₋₆alkyl optionally substituted independently with one, two or three substituents selected from halo, C₁₋₆alkoxyaryl and Het; or with a cyano, polyhaloC₁₋₆alkoxy or C₃₋₇cycloalkyl.

In one embodiment, the invention relates to the use of the compounds of formula (Ia) for the manufacture of a medicament useful for inhibiting HCV activity in a mammal infected with HCV, said compounds being acylated benzodiazepines of the formula (Ia):

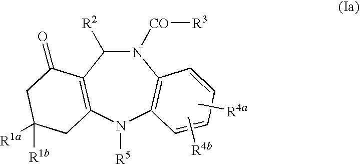

and the salts, stereoisomeric forms, and racemic mixtures thereof in which
R^{1a} and R^{1b} are independently, hydrogen; C₃₋₇cycloalkyl; aryl; Het; or C₁₋₆alkyl optionally substituted independently with one, two or three substituents selected from halo, C₁₋₆alkoxy, aryl and Het; or with a cyano, polyhaloC₁₋₆alkoxy or C₃₋₇cycloalkyl;
R² is hydrogen;
   C₁₋₆alkyl optionally substituted independently with one, two or three substituents selected from halo, C₁₋₆alkoxy, aryl and Het; or with a cyano, polyhaloC₁₋₆alkoxy or C₃₋₇cycloalkyl;
   C₃₋₇cycloalkyl optionally substituted independently with one, two or three substituents selected from halo, C₁₋₆alkoxy, aryl and Het; or with a cyano, polyhaloC₁₋₆alkoxy or C₃₋₇cycloalkyl,
   C₃₋₇cycloalkylC₁₋₆alkyl optionally substituted independently with one, two or three substituents selected from halo, C₁₋₆alkoxy, aryl and Het; or with a cyano, polyhaloC₁₋₆alkoxy or C₃₋₇cycloalkyl;
   C₂₋₆alkenyl optionally substituted independently with one, two or three substituents selected from halo, C₁₋₆alkoxy, aryl and Het; or with a cyano, polyhaloC₁₋₆alkoxy or C₃₋₇cycloalkyl;
   C₄₋₇cycloalkenyl optionally substituted independently with one, two or three substituents selected from halo, C₁₋₆alkoxy, aryl and Het; or with a cyano, polyhaloC₁₋₆alkoxy or C₃₋₇cycloalkyl;
   C₄₋₈cycloalkenylC₁₋₆alkyl optionally substituted independently with one, two or three substituents selected from halo, C₁₋₆alkoxy, aryl and Het; or with a cyano, polyhaloC₁₋₆alkoxy or C₃₋₇cycloalkyl;
   aryl²; or
   Het²;
R³ is C₁₋₇alkyl optionally substituted independently with one, two or three substituents selected from halo, C₁₋₆alkoxy, aryl, and Het; or with a cyano, polyhaloC₁₋₆alkoxy, C₃₋₇cycloalkyl or carboxyl;
   C₃₋₇cycloalkyl optionally substituted independently with one, two or three substituents selected from halo, C₁₋₆alkoxy, aryl and Het; or with a cyano, polyhaloC₁₋₆alkoxy, C₃₋₇cycloalkyl,
   aryl;
   Het;
   NR^{12a}R^{12b}, OR^{13a};
      in which each R^{12a} and R^{12b} is, independently, hydrogen, C₃₋₇cycloalkyl, aryl, Het, or C₁₋₆alkyl optionally substituted independently with one, two or three substituents selected from halo, C₁₋₆alkoxy, aryl and Het; or with a cyano, polyhaloC₁₋₆alkoxy or C₃₋₇cycloalkyl;
      R¹³ is hydrogen, C₂₋₆alkenyl, C₃₋₇cycloalkyl, Het, or C₁₋₆alkyl optionally substituted with a C₃₋₇cycloalkyl or Het;
   C₁₋₆alkyloxycarbonylC₁₋₆alkyl;
   Het-thioC₁₋₆alkyl; or
   Het-oxyC₁₋₆alkyl; or
R² and R³, together with the intervening grouping in formula (I) of sub-formula:

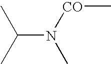

form a ring of formula:

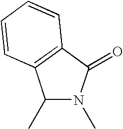

R^{4a} and R^{4b} are independently hydrogen, halo, cyano, C₁₋₆alkyl, C₁₋₆alkoxy, arylcarbonyl or —NR^{14a}R^{14b};
   in which each R^{14a} and R^{14b} is, independently, hydrogen; C₃₋₇cycloalkyl; aryl; Het; or C₁₋₆alkyl optionally substituted independently with one, two or three substituents selected from halo, C₁₋₆alkoxy, aryl, Het, cyano, polyhalo-C₁₋₆alkoxy, and C₃₋₇cycloalkyl;
R⁵ is hydrogen; C₃₋₇cycloalkyl; or C₁₋₆alkyl optionally substituted with a C₃₋₇cyclo-alkyl, aryl, Het, —C(═O)NR^{15a}R^{15b}, NR^{15a}R^{15b}, —C(═O)R¹⁷, —NR^{15a}C(═O)R¹⁷, —NR^{15a}SOₚR¹⁸, —SOₚR¹⁸, —SOₚNR^{15a}R^{15b}, —C(═O)OR¹⁶, or —NR^{15a}C(═O)OR¹⁶ in which
   p is 0, 1 or 2;
   each R^{15a} and R^{15b} is, independently, hydrogen; C₃₋₇cycloalkyl; aryl; Het; or C₁₋₆alkyl optionally substituted independently with one, two or three substituents selected from halo, C₁₋₆alkoxy, aryl and Het; or with a cyano, polyhaloC₁₋₆alkoxy or C₃₋₇cycloalkyl;
   R¹⁶ is hydrogen; C₂₋₆alkenyl; C₃₋₇cycloalkyl; Het; or C₁₋₆alkyl optionally substituted with a C₃₋₇cycloalkyl or Het;
   R^{16a} is C₂₋₆alkenyl; C₃₋₇cycloalkyl; Het; or C₁₋₆alkyl optionally substituted with a C₃₋₇cycloalkyl or Het;
   R¹⁷ is hydrogen, C₁₋₆alkyl, C₃₋₇cycloalkyl or aryl;
   R¹⁸ is hydrogen; polyhaloC₁₋₆alkyl; C₃₋₇cycloalkyl; aryl; Het; or C₁₋₆alkyl optionally substituted with a C₃₋₇cycloalkyl, aryl or Het;
      aryl as a group or part of a group is phenyl, naphthyl, indanyl, or 1,2,3,4-tetrahydro-naphthyl, each of which may be optionally independently substituted with
(a) one, two or three substituents selected from halo, C₁₋₆alkyl, polyhaloC₁₋₆alkyl, hydroxy, trifluoromethyl, alkylenedioxy, C₁₋₆alkoxy, polyhaloC₁₋₆alkoxy, C₁₋₆alkoxyC₁₋₆alkyl, carboxyl, C₁₋₆alkylcarbonyl, cyano, nitro, amino, mono- or diC₁₋₆alkylamino, azido, mercapto, C₃₋₇cycloalkyl, pyrrolidinyl, piperidinyl, piperazinyl, 4-C₁₋₆alkylpiperazinyl, 4-C₁₋₆alkylcarbonyl-piperazinyl, and morpholinyl; or
(b) phenyl- or naphthyl-alkoxy optionally substituted with one, two or three substituents defined for (a) above; or
(c) phenyl- or naphthyl-carbonyloxy optionally substituted with one, two or three substituents defined for (a) above; and
   Het as a group or part of a group is a 5 or 6 membered saturated, partially unsaturated or completely unsaturated heterocyclic ring containing 1 to 4 heteroatoms each independently selected from nitrogen, oxygen and sulfur, being optionally condensed with a benzene ring, and wherein the group Het as a whole may be optionally substituted with one, two or three substituents each independently selected from the group consisting of halo, C₁₋₆alkyl, polyhaloC₁₋₆alkyl, hydroxy, aryl, C₁₋₆alkoxy, polyhaloC₁₋₆alkoxy, C₁₋₆alkoxyC₁₋₆alkyl, carboxyl, C₁₋₆alkylcarbonyl, cyano, nitro, amino, mono- or diC₁₋₆alkylamino, C₃₋₇cycloalkyl, pyrrolidinyl, piperidinyl, piperazinyl, 4-C₁₋₆alkylpiperazinyl, 4-C₁₋₆alkylcarbonyl-piperazinyl, and morpholinyl;
   aryl² as a group or part of a group is phenyl, naphthyl, indanyl, or 1,2,3,4-tetrahydro-naphthyl, each of which may be optionally substituted with one, two or three substituents selected from halo, C₁₋₆alkyl, polyhaloC₁₋₆alkyl, hydroxy, trifluoromethyl, alkylenedioxy, C₁₋₆alkoxy, phenyl- or napthyl-alkoxy optionally substituted with halogen; mono- or di-alkylamino; phenyl- or naphthyl-carbonyloxy optionally substituted with halogen, polyhaloC₁₋₆alkoxy, C₁₋₆alkoxyC₁₋₆alkyl, carboxyl, C₁₋₆alkylcarbonyl, mono or dialkylamino, cyano, nitro, amino, mono- or diC₁₋₆alkyl-amino, azido, mercapto, C₃₋₇cycloalkyl, pyrrolidinyl, piperidinyl, piperazinyl, 4-C₁₋₆alkylpiperazinyl, 4-C₁₋₆alkylcarbonyl-piperazinyl, morpholinyl; or aryl² is substituted with a radical of formula —(X)ₙ-aryl or —(X)ₙ-Het in which n is 0 or 1 and X is —C₁₋₆alkanediyl-, C₁₋₆alkenediyl-, —NR²⁰—, —NR²⁰—C₁₋₆alkanediyl-, —NR²⁰—CO—C₁₋₆alkanediyl-, —CO—NR^{1e}—C₁₋₆alkanediyl-, —O—, —O—C₁₋₆alkanediyl-, —O—CO—, —O—CO—C₁₋₆alkanediyl-, —S— or —S—C₁₋₆alkanediyl-
   in which R²⁰ is hydrogen, C₃₋₇cycloalkyl, aryl, Het, C₁₋₆alkyl optionally substituted independently with one, two or three substituents selected from halo, C₁₋₆alkoxy, aryl, Het, cyano, polyhaloC₁₋₆alkoxy, and C₃₋₇cycloalkyl;
      Het² as a group or part of a group is a 5 or 6 membered saturated, partially unsaturated or completely unsaturated heterocyclic ring containing 1 to 4 heteroatoms each independently selected from nitrogen, oxygen and sulfur, being optionally condensed with a benzene ring, and wherein the group Het as a whole may be optionally substituted with one, two or three substituents each independently selected from the group consisting of halo, C₁₋₆alkyl, polyhaloC₁₋₆alkyl, hydroxy, aryl, C₁₋₆alkoxy, polyhaloC₁₋₆alkoxy, C₁₋₆alkoxyC₁₋₆alkyl, carboxyl, C₁₋₆alkylcarbonyl, cyano, nitro, amino, mono- or diC₁₋₆alkylamino, cycloalkyl, pyrrolidinyl, piperidinyl, piperazinyl, 4-C₁₋₆alkylpiperazinyl, 4-C₁₋₆alkylcarbonyl-piperazinyl, morpholinyl; or Het² is substituted with a radical of formula —(X)ₙ-aryl or —(X)ₙ-Het in which n is 0 or 1 and X is —C₁₋₆alkanediyl-, C₁₋₆alkenediyl-, —NR²⁰—, —NR²⁰—C₁₋₆alkanediyl-, —NR²¹—CO—C₁₋₆alkanediyl-, —CO—NR²¹—C₁₋₆alkanediyl-, —O—, —O—C₁₋₆alkanediyl-, —O—CO—, —O—CO—C₁₋₆alkanediyl-, —S—, —S—C₁₋₆alkanediyl-
   in which R²¹ is hydrogen, C₃₋₇cycloalkyl, aryl, Het, C₁₋₆alkyl optionally substituted independently with one, two or three substituents selected from halo, C₁₋₆alkoxyaryl and Het; or with a cyano, polyhaloC₁₋₆alkoxy or C₃₋₇cycloalkyl.

In a further embodiment, the present invention relates to the following novel compounds of formula (Ia) per se,

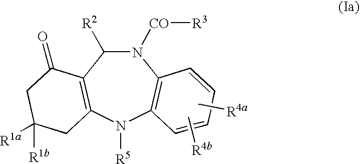

and the salts, stereoisomeric forms, and racemic mixtures thereof in which
R^{1a} and R^{1b} are independently, hydrogen, aryl, Het, or C₁₋₆alkyl;
R² is C₂₋₆alkenyl optionally substituted independently with one or two substituents selected from halo, and aryl;
   aryl²; or
   Het²;
R³ is C₁₋₇alkyl optionally substituted independently with one, two or three substituents selected from halo, aryl, and cyano;
   C₂₋₆alkenyl;
   aryl;
   Het;
   —NR^{12a}R^{12b},
   in which each R^{12a} and R^{12b} is, independently, hydrogen, aryl, or C₁₋₆alkyl optionally substituted independently with one or two substituents selected from aryl and Het;
R^{4a} and R^{4b} are independently hydrogen; halo; cyano; C₁₋₆alkyl optionally substituted with halo, hydroxy, Het, —OR^{14a}, or —NR^{14a}R^{14b}; C₁₋₆alkoxy optionally substituted with amino, hydroxy, C₁₋₆alkoxy, hydroxycarbonyl, aryl, or Het; aryloxy; Het-oxy; carboxyl; C₁₋₆alkylcarbonyloxy; C₁₋₆alkoxycarbonyl; —NR^{14a}R^{14b}; or —C(═O)—NR^{14a}R^{14b};
   in which each R^{14a} and R^{14b} is, independently, hydrogen; or C₁₋₆alkyl optionally substituted independently with one or two substituents selected from mono- or diC₁₋₆alkylamino, and Het;
R⁵ is hydrogen; or C₁₋₆alkyl optionally substituted with aryl; aryl as a group or part of a group is phenyl or naphthyl, each of which may be optionally independently substituted with
(a) one, two or three substituents selected from halo, C₁₋₆alkyl, trifluoromethyl, C₁₋₆alkoxy, carboxyl, C₁₋₆alkylcarbonyl, cyano, cyanoC₁₋₆alkyl, nitro, mono- or diC₁₋₆alkylamino; or
(b) phenyl-alkoxy optionally substituted with one, two or three substituents defined for (a) above;
   Het as a group or part of a group is a 5 or 6 membered saturated, partially unsaturated or completely unsaturated heterocyclic ring containing 1 to 4 heteroatoms each independently selected from nitrogen, oxygen and sulfur, being optionally condensed with one or two benzene rings, and wherein the group Het as a whole may be optionally substituted with one, two or three substituents each independently selected from the group consisting of C₁₋₆alkyl, and aminocarbonyl;
   aryl² as a group or part of a group is phenyl or naphthyl, each of which may be optionally independently substituted with one, two or three substituents selected from
(c) halo, C₁₋₆alkyl, hydroxy, trifluoromethyl, C₁₋₆alkoxy, C₁₋₆alkylcarbonyloxy, polyhaloC₁₋₆alkoxy, nitro, mono- or diC₁₋₆alkylamino; or
(d) a radical of formula —(X)ₙ-aryl or —(X)ₙ-Het in which n is 1 and X is —O—, —CO—NH—, —NH—CO—, —NH—SO₂—, —SO₂—NH—, —O—C₁₋₆alkanediyl-, —O—CO—, —CO—;
   Het² as a group or part of a group is a 5 or 6 membered saturated, partially unsaturated or completely unsaturated heterocyclic ring containing 1 to 4 heteroatoms each independently selected from nitrogen, oxygen and sulfur, being optionally condensed with one or two benzene rings, and wherein the group Het as a whole may be optionally substituted with one, two or three substituents each independently selected from the group consisting of halo, C₁₋₆alkyl, aryl, and nitro.

In a further embodiment, the present invention relates to the following novel compounds of formula (Ia) per se, namely Compound Nos. 101, 128, 129, 131, 132, 134, 210, 223 and 224, referred to in the Tables below, and the salts, stereoisomeric forms, and racemic mixtures thereof.

In one embodiment, the invention relates to the use of the compounds of formula (Ib) for the manufacture of a medicament useful for inhibiting HCV activity in a mammal infected with HCV, said compounds being benzodiazepines of the formula (Ib):

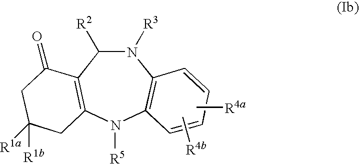

and the salts, stereoisomeric forms, and racemic mixtures thereof in which
R^{1a} and R^{1b} are independently, hydrogen; C₃₋₇cycloalkyl; aryl; Het; or C₁₋₆alkyl optionally substituted independently with one, two or three substituents selected from halo, C₁₋₆alkoxy, aryl and Het; or with a cyano, polyhaloC₁₋₆alkoxy or C₃₋₇cycloalkyl;
R² is hydrogen;
   C₁₋₆alkyl optionally substituted independently with one, two or three substituents selected from halo, C₁₋₆alkoxy, aryl and Het; or with a cyano, polyhaloC₁₋₆alkoxy or C₃₋₇cycloalkyl;
   C₃₋₇cycloalkyl optionally substituted independently with one, two or three substituents selected from halo, C₁₋₆alkoxy, aryl and Het; or with a cyano, polyhaloC₁₋₆alkoxy or C₃₋₇cycloalkyl,
   C₃₋₇cycloalkylC₁₋₆alkyl optionally substituted independently with one, two or three substituents selected from halo, C₁₋₆alkoxy, aryl and Het; or with a cyano, polyhaloC₁₋₆alkoxy or C₃₋₇cycloalkyl;
   C₂₋₆alkenyl optionally substituted independently with one, two or three substituents selected from halo, C₁₋₆alkoxy, aryl and Het; or with a cyano, polyhaloC₁₋₆alkoxy or C₃₋₇cycloalkyl;
   C₄₋₇cycloalkenyl optionally substituted independently with one, two or three substituents selected from halo, C₁₋₆alkoxy, aryl and Het; or with a cyano, polyhaloC₁₋₆alkoxy or C₃₋₇cycloalkyl;
   C₄₋₈cycloalkenylC₁₋₆alkyl optionally substituted independently with one, two or three substituents selected from halo, C₁₋₆alkoxy, aryl and Het; or with a cyano, polyhaloC₁₋₆alkoxy or C₃₋₇cycloalkyl;
   aryl²; or
   Het²;
R³ is hydrogen; or C₁₋₆alkyl optionally substituted with carboxyl, C₁₋₆alkylcarbonyl, C₁₋₆alkoxycarbonyl, or Het-C₁₋₆alkylaminocarbonyl;
R^{4a} and R^{4b} are independently hydrogen; halo; cyano; C₁₋₆alkyl optionally substituted with halo, hydroxy or —NR^{14a}R^{14b}; C₁₋₆alkoxy; carboxyl; C₁₋₆alkoxycarbonyl; arylcarbonyl; or —NR^{14a}R^{14b};
   in which each R^{14a} and R^{14b} is, independently, hydrogen; C₃₋₇cycloalkyl; aryl; Het; or C₁₋₆alkyl optionally substituted independently with one, two or three substituents selected from halo, C₁₋₆alkoxy, aryl, Het, cyano, polyhalo-C₁₋₆alkoxy, and C₃₋₇cycloalkyl;
R⁵ is hydrogen; C₃₋₇cycloalkyl; or C₁₋₆alkyl optionally substituted with a C₃₋₇cyclo-alkyl, aryl, Het, —C(═O)NR^{15a}R^{15b}, —NR^{15a}R^{15b}, —C(═O)R¹⁷, —NR^{15a}C(═O)R¹⁷, —NR^{15a}SOₚR¹⁸, —SOₚR¹⁸, —SOₚNR^{15a}R^{15b}, —C(═O)OR¹⁶, or —NR^{15a}C(═O)OR^{16a} in which
   p is 0, 1 or 2;
   each R^{15a} and R^{15b} is, independently, hydrogen; C₃₋₇cycloalkyl; aryl; Het; or C₁₋₆alkyl optionally substituted independently with one, two or three substituents selected from halo, C₁₋₆alkoxy, aryl and Het; or with a cyano, polyhaloC₁₋₆alkoxy or C₃₋₇cycloalkyl;
   R¹⁶ is hydrogen; C₂₋₆alkenyl; C₃₋₇cycloalkyl; Het; or C₁₋₆alkyl optionally substituted with a C₃₋₇cycloalkyl or Het;
   R^{16a} is C₂₋₆alkenyl; C₃₋₇cycloalkyl; Het; or C₁₋₆alkyl optionally substituted with a C₃₋₇cycloalkyl or Het;
   R¹⁷ is hydrogen, C₁₋₆alkyl, C₃₋₇cycloalkyl or aryl;
   R¹⁸ is hydrogen; polyhaloC₁₋₆alkyl; C₃₋₇cycloalkyl; aryl; Het; or C₁₋₆alkyl optionally substituted with a C₃₋₇cycloalkyl, aryl or Het;
      aryl as a group or part of a group is phenyl, naphthyl, indanyl, or 1,2,3,4-tetrahydro-naphthyl, each of which may be optionally independently substituted with
(a) one, two or three substituents selected from halo, C₁₋₆alkyl, polyhaloC₁₋₆alkyl, hydroxy, trifluoromethyl, alkylenedioxy, C₁₋₆alkoxy, C₁₋₆alkylthio, polyhalo-C₁₋₆alkoxy, C₁₋₆alkoxyC₁₋₆alkyl, carboxyl, C₁₋₆alkylcarbonyl, cyano, nitro, amino, mono- or diC₁₋₆alkylamino, azido, mercapto, C₃₋₇cycloalkyl, pyrrolidinyl, piperidinyl, piperazinyl, 4-C₁₋₆alkylpiperazinyl, 4-C₁₋₆alkylcarbonyl-piperazinyl, and morpholinyl; or
(b) phenyl- or naphthyl-alkoxy optionally substituted with one, two or three substituents defined for (a) above; or
(c) phenyl- or naphthyl-carbonyloxy optionally substituted with one, two or three substituents defined for (a) above; and
   Het as a group or part of a group is a 5 or 6 membered saturated, partially unsaturated or completely unsaturated heterocyclic ring containing 1 to 4 heteroatoms each independently selected from nitrogen, oxygen and sulfur, being optionally condensed with one or two benzene rings, and wherein the group Het as a whole may be optionally substituted with one, two or three substituents each independently selected from the group consisting of halo, C₁₋₆alkyl, polyhaloC₁₋₆alkyl, hydroxy, aryl, C₁₋₆alkoxy, polyhaloC₁₋₆alkoxy, C₁₋₆alkoxyC₁₋₆alkyl, carboxyl, C₁₋₆alkylcarbonyl, cyano, nitro, amino, mono- or diC₁₋₆alkylamino, C₃₋₇cycloalkyl, pyrrolidinyl, piperidinyl, piperazinyl, 4-C₁₋₆alkylpiperazinyl, 4-C₁₋₆alkylcarbonyl-piperazinyl, and morpholinyl;
   aryl² as a group or part of a group is phenyl, naphthyl, indanyl, or 1,2,3,4-tetrahydro-naphthyl, each of which may be optionally independently substituted with one, two or three substituents selected from
(e) halo, C₁₋₆alkyl, polyhaloC₁₋₆alkyl, hydroxy, trifluoromethyl, alkylenedioxy, C₁₋₆alkoxy, C₁₋₆alkylthio, polyhalo-C₁₋₆alkoxy, C₁₋₆alkoxyC₁₋₆alkyl, carboxyl, C₁₋₆alkylcarbonyl, cyano, nitro, amino, mono- or diC₁₋₆alkylamino, azido, mercapto, C₃₋₇cycloalkyl, pyrrolidinyl, piperidinyl, piperazinyl, 4-C₁₋₆alkylpiperazinyl, 4-C₁₋₆alkylcarbonyl-piperazinyl and morpholinyl; or
(f) a radical of formula —(X)ₙ-aryl or —(X)ₙ-Het in which n is 0 or 1 and X is —C₁₋₆alkanediyl-, C₁₋₆alkenediyl-, —NR²⁰—, —NR²⁰—C₁₋₆alkanediyl-, —NR²⁰—CO—C₁₋₆alkanediyl-, —CO—NR²⁰—C₁₋₆alkanediyl-, —O—, —CO—NR²⁰—, —SO₂—NR²⁰—, —O—C₁₋₆alkanediyl-, —O—CO—, —CO—, —O—CO—C₁₋₆alkanediyl-, —S— or —S—C₁₋₆alkanediyl-
   in which R²⁰ is hydrogen, C₃₋₇cycloalkyl, aryl, Het, C₁₋₆alkyl optionally substituted independently with one, two or three substituents selected from halo, C₁₋₆alkoxy, aryl, Het, cyano, polyhaloC₁₋₆alkoxy, and C₃₋₇cycloalkyl;
      Het² as a group or part of a group is a 5 or 6 membered saturated, partially unsaturated or completely unsaturated heterocyclic ring containing 1 to 4 heteroatoms each independently selected from nitrogen, oxygen and sulfur, being optionally condensed with one or two benzene rings, and wherein the group Het as a whole may be optionally substituted with one, two or three substituents each independently selected from the group consisting of halo, C₁₋₆alkyl, polyhaloC₁₋₆alkyl, hydroxy, oxo, aryl, C₁₋₆alkoxy, polyhaloC₁₋₆alkoxy, C₁₋₆alkoxyC₁₋₆alkyl, carboxyl, C₁₋₆alkylcarbonyl, cyano, nitro, amino, mono- or diC₁₋₆alkylamino, cycloalkyl, pyrrolidinyl, piperidinyl, piperazinyl, 4-C₁₋₆alkylpiperazinyl, 4-C₁₋₆alkylcarbonyl-piperazinyl, morpholinyl; or Het² is substituted with a radical of formula —(X)ₙ-aryl or —(X)ₙ-Het in which n is 0 or 1 and X is —C₁₋₆alkanediyl-, C₁₋₆alkenediyl-, —NR²¹—, —NR²¹—C₁₋₆alkanediyl-, —NR²¹—CO—C₁₋₆alkanediyl-, —CO—NR²¹—C₁₋₆alkanediyl-, —O—, —O—C₁₋₆alkanediyl-, —O—CO—, —O—CO—C₁₋₆alkanediyl-, —S—, or —S—C₁₋₆alkanediyl-
   in which R²¹ is hydrogen, C₃₋₇cycloalkyl, aryl, Het, C₁₋₆alkyl optionally substituted independently with one, two or three substituents selected from halo, C₁₋₆alkoxyaryl and Het; or with a cyano, polyhaloC₁₋₆alkoxy or C₃₋₇cycloalkyl.

In one embodiment, the invention relates to the use of the compounds of formula (Ib) for the manufacture of a medicament useful for inhibiting HCV activity in a mammal infected with HCV, said compounds being benzodiazepines of the formula (Ib):

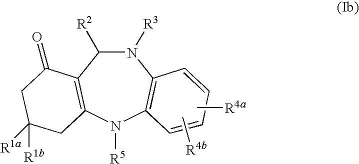

and the salts, stereoisomeric forms, and racemic mixtures thereof in which
R^{1a} and R^{1b} are independently, hydrogen; C₃₋₇cycloalkyl; aryl; Het; or C₁₋₆alkyl optionally substituted independently with one, two or three substituents selected from halo, C₁₋₆alkoxy, aryl and Het; or with a cyano, polyhaloC₁₋₆alkoxy or C₃₋₇cycloalkyl;
R² is hydrogen;
   C₁₋₆alkyl optionally substituted independently with one, two or three substituents selected from halo, C₁₋₆alkoxy, aryl and Het; or with a cyano, polyhaloC₁₋₆alkoxy or C₃₋₇cycloalkyl;
   C₃₋₇cycloalkyl optionally substituted independently with one, two or three substituents selected from halo, C₁₋₆alkoxy, aryl and Het; or with a cyano, polyhaloC₁₋₆alkoxy or C₃₋₇cycloalkyl,
   C₃₋₇cycloalkylC₁₋₆alkyl optionally substituted independently with one, two or three substituents selected from halo, C₁₋₆alkoxy, aryl and Het; or with a cyano, polyhaloC₁₋₆alkoxy or C₃₋₇cycloalkyl;
   C₂₋₆alkenyl optionally substituted independently with one, two or three substituents selected from halo, C₁₋₆alkoxy, aryl and Het; or with a cyano, polyhaloC₁₋₆alkoxy or C₃₋₇cycloalkyl;
   C₄₋₇cycloalkenyl optionally substituted independently with one, two or three substituents selected from halo, C₁₋₆alkoxy, aryl and Het; or with a cyano, polyhaloC₁₋₆alkoxy or C₃₋₇cycloalkyl;
   C₄₋₈cycloalkenylC₁₋₆alkyl optionally substituted independently with one, two or three substituents selected from halo, C₁₋₆alkoxy, aryl and Het; or with a cyano, polyhaloC₁₋₆alkoxy or C₃₋₇cycloalkyl;
   aryl²; or
   Het²;
R³ is hydrogen or C₁₋₆alkyl;
R^{4a} and R^{4b} are independently hydrogen, halo, cyano, C₁₋₆alkyl, C₁₋₆alkoxy, arylcarbonyl or —NR^{14a}R^{14b};
   in which each R^{14a} and R^{14b} is, independently, hydrogen; C₃₋₇cycloalkyl; aryl; Het; or C₁₋₆alkyl optionally substituted independently with one, two or three substituents selected from halo, C₁₋₆alkoxy, aryl, Het, cyano, polyhalo-C₁₋₆alkoxy, and C₃₋₇cycloalkyl;
R⁵ is hydrogen; C₃₋₇cycloalkyl; or C₁₋₆alkyl optionally substituted with a C₃₋₇cyclo-alkyl, aryl, Het, —C(═O)NR^{15a}R^{15b}, —NR^{15a}R^{15b}, —C(═O)R¹⁷, —NR^{15a}C(O)R¹⁷, —NR^{15a}SOₚR¹⁸, —SOₚR¹⁸, —SOₚNR^{15a}R^{15b}, —C(═O)OR¹⁶, or —NR^{15a}C(═O)OR^{16a} in which
   p is 0, 1 or 2;
   each R^{15a} and R^{15b} is, independently, hydrogen; C₃₋₇cycloalkyl; aryl; Het; or C₁₋₆alkyl optionally substituted independently with one, two or three substituents selected from halo, C₁₋₆alkoxy, aryl and Het; or with a cyano, polyhaloC₁₋₆alkoxy or C₃₋₇cycloalkyl;
   R¹⁶ is hydrogen; C₂₋₆alkenyl; C₃₋₇cycloalkyl; Het; or C₁₋₆alkyl optionally substituted with a C₃₋₇cycloalkyl or Het;
   R^{16a} is C₂₋₆alkenyl; C₃₋₇cycloalkyl; Het; or C₁₋₆alkyl optionally substituted with a C₃₋₇cycloalkyl or Het;
   R¹⁷ is hydrogen, C₁₋₆alkyl, C₃₋₇cycloalkyl or aryl;
   R¹⁸ is hydrogen; polyhaloC₁₋₆alkyl; C₃₋₇cycloalkyl; aryl; Het; or C₁₋₆alkyl optionally substituted with a C₃₋₇cycloalkyl, aryl or Het;
      aryl as a group or part of a group is phenyl, naphthyl, indanyl, or 1,2,3,4-tetrahydro-naphthyl, each of which may be optionally independently substituted with
(a) one, two or three substituents selected from halo, C₁₋₆alkyl, polyhaloC₁₋₆alkyl, hydroxy, trifluoromethyl, alkylenedioxy, C₁₋₆alkoxy, C₁₋₆alkylthio, polyhalo-C₁₋₆alkoxy, C₁₋₆alkoxyC₁₋₆alkyl, carboxyl, C₁₋₆alkylcarbonyl, cyano, nitro, amino, mono- or diC₁₋₆alkylamino, azido, mercapto, C₃₋₇cycloalkyl, pyrrolidinyl, piperidinyl, piperazinyl, 4-C₁₋₆alkylpiperazinyl, 4-C₁₋₆alkylcarbonyl-piperazinyl, and morpholinyl; or
(b) phenyl- or naphthyl-alkoxy optionally substituted with one, two or three substituents defined for (a) above; or
(c) phenyl- or naphthyl-carbonyloxy optionally substituted with one, two or three substituents defined for (a) above; and
   Het as a group or part of a group is a 5 or 6 membered saturated, partially unsaturated or completely unsaturated heterocyclic ring containing 1 to 4 heteroatoms each independently selected from nitrogen, oxygen and sulfur, being optionally condensed with one or two benzene rings, and wherein the group Het as a whole may be optionally substituted with one, two or three substituents each independently selected from the group consisting of halo, C₁₋₆alkyl, polyhaloC₁₋₆alkyl, hydroxy, aryl, C₁₋₆alkoxy, polyhaloC₁₋₆alkoxy, C₁₋₆alkoxyC₁₋₆alkyl, carboxyl, C₁₋₆alkylcarbonyl, cyano, nitro, amino, mono- or diC₁₋₆alkylamino, C₃₋₇cycloalkyl, pyrrolidinyl, piperidinyl, piperazinyl, 4-C₁₋₆alkylpiperazinyl, 4-C₁₋₆alkylcarbonyl-piperazinyl, and morpholinyl;
   aryl² as a group or part of a group is phenyl, naphthyl, indanyl, or 1,2,3,4-tetrahydro-naphthyl, each of which may be optionally independently substituted with
(g) one, two or three substituents selected from halo, C₁₋₆alkyl, polyhaloC₁₋₆alkyl, hydroxy, trifluoromethyl, alkylenedioxy, C₁₋₆alkoxy, C₁₋₆alkylthio, polyhalo-C₁₋₆alkoxy, C₁₋₆alkoxyC₁₋₆alkyl, carboxyl, C₁₋₆alkylcarbonyl, cyano, nitro, amino, mono- or diC₁₋₆alkylamino, azido, mercapto, C₃₋₇cycloalkyl, pyrrolidinyl, piperidinyl, piperazinyl, 4-C₁₋₆alkylpiperazinyl, 4-C₁₋₆alkyl-carbonyl-piperazinyl and morpholinyl; or
(h) a radical of formula —(X)ₙ-aryl or —(X)ₙ-Het in which n is 0 or 1 and X is —C₁₋₆alkanediyl-, C₁₋₆alkenediyl-, —NR²⁰—, —NR²⁰—C₁₋₆alkanediyl-, —NR²⁰—CO—C₁₋₆alkanediyl-, —CO—NR²⁰—C₁₋₆alkanediyl-, —O—, —O—C₁₋₆alkanediyl-, —O—CO—, —O—CO—C₁₋₆alkanediyl-, —S— or —S—C₁₋₆alkanediyl-
   in which R²⁰ is hydrogen, C₃₋₇cycloalkyl, aryl, Het, C₁₋₆alkyl optionally substituted independently with one, two or three substituents selected from halo, C₁₋₆alkoxy, aryl, Het, cyano, polyhaloC₁₋₆alkoxy, and C₃₋₇cycloalkyl;
      Het² as a group or part of a group is a 5 or 6 membered saturated, partially unsaturated or completely unsaturated heterocyclic ring containing 1 to 4 heteroatoms each independently selected from nitrogen, oxygen and sulfur, being optionally condensed with one or two benzene rings, and wherein the group Het as a whole may be optionally substituted with one, two or three substituents each independently selected from the group consisting of halo, C₁₋₆alkyl, polyhaloC₁₋₆alkyl, hydroxy, oxo, aryl, C₁₋₆alkoxy, polyhaloC₁₋₆alkoxy, C₁₋₆alkoxyC₁₋₆alkyl, carboxyl, C₁₋₆alkylcarbonyl, cyano, nitro, amino, mono- or diC₁₋₆alkylamino, cycloalkyl, pyrrolidinyl, piperidinyl, piperazinyl, 4-C₁₋₆alkylpiperazinyl, 4-C₁₋₆alkylcarbonyl-piperazinyl, morpholinyl; or Het² is substituted with a radical of formula —(X)ₙ-aryl or —(X)ₙ-Het in which n is 0 or 1 and X is —C₁₋₆alkanediyl-, C₁₋₆alkenediyl-, —NR²¹—, —NR²¹—C₁₋₆alkanediyl-, —NR²¹—CO—C₁₋₆alkanediyl-, —CO—NR²¹—C₁₋₆alkanediyl-, —O—, —O—C₁₋₆alkanediyl-, —O—CO—, —O—CO—C₁₋₆alkanediyl-, —S—, or —S—C₁₋₆alkanediyl-
   in which R²¹ is hydrogen, C₃₋₇cycloalkyl, aryl, Het, C₁₋₆alkyl optionally substituted independently with one, two or three substituents selected from halo, C₁₋₆alkoxyaryl and Het; or with a cyano, polyhaloC₁₋₆alkoxy or C₃₋₇cycloalkyl.

In a further embodiment, the present invention relates to the following novel compounds of formula (Ib) per se,

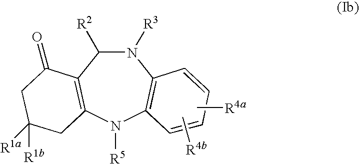

and the salts, stereoisomeric forms, and racemic mixtures thereof in which
R^{1a} and R^{1b} are independently, hydrogen, aryl, or C₁₋₆alkyl;
R² is C₂₋₆alkenyl optionally substituted independently with one or two substituents selected from halo, and aryl;
   aryl²; or
   Het²;
R³ is hydrogen;
   C₁₋₆alkyl optionally substituted with carboxyl, C₁₋₆alkylcarbonyl,
   C₁₋₆alkoxycarbonyl, Het-C₁₋₆alkylaminocarbonyl;
R^{4a} and R^{4b} are independently hydrogen; halo; cyano; C₁₋₆alkyl optionally substituted with halo, hydroxy, or —NR^{14a}R^{14b}; C₁₋₆alkoxy optionally substituted with C₁₋₆alkoxy; carboxyl; or —NR^{14a}R^{14b};
   in which each R^{14a} and R^{14b} is, independently, hydrogen; or C₁₋₆alkyl;
R⁵ is hydrogen;
   aryl as a group or part of a group is phenyl or naphthyl, each of which may be optionally independently substituted with
(a) one, two or three substituents selected from halo, and C₁₋₆alkoxy; or
(b) phenyl-alkoxy optionally substituted with one, two or three substituents defined for (a) above;
   Het as a group or part of a group is a 5 or 6 membered saturated, partially unsaturated or completely unsaturated heterocyclic ring containing 1 to 4 heteroatoms each independently selected from nitrogen, oxygen and sulfur, being optionally condensed with one or two benzene rings;
   aryl² as a group or part of a group is phenyl or naphthyl, each of which may be optionally independently substituted with one, two or three substituents selected from
a) halo, hydroxy, polyhalo-C₁₋₆alkoxy, carboxyl, nitro; or
b) a radical of formula —(X)ₙ-aryl in which n is 1 and
   X is —O—, —CO—NH—, —SO₂—NH—, —O—C₁₋₆alkanediyl-, —O—CO—, —CO—;
      Het² as a group or part of a group is a 5 or 6 membered saturated, partially unsaturated or completely unsaturated heterocyclic ring containing 1 to 4 heteroatoms each independently selected from nitrogen, oxygen and sulfur, being optionally condensed with one or two benzene rings, and wherein the group Het as a whole may be optionally substituted with one, two or three halo.

In a further embodiment, the present invention relates to the following novel compounds of formula (Ib) per se, namely Compound Nos. 94, 95, 96, 97, 98, 124, 154, 156, 157, 158 and 159, referred to in the Tables below, and the salts, stereoisomeric forms, and racemic mixtures thereof.

The term “C₁₋₆alkyl” as a group or part of a group defines straight and branched chained saturated hydrocarbon radicals having from 1 to 6 carbon atoms, such as, for example methyl, ethyl, propyl, butyl, 2-methyl-propyl, pentyl, 2-methylbutyl, hexyl, 3-methylpentyl and the like.

The term “C₁₋₇alkyl” as a group or part of a group defines straight and branched chained saturated hydrocarbon radicals having from 1 to 7 carbon atoms, such as, for example methyl, ethyl, propyl, butyl, 2-methyl-propyl, pentyl, 2-methylbutyl, hexyl, 3-methylpentyl, heptyl and the like.

The term “C₁₋₆alkoxy” means C₁₋₆alkyloxy wherein C₁₋₆alkyl is as defined above.

The term “C₃₋₇cycloalkyl” as a group or part of a group defines cyclic saturated hydrocarbon radicals having from 3 to 7 carbon atoms, such as, for example cyclopropyl, cyclobutyl, cyclopentyl, cyclohexyl and cycloheptyl.

The term “C₂₋₆alkenyl” as a group or part of a group defines straight and branched chained hydrocarbon radicals having at least one double bond, and from 2 to 6 carbon atoms, such as, for example, ethenyl, prop-1-enyl, but-1-enyl, but-2-enyl, pent-1-enyl, pent-2-enyl, hex-1-enyl, hex-2-enyl, hex-3-enyl, 1-methyl-pent-2-enyl and the like. Preferred are C₂₋₆alkenyls having one double bond.

The term “C₄₋₈cycloalkenyl” as a group or part of a group defines cyclic hydrocarbon radicals having at least one double bond, and from 4 to 8 carbon atoms, such as, for example cyclobutenyl, cyclopentenyl, cyclohexenyl or cycloheptenyl and the like, and including alkyl substitution on the ring, such as for example 2,2-dimethyl-3-methyl-cyclopent-3-enyl. Preferred are C₄₋₈cycloalkenyls having one double bond.

The term “C₁₋₆alkanediyl” as a group or part of a group defines bivalent straight and branched chained hydrocarbon radicals having from 1 to 6 carbon atoms such as, for example, methanediyl, 1,2-ethanediyl, or 1,1-ethanediyl, 1,3-propanediyl, 1,3-butanediyl, 1,4-butanediyl, 1,3-pentanediyl, 1,5-pentanediyl, 1,4-hexanediyl, 1,6-hexanediyl, and the like.

The term “halo” is generic to fluoro, chloro, bromo or iodo.

As used in the foregoing and hereinafter “polyhaloC₁₋₆alkyl” as a group or part of a group is defined as mono- or polyhalosubstituted C₁₋₆alkyl, for example, 1,1,1-trifluoroethyl, 1,1-difluoro-ethyl, the polyhalomethyl groups mentioned hereinafter, and the like. A preferred subgroup of polyhaloC₁₋₆alkyl is polyhalomethyl, wherein the latter as a group or part of a group is defined as mono- or polyhalo-substituted methyl, in particular methyl with one or more fluoro atoms, for example, difluoromethyl or trifluoromethyl. In case more than one halogen atom is attached to an alkyl group within the definition of polyhalomethyl or polyhaloC₁₋₄alkyl, they may be the same or different.

It should also be noted that the radical positions on any molecular moiety used in the definitions, unless indicated otherwise, may be anywhere on such moiety as long as it is chemically stable. For instance pyridyl includes 2-pyridyl, 3-pyridyl and 4-pyridyl; pentyl includes 1-pentyl, 2-pentyl and 3-pentyl.

When any variable (e.g. halogen or C₁₋₄alkyl) occurs more than one time in any constituent, each definition is independent.

The N-oxide forms of the present compounds are meant to comprise any one of the compounds of the present invention wherein one or several nitrogen atoms are oxidized to the so-called N-oxide.

For therapeutic use, the salts of the compounds of the present invention are those wherein the counter-ion is pharmaceutically or physiologically acceptable. However, salts having a pharmaceutically unacceptable counter-ion may also find use, for example, in the preparation or purification of a pharmaceutically acceptable compound of formula (I). All salts, whether pharmaceutically acceptable or not are included within the ambit of the present invention.

The pharmaceutically acceptable or physiologically tolerable addition salt forms which the compounds of the present invention are able to form can conveniently be prepared using the appropriate acids, such as, for example, inorganic acids such as hydrohalic acids, e.g. hydrochloric or hydrobromic acid, sulfuric, hemisulphuric, nitric, phosphoric and the like acids; or organic acids such as, for example, acetic, aspartic, dodecyl-sulphuric, heptanoic, hexanoic, benzoic, nicotinic, propanoic, hydroxyacetic, lactic, pyruvic, oxalic, malonic, succinic, maleic, fumaric, malic, tartaric, citric, methane-sulfonic, ethanesulfonic, benzenesulfonic, p-toluenesulfonic, cyclamic, salicylic, p-amino-salicylic, pamoic and the like acids.

Conversely said acid addition salt forms can be converted by treatment with an appropriate base into the free base form.

The compounds of formula (I) containing an acidic proton may also be converted into their non-toxic metal or amine addition base salt form by treatment with appropriate organic and inorganic bases. Appropriate base salt forms comprise, for example, the ammonium salts, the alkali and earth alkaline metal salts, e.g. the lithium, sodium, potassium, magnesium, calcium salts and the like, salts with organic bases, e.g. the benzathine, N-methyl-D-glucamine, hydrabamine salts, and salts with amino acids such as, for example, arginine, lysine and the like. Alternatively, when a carboxyl moiety is present on the compound of formula (I), the compound may also be supplied as a salt with a pharmaceutically acceptable cation.

Conversely said base addition salt forms can be converted by treatment with an appropriate acid into the free acid form.

The term “salts” also comprises the hydrates and the solvent addition forms that the compounds of the present invention are able to form. Examples of such forms are e.g. hydrates, alcoholates and the like.

In the event that any of the substituents of formula (I) contain chiral centers, as some, indeed, do, the compounds of formulas (I) include all stereoisomeric forms thereof, both as isolated stereoisomers and mixtures of these stereoisomeric forms.

The term stereochemically isomeric forms of compounds of the present invention, as used hereinbefore, defines all possible compounds made up of the same atoms bonded by the same sequence of bonds but having different three-dimensional structures which are not interchangeable, which the compounds of the present invention may possess. Unless otherwise mentioned or indicated, the chemical designation of a compound encompasses the mixture of all possible stereochemically isomeric forms which said compound may possess. Said mixture may contain all diastereomers and/or enantiomers of the basic molecular structure of said compound. All stereochemically isomeric forms of the compounds of the present invention both in pure form or in admixture with each other are intended to be embraced within the scope of the present invention.

Pure stereoisomeric forms of the compounds as mentioned herein are defined as isomers substantially free of other enantiomeric or diastereomeric forms of the same basic molecular structure of said compounds or intermediates. In particular, the term ‘stereoisomerically pure’ concerns compounds or intermediates having a stereoisomeric excess of at least 80% (i.e. minimum 90% of one isomer and maximum 10% of the other possible isomers) up to a stereoisomeric excess of 100% (i.e. 100% of one isomer and none of the other), more in particular, compounds or intermediates having a stereoisomeric excess of 90% up to 100%, even more in particular having a stereoisomeric excess of 94% up to 100% and most in particular having a stereoisomeric excess of 97% up to 100%. The terms ‘enantiomerically pure’ and ‘diastereomerically pure’ should be understood in a similar way, but then having regard to the enantiomeric excess, respectively the diastereomeric excess of the mixture in question.

Pure stereoisomeric forms of the compounds of this invention may be obtained by the application of art-known procedures. For instance, enantiomers may be separated from each other by the selective crystallization of their diastereomeric salts with optically active acids or bases. Examples thereof are tartaric acid, dibenzoyl-tartaric acid, ditoluoyltartaric acid and camphosulfonic acid. Alternatively, enantiomers may be separated by chromatographic techniques using chiral stationary phases. Said pure stereochemically isomeric forms may also be derived from the corresponding pure stereochemically isomeric forms of the appropriate starting materials, provided that the reaction occurs stereospecifically. Preferably, if a specific stereoisomer is desired, said compound will be synthesized by stereospecific methods of preparation. These methods will advantageously employ enantiomerically pure starting materials.

The diastereomeric racemates of formula (I) can be obtained separately by conventional methods. Appropriate physical separation methods that may advantageously be employed are, for example, selective crystallization and chromatography, e.g. column chromatography.

The present compounds may also exist in their tautomeric forms. Such forms, although not explicitly indicated in the above formula are intended to be included within the scope of the present invention. For example, within the definition of Het, for example an 1,2,4-oxadiazole may be substituted with a hydroxy group in the 5-position, thus being in equilibrium with its respective tautomeric form as depicted below.

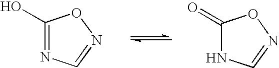

The term “prodrug” as used throughout this text means the pharmacologically acceptable derivatives such as esters, amides and phosphates, such that the resulting in vivo biotransformation product of the derivative is the active drug as defined in the compounds of formula (I). The reference by Goodman and Gilman (The Pharmacological Basis of Therapeutics, 8^{th} ed, McGraw-Hill, Int. Ed. 1992, “Biotransformation of Drugs”, p 13-15) describing prodrugs generally is hereby incorporated. Prodrugs of a compound of the present invention are prepared by modifying functional groups present in the compound in such a way that the modifications are cleaved, either by routine manipulation or in vivo, to the parent compound. For example, a substituent containing sulfhydryl could be coupled to a carrier which renders the compound biologically inactive until removed by endogenous enzymes or, for example, by enzymes targeted to a particular receptor or location in the subject.

Prodrugs are characterized by excellent aqueous solubility, increased bioavailability and are readily metabolized into the active inhibitors in vivo.

The present invention is also intended to include all isotopes of atoms occurring on the present compounds. Isotopes include those atoms having the same atomic number but different mass numbers. By way of general example and without limitation, isotopes of hydrogen include tritium and deuterium. Isotopes of carbon include C-13 and C-14.

Whenever used hereinafter, the term “compounds of formula (I)”, or similar term is meant to include the compounds of general formula (I), (Ia), (Ib), their N-oxides, salts, stereoisomeric forms, racemic mixtures, prodrugs and esters. An interesting subgroup of the compounds of the present invention or any subgroup thereof are the N-oxides, salts and all the stereoisomeric forms thereof.

Examples of compounds of formula (J) include those wherein the aryl or aryl² group is phenyl or naphthyl optionally substituted with halogen; alkoxy; phenyl- or naphthyl-oxy optionally substituted with halo; mono- or di C₁₋₆alkylamino; nitro; hydroxy; or phenyl- or naphthyl-carbonyloxy optionally substituted with halo. Especially preferred substituents include halo such as fluoro, chloro, bromo; alkoxy such as methoxy, ethoxy, isopropoxy, n-butoxy or n-pentoxy; and mono- or di C₁₋₆alkylamino such as dimethylamino or diethylamino.

Examples of compounds of formula (J) include those wherein the Het or Het² group is a 5 or 6 membered heterocyclic ring containing 1, 2 or 3, preferably 1 or 2 heteroatoms selected from nitrogen, oxygen and sulphur, for example, furanyl, thienyl, pyrrolyl, pyrrolidinyl, piperidinyl, morpholinyl, thiomorpholinyl, piperazinyl, pyrrolyl, pyrazolyl, imidazolyl, oxazolyl, isoxazolyl, thiazinolyl, isothiazinolyl, thiazolyl, isothiazolyl, oxadiazolyl, thiadiazolyl, triazolyl (including 1,2,3-triazolyl, 1,2,4-triazolyl), tetrazolyl, pyridyl, pyrimidyl, pyridazinyl, pyrazolyl, triazinyl, and the like. Such Het or Het² groups may be optionally substituted with halogen, C₁₋₆alkyl, nitro or aryl optionally substituted with halo. In the compounds of formula (Ib), such heterocyclic groups may be optionally condensed with one or two benzene rings to form for example a carbazolyl, indolyl or cromenyl group.

The above groups which may be optionally substituted with one, two or three substituents are generally preferably either unsubstituted or substituted with one or two substituents.

Further embodiments of the present invention include compounds of formula (I) or any subgroup thereof, wherein at least one of R^{1a} and R^{1b} is hydrogen, halo, C₁₋₆alkyl, aryl or Het. In a preferred embodiment, both R^{1a} and R^{1b} are methyl. In another preferred embodiment, R^{1a} is hydrogen and R^{1b} is aryl substituted with one or two substituents selected from C₁₋₆alkoxy and phenylC₁₋₆alkoxy. In particular, R^{1a} is hydrogen and R^{1b} is phenyl substituted with one or two substituents selected from methoxy, ethoxy, and phenylmethoxy.

Further embodiments of the present invention include compounds of formula (I) or any subgroup thereof, wherein R² is hydrogen; C₂₋₆alkenyl optionally substituted with aryl or halo; C₄₋₈cycloalkenylC₁₋₆alkyl; aryl²; or Het². In a preferred embodiment, R² is aryl² substituted with one or two substituents selected from halo, C₁₋₆alkoxy, and —(X)ₙ-aryl, wherein n is 1 and X is —O—C₁₋₆alkanediyl. In particular, R² is phenyl substituted with two substituents selected from halo, methoxy, 1-methyl-propoxy, and —(X)ₙ-phenyl, wherein n is 1 and X is —O-methanediyl. In another preferred embodiment, R² is C₂₋₆alkenyl substituted with aryl and halo, in particular ethenyl substituted with halo and phenyl.

Further embodiments of the present invention include compounds of formula (Ia) or any subgroup thereof, wherein R³ is C₁₋₇alkyl optionally substituted with halo, aryl or carboxyl; C₃₋₇cycloalkyl; aryl; Het; Het-thioC₁₋₆alkyl; or —NR^{12a}R^{12b}. In a preferred embodiment of the compounds of formula (Ia) or any subgroup thereof, R³ is C₁₋₆alkyl or polyhaloC₁₋₆alkyl, in particular methyl, pentyl, or trifluoromethyl.

Further embodiments of the present invention include compounds of formula (Ib) or any subgroup thereof, wherein R³ is hydrogen. In a preferred embodiment of the compounds of formula (Ib) or any subgroup thereof, R³ is hydrogen or C₁₋₆alkyl, in particular propyl.

Further embodiments of the present invention include compounds of formula (Ia) or any subgroup thereof, wherein at least one of R^{4a} and R^{4b} is hydrogen or arylcarbonyl.

Further embodiments of the present invention include compounds of formula (Ib) or any subgroup thereof, wherein at least one of R^{4a} and R^{4b} is hydrogen, halo, C₁₋₆alkyl or arylcarbonyl. In a preferred embodiment, both R^{4a} and R^{4b} are hydrogen.

Further embodiments of the present invention include compounds of formula (I) or any subgroup thereof, wherein R⁵ is hydrogen.

Further embodiments of the present invention include compounds of formula (Ia) or any subgroup thereof, wherein at least one of R^{1a} and R^{1b} is hydrogen, chloro; methyl; phenyl optionally substituted with halo, C₁₋₆alkoxy (for example methoxy, ethoxy or n-propoxy), nitro or mono- or di-C₁₋₆alkylamino; or at least one of R^{1a} and R^{1b} is furanyl or thienyl.

Further embodiments of the present invention include compounds of formula (Ib) or any subgroup thereof, wherein at least one of R^{1a} and R^{1b} is hydrogen, chloro; methyl; phenyl optionally substituted with halo, alkylenedioxy, C₁₋₆alkoxy (for example methoxy, ethoxy or n-propoxy), nitro, mono- or di-C₁₋₆alkylamino or benzyloxy; or at least one of R^{1a} and R^{1b} is furanyl or thienyl.

Further embodiments of the present invention include compounds of formula (Ia) or any subgroup thereof, wherein both of R^{1a} and R^{1b} are hydrogen.

Further embodiments of the present invention include compounds of formula (Ib) or any subgroup thereof, wherein both of R^{1a} and R^{1b} are hydrogen or both are methyl.

Further embodiments of the present invention include compounds of formula (Ia) or any subgroup thereof, wherein R² is hydrogen, phenyl optionally substituted with halo, C₁₋₆alkyl, polyhalo-C₁₋₆alkyl, C₁₋₆alkoxy, alkylenedioxy, nitro, hydroxy, mono- or di C₁₋₆alkylamino or with benzyloxy optionally substituted with halo (for example fluoro), or R² is phenyl optionally substituted with benzoyloxy optionally substituted with halo (for example chloro), or R² is furanyl, thienyl or pyrrolyl optionally substituted with halo, C₁₋₆alkyl, nitro, or R² is C₂₋₆alkenyl optionally substituted with aryl especially phenyl, or with aryl, especially phenyl, and halogen, especially bromo; or R² is cyclopentenylmethyl optionally substituted on the cyclopentenyl ring with C₁₋₆ alkyl for example methyl, especially cyclopent-3-enyl, substituted for example with 1, 2 or 3 methyl groups especially 2,2,3-trimethyl.

Further embodiments of the present invention include compounds of formula (Ib) or any subgroup thereof, wherein R² is hydrogen, phenyl optionally substituted with halo, C₁₋₆alkyl, polyhaloC₁₋₆alkyl, C₁₋₆alkoxy, C₁₋₆alkylthio, alkylenedioxy, nitro, hydroxy, mono- or di-C₁₋₆alkylamino or with benzyloxy optionally substituted with halo (for example fluoro), or R² is phenyl or naphthyl, each optionally substituted with benzoyloxy optionally substituted with halo (for example chloro), or R² is pyridyl, thienyl, carbazoyl, indolyl or cromenyl, each optionally substituted with C₁₋₆alkyl; or R² is C₂₋₆alkenyl optionally substituted with aryl especially phenyl, or with aryl, especially phenyl, and halogen, especially bromo.

Further embodiments of the present invention include compounds of formula (Ia) or any subgroup thereof, wherein R³ is methyl, ethyl, isopropyl, n-butyl, sec-butyl, pentyl, heptyl; polyhalomethyl; cyclopropyl; phenyl optionally substituted with halo for example fluoro or with carboxy; benzyl optionally substituted with halo for example fluoro; or R³ is arylamino for example dichlorophenylamino; or benzothiazolylthio-alkyl (for example -methyl) optionally substituted with C₁₋₆alkoxy for example methoxy.

Further embodiments of the present invention include compounds of formula (J) or any subgroup thereof, wherein at least one of R^{4a} and R^{4b} is hydrogen, for example wherein R^{4a} and R^{4b} are both hydrogen.

Further embodiments of the invention include compounds of formula (Ia) or any subgroup thereof, containing one of more of the following groups:

R^{1a} and R^{1b} are both methyl;
R² is 2,4-dichlorophenyl, 3-methoxy-4-benzyloxy-phenyl or 1-bromo-2-phenylethenyl;
R³ is methyl, phenyl, trifluoromethyl or cyclopropyl;
R^{4a} and R^{4b} are both hydrogen; and
R⁵ is hydrogen.

Further embodiments of the invention include compounds of formula (Ib) or any subgroup thereof, containing one of more of the following groups:

R^{1a} and R^{1b} are both methyl or one of R^{1a} and R^{1b} is hydrogen and the other is phenyl substituted with one or two C₁₋₆alkoxy substituents or by a benzyloxy substituent;
R² is phenyl substituted with one or two halo or C₁₋₆alkoxy substituents or with a nitro or benzyloxy substituent;
R³ is hydrogen;
R^{4a} and R^{4b} are both hydrogen or one of R^{4a} and R^{4b} is hydrogen and the other is benzoyl; and
R⁵ is hydrogen.

Examples of specific compounds of formula (Ia) in accordance with the invention include Compound Nos. 35, 38, 42, 45, 48, 51, 53 and 193, referred to in the Tables below, and the salts, stereoisomeric forms, and racemic mixtures thereof.

Examples of specific compounds of formula (Ib) in accordance with the invention include Compound Nos. 78, 97, 108, 116, 156 and 157 referred to in the Tables below, and the salts, stereoisomeric forms, and racemic mixtures thereof.

### Pharmacology

Due to their favorable antiviral properties, as will be apparent from the examples, the compounds of the present invention are useful in the treatment of individuals infected by HCV and for the prophylaxis of these individuals. In general, the compounds of the present invention may be useful in the treatment of warm-blooded animals infected with flaviviruses. Conditions which may be prevented or treated with the compounds of the present invention, especially conditions associated with HCV and other pathogenic flaviviruses, such as Yellow fever, Dengue fever (types 1-4), St. Louis encephalitis, Japanese encephalitis, Murray valley encephalitis, West Nile virus and Kunjin virus. The conditions associated with HCV include progressive liver fibrosis, inflammation and necrosis leading to cirrhosis, end-stage liver disease, and HCC; and for the other pathogenic flaviruses the conditions include yellow fever, dengue fever, hemorrhagic fever and encephalitis.

The compounds of the present invention or any subgroup thereof may therefore be used as medicines against the above-mentioned conditions. Said use as a medicine or method of treatment comprises the systemic administration to HCV-infected subjects of an amount effective to combat the conditions associated with HCV and other pathogenic flaviviruses. Consequently, the compounds of the present invention can be used in the manufacture of a medicament useful for treating conditions associated with HCV and other pathogenic flaviviruses.

In an embodiment, the invention relates to the use of a compound of formula (I) or any subgroup thereof as defined herein in the manufacture of a medicament for treating or combating infection or disease associated with HCV infection in a mammal. The invention also relates to a method of treating a flaviviral infection, in particular an HCV infection, or a disease associated with flavivirus infection comprising administering to a mammal in need thereof an effective amount of a compound of formula (I) or a subgroup thereof as defined herein.

In another embodiment, the present invention relates to the use of formula (I) or any subgroup thereof as defined herein for the manufacture of a medicament useful for inhibiting HCV activity in a mammal infected with flaviviruses, in particular HCV.

In another embodiment, the present invention relates to the use of formula (I) or any subgroup thereof as defined herein for the manufacture of a medicament useful for inhibiting HCV activity in a mammal infected with flaviviruses, wherein said HCV is inhibited in its replication.

In a further aspect, the present invention concerns a pharmaceutical composition comprising a therapeutically effective amount of a novel compound of formula (I) as specified herein, and a pharmaceutically acceptable carrier. A therapeutically effective amount in this context is an amount sufficient to prophylactically act against, to stabilize or to reduce viral infection, and in particular HCV viral infection, in infected subjects or subjects being at risk of being infected. In still a further aspect, this invention relates to a process of preparing a pharmaceutical composition as specified herein, which comprises intimately mixing a pharmaceutically acceptable carrier with a therapeutically effective amount of a said compound of formula (I), as specified herein.

Therefore, the compounds of the present invention may be formulated into various pharmaceutical forms for administration purposes. As appropriate compositions there may be cited all compositions usually employed for systemically administering drugs. To prepare the pharmaceutical compositions of this invention, an effective amount of the particular compound, optionally in addition salt form or metal complex, as the active ingredient is combined in intimate admixture with a pharmaceutically acceptable carrier, which carrier may take a wide variety of forms depending on the form of preparation desired for administration. These pharmaceutical compositions are desirable in unitary dosage form suitable, particularly, for administration orally, rectally, percutaneously, or by parenteral injection. For example, in preparing the compositions in oral dosage form, any of the usual pharmaceutical media may be employed such as, for example, water, glycols, oils, alcohols and the like in the case of oral liquid preparations such as suspensions, syrups, elixirs, emulsions and solutions; or solid carriers such as starches, sugars, kaolin, lubricants, binders, disintegrating agents and the like in the case of powders, pills, capsules, and tablets. Because of their ease in administration, tablets and capsules represent the most advantageous oral dosage unit forms, in which case solid pharmaceutical carriers are obviously employed. For parenteral compositions, the carrier will usually comprise sterile water, at least in large part, though other ingredients, for example, to aid solubility, may be included. Injectable solutions, for example, may be prepared in which the carrier comprises saline solution, glucose solution or a mixture of saline and glucose solution. Injectable suspensions may also be prepared in which case appropriate liquid carriers, suspending agents and the like may be employed. Also included are solid form preparations which are intended to be converted, shortly before use, to liquid form preparations. In the compositions suitable for percutaneous administration, the carrier optionally comprises a penetration enhancing agent and/or a suitable wetting agent, optionally combined with suitable additives of any nature in minor proportions, which additives do not introduce a significant deleterious effect on the skin.

The compounds of the present invention may also be administered via oral inhalation or insufflation by means of methods and formulations employed in the art for administration via this way. Thus, in general the compounds of the present invention may be administered to the lungs in the form of a solution, a suspension or a dry powder, a solution being preferred. Any system developed for the delivery of solutions, suspensions or dry powders via oral inhalation or insufflation are suitable for the administration of the present compounds.

Thus, the present invention also provides a pharmaceutical composition adapted for administration by inhalation or insufflation through the mouth comprising a compound of formula (I) and a pharmaceutically acceptable carrier. Preferably, the compounds of the present invention are administered via inhalation of a solution in nebulized or aerosolized doses.

It is especially advantageous to formulate the aforementioned pharmaceutical compositions in unit dosage form for ease of administration and uniformity of dosage.

Unit dosage form as used herein refers to physically discrete units suitable as unitary dosages, each unit containing a predetermined quantity of active ingredient calculated to produce the desired therapeutic effect in association with the required pharmaceutical carrier. Examples of such unit dosage forms are tablets (including scored or coated tablets), capsules, pills, suppositories, powder packets, wafers, injectable solutions or suspensions and the like, and segregated multiples thereof.

The dosages of the compounds of the invention will depend on a number of factors which will vary from patient to patient. However, it is believed that generally, the daily oral dosage will utilize 0.001-100 mg/kg total body weight, preferably from 0.01-50 mg/kg and more preferably about 0.01 mg/kg-10 mg/kg. The dose regimen will vary, however, depending on the conditions being treated and the judgment of the practitioner.

It should be noted that the compounds of the invention can be administered as individual active ingredients, or as mixtures of several embodiments of this formula. In addition, the compounds of the invention may be used as single therapeutic agents or in combination with other therapeutic agents.

Also, the combination of previously known anti-HCV compound, such as, for instance, interferon-α (IFN-α), pegylated interferon-α and/or ribavirin, and a compound of the present invention can be used as a medicine in a combination therapy. The term “combination therapy” relates to a product containing mandatory (a) a compound of the present invention, and (b) optionally another anti-HCV compound, as a combined preparation for simultaneous, separate or sequential use in treatment of HCV infections, in particular, in the treatment of infections with HCV type 1. Thus, to combat or treat HCV infections, the compounds of this invention may be co-administered in combination with for instance, interferon-α (IFN-α), pegylated interferon-α and/or ribavirin, as well as therapeutics based on antibodies targeted against HCV epitopes, small interfering RNA (Si RNA), ribozymes, DNAzymes, antisense RNA, small molecule antagonists of for instance NS3 protease, NS3 helicase and NS5B polymerase.

Accordingly, the present invention relates to the use of a compound of formula (I) or any subgroup thereof as defined above for the manufacture of a medicament useful for inhibiting HCV activity in a mammal infected with HCV viruses, wherein said medicament is used in a combination therapy, said combination therapy preferably comprising a compound of formula (I) and (pegylated) IFN-α and/or ribavirin.

### Preparation

The compounds according to the invention are either commercially available or can be prepared in accordance with conventional procedures for example as described in the patent and literature references identified above or in accordance with the synthetic routes described below.

Compounds of formulae (Ia) and (Ib) in which R⁵ is hydrogen, represented by formulae (Ia′) and (Ib′) below, can be prepared in accordance with the synthetic route set out in Scheme 1 below:

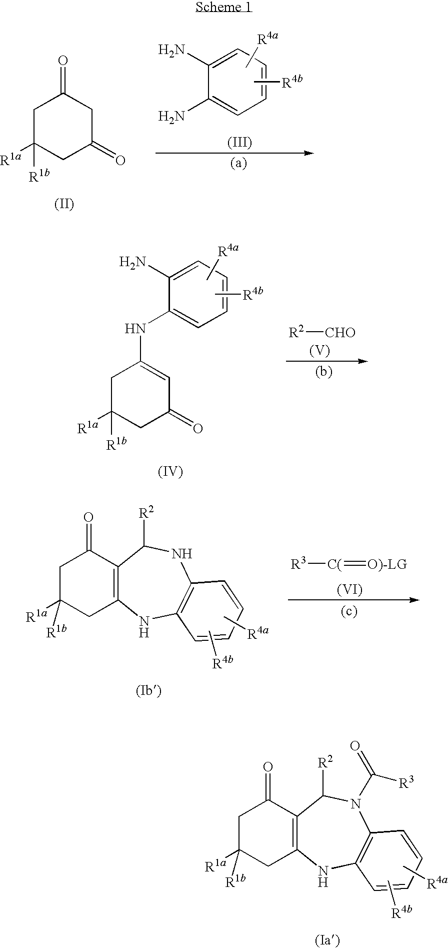

Step (a) a cyclohexane-1,3-dione of formula (JJ) is reacted with an o-phenylene-diamine of formula (III), to give an adduct of formula (IV); the reaction being generally effected in an organic solvent for example toluene for example at reflux.

Step (b): an adduct of formula (IV) is reacted with an aldehyde of formula (V) for example in an anhydrous organic solvent such as ethanol under acid conditions for example in the presence of acetic acid, advantageously at an elevated temperature for example 40° C. to 130° C. preferably at 75° C. for about 5 hours.

Step (c): a compound of formula (Ib′) is reacted with an acylating agent of formula (VI), namely R³—C(═O)-LG, in which LG represent a leaving group; examples of such acylating agents include acyl halides for example acyl chlorides and acyl anhydrides, the acylating reaction being effected in a basic organic solvent such as pyridine for example at a temperature of −20° C. to 50° C. preferably about 0° C.

Compounds of formula (Ia′) in which R⁵ is other than hydrogen can be prepared by reacting a corresponding compound of formula (Ia′) in which R⁵ is hydrogen with a compound of formula R^{5a}-LG¹ in which R^{5a} is as defined for R⁵ other than hydrogen and LG¹ is a leaving group, such as an halogen atom, the reaction being generally effected in the presence of a base such sodium hydride, and in an appropriate organic solvent for example tetrahydrofuran or dimethylformamide.

Compounds of formula (Ib) in which R³ is C₁₋₆alkyl may be prepared from a corresponding compound of formula (Ib) in which R³ is hydrogen by treatment with an alkylating agent for example a C₁₋₆alkyl halide for example an iodide, generally in the presence of a base such as potassium carbonate, and in an appropriate solvent such as acetone, conveniently at room temperature.

Compounds of formula (J) in which R⁵ is other than hydrogen can be prepared by reacting a corresponding compound of formula (I), (Ia) or (Ib) in which R⁵ is hydrogen with a compound of formula R^{5a}-LG¹ in which R^{5a} is as defined for R⁵ other than hydrogen and LG¹ is a leaving group, such as an halogen atom, the reaction being generally effected in the presence of a base such sodium hydride, and in an appropriate organic solvent for example tetrahydrofuran or dimethylformamide.

The starting materials of formula (JJ) are either commercially available or can be prepared in accordance with conventional procedures. For example compounds of Formula (II) in which R^{1b} is H, represented by formula (IIa) below can be prepared in accordance with the synthetic route set out in Scheme 2 below:

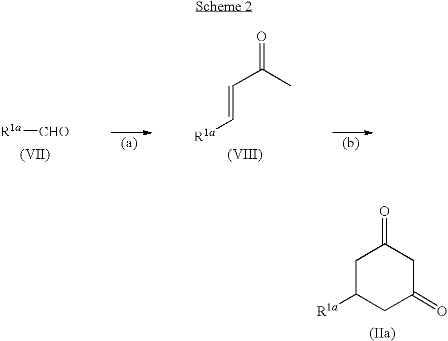

Step a): an aldehyde of formula (VII) is reacted with acetone, in presence of a base such as aqueous sodium hydroxide, to give a ketone of formula (VIII);

Step b): a ketone of formula (VIII) is cyclized to the corresponding cyclohexane-1,3-dione of formula (Ia) by reaction with diethyl malonate in presence of a base, such as potassium tert-butoxide in an appropriate solvent such as ethanol.

Other starting materials of formula (II) are commercially available for example the compound of formula (II) in which R^{1a} and R^{1b} are both methyl is widely available under the name of dimedone.

Alternatively compounds of formula (II) can be prepared from an alpha alkene ketone of Formula (IX) by condensation with diethyl malonate in accordance with Scheme 3 below:

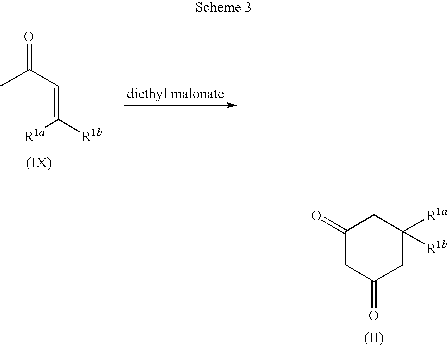

Accordingly, a ketone of Formula (IX) can react with one equivalent or an excess of diethylmalonate, optionally in presence of a solvent such as ethanol or isopropanol.

The present invention further includes the novel compounds of formula (IV) and (Ib′) for example for use as intermediates in the preparation of the compounds of formula (Ia). The present invention further includes the novel compounds of formula (IV) for example for use as intermediates in the preparation of the compounds of formula (Ib).

### EXAMPLES

The following Examples are intended to illustrate, but not to limit, the present invention.

Some of the compounds prepared in the Examples have been analysed by LC/MS on one of the following equipments:
LCT: method XterragradPOS@V1002V1003.olp
electrospray ionisation in positive mode, scanning mode from 100 to 900 amu
Xterra MS C18 (Waters, Milford, Mass.) 5 μm, 3.9×150 mm); Flow rate 1 ml/min. Two mobile phases (mobile phase A: 85% 6.5 mM ammonium acetate+15% acetonitrile; mobile phase B: 20% 6.5 mM ammonium acetate+80% acetonitrile) were employed to run a gradient from 100% A for 3 min to 100% B in 5 min., 100% B for 6 min to 100% A in 3 min, and equilibrate again with 100% A for 3 min).
ZQ: method Xterra_{—}15 cm@V2001V2001.olp
electrospray ionisation in both positive and negative (pulsed) mode scanning from 100 to 1000 amu
Xterra RP C18 (Waters, Milford, Mass.) 5 μm, 3.9×150 mm); Flow rate 1 ml/min. Two mobile phases (mobile phase A: 85% 6.5 mM ammonium acetate+15% acetonitrile; mobile phase B: 20% 6.5 mM ammonium acetate+80% acetonitrile) were employed to run a gradient condition from 100% A for 3 min to 100% B in 5 min., 100% B for 6 min to 100% A in 3 min, and equilibrate again with 100% A for 3 min).

In the Examples the following abbreviations are used:

(M+H)⁺: molecular ion: Å: Angstrom (10⁻¹⁰ m); Ac₂O: acetic acid anhydride; AcOH: acetic acid; Et₂O: diethyl ether; EtOAc: ethyl acetate; EtOH: ethanol; i-Pr₂O: diisopropyl ether; M: molar; mol·L⁻¹; m/z: mass to charge ratio; MeOH: methanol; N: normal; TLC: Thin Layer Chromatography; DIPE: diisopropyl ether; THF: tetrahydrofuran; DMAP: 4-dimethylamino pyridine; DMF: dimethylformamide; EDCI: 1-(3-dimethylaminopropyl)-3-ethylcarbodiimide hydrochloride; HOBT: 1-hydroxy-benzotriazole; DMA: N,N-dimethylaniline.

### Example 1

### 10-Acetyl-3-(2-benzyloxyphenyl)-11-(2,4-dichlorophenyl)-2,3,4,5,10,11-hexahydro-dibenzo[b,e][1,4]diazepin-1-one Compound No. 186 diastereomer A

### Step A

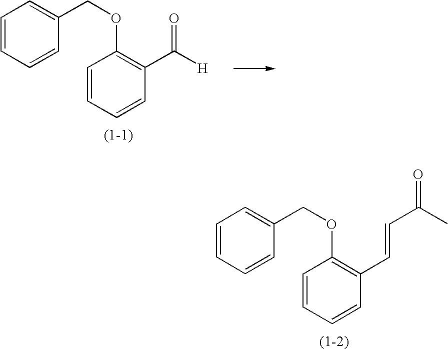

2-Benzyloxybenzaldehyde (30 g, 141.3 mmol) (Intermediate (1-1)) was stirred for 1 week in a mixture of 80 mL acetone and 500 mL of an aqueous NaOH 5% solution. The white precipitate was filtered off, thoroughly washed with water and dried, yielding 35.2 gram (98.9%) of Intermediate (1-2): m/z=253 (M+H)⁺.

### Step B

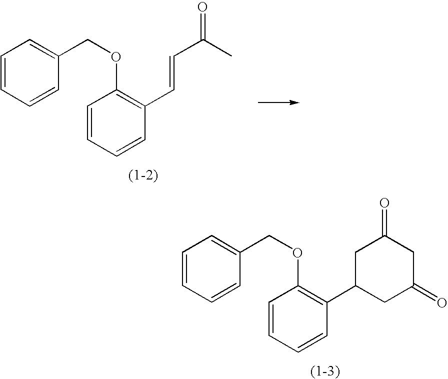

Potassium tert-butoxide (2.22 g, 19.8 mmol) and diethyl malonate (3.01 mL, 19.8 mmol) were added to 50 mL EtOH (dried on 3 Å molecular sieves). To this mixture, Intermediate (1-2) (5 g, 19.8 mmol) and another 10 mL of dry EtOH was added. The reaction mixture was refluxed overnight. The EtOH was evaporated, and the residue was refluxed in 100 mL 2M NaOH for 2 h. The solution was cooled in an ice-bath, 100 mL 5M H₂SO₄ were added, and the mixture was refluxed for 4 h. Two layers were formed, and the aqueous layer was decanted from the oily layer. The oil solidified after cooling to room temperature and was extracted with Et₂O. The Et₂O layer was dried (Na₂SO₄) and evaporated to give 4.21 g (72.2%) of Intermediate (1-3): m/z=295 (M+H)⁺.

### Step C

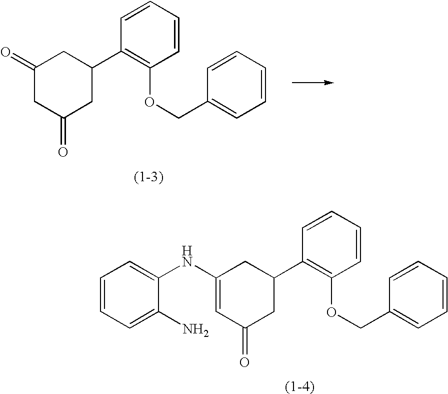

A mixture of Intermediate (1-3) (3.47 g, 11.78 mmol) and o-phenylenediamine (1.27 g, 11.74 mmol) in 100 mL of dry toluene was reacted in a Dean-Stark apparatus overnight. The reaction was cooled to room temperature and the toluene was evaporated. The residue was stirred in i-Pr₂O and filtered off to yield 4.26 g (77.6%) of Intermediate (1-4).

### Step D

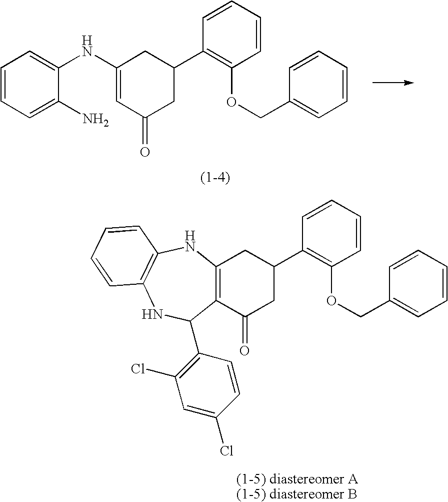

A solution of Intermediate (1-4) (200 mg, 0.520 mmol) and 2,4-dichlorobenzaldehyde (91 mg, 0.520 mmol) in 10 mL dry EtOH and 1 mL AcOH was heated at 75° C. for 5 h. Solvents were evaporated. The residue was dissolved in EtOAc and stirred for 1.5 h with saturated aqueous NaHCO₃, and dried (Na₂SO₄). Two diastereomers were obtained, and purified by silica flash column chromatography (gradient elution from heptane/EtOAc 4:1 to 2:1) to give Intermediate (1-5) diastereomer A, (yield: 118 mg, 41.1%): m/z=542 (M+H)⁺, and Intermediate (1-5) diastereomer B (yield: 57 mg, 20.2%): m/z=542 (M+H)⁺.

### Step E

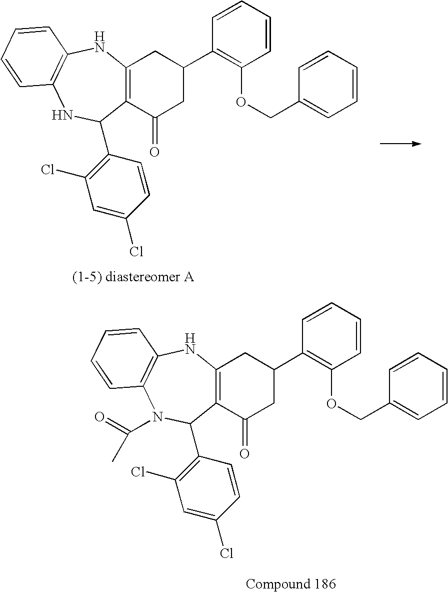

Acetic anhydride (211 μL) was added at 0° C. to a solution of Intermediate (1-5) diastereomer A (52 mg, 0.096 mmol) in pyridine (3 mL). The mixture was stirred at 0° C. during 3 days. Then, water was added to the reaction mixture and the solid was filtered off and washed with water. Purification by preparative TLC (Gradient EtOAc/Heptane 2:1 to 3:1; followed by CH₂Cl₂/MeOH 9:1) provided 41 mg (73.2%) of the final Compound No. 186: m/z=583 (M+H)⁺.

### Example 2

### 10-Acetyl-3-(2-benzyloxyphenyl)-11-(2,4-dichlorophenyl)-2,3,4,5,10,11-hexahydro-dibenzo[b,e][1,4]diazepin-1-one Compound No. 187 diastereomer B

The title product was prepared from Intermediate (1-5) diastereomer B (52 mg, 0.096 mmol) following the procedure described in Example 1.

### Example 3

### 10-Acetyl-3,11-bis-(2-benzyloxyphenyl)-1-(3-benzyloxyphenyl)-2,3,4,5,10,11-hexahydro-dibenzo[b,e][1,4]diazepin-1-one Compound No. 189 diastereomer A

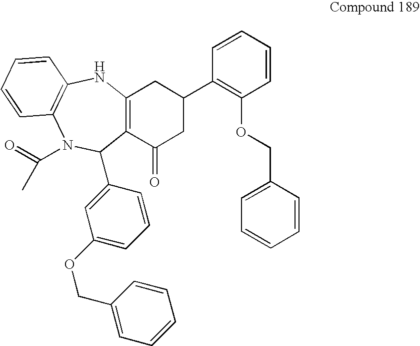

The title product was prepared from Intermediate (1-4) (300 mg, 0.780 mmol) and 3-benzyloxybenzaldehyde (199 mg, 0.938 mmol) following the procedure described in Example 1.

### Example 4

### 10-Acetyl-3,11-bis-(2-benzyloxyphenyl)-11-(3-benzyloxyphenyl)-2,3,4,5,10,11-hexahydro-dibenzo[b,e][1,4]diazepin-1-one Compound 190 diastereomer B

The title product was prepared from Intermediate (1-4) and 3-benzyloxybenzaldehyde following the procedure described in Example 1.

### Example 5

### 10-Acetyl-11-(2,4-dichlorophenyl)-2,3,4,5,10,11-hexahydro-3,3-dimethyl-1H-dibenzo[b,e][1,4]diazepin-1-one Compound No. 38 and enantiomer A Compound No. 36 and enantiomer B Compound No. 35

### Step A

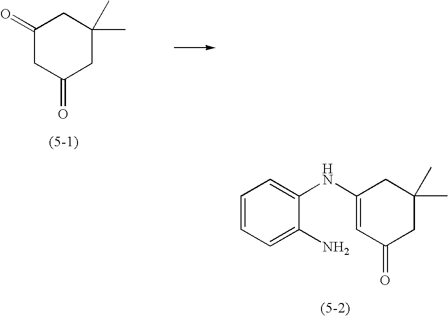

A solution of dimedone (Intermediate (5-1)), 5.0 g, 35.67 mmol) and o-phenylene-diamine (3.86 g, 35.69 mmol) in 150 mL dry toluene were refluxed overnight in a Dean-Stark trap. After 24 h, the solvent was evaporated to give Intermediate (5-2) as an orange foam which was used without further purification in the next step.

Step B

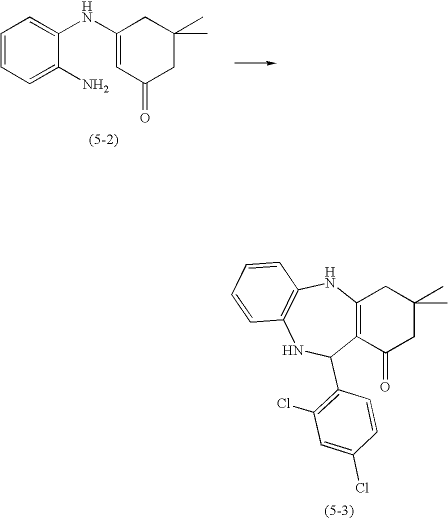

A solution of Intermediate (5-2) (35.7 mmol) and 2,4-dichlorobenzaldehyde (6.24 g, 35.65 mmol) in a mixture of 100 mL dry EtOH and 10 mL AcOH was heated at 75° C. overnight. The reaction mixture was cooled to room temperature and the solvents evaporated. The residue was dissolved in EtOAc and stirred with saturated aqueous NaHCO₃ for 1.5 h. Then, the water layer was removed in a separating funnel and the organic layer was filtered off, the filtrate was washed twice with EtOAc. Organic layers were dried (Na₂SO₄), evaporated and the residue dried under high vacuum, yielding 9.45 g (68.4%) of Intermediate (5-3): m/z=387 (M+H)⁺.

### Step C

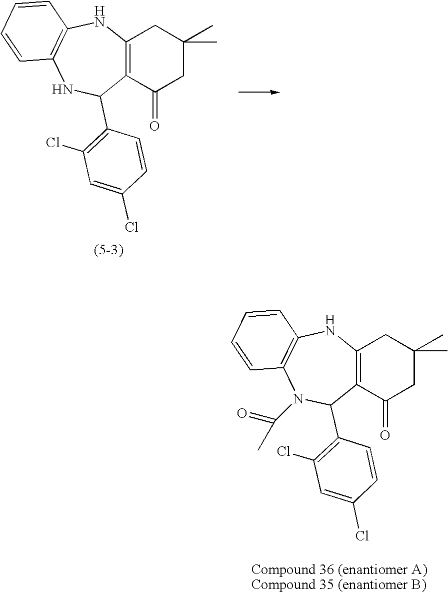

Intermediate (5-3) (1.0 g, 2.582 mmol) was dissolved in 25 mL Pyridine, cooled to 0° C., and 1 mL acetic anhydride was added. The temperature was allowed to warm to room temperature. After 12 h, the reaction mixture was cooled to 0° C., and another 1 mL of acetic anhydride was added. After 12 h, the reaction mixture was filtered off, washed with water and dried overnight at 40° C. under high vacuum. Then, the material was stirred for 1 h in 0.5 N KHSO₄ and extracted with CH₂Cl₂. The organic layer was washed with 0.5 N KHSO₄, dried (Na₂SO₄) and evaporated. The product was finally sonicated in i-Pr₂O, filtered off and dried to give 834 mg (75.2%) of Compound No. 38 as mixture of Compound No. 36 enantiomer A and Compound No. 35 enantiomer B.

### Step D: Separation of Compound No. 36 Enantiomer A and Compound No. 35 Enantiomer B.

Compound No. 36 enantiomer A and Compound No. 35 enantiomer B obtained above in admixture were separated by chiral HPLC using a Berger Minigram SFC, Knauer K2501 UV detector apparatus equipped with a Daicel AD-H 4.6×250 mm column. The mobile phase was 80% CO₂/20% MeOH, the flow of 5 mL/min and the pressure 100 bars. Detection was performed at 220 nm. Several 100 microL injections of a 5 mg/mL solution were performed. Compound No. 36 enantiomer A or the “front enantiomer” is the enantiomer which was eluted from the column first followed by Compound No. 35 enantiomer B or the “back enantiomer”, which was the enantiomer which was eluted from the column second: m/z=430 (M+H)⁺.

### Example 6

### 10-Acetyl-11-(1-bromo-2-phenylvinyl)-3,3-dimethyl-2,3,4,5,10,11-hexahydro-dibenzo[b,e][1,4]diazepin-1-one Compound No 274

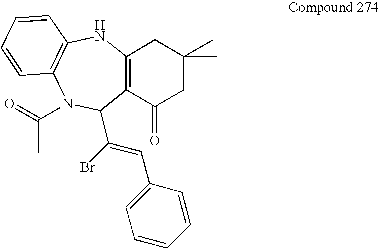

The title compound was prepared from Intermediate (5-2) and 2-bromo-3-phenyl-acroleine following the procedure described in Example 5m/z=466 (M+H)⁺.

### Example 7

### 10-Acetyl-11-(1-chloro-2-phenylvinyl)-3,3-dimethyl-2,3,4,5,10,11-hexahydro-dibenzo[b,e][1,4]diazepin-1-one Compound No. 273

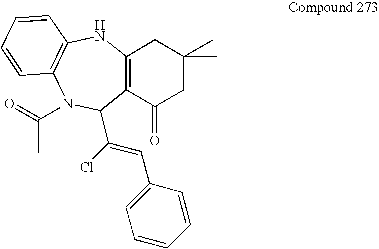

The title compound was prepared from Intermediate (5-2) and 2-chloro-3-phenyl-acroleine following the procedure described in Example 5m/z=421 (M+H)⁺.

### Example 8

### 10-Acetyl-3,3-dimethyl-11-[3-(4-chlorobenzoyloxy)phenyl]-2,3,4,5,10,11-hexahydro-dibenzo[b,e][1,4]diazepin-1-one Compound No. 101

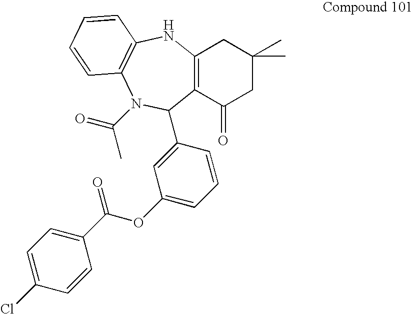

The title compound was prepared from Intermediate (5-2) and 3-[(4-chlorobenzoyl)-oxy]benzaldehyde following the described in Example 5: m/z=515 (M+H)⁺.

### Example 9

### 10-Acetyl-11-(2,4-dichlorophenyl)-3,3,7,8-tetramethyl-2,3,4,5,10,11-hexahydro-dibenzo[b,e][1,4]diazepin-1-one Compound No. 308

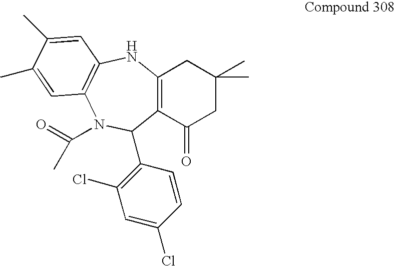

The title compound was prepared from 4,5-dimethyl-o-phenylenediamine and 2,4-dichlorobenzaldehyde following the procedure described in Example 5m/z=457 (M+H)⁺.

### Example 10

### 10-Acetyl-3-(2-benzyloxyphenyl)-11-[3-(4-chlorobenzoyloxy)phenyl]-2,3,4,5,10,11-hexahydro-dibenzo[b,e][1,4]diazepin-1-one Compound No. 192 diastereomer A

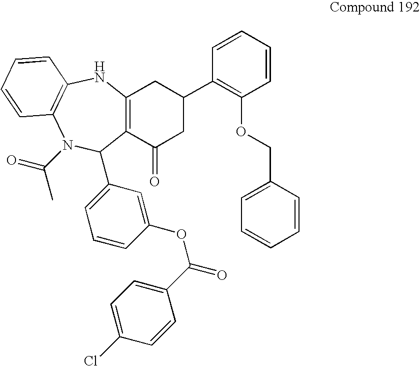

The title compound was prepared from Intermediate (1-4) and 3-[(4-chlorobenzoyl)-oxy]benzaldehyde following the procedure described in Example 1: m/z=669 (M+H)+.

### Example 11

### 10-Acetyl-3-(2-benzyloxyphenyl)-11-[3-(4-chlorobenzoyloxy)phenyl]-2,3,4,5,10,11-hexahydro-dibenzo[b,e][1,4]diazepin-1-one—Compound No. 191 diastereomer B

The title compound was prepared from Intermediate (1-4) and 3-[(4-chlorobenzoyl)oxy]benzaldehyde following the procedure described in Example 2m/z=669 (M+H)⁺.

### Example 12

### 10-Acetyl-11-(2,4-dichlorophenyl)-2,3,4,5,10,11-hexahydro-1H-dibenzo[b,e][1,4]-diazepin-1-one Compound No. 291.

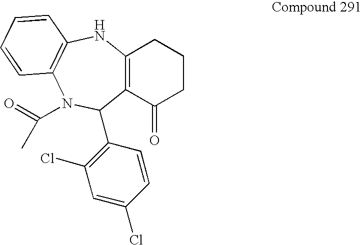

The title compound was prepared from cyclohexan-1,3-dianone, o-phenylenediamine and 2,4-dichlorobenzaldehyde following the procedure described in Example 5: m/z=401 (M+H)⁺.

### Example 13

### 10-Acetyl-7,8-dichloro-11-(2,4-dichlorophenyl)-2,3,4,5,10,11-hexahydro-3,3-dimethyl-1H-dibenzo[b,e][1,4]diazepin-1-one Compound No. 307.

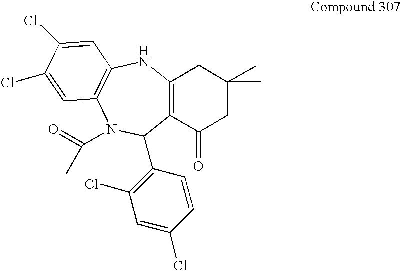

The title compound was prepared from 4,5-dichloro-o-phenylenediamine and 2,4-dichlorobenzaldehyde following the procedure described in Example 5m/z=497 (M+H)⁺.

### Example 14

### 3,3-dimethyl-11-(4-hydroxyphenyl)-2,3,4,5,10,11-hexahydro-1H-dibenzo[b,e][1,4]diazepin-1-one Compound No. 440

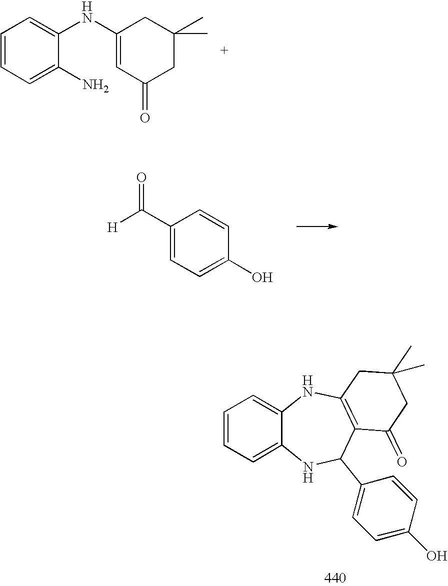

The title compound was prepared from intermediate 5-2 and 4-hydroxybenzaldehyde following the procedure described for 11-(2,4-dichlorophenyl)-2,3,4,5,10,11-hexa-hydro-3,3-dimethyl-1H-dibenzo[b,e][1,4]diazepin-1-one 5-3: m/z=335 (M+H)⁺.

### Example 15

### 11-(4-acetoxyphenyl)-10-acetyl-3,3-dimethyl-2,3,4,5,10,11-hexahydro-1H-dibenzo[b,e][1,4]diazepin-1-one Compound No. 1001

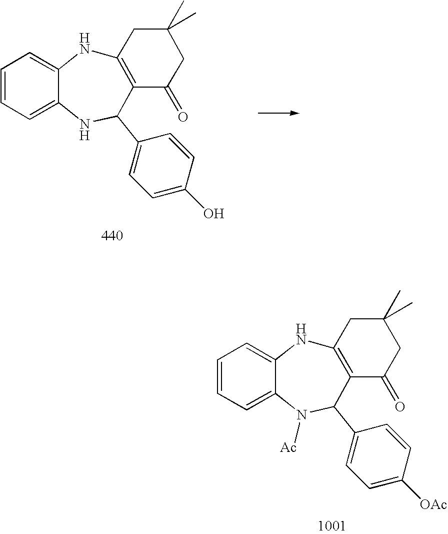

Ac₂O (5 mL) was added at 0° C. to a solution of 3,3-dimethyl-11-(4-hydroxyphenyl)-2,3,4,5,10,11-hexahydro-1H-dibenzo[b,e][1,4]diazepin-1-one 440 in pyridine (50 mL). After 7 days, the reaction mixture was quenched with water (250 mL). Then, the solid was filtered off and washed with water. The solid was successively re-dissolved in CH₂Cl₂, washed with 0.5 N KHSO₄ (twice), dried (Na₂SO₄) and evaporated. The residue was sonicated in Et₂O and filtered off to give 4.36 g (71%) of the target compound 1001: m/z=419 (M+H)+.

### Example 16

### 10-acetyl-11-(4-hydroxyphenyl)-3,3-dimethyl-2,3,4,5,10,11-hexahydro-1H-dibenzo[b,e][1,4]diazepin-1-one Compound N o. 137

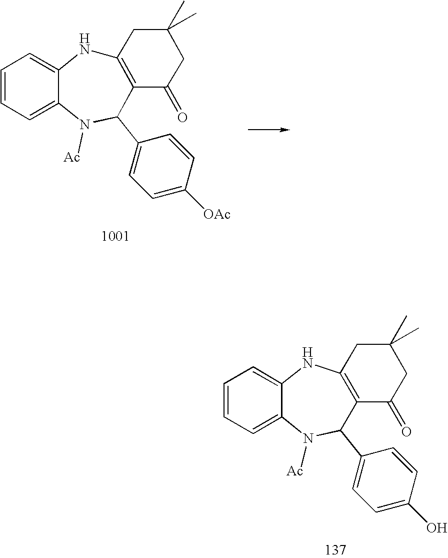

A solution of lithium hydroxide hydrate (672 mg) in water (5 mL) was added to a stirred suspension of 11-(4-acetoxyphenyl)-10-acetyl-3,3-dimethyl-2,3,4,5,10,11-hexa-hydro-1H-dibenzo[b,e][1,4]diazepin-1-one 1001 (4.26 g, 10.2 mmol) in MeOH/THF/H₂O 2.5:0.5:1 (70 mL). After 30 minutes, 1N HCl (20 mL) was added. Then, the reaction mixture was diluted with water (100 mL) and concentrated under reduced pressure. The precipitate was successively filtered off, washed with water and dried to give 3.70 g (97%) of the title product 137 as a white powder: m/z=377 (M+H)⁺.

### Example 17

### 10-acetyl-3,3-dimethyl-11-[4-(2-pyridylmethoxy)phenyl]-2,3,4,5,10,11-hexahydro-1H-dibenzo[b,e][1,4]diazepin-1-one Compound No. 141

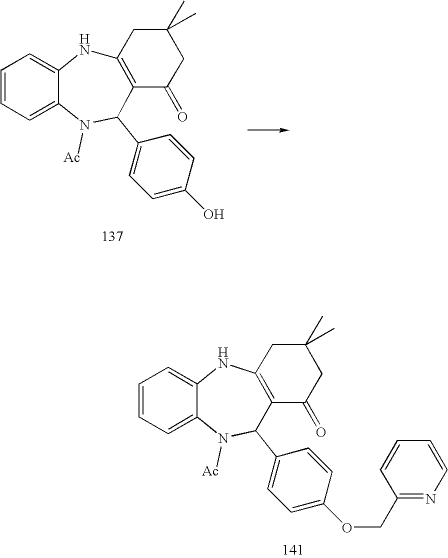

A mixture of 10-acetyl-3,3-dimethyl-11-(4-hydroxyphenyl)-2,3,4,5,10,11-hexahydro-1H-dibenzo[b,e][1,4]diazepin-1-one 137 (250 mg, 0.664 mmol), 2-picolylchloride hydrochloride (109 mg, 1 eq.), cesium carbonate (476 mg, 2.2 eq.) in dry DMF (10 mL) were stirred at room temperature for 78 h. Then, the reaction mixture was diluted with water (300 mL) and the precipitate was successively filtered off, washed with water, dried and triturated in isopropylether to give 79 mg of the target product 141: m/z=468 (M+H)⁺.

### Example 18

### 10-acetyl-11-[4-(2-chlorobenzyloxy)phenyl]-3,3-dimethyl-2,3,4,5,10,11-hexahydro-1H-dibenzo[b,e][1,4]diazepin-1-one Compound No. 148.

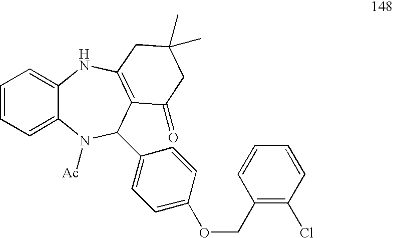

The title compound was prepared from 10-acetyl-11-(4-hydroxyphenyl)-3,3-dimethyl-2,3,4,5,10,11-hexahydro-1H-dibenzo[b,e][1,4]diazepin-1-one 137 and 2-chlorobenzyl-bromide following the procedure described for example 17: m/z=501 (M+H)⁺.

### Example 19

### 10-acetyl-3,3-dimethyl-11-[4-(4-pyridylmethoxy)phenyl]-2,3,4,5,10,11-hexahydro-1H-dibenzo[b,e][1,4]diazepin-1-one Compound No. 145

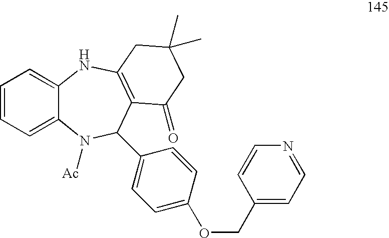

The title compound was prepared from 10-acetyl-11-(4-hydroxyphenyl)-3,3-dimethyl-2,3,4,5,10,11-hexahydro-1H-dibenzo[b,e][1,4]diazepin-1-one 137 and 4-picolyl-chloride hydrochloride following the procedure described for example 17: m/z=468 (M+H)⁺.

### Example 20

### 10-acetyl-3,3-dimethyl-11-[4-(3-pyridylmethoxy)phenyl]-2,3,4,5,10,11-hexahydro-1H-dibenzo[b,e][1,4]diazepin-1-one Compound No. 140.

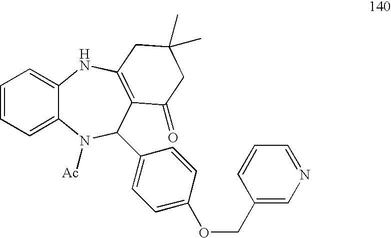

The title compound was prepared from 10-acetyl-11-(4-hydroxyphenyl)-3,3-dimethyl-2,3,4,5,10,11-hexahydro-1H-dibenzo[b,e][1,4]diazepin-1-one 137 and 3-picolyl-chloride hydrochloride following the procedure described for example 17: m/z=468 (M+H)⁺.

### Example 21

### 10-acetyl-11-(2,4-dichlorophenyl)-3,3-dimethyl-1-oxo-2,3,4,5,10,11-hexahydro-1H-dibenzo[b,e][1,4]diazepine-7-carboxylic acid methyl ester Compound no. 520

### Step A.

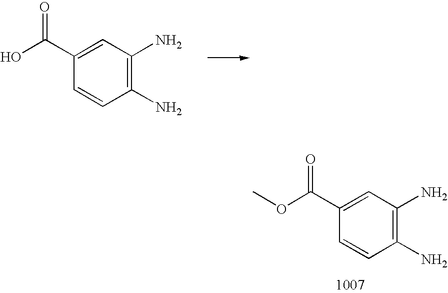

Thionyl chloride (16.0 mL, 220 mmol) was added to a suspension of 3,4-diamino-benzoic acid (16.8 g, 110 mmol) in dry MeOH (200 mL). The resulting mixture was heated at reflux. After 12 h, the solution was successively cooled down to room temperature and concentrated under reduced pressure. The residue was triturated in diluted NaHCO₃. Then, the precipitate was filtered off and dried to give 10.1 g (55%) of the target compound 1007.

### Step B.

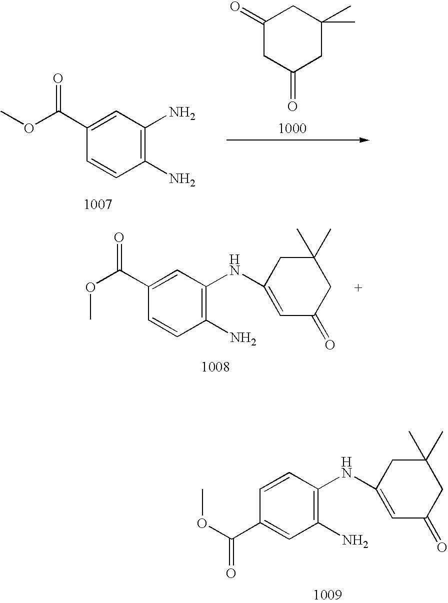

The intermediates 1008 and 1009 were prepared from 3,4-diaminobenzoic acid methyl ester 1007 and dimedone 1000 following the procedure (Step A) described for the synthesis of 10-acetyl-11-(2,4-dichlorophenyl)-2,3,4,5,10,11-hexahydro-3,3-dimethyl-1H-dibenzo[b,e][1,4]diazepin-1-one (compound no. 38).

### Step C.

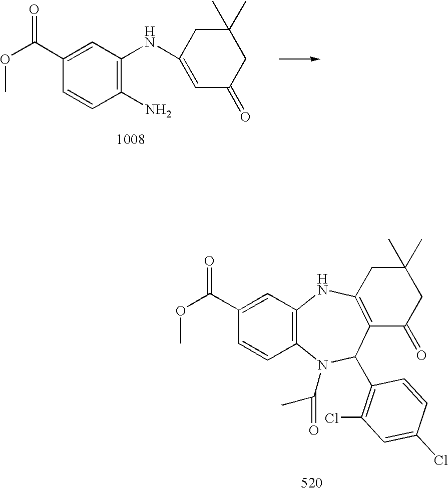

The title compound 520 was prepared from 1008 and 2,4-dichlorobenzaldehyde following the procedure described for 10-acetyl-11-(2,4-dichlorophenyl)-2,3,4,5,10,11-hexahydro-3,3-dimethyl-1H-dibenzo[b,e][1,4]diazepin-1-one (compound no. 38): m/z=487 (M+H)⁺.

### Example 22

### 10-acetyl-11-(2,4-dichlorophenyl)-3,3-dimethyl-1-oxo-2,3,4,5,10,11-hexahydro-1H-dibenzo[b,e][1,4]diazepine-8-carboxylic acid methyl ester Compound no. 521

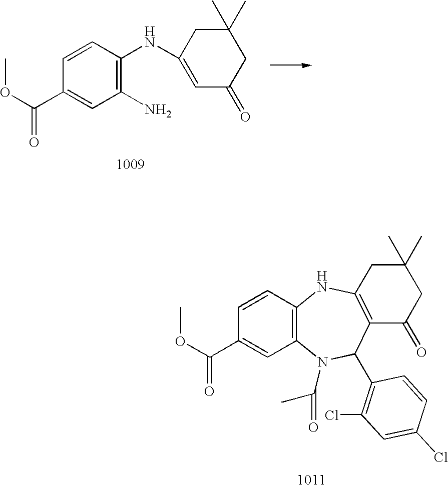

The title compound 521 was prepared from 1009 and 2,4-dichlorobenzaldehyde following the procedure described for 10-acetyl-1-(2,4-dichlorophenyl)-2,3,4,5,10,11-hexahydro-3,3-dimethyl-1H-dibenzo[b,e][1,4]diazepin-1-one (compound no. 38): m/z=487 (M+H)⁺.

### Example 23

### 10-acetyl-11-(2,4-dichlorophenyl)-3,3-dimethyl-1-oxo-2,3,4,5,10,11-hexahydro-1H-dibenzo[b,e][1,4]diazepine-7-carboxylic acid Compound no. 1012

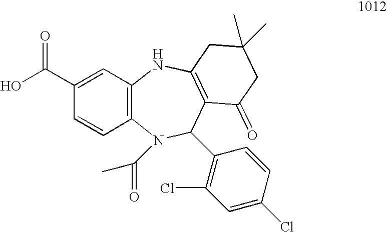

A solution of lithium hydroxide hydrate (354 mg, 8.2 mmol) was added to a suspension of 10-acetyl-11-(2,4-dichlorophenyl)-3,3-dimethyl-1-oxo-2,3,4,5,10,11-hexahydro-1H-dibenzo[b,e][1,4]diazepine-7-carboxylic acid methyl ester 520 (2.0 g, 4.10 mmol) in water (25 mL) and THF (25 mL). After 12 h, the pH of the reaction mixture was adjusted to 3 with 1 N HCl. The precipitate was collected by filtration, the washed with water and dried to afford 1.87 g (96.4%) of the title product 1012: m/z=473 (M+H)⁺.

### Example 24

### 10-acetyl-11-(2,4-dichlorophenyl)-3,3-dimethyl-1-oxo-2,3,4,5,10,11-hexahydro-1H-dibenzo[b,e][1,4]diazepine-8-carboxylic acid Compound no. 522.

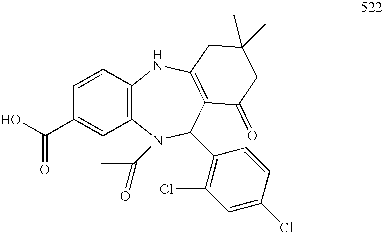

The title compound 522 was prepared from 10-acetyl-11-(2,4-dichlorophenyl)-3,3-dimethyl-1-oxo-2,3,4,5,10,11-hexahydro-1H-dibenzo[b,e][1,4]diazepine-8-carboxylic acid methyl ester 521 following the procedure described for the preparation of 10 acetyl-11-(2,4-dichlorophenyl)-3,3-dimethyl-1-oxo-2,3,4,5,10,11-hexahydro-1H-dibenzo[b,e][1,4]diazepine-7-carboxylic acid 1012: m/z=473 (M+H)⁺.

### Example 25

### 10-acetyl-N-(morpholin-4-ylethyl)-11-(2,4-dichlorophenyl)-3,3-dimethyl-1-oxo-2,3,4,5,10,11-hexahydro-1H-dibenzo[b,e][1,4]diazepine-7-carboxamide Compound no. 321

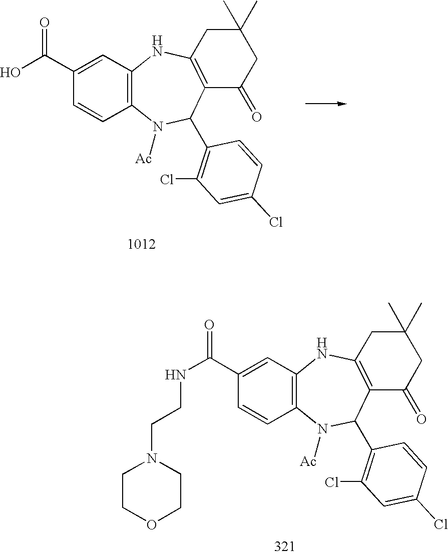

A solution of 10-acetyl-11-(2,4-dichlorophenyl)-3,3-dimethyl-1-oxo-2,3,4,5,10,11-hexahydro-1H-dibenzo[b,e][1,4]diazepine-7-carboxylic acid 1012 (250 mg, 0.53 mmol), 4-(2-aminoethyl)morpholine, EDCI.HCl (203 mg, 1.06 mmol), HOAT (144 mg, 1.06 mmol), and DIPEA (185 μL, 1.06 mmol) in dry DMF (5 mL) was stirred overnight at room temperature. Then, the reaction mixture was diluted with water (75 mL), and the precipitate formed was collected by filtration, then washed with water and dried to give 120 mg (39%) of the title product 321: m/z=585 (M+H)⁺.

### Example 26

### 10-acetyl-N—(N,N-dimethylaminopropyl)-11-(2,4-dichlorophenyl)-3,3-dimethyl-1-oxo-2,3,4,5,10,11-hexahydro-1H-dibenzo[b,e][1,4]diazepine-7-carboxamide Compound no. 1015

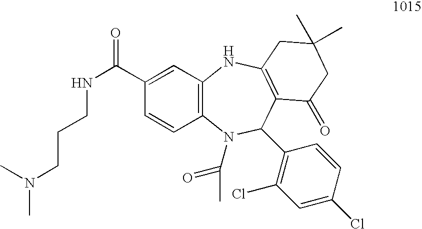

The title compound 1015 was prepared in 73% yield from 10-acetyl-11-(2,4-dichloro-phenyl)-3,3-dimethyl-1-oxo-2,3,4,5,10,11-hexahydro-1H-dibenzo[b,e][1,4]diazepine-7-carboxylic acid 1012 and 3-(N,N-dimethylamino)propylamine following the procedure reported for the preparation of N-(morpholin-4-ylethyl)-11-(2,4-dichlorophenyl)-3,3-dimethyl-1-oxo-2,3,4,5,10,11-hexahydro-1H-dibenzo[b,e][1,4]diazepine-7-carboxamide 321: m/z=557 (M+H)⁺.

### Example 27

### 10-acetyl-N-(4-pyridylethyl)-11-(2,4-dichlorophenyl)-3,3-dimethyl-1-oxo-2,3,4,5,10,11-hexahydro-1H-dibenzo[b,e][1,4]diazepine-7-carboxamide Compound no. 1016.

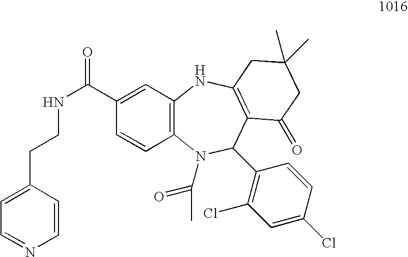

The title compound 1016 was prepared in 53% yield from 10-acetyl-11-(2,4-dichloro-phenyl)-3,3-dimethyl-1-oxo-2,3,4,5,10,11-hexahydro-1H-dibenzo[b,e][1,4]diazepine-7-carboxylic acid 1012 and 4-pyridylethylamine following the procedure reported for the preparation of 10-acetyl-N-(morpholin-4-ylethyl)-11-(2,4-dichlorophenyl)-3,3-dimethyl-1-oxo-2,3,4,5,10,11-hexahydro-1H-dibenzo[b,e][1,4]diazepine-7-carboxamide 321: m/z=577 (M+H)⁺.

### Example 28

### 10-acetyl-N—(N,N-dimethylaminoethyl)-11-(2,4-dichlorophenyl)-3,3-dimethyl-1-oxo-2,3,4,5,10,11-hexahydro-1H-dibenzo[b,e][1,4]diazepine-7-carboxamide Compound no. 1018

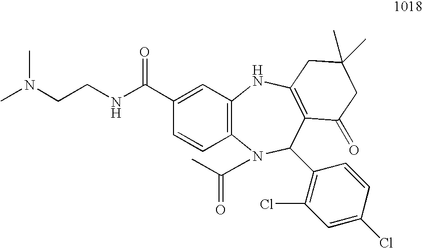

The title compound 1018 was prepared from 10-acetyl-11-(2,4-dichlorophenyl)-3,3-dimethyl-1-oxo-2,3,4,5,10,11-hexahydro-1H-dibenzo[b,e][1,4]diazepine-7-carboxylic acid 1012 and 2-(N,N-dimethylamino)ethylamine following the procedure reported for the preparation of 10-acetyl-N-(morpholin-4-ylethyl)-11-(2,4-dichlorophenyl)-3,3-dimethyl-1-oxo-2,3,4,5,10,11-hexahydro-1H-dibenzo[b,e][1,4]diazepine-7-carboxamide 321: m/z=543 (M+H)⁺.

### Example 29

### 10-acetyl-N-(2-piperidin-1-ylethyl)-11-(2,4-dichlorophenyl)-3,3-dimethyl-1-oxo-2,3,4,5,10,11-hexahydro-1H-dibenzo[b,e][1,4]diazepine-7-carboxamide Compound no. 1019.

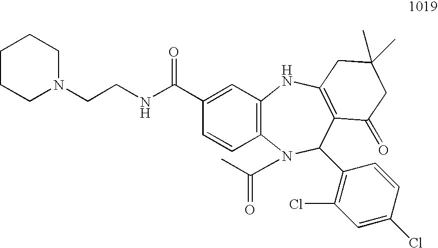

The title compound 1019 was prepared from 10-acetyl-11-(2,4-dichlorophenyl)-3,3-dimethyl-1-oxo-2,3,4,5,10,11-hexahydro-1H-dibenzo[b,e][1,4]diazepine-7-carboxylic acid 1012 and 2-(piperidin-1-yl)ethylamine following the procedure reported for the preparation of 10-acetyl-N-(morpholin-4-ylethyl)-11-(2,4-dichlorophenyl)-3,3-dimethyl-1-oxo-2,3,4,5,10,11-hexahydro-1H-dibenzo[b,e][1,4]diazepine-7-carboxamide 321: m/z=583 (M+H)⁺.

### Example 30

### 10-acetyl-N-(2-cyanoethyl)-11-(2,4-dichlorophenyl)-3,3-dimethyl-1-oxo-2,3,4,5,10,11-hexahydro-1H-dibenzo[b,e][1,4]diazepine-7-carboxamide Compound no. 1020.

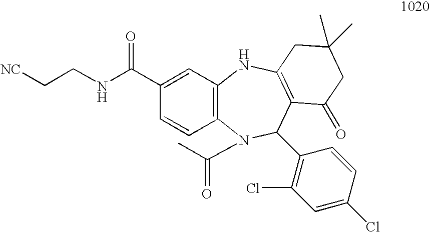

The title compound 1020 was prepared from 10-acetyl-11-(2,4-dichlorophenyl)-3,3-dimethyl-1-oxo-2,3,4,5,10,11-hexahydro-1H-dibenzo[b,e][1,4]diazepine-7-carboxylic acid 1012 and 2-cyanoethylamine following the procedure reported for the preparation of 10-acetyl-N-(morpholin-4-ylethyl)-11-(2,4-dichlorophenyl)-3,3-dimethyl-1-oxo-2,3,4,5,10,11-hexahydro-1H-dibenzo[b,e][1,4]diazepine-7-carboxamide 321: m/z=525 (M+H)⁺.

### Example 31

### 10-acetyl-11-(2,4-dichlorophenyl)-7-hydroxymethyl-3,3-dimethyl-2,3,4,5,10,11-hexahydro-1H-dibenzo[b,e][1,4]diazepin-1-one Compound no. 523

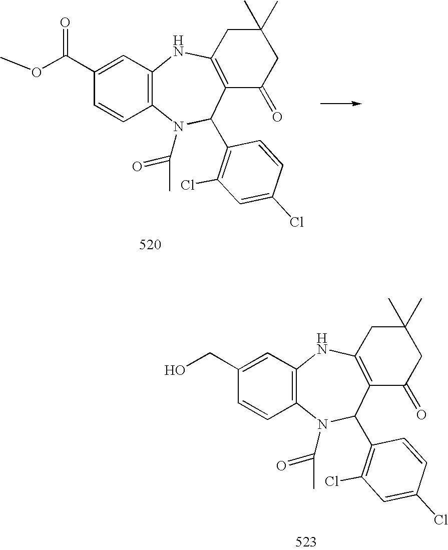

Sodium borohydride (824 mg, 21.8 mmol) was added portion wise to a solution of 10-acetyl-11-(2,4-dichlorophenyl)-3,3-dimethyl-1-oxo-2,3,4,5,10,11-hexahydro-1H-dibenzo[b,e][1,4]diazepine-7-carboxylic acid methyl ester 520 (5.3 g, 10.9 mmol) in absolute ethanol (100 mL). After 24 h, sodium borohydride (824 mg, 21.8 mmol) was added to the reaction mixture. This operation was repeated 3 times (total: 14 eq. of NaBH₄ were used). The reaction mixture was added dropwise to a solution of 2N HCl (500 mL). The precipitate was collected by filtration, washed with water and dried to give 3.83 g (77%) of the title product 523 as a white powder: m/z=459 (M+H)⁺.

### Example 32

### 10-acetyl-7-bromomethyl-11-(2,4-dichlorophenyl)-3,3-dimethyl-2,3,4,5,10,11-hexahydro-1H-dibenzo[b,e][1,4]diazepin-1-one Compound no. 1022

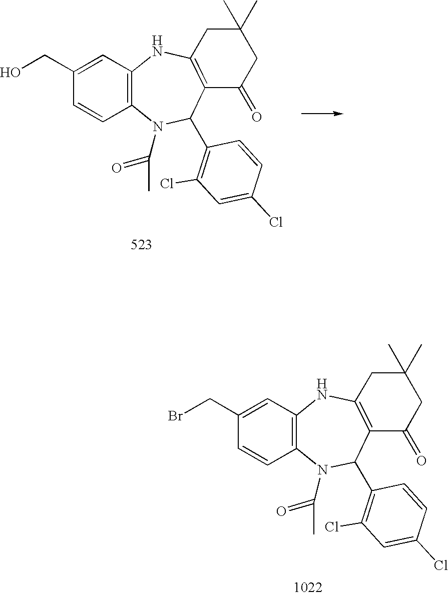

Phosphorous tribromide (118 μL, 1.25 mmol) was gradually added under nitrogen at 0° C. to a stirred solution of 10-acetyl-11-(2,4-dichlorophenyl)-7-hydroxymethyl-3,3-dimethyl-2,3,4,5,10,11-hexahydro-1H-dibenzo[b,e][1,4]diazepin-1-one 523 in DCE (2 mL). The resulting solution was stirred at room temperature for 1 h. Then, a diluted aqueous solution of sodium bicarbonate was added. The reaction mixture was extracted with AcOEt, dried (Na₂SO₄) and evaporated to give 157 mg (68%) of the title product 1022: m/z=522 (M+H)⁺.

### Example 33

### 10-acetyl-7-chloromethyl-11-(2,4-dichlorophenyl)-3,3-dimethyl-2,3,4,5,10,11-hexahydro-1H-dibenzo[b,e][1,4]diazepin-1-one Compound no. 1023.

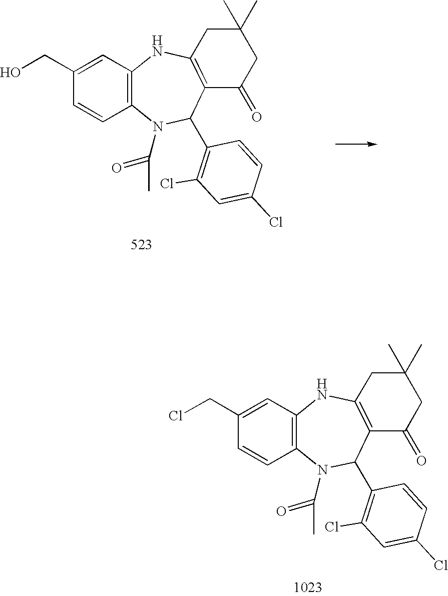

Thionylchloride (238 μL, 3.26 mmol) was added dropwise under nitrogen at 0° C. to a stirred solution of 10-acetyl-11-(2,4-dichlorophenyl)-7-hydroxymethyl-3,3-dimethyl-2,3,4,5,10,11-hexahydro-1H-dibenzo[b,e][1,4]diazepin-1-one 523 (500 mg, 1.09 mmol) in DCE (10 mL). The resulting solution was stirred at room temperature for 2 h. Then, ice-cold water was added. The reaction mixture was extracted with DCM, dried (Na₂SO₄) and evaporated to give 410 mg (79%) of the title product 1023: m/z=477 (M+H)⁺.

### Example 34

### 10-acetyl-11-(2,4-dichlorophenyl)-3,3-dimethyl-1-oxo-2,3,4,5,10,11-hexahydro-1H-dibenzo[b,e][1,4]diazepine-7-carboxaldehyde Compound no. 1024.

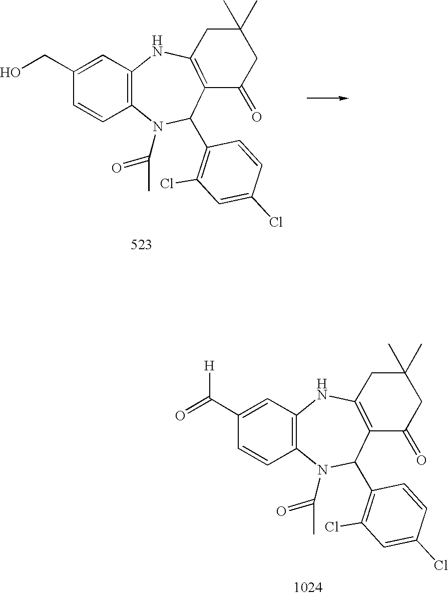

Manganese (IV) oxide was added to a stirred solution of 10-acetyl-11-(2,4-dichloro-phenyl)-7-hydroxymethyl-3,3-dimethyl-2,3,4,5,10,11-hexahydro-1H-dibenzo[b,e][1,4]diazepin-1-one 523 in acetone (10 mL). The resulting solution was heated to reflux. After 2 days, the reaction mixture was cooled down to room temperature, filtered over kieselguhr, and evaporated to give 400 mg (40%) of the title product 1024: m/z=457 (M+H)⁺.

### Example 35

### 10-acetyl-11-(2,4-dichlorophenyl)-3,3-dimethyl-7-(2-morpholin-4-ylethylaminomethyl)-2,3,4,5,10,11-hexahydro-1H-dibenzo[b,e][1,4]diazepin-1-one Compound no. 1025.

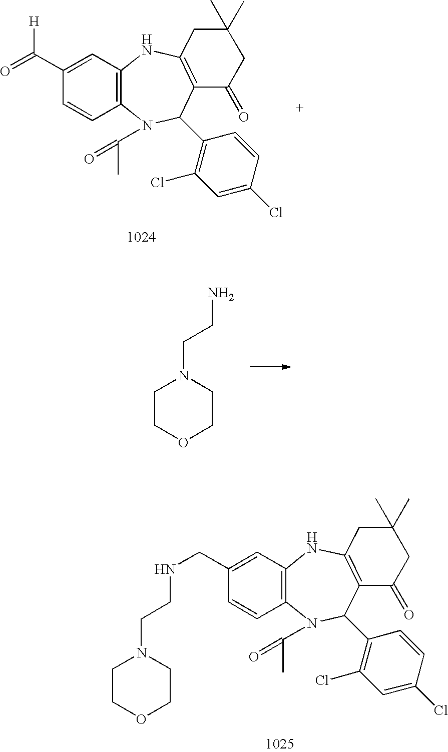

A solution of 10-acetyl-11-(2,4-dichlorophenyl)-3,3-dimethyl-1-oxo-2,3,4,5,10,11-hexahydro-1H-dibenzo[b,e][1,4]diazepine-7-carboxaldehyde 1024 (200 mg, 0.44 mmol) and 4-(2-aminoethyl)morpholine (53 μL, 0.40 mmol) in DCM (5 mL) was stirred at room temperature for 30 minutes. Then, NaBH(OAc)₃ (122 mg, 0.57 mmol) and acetic acid (26.3 μL, 1.2 eq.) were added. The resulting reaction mixture was stirred overnight at room temperature, then quenched with a saturated solution of sodiumbicarbonate, extracted with AcOEt, dried (Na₂SO₄) and evaporated to give 190 mg (87%) of the title product 1025: m/z=571 (M+H)⁺.

### Example 36

### 10-acetyl-11-(2,4-dichlorophenyl)-3,3-dimethyl-7-[3-(N,N-dimethyl-amino)propylaminomethyl]-2,3,4,5,10,11-hexahydro-1H-dibenzo[b,e][1,4]diazepin-1-one Compound no. 1026.

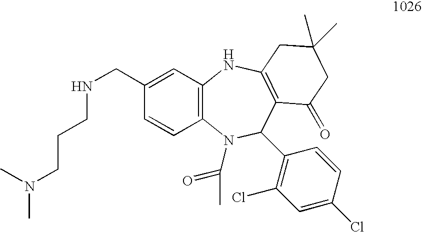

The title compound 1026 was prepared from 10-acetyl-11-(2,4-dichlorophenyl)-3,3-dimethyl-1-oxo-2,3,4,5,10,11-hexahydro-1H-dibenzo[b,e][1,4]diazepine-7-carboxaldehyde 1024 and 3-(N,N-dimethylaminopropylamine following the procedure reported for the preparation of 10-acetyl-11-(2,4-dichlorophenyl)-3,3-dimethyl-7-(2-morpholin-4-ylethylaminomethyl)-2,3,4,5,10,11-hexahydro-1H-dibenzo[b,e][1,4]diazepin-1-one 1025: m/z=543 (M+H)⁺.

### Example 37

### 10-acetyl-11-(2,4-dichlorophenyl)-3,3-dimethyl-7-[2-(4-pyridyl)ethyl-aminomethyl]-2,3,4,5,10,11-hexahydro-1H-dibenzo[b,e][1,4]diazepin-1-one Compound no. 1027.

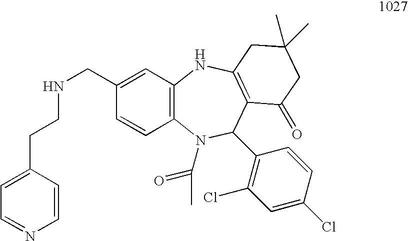

The title compound 1027 was prepared from 10-acetyl-11-(2,4-dichlorophenyl)-3,3-dimethyl-1-oxo-2,3,4,5,10,11-hexahydro-1H-dibenzo[b,e][1,4]diazepine-7-carboxaldehyde 1024 and 4-pyridylethylamine following the procedure reported for the preparation of 10-acetyl-11-(2,4-dichlorophenyl)-3,3-dimethyl-7-(2-morpholin-4-yl-ethylaminomethyl)-2,3,4,5,10,11-hexahydro-1H-dibenzo[b,e][1,4]diazepin-1-one 1025: m/z=563 (M+H)⁺.

### Example 38

### 10-acetyl-11-(2,4-dichlorophenyl)-3,3-dimethyl-7-[2-(N,N-dimethyl-amino)ethylaminomethyl]-2,3,4,5,10,11-hexahydro-1H-dibenzo[b,e][1,4]diazepin-1-one Compound no. 1028.

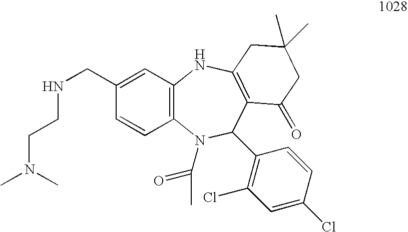

The title compound 1028 was prepared from 10-acetyl-11-(2,4-dichlorophenyl)-3,3-dimethyl-1-oxo-2,3,4,5,10,11-hexahydro-1H-dibenzo[b,e][1,4]diazepine-7-carboxaldehyde 1024 and 2-(N,N-dimethylamino)ethylamine following the procedure reported for the preparation of 10-acetyl-11-(2,4-dichlorophenyl)-3,3-dimethyl-7-(2-morpholin-4-ylethylaminomethyl)-2,3,4,5,10,11-hexahydro-1H-dibenzo[b,e][1,4]diazepin-1-one 1025: m/z=529 (M+H)⁺.

### Example 39

### 10-acetyl-11-(2,4-dichlorophenyl)-3,3-dimethyl-7-[2-(piperidin-1-yl)-ethylaminomethyl]-2,3,4,5,10,11-hexahydro-1H-dibenzo[b,e][1,4]diazepin-1-one Compound no. 1030

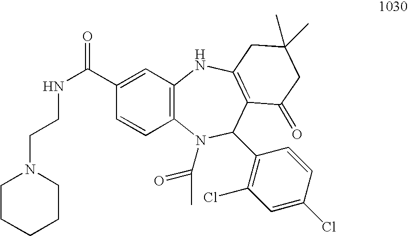

The title compound 1030 was prepared from 10-acetyl-11-(2,4-dichlorophenyl)-3,3-dimethyl-1-oxo-2,3,4,5,10,11-hexahydro-1H-dibenzo[b,e][1,4]diazepine-7-carboxaldehyde 1024 and 2-(piperidin-1-yl)ethylamine following the procedure reported for the preparation of 10-acetyl-11-(2,4-dichlorophenyl)-3,3-dimethyl-7-(2-morpholin-4-ylethylaminomethyl)-2,3,4,5,10,11-hexahydro-1H-dibenzo[b,e][1,4]diazepin-1-one 1025: m/z=569 (M+H)⁺.

### Example 40

### 10-acetyl-11-(2,4-dichlorophenyl)-3,3-dimethyl-7-(2-cyanoethylamino-methyl)-2,3,4,5,10,11-hexahydro-1H-dibenzo[b,e][1,4]diazepin-1-one Compound no. 1031.

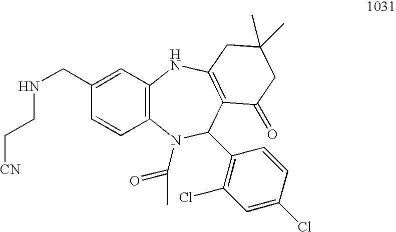

The title compound 1031 was prepared from 10-acetyl-11-(2,4-dichlorophenyl)-3,3-dimethyl-1-oxo-2,3,4,5,10,11-hexahydro-1H-dibenzo[b,e][1,4]diazepine-7-carboxaldehyde 1024 and 2-cyanoethylamine following the procedure reported for the preparation of 10-acetyl-11-(2,4-dichlorophenyl)-3,3-dimethyl-7-(2-morpholin-4-ylethylaminomethyl)-2,3,4,5,10,11-hexahydro-1H-dibenzo[b,e][1,4]diazepin-1-one 1025: m/z=511 (M+H)⁺.

### Example 41

### 10-acetyl-11-(2,4-dichlorophenyl)-3,3-dimethyl-7-(morpholin-4-ylmethyl)-2,3,4,5,10,11-hexahydro-1H-dibenzo[b,e][1,4]diazepin-1-one Compound no. 1032.

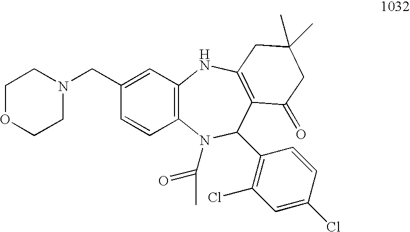

The title compound 1032 was prepared from 10-acetyl-11-(2,4-dichlorophenyl)-3,3-dimethyl-1-oxo-2,3,4,5,10,11-hexahydro-1H-dibenzo[b,e][1,4]diazepine-7-carboxaldehyde 1024 and morpholine following the procedure reported for the preparation of 10-acetyl-11-(2,4-dichlorophenyl)-3,3-dimethyl-7-(2-morpholin-4-ylethylaminomethyl)-2,3,4,5,10,11-hexahydro-1H-dibenzo[b,e][1,4]diazepin-1-one 1025: m/z=528 (M+H)⁺.

### Example 42

### 10-acetyl-11-(2,4-dichlorophenyl)-3,3-dimethyl-7-(N-methyl-N-propyl-aminomethyl)-2,3,4,5,10,11-hexahydro-1H-dibenzo[b,e][1,4]diazepin-1-one Compound no. 1033

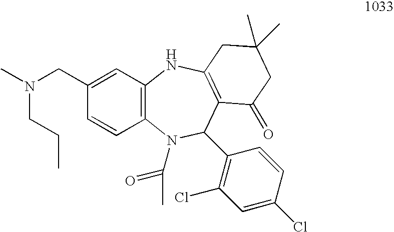

The title compound 1033 was prepared from 10-acetyl-11-(2,4-dichlorophenyl)-3,3-dimethyl-1-oxo-2,3,4,5,10,11-hexahydro-1H-dibenzo[b,e][1,4]diazepine-7-carboxaldehyde 1024 and N-methylpropylamine following the procedure reported for the preparation of 10-acetyl-11-(2,4-dichlorophenyl)-3,3-dimethyl-7-(2-morpholin-4-ylethylaminomethyl)-2,3,4,5,10,11-hexahydro-1H-dibenzo[b,e][1,4]diazepin-1-one 1025: m/z=514 (M+H)⁺.

### Example 43

### 10-acetyl-11-(2,4-dichlorophenyl)-3,3-dimethyl-7-[4-(aminocarbonyl)-piperidin-1-ylmethyl]-2,3,4,5,10,11-hexahydro-1H-dibenzo[b,e][1,4]diazepin-1-one Compound no. 1034.

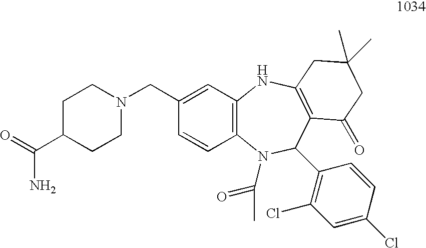

The title compound 1034 was prepared from 10-acetyl-11-(2,4-dichlorophenyl)-3,3-dimethyl-1-oxo-2,3,4,5,10,11-hexahydro-1H-dibenzo[b,e][1,4]diazepine-7-carboxaldehyde 1024 and 4-(aminocarbonyl)piperidine following the procedure reported for the preparation of 10-acetyl-11-(2,4-dichlorophenyl)-3,3-dimethyl-7-(2-morpholin-4-ylethylaminomethyl)-2,3,4,5,10,11-hexahydro-1H-dibenzo[b,e][1,4]-diazepin-1-one 1025: m/z=569 (M+H)⁺.

### Example 44

### 10-acetyl-11-(2,4-dichlorophenyl)-3,3-dimethyl-7-(4-methylpiperazin-1-ylmethyl)-2,3,4,5,10,11-hexahydro-1H-dibenzo[b,e][1,4]diazepin-1-one Compound no. 1035.

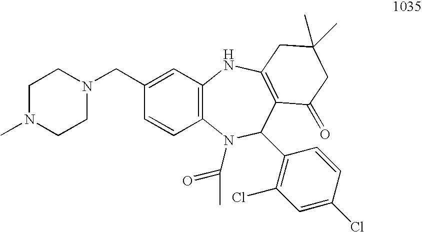

The title compound 1035 was prepared from 10-acetyl-11-(2,4-dichlorophenyl)-3,3-dimethyl-1-oxo-2,3,4,5,10,11-hexahydro-1H-dibenzo[b,e][1,4]diazepine-7-carboxaldehyde 1024 and 4-methylpiperazine following the procedure reported for the preparation of 10-acetyl-11-(2,4-dichlorophenyl)-3,3-dimethyl-7-(2-morpholin-4-ylethylaminomethyl)-2,3,4,5,10,11-hexahydro-1H-dibenzo[b,e][1,4]diazepin-1-one 1025: m/z=541 (M+H)⁺.

### Example 45

### 10-acetyl-11-(2,4-dichlorophenyl)-3,3-dimethyl-7-(piperidin-1-ylmethyl)-2,3,4,5,10,11-hexahydro-1H-dibenzo[b,e][1,4]diazepin-1-one Compound no. 1037

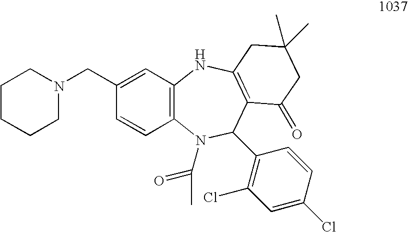

The title compound 1037 was prepared from 10-acetyl-11-(2,4-dichlorophenyl)-3,3-dimethyl-1-oxo-2,3,4,5,10,11-hexahydro-1H-dibenzo[b,e][1,4]diazepine-7-carboxaldehyde 1024 and piperidine following the procedure reported for the preparation of 10-acetyl-11-(2,4-dichlorophenyl)-3,3-dimethyl-7-(2-morpholin-4-ylethylaminomethyl)-2,3,4,5,10,11-hexahydro-1H-dibenzo[b,e][1,4]diazepin-1-one 1025: m/z=526 (M+H)⁺.

### Example 46

### 10-acetyl-11-(2,4-dichlorophenyl)-3,3-dimethyl-7-(pyrrolidin-1-yl-methyl)-2,3,4,5,10,11-hexahydro-1H-dibenzo[b,e][1,4]diazepin-1-one Compound no. 1038

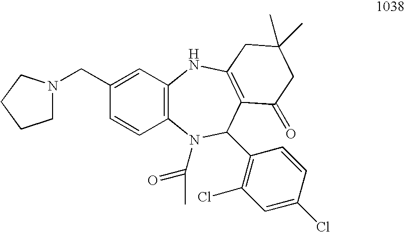

The title compound 1038 was prepared from 10-acetyl-11-(2,4-dichlorophenyl)-3,3-dimethyl-1-oxo-2,3,4,5,10,11-hexahydro-1H-dibenzo[b,e][1,4]diazepine-7-carboxaldehyde 1024 and pyrrolidine following the procedure reported for the preparation of 10-acetyl-11-(2,4-dichlorophenyl)-3,3-dimethyl-7-(2-morpholin-4-ylethylaminomethyl)-2,3,4,5,10,11-hexahydro-1H-dibenzo[b,e][1,4]diazepin-1-one 1025: m/z=512 (M+H)⁺.

### Example 47

### 10-acetyl-11-(2,4-dichlorophenyl)-3,3-dimethyl-7-[2-(piperidin-1-yl)-ethoxymethyl]-2,3,4,5,10,11-hexahydro-1H-dibenzo[b,e][1,4]diazepin-1-one Compound no. 1039.

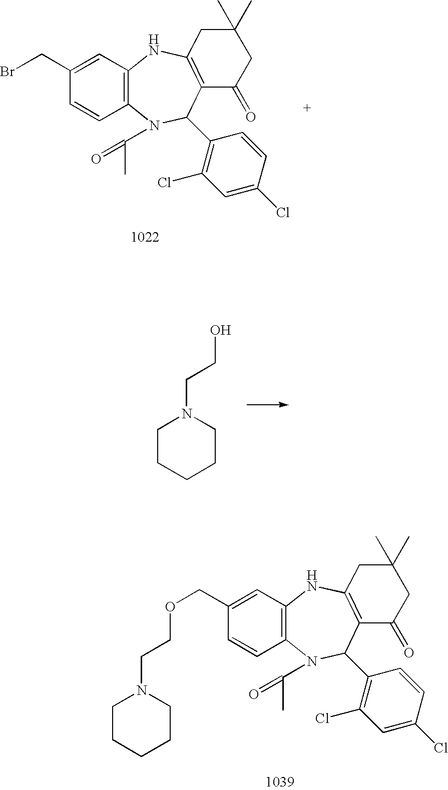

Sodium hydride (17 mg, 60% in mineral oil, 0.42 mmol) was added at 0° C. under argon to a solution of N-piperidineethanol (53 μL, 0.4 mmol) in dry DMF (4 mL). The resulting solution was added at 0° C. under argon to a solution of 10-acetyl-7-bromo-methyl-11-(2,4-dichlorophenyl)-3,3-dimethyl-2,3,4,5,10,11-hexahydro-1H-dibenzo-[b,e][1,4]diazepin-1-one 1022 (200 mg, 0.38 mmol) in dry DMF (2 mL). After 2 h, the reaction mixture was diluted with ice-cold water (70 mL). The pH of the resulting solution was adjusted to 7 with 2N aqueous NaOH. Then, the reaction mixture was successively extracted with AcOEt (3 times), THF (3 times). The combined organic extracts were washed with brine, dried (Na₂SO₄) and evaporated. The residue was triturated in toluene, the evaporated. The residue was triturated in DCM and methanol, filtered and concentrated under vacuum. The residue was purified by column chromatography on alumina (CH₂Cl₂/MeOH, gradient 1:0 to 92:8) to give 85 mg (39%) of the target compound 1039: m/z=570 (M+H)⁺.

### Example 48

### 10-acetyl-11-(2,4-dichlorophenyl)-3,3-dimethyl-7-[3-(N,N-dimethyl-amino)propoxymethyl]-2,3,45,10,11-hexahydro-1H-dibenzo[b,e][1,4]diazepin-1-one Compound no. 1040.

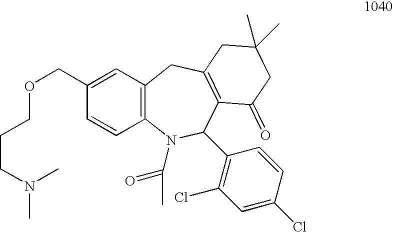

The title compound 1040 was prepared from 10-acetyl-7-bromomethyl-11-(2,4-dichlorophenyl)-3,3-dimethyl-2,3,4,5,10,11-hexahydro-1H-dibenzo[b,e][1,4]diazepin-1-one 1022 and 3-(N,N-dimethylamino)propanol following the procedure reported for the preparation of 10-acetyl-11-(2,4-dichlorophenyl)-3,3-dimethyl-7-[2-(piperidin-1-yl)ethoxymethyl]-2,3,4,5,10,11-hexahydro-1H-dibenzo[b,e][1,4]diazepin-1-one 1039: m/z=544 (M+H)⁺.

### Example 49

### 10-acetyl-11-[4-(phenalaminocarbonyl)phenyl]-33-dimethyl-2,3,4,5,10,11-hexahydro-1H-dibenzo[b,e][1,4]diazepin-1-one Compound no. 1041.

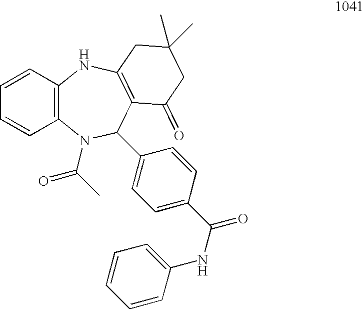

The title compound 1041 was prepared from 4-(phenylaminocarbonyl)benzaldehyde following the procedure reported for the synthesis of 10-acetyl-11-(2,4-dichloro-phenyl)-2,3,4,5,10,11-hexahydro-3,3-dimethyl-1H-dibenzo[b,e][1,4]diazepin-1-one (compound no. 38): m/z=480 (M+H)⁺.

### Example 50

### 10-acetyl-11-[4-(N-acetyl-N-phenalaminosulfonyl)phenyl]-3,3-dimethyl-2,3,4,5,10,11-hexahydro-1H-dibenzo[b,e][1,4]diazepin-1-one Compound no. 1042.

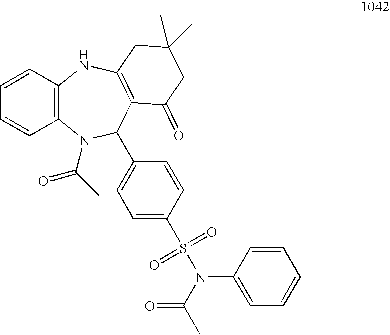

The title compound 1042 was prepared from 4-(N-phenylaminosulfonyl)benzaldehyde following the procedure reported for the synthesis of 10-acetyl-11-(2,4-dichloro-phenyl)-2,3,4,5,10,11-hexahydro-3,3-dimethyl-1H-dibenzo[b,e][1,4]diazepin-1-one (compound no. 38): m/z=558 (M+H)⁺.

### Example 51

### 10-acetyl-11-[4-(N-phenylaminosulfonyl)phenyl]-3,3-dimethyl-2,3,4,5,10,11-hexahydro-1H-dibenzo[b,e][1,4]diazepin-1-one Compound no. 1043

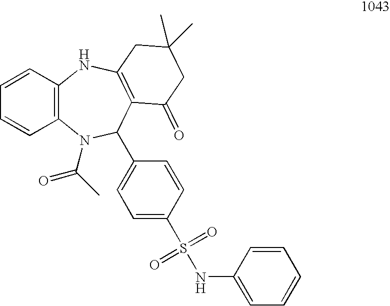

A solution of lithium hydroxide hydrate (11 mg, 0.26 mmol) in water (0.5 mL) was added at 0° C. to a stirred solution of 10-acetyl-11-[4-(N-acetyl-N-phenylamino-sulfonyl)phenyl]-3,3-dimethyl-2,3,4,5,10,11-hexahydro-1H-dibenzo[b,e][1,4]diazepin-1-one 1042 (133 mg, 0.26 mmol). After 12 h at room temperature, the reaction mixture was diluted with a saturated solution of ammonium chloride, extracted twice with AcOEt, washed with brine, dried (Na₂SO₄) and evaporated to give 100 mg of the title product: m/z=516 (M+H)⁺.

### Example 52

### 10-acetyl-11-(4-nitrophenyl)-3,3-dimethyl-2,3,4,5,10,11-hexahydro-1H-dibenzo[b,e][1,4]diazepin-1-one Compound no. 1044

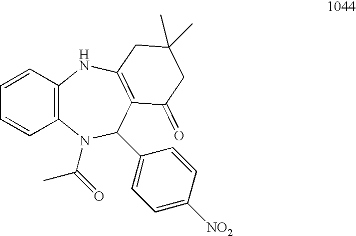

The title compound 1044 was prepared from intermediate 5-2 and 4-nitrobenzaldehyde following the procedure described for 10-acetyl-11-(2,4-dichlorophenyl)-2,3,4,5,10,11-hexahydro-3,3-dimethyl-1H-dibenzo[b,e][1,4]diazepin-1-one (compound no. 38): m/z=406 (M+H)⁺.

### Example 53

### 10-acetyl-11-(4-aminophenyl)-3,3-dimethyl-2,3,4,5,10,11-hexahydro-1H-dibenzo[b,e][1,4]diazepin-1-one Compound no. 1045

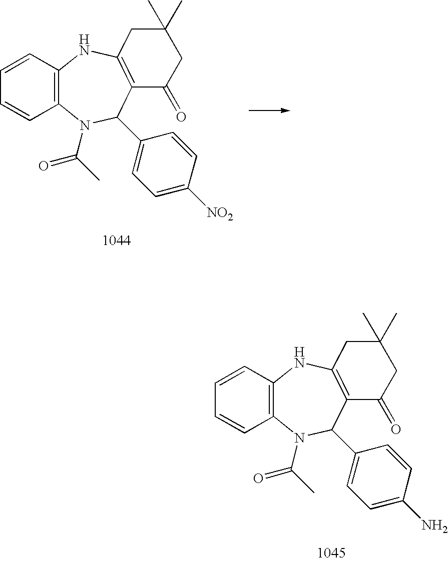

A solution of 10-acetyl-11-(4-nitrophenyl)-3,3-dimethyl-2,3,4,5,10,11-hexahydro-1H-dibenzo[b,e][1,4]diazepin-1-one 1044 (862 mg, 2.13 mmol) in MeOH (3 mL) and THF (3 mL) was added to a suspension of iron (476 mg, 8.52 mmol) and ammonium chloride (460 mg, 8.52 mmol) in water (3 mL). The resulting mixture was heated at 70° C. After 2 h, the reaction mixture was filtered on kieselguhr and extensively washed with AcOEt. The combine organic extracts were washed with brine, the dried (Na₂SO₄) and evaporated to give 272 mg (35%) of the title product 1045: m/z=376 (M+H)⁺.

### Example 54

### 10-acetyl-11-[4-(phenylsulfonylamino)phenyl]-3,3-dimethyl-2,3,4,5,10,11-hexahydro-1H-dibenzo[b,e][1,4]diazepin-1-one Compound no. 1046.

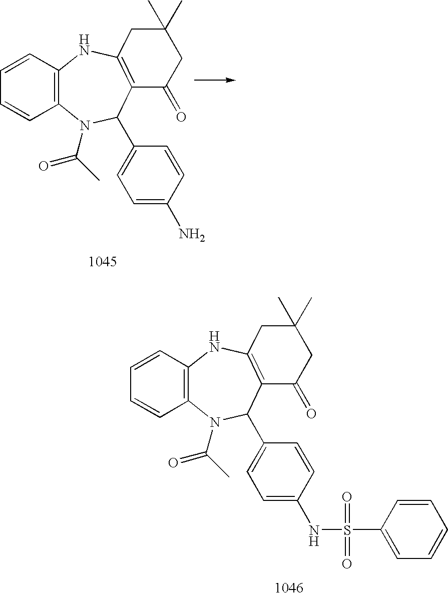

A solution of 10-acetyl-11-(4-aminophenyl)-3,3-dimethyl-2,3,4,5,10,11-hexahydro-1H-dibenzo[b,e][1,4]diazepin-1-one 1045 (127 mg, 0.34 mmol), benzensulfonyl chloride (45.5 μL, 0.36 mmol) in pyridine (2 mL) was stirred at room temperature for 12 h. The reaction mixture was successively added dropwise to 10 mL of water, extracted with AcOEt, washed with brine, dried (Na₂SO₄) and evaporated to afford 78 mg of the title product 1046: m/z=516 (M+H)⁺.

### Example 55

### 10-acetyl-11-[4-(phenylcarbonylamino)henyl]-33-dimethyl-2,3,4,5,1011-hexahydro-1H-dibenzo[b,e][1,4]diazenin-1-one Compound no. 1047.

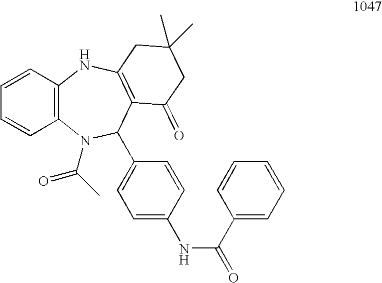

The title compound 1047 was prepared from 10-acetyl-11-(4-aminophenyl)-3,3-dimethyl-2,3,4,5,10,11-hexahydro-1H-dibenzo[b,e][1,4]diazepin-1-one 1045 and benzoyl chloride following the procedure described for 10-acetyl-11-[4-(phenyl-sulfonylamino)phenyl]-3,3-dimethyl-2,3,4,5,10,11-hexahydro-1H-dibenzo[b,e][1,4]-diazepin-1-one 1046: m/z=480 (M+H)⁺.

### Example 56

### 11-[4-(phenylcarbonyl)phenyl]-3,3-dimethyl-2,3,4,5,10,11-hexahydro-1H-dibenzo[b,e][1,4]diazepin-1-one Compound no. 1048.

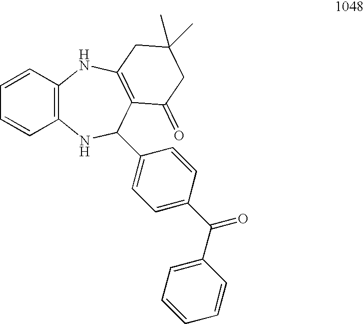

The title compound was prepared from intermediate 5-2 and 4-(phenylcarbonyl)-benzaldehyde following the procedure described for 11-(2,4-dichlorophenyl)-2,3,4,5,10,11-hexahydro-3,3-dimethyl-1H-dibenzo[b,e][1,4]diazepin-1-one 5-3: m/z=423 (M+H)⁺.

### Example 57

### 10-acetyl-11-[4-(phenylcarbonyl)phenyl]-3,3-dimethyl-2,3,4,5,10,11-hexahydro-1H-dibenzo[b,e][1,4]diazepin-1-one Compound no. 1049.

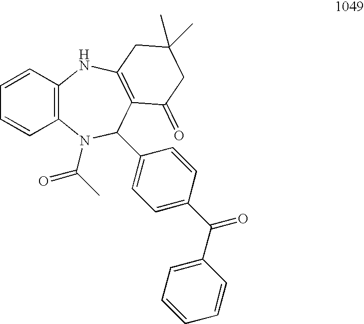

The title compound 1049 was prepared from 11-[4-(phenylcarbonyl)phenyl]-3,3-dimethyl-2,3,4,5,10,11-hexahydro-1H-dibenzo[b,e][1,4]diazepin-1-one 1048 following the procedure described for 10-acetyl-11-(2,4-dichlorophenyl)-2,3,4,5,10,11-hexa-hydro-3,3-dimethyl-1H-dibenzo[b,e][1,4]diazepin-1-one (compound no. 38): m/z=465 (M+H)⁺.

### Example 58

### 11-[4-benzyloxycarbonyl-2-chlorophenyl]-3,3-dimethyl-2,3,4,5,10,11-hexahydro-1H-dibenzo[b,e][1,4]diazepin-1-one Compound no. 443.

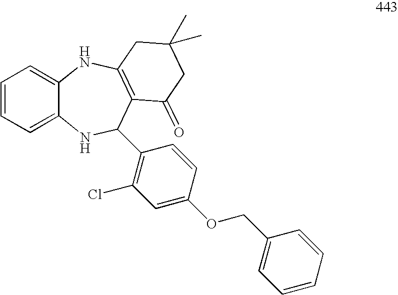

The title compound 443 was prepared from intermediate 5-2 and 4-benzyloxy-2-chlorobenzaldehyde following the procedure described for 11-(2,4-dichlorophenyl)-2,3,4,5,10,11-hexahydro-3,3-dimethyl-1H-dibenzo[b,e][1,4]diazepin-1-one 5-3: m/z=459 (M+H)⁺.

### Example 59

### 10-acetyl-11-[4-benzyloxycarbonyl-2-chlorophenyl]-3,3-dimethyl-2,3,4,5,10,11-hexahydro-1H-dibenzo[b,e][1,4]diazepin-1-one Compound no. 142.

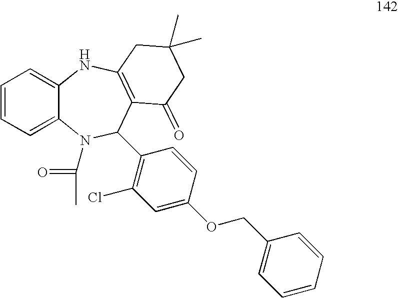

The title compound 142 was prepared from 11-[4-benzyloxycarbonyl-2-chlorophenyl]-3,3-dimethyl-2,3,4,5,10,11-hexahydro-1H-dibenzo[b,e][1,4]diazepin-1-one 443 following the procedure described for 10-acetyl-11-(2,4-dichlorophenyl)-2,3,4,5,10,11-hexahydro-3,3-dimethyl-1H-dibenzo[b,e][1,4]diazepin-1-one (compound no. 38): m/z=501 (M+H)⁺.

### Example 60

### 11-[3,5-dichlorophenyl]-3,3-dimethyl-2,3,4,5,10,11-hexahydro-1H-dibenzo[b,e][1,4]diazepin-1-one Compound no. 436.

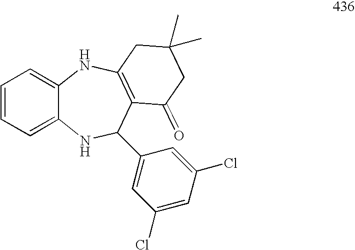

The title compound 436 was prepared from intermediate 5-2 and 2,5-dichloro-benzaldehyde following the procedure described for 11-(2,4-dichlorophenyl)-2,3,4,5,10,11-hexahydro-3,3-dimethyl-1H-dibenzo[b,e][1,4]diazepin-1-one 5-3: m/z=387 (M+H)⁺.

### Example 61

### 10-acetyl-11-[3,5-dichlorophenyl]-3,3-dimethyl-2,3,4,5,10,11-hexahydro-1H-dibenzo[b,e][1,4]diazepin-1-one Compound no. 133.

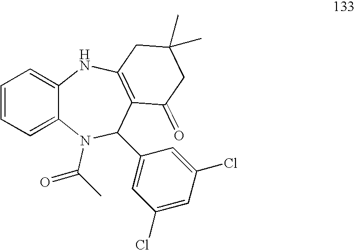

The title compound 133 was prepared from 11-[3,5-dichlorophenyl]-3,3-dimethyl-2,3,4,5,10,11-hexahydro-1H-dibenzo[b,e][1,4]diazepin-1-one 436 following the procedure described for 10-acetyl-11-(2,4-dichlorophenyl)-2,3,4,5,10,11-hexahydro-3,3-dimethyl-1H-dibenzo[b,e][1,4]diazepin-1-one (compound no. 38): m/z=429 (M+H)⁺.

### Example 62

### 11-[4-benzyloxy-3-chlorophenyl]-3,3-dimethyl-2,3,4,5,10,11-hexahydro-1H-dibenzo[b,e][1,4]diazepin-1-one Compound no. 439.

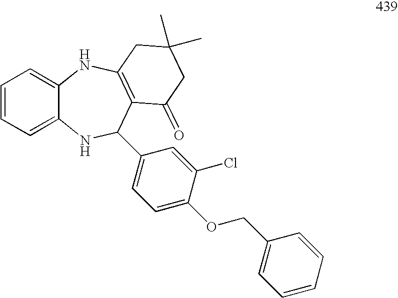

The title compound 439 was prepared from intermediate 5-2 and 3-chloro-4-benzyloxy-benzaldehyde following the procedure described for 11-(2,4-dichlorophenyl)-2,3,4,5,10,11-hexahydro-3,3-dimethyl-1H-dibenzo[b,e][1,4]diazepin-1-one 5-3: m/z=459 (M+H)⁺.

### Example 63

### 10-acetyl-11-[4-benzyloxy-3-chlorophenyl]-3,3-dimethyl-2,3,4,5,10,11-hexahydro-1H-dibenzo[b,e][1,4]diazepin-1-one Compound no. 139

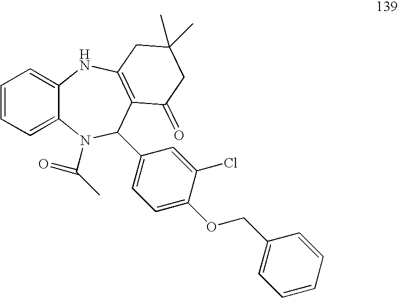

The title compound 139 was prepared from 11-[4-benzyloxy-3-chlorophenyl]-3,3-dimethyl-2,3,4,5,10,11-hexahydro-1H-dibenzo[b,e][1,4]diazepin-1-one 439 following the procedure described for 10-acetyl-11-(2,4-dichlorophenyl)-2,3,4,5,10,11-hexahydro-3,3-dimethyl-1H-dibenzo[b,e][1,4]diazepin-1-one (compound no. 38): m/z=501 (M+H)⁺.

### Example 64

### 11-[4-benzyloxy-3,5-dichlorophenyl]-3,3-dimethyl-2,3,4,5,10,11-hexahydro-1H-dibenzo[b,e][1,4]diazepin-1-one Compound no. 444.

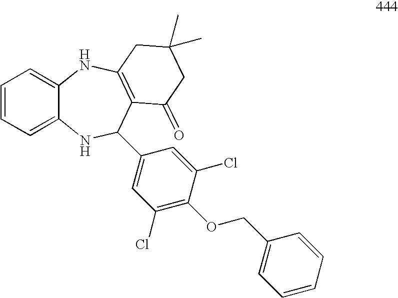

The title compound 444 was prepared from intermediate 5-2 and 3,5-dichloro-4-benzyloxybenzaldehyde following the procedure described for 11-(2,4-dichloro-phenyl)-2,3,4,5,10,11-hexahydro-3,3-dimethyl-1H-dibenzo[b,e][1,4]diazepin-1-one 5-3: m/z=493 (M+H)⁺.

### Example 65

### 10-acetyl-11-[4-benzyloxy-3,5-dichlorophenyl]-3,3-dimethyl-2,3,4,5,10,11-hexahydro-1H-dibenzo[b,e][1,4]diazepin-1-one Compound no. 143

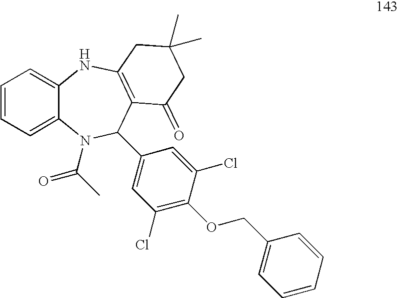

The title compound 143 was prepared from 11-[4-benzyloxy-3,5-dichlorophenyl]-3,3-dimethyl-2,3,4,5,10,11-hexahydro-1H-dibenzo[b,e][1,4]diazepin-1-one 444 following the procedure described for 10-acetyl-11-(2,4-dichlorophenyl)-2,3,4,5,10,11-hexahydro-3,3-dimethyl-1H-dibenzo[b,e][1,4]diazepin-1-one (compound no. 38): m/z=535 (M+H)⁺.

### Example 66

### 11-[2,5-dichlorophenyl]-3,3-dimethyl-2,3,4,5,10,11-hexahydro-1H-dibenzo[b,e][1,4]diazepin-1-one Compound no. 438

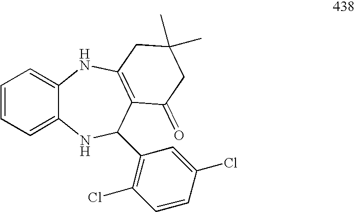

The title compound 438 was prepared from intermediate 5-2 and 2,5-dichloro-benzaldehyde following the procedure described for 11-(2,4-dichlorophenyl)-2,3,4,5,10,11-hexahydro-3,3-dimethyl-1H-dibenzo[b,e][1,4]diazepin-1-one 5-3: m/z=387 (M+H)⁺.

### Example 67

### 10-acetyl-11-[2,5-dichlorophenyl]-3,3-dimethyl-2,3,4,5,10,11-hexahydro-1H-dibenzo[b,e][1,4]diazepin-1-one Compound no. 138

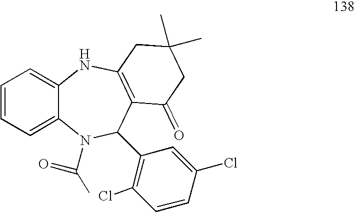

The title compound 138 was prepared from 11-[2,5-dichlorophenyl]-3,3-dimethyl-2,3,4,5,10,11-hexahydro-1H-dibenzo[b,e][1,4]diazepin-1-one 438 following the procedure described for 10-acetyl-11-(2,4-dichlorophenyl)-2,3,4,5,10,11-hexahydro-3,3-dimethyl-1H-dibenzo[b,e][1,4]diazepin-1-one (compound no. 38): m/z=429 (M+H)⁺.

### Example 68

### 11-[2,4-dibenzyloxyphenyl]-3,3-dimethyl-2,3,4,5,10,11-hexahydro-1H-dibenzo[b,e][1,4]diazepin-1-one Compound no. 445.

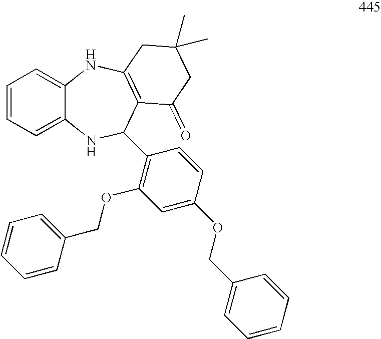

The title compound 445 was prepared from intermediate 5-2 and 2,4-dibenzyloxy-benzaldehyde following the procedure described for 11-(2,4-dichlorophenyl)-2,3,4,5,10,11-hexahydro-3,3-dimethyl-1H-dibenzo[b,e][1,4]diazepin-1-one 5-3: m/z=531 (M+H)⁺.

### Example 69

### 10-acetyl-11-[2,4-dibenzyloxyphenyl]-3,3-dimethyl-2,3,4,5,10,11-hexahydro-1H-dibenzo[b,e][1,4]diazepin-1-one Compound no. 144.

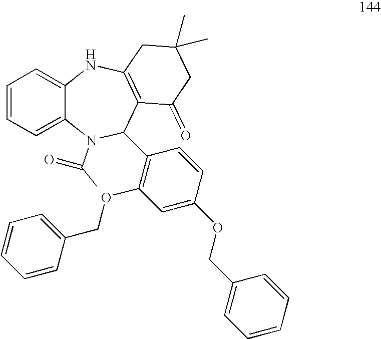

The title compound 144 was prepared from 11-[2,4-dibenzyloxyphenyl]-3,3-dimethyl-2,3,4,5,10,11-hexahydro-1H-dibenzo[b,e][1,4]diazepin-1-one 445 following the procedure described for 10-acetyl-11-(2,4-dichlorophenyl)-2,3,4,5,10,11-hexahydro-3,3-dimethyl-1H-dibenzo[b,e][1,4]diazepin-1-one (compound no. 38): m/z=573 (M+H)⁺.

### Example 70

### 11-(2,4-difluorophenyl)-3,3-dimethyl-2,3,4,5,10,11-hexahydro-1H-dibenzo[b,e][1,4]diazepin-1-one Compound no. 441

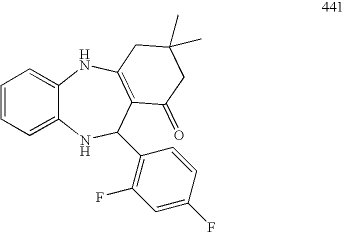

The title compound 441 was prepared from intermediate 5-2 and 2,4-difluoro-benzaldehyde following the procedure described for 11-(2,4-dichlorophenyl)-2,3,4,5,10,11-hexahydro-3,3-dimethyl-1H-dibenzo[b,e][1,4]diazepin-1-one 5-3: m/z=355 (M+H)⁺.

### Example 71

### 10-acetyl-11-(2,4-difluorophenyl)-3,3-dimethyl-2,3,4,5,10,11-hexahydro-1H-dibenzo[b,e][1,4]diazepin-1-one Compound no. 146

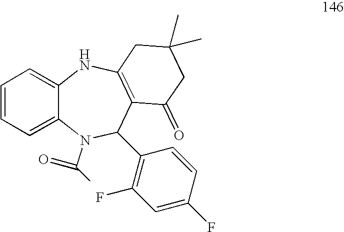

The title compound 146 was prepared from 11-(2,4-difluorophenyl)-3,3-dimethyl-2,3,4,5,10,11-hexahydro-1H-dibenzo[b,e][1,4]diazepin-1-one 441 following the procedure described for 10-acetyl-11-(2,4-dichlorophenyl)-2,3,4,5,10,11-hexahydro-3,3-dimethyl-1H-dibenzo[b,e][1,4]diazepin-1-one (compound no. 38): m/z=397 (M+H)⁺.

### Example 72

### 11-(4-trifluoromethyloxyphenyl)-3,3-dimethyl-2,3,4,5,10,11-hexahydro-1H-dibenzo[b,e][1,4]diazepin-1-one Compound no. 434

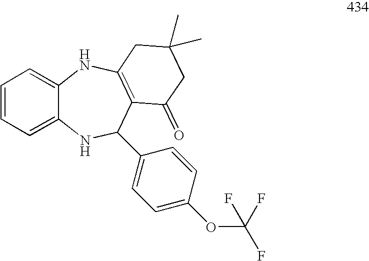

The title compound 434 was prepared from intermediate 5-2 and 4-trifluoromethyloxy-benzaldehyde following the procedure described for 11-(2,4-dichlorophenyl)-2,3,4,5,10,11-hexahydro-3,3-dimethyl-1H-dibenzo[b,e][1,4]diazepin-1-one 5-3: m/z=403 (M+H)⁺.

### Example 73

### 10-acetyl-11-(4-trifluoromethyloxyphenyl)-3,3-dimethyl-2,3,4,5,10,11-hexahydro-1H-dibenzo[b,e][1,4]diazepin-1-one Compound no. 1064.

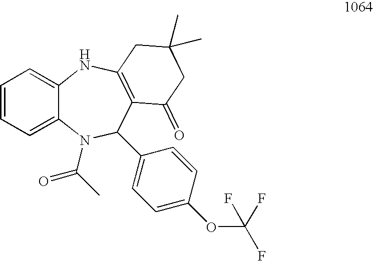

The title compound 1064 was prepared from 11-(4-trifluoromethyloxyphenyl)-3,3-dimethyl-2,3,4,5,10,11-hexahydro-1H-dibenzo[b,e][1,4]diazepin-1-one 434 following the procedure described for 10-acetyl-11-(2,4-dichlorophenyl)-2,3,4,5,10,11-hexahydro-3,3-dimethyl-1H-dibenzo[b,e][1,4]diazepin-1-one (compound no. 38): m/z=445 (M+H)⁺.

### Example 74

### 11-(4-benzyloxy-2,6-dichlorophenyl)-3,3-dimethyl-2,3,4,5,10,11-hexahydro-1H-dibenzo[b,e][1,4]diazepin-1-one Compound no. 447.

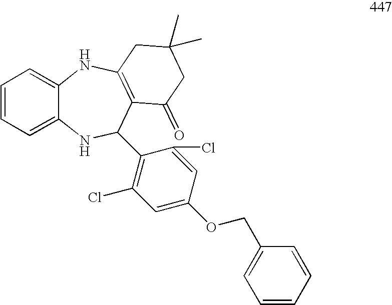

The title compound 447 was prepared from intermediate 5-2 and 4-benzyloxy-2,6-dichlorobenzaldehyde following the procedure described for 11-(2,4-dichlorophenyl)-2,3,4,5,10,11-hexahydro-3,3-dimethyl-1H-dibenzo[b,e][1,4]diazepin-1-one 5-3: m/z=493 (M+H)⁺.

### Example 75

### 10-acetyl-11-(4-benzyloxy-2,6-dichlorophenyl)-3,3-dimethyl-2,3,4,5,10,11-hexahydro-1H-dibenzo[b,e][1,4]diazepin-1-one Compound no. 150.

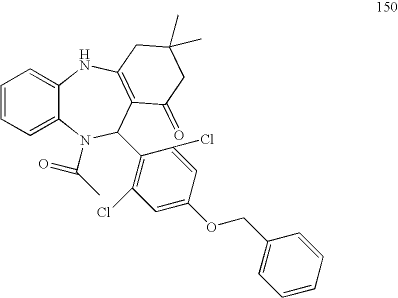

The title compound 150 was prepared from 11-(4-benzyloxy-2,6-dichlorophenyl)-3,3-dimethyl-2,3,4,5,10,11-hexahydro-1H-dibenzo[b,e][1,4]diazepin-1-one 447 following the procedure described for 10-acetyl-11-(2,4-dichlorophenyl)-2,3,4,5,10,11-hexahydro-3,3-dimethyl-1H-dibenzo[b,e][1,4]diazepin-1-one (compound no. 38): m/z=535 (M+H)⁺.

### Example 76

### 11-(3-benzyloxyphenyl)-3,3-dimethyl-2,3,4,5,10,11-hexahydro-1H-dibenzo[b,e][1,4]diazepin-1-one Compound no. 437

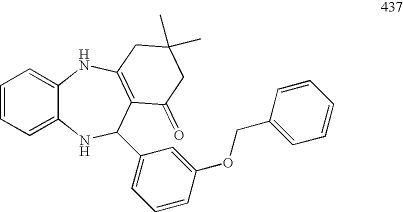

The title compound 437 was prepared from intermediate 5-2 and 3-benzyloxy-benzaldehyde following the procedure described for 11-(2,4-dichlorophenyl)-2,3,4,5,10,11-hexahydro-3,3-dimethyl-1H-dibenzo[b,e][1,4]diazepin-1-one 5-3: m/z=425 (M+H)⁺.

### Example 77

### 10-acetyl-11-(3-benzyloxyphenyl)-3,3-dimethyl-2,3,4,5,10,11-hexahydro-1H-dibenzo[b,e][1,4]diazepin-1-one Compound no. 136.

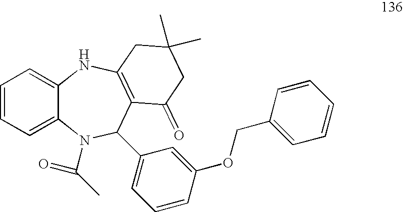

The title compound 136 was prepared from 11-(3-benzyloxyphenyl)-3,3-dimethyl-2,3,4,5,10,11-hexahydro-1H-dibenzo[b,e][1,4]diazepin-1-one 437 following the procedure described for 10-acetyl-11-(2,4-dichlorophenyl)-2,3,4,5,10,11-hexahydro-3,3-dimethyl-1H-dibenzo[b,e][1,4]diazepin-1-one (compound no. 38): m/z=467 (M+H)⁺.

### Example 78

### 11-(4-phenoxyphenyl)-3,3-dimethyl-2,3,4,5,10,11-hexahydro-1H-dibenzo[b,e][1,4]diazepin-1-one Compound no. 435

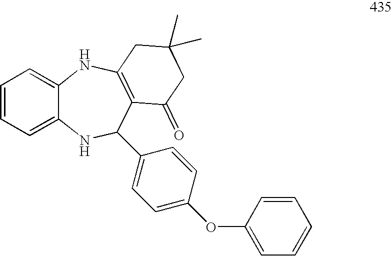

The title compound 435 was prepared from intermediate 5-2 and 4-phenoxy-benzaldehyde following the procedure described for 11-(2,4-dichlorophenyl)-2,3,4,5,10,11-hexahydro-3,3-dimethyl-1H-dibenzo[b,e][1,4]diazepin-1-one 5-3: m/z=411 (M+H)⁺.

### Example 79

### 10-acetyl-11-(4-phenoxyphenyl)-3,3-dimethyl-2,3,4,5,10,11-hexahydro-1H-dibenzo[b,e][1,4]diazepin-1-one Compound no. 134

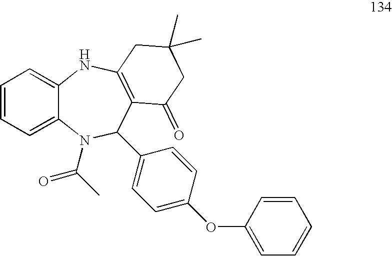

The title compound 134 was prepared from 11-(4-phenoxyphenyl)-3,3-dimethyl-2,3,4,5,10,11-hexahydro-1H-dibenzo[b,e][1,4]diazepin-1-one 435 following the procedure described for 10-acetyl-1-(2,4-dichlorophenyl)-2,3,4,5,10,11-hexahydro-3,3-dimethyl-1H-dibenzo[b,e][1,4]diazepin-1-one (compound no. 38): m/z=453 (M+H)⁺.

### Example 80

### 11-[4-(2-bromophenoxy)phenyl]-3,3-dimethyl-2,3,4,5,10,11-hexahydro-1H-dibenzo[b,e][1,4]diazepin-1-one Compound no. 442

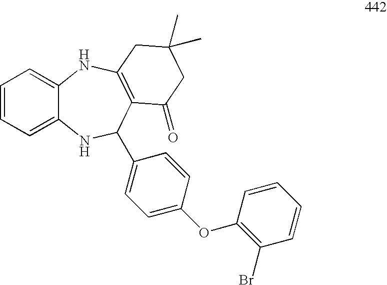

The title compound 442 was prepared from intermediate 5-2 and 4-(2-bromophenoxy)-benzaldehyde following the procedure described for 11-(2,4-dichlorophenyl)-2,3,4,5,10,11-hexahydro-3,3-dimethyl-1H-dibenzo[b,e][1,4]diazepin-1-one 5-3: m/z=490 (M+H)⁺.

### Example 81

### 10-acetyl-11-[4-(2-bromophenoxy)phenyl]-3,3-dimethyl-2,3,4,5,10,11-hexahydro-1H-dibenzo[b,e][1,4]diazepin-1-one Compound no. 147

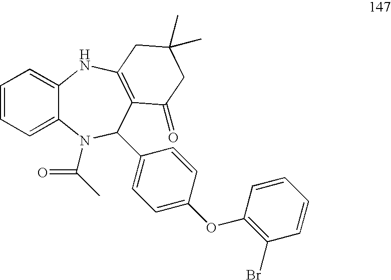

The title compound 147 was prepared from 11-[4-(2-bromophenoxy)phenyl]-3,3-dimethyl-2,3,4,5,10,11-hexahydro-1H-dibenzo[b,e][1,4]diazepin-1-one 442 following the procedure described for 10-acetyl-1-(2,4-dichlorophenyl)-2,3,4,5,10,11-hexahydro-3,3-dimethyl-1H-dibenzo[b,e][1,4]diazepin-1-one (compound no. 38): m/z=532 (M+H)⁺.

### Example 82

### 11-(3-phenoxyphenyl)-3,3-dimethyl-2,3,4,5,10,11-hexahydro-1H-dibenzo[b,e][1,4]diazepin-1-one Compound no. 433

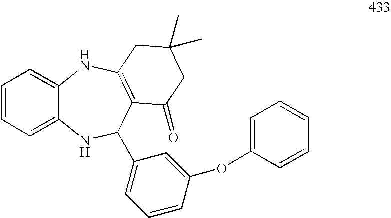

The title compound 433 was prepared from intermediate 5-2 and 3-phenoxy-benzaldehyde following the procedure described for 11-(2,4-dichlorophenyl)-2,3,4,5,10,11-hexahydro-3,3-dimethyl-1H-dibenzo[b,e][1,4]diazepin-1-one 5-3: m/z=411 (M+H)⁺.

### Example 83

### 11-(3-phenoxyphenyl)-3,3-dimethyl-2,3,4,5,10,11-hexahydro-1H-dibenzo[b,e][1,4]diazepin-1-one Compound no. 135

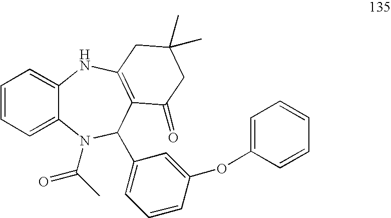

The title compound 135 was prepared from 11-(3-phenoxyphenyl)-3,3-dimethyl-2,3,4,5,10,11-hexahydro-1H-dibenzo[b,e][1,4]diazepin-1-one 433 following the procedure described for 10-acetyl-1-(2,4-dichlorophenyl)-2,3,4,5,10,11-hexahydro-3,3-dimethyl-1H-dibenzo[b,e][1,4]diazepin-1-one (compound no. 38): m/z=453 (M+H)⁺.

### Example 84

### 11-[3-(2-bromophenoxy)phenyl]-3,3-dimethyl-2,3,4,5,10,11-hexahydro-1H-dibenzo[b,e][1,4]diazepin-1-one Compound no. 446

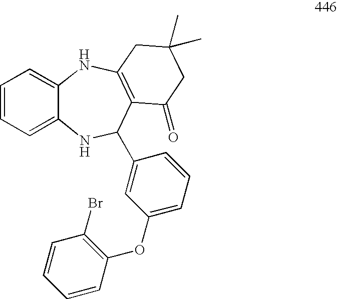

The title compound 446 was prepared from intermediate 5-2 and 3-(2-bromophenoxy)-benzaldehyde following the procedure described for 11-(2,4-dichlorophenyl)-2,3,4,5,10,11-hexahydro-3,3-dimethyl-1H-dibenzo[b,e][1,4]diazepin-1-one 5-3: m/z=490 (M+H)⁺.

### Example 85

### 10-acetyl-11-[3-(2-bromophenoxy)phenyl]-3,3-dimethyl-2,3,4,5,10,11-hexahydro-1H-dibenzo[b,e][1,4]diazepin-1-one Compound no. 149

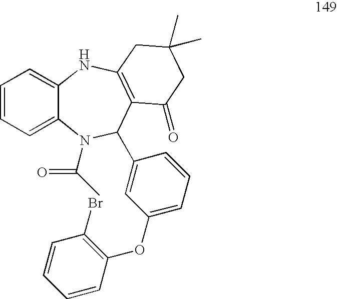

The title compound 149 was prepared from 11-[3-(2-bromophenoxy)phenyl]-3,3-dimethyl-2,3,4,5,10,11-hexahydro-1H-dibenzo[b,e][1,4]diazepin-1-one 446 following the procedure described for 10-acetyl-11-(2,4-dichlorophenyl)-2,3,4,5,10,11-hexahydro-3,3-dimethyl-1H-dibenzo[b,e][1,4]diazepin-1-one (compound no. 38): m/z=532 (M+H)⁺.

### Example 86

### 7,8-dimethoxy-11-(2,4-dichlorophenyl)-3,3-dimethyl-2,3,4,5,10,11-hexahydro-1H-dibenzo[b,e][1,4]diazepin-1-one Compound no. 467

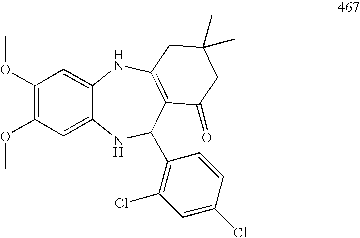

The title compound 467 was prepared from 2,4-dichlorobenzaldehyde following the procedure described for 11-(2,4-dichlorophenyl)-2,3,4,5,10,11-hexahydro-3,3-dimethyl-1H-dibenzo[b,e][1,4]diazepin-1-one 5-3: m/z=447 (M+H)⁺.

### Example 87

### 10-acetyl-7,8-dimethoxy-11-(2,4-dichlorophenyl)-3,3-dimethyl-2,3,4,5,10,11-hexahydro-1H-dibenzo[b,e][1,4]diazepin-1-one Compound no. 309

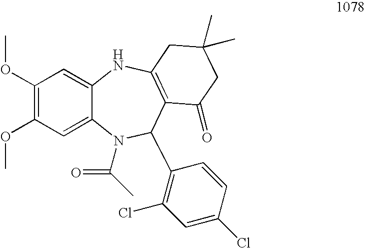

The title compound 309 was prepared from 7,8-dimethoxy-11-(2,4-dichlorophenyl)-3,3-dimethyl-2,3,4,5,10,11-hexahydro-1H-dibenzo[b,e][1,4]diazepin-1-one 467 following the procedure described for 10-acetyl-11-(2,4-dichlorophenyl)-2,3,4,5,10,11-hexahydro-3,3-dimethyl-1H-dibenzo[b,e][1,4]diazepin-1-one (compound no. 38): m/z=489 (M+H)⁺.

### Example 88

### 7,8-difluoro-11-(2,4-dichlorophenyl)-3,3-dimethyl-2,3,4,5,10,11-hexahydro-1H-dibenzo[b,e][1,4]diazepin-1-one Compound no. 466

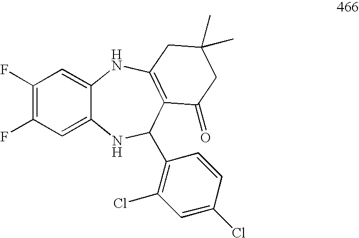

The title compound 466 was prepared from 2,4-dichlorobenzaldehyde following the procedure described for 11-(2,4-dichlorophenyl)-2,3,4,5,10,11-hexahydro-3,3-dimethyl-1H-dibenzo[b,e][1,4]diazepin-1-one 5-3: m/z=423 (M+H)⁺.

### Example 89

### 10-acetyl-7,8-difluoro-11-(2,4-dichlorophenyl)-3,3-dimethyl-2,3,4,5,10,11-hexahydro-1H-dibenzo[b,e][1,4]diazepin-1-one Compound no. 310

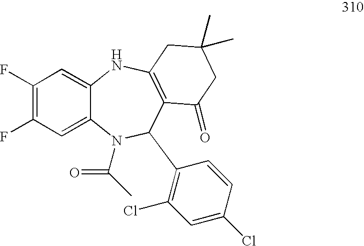

The title compound 310 was prepared from 7,8-difluoro-11-(2,4-dichlorophenyl)-3,3-dimethyl-2,3,4,5,10,11-hexahydro-1H-dibenzo[b,e][1,4]diazepin-1-one 466 following the procedure described for 10-acetyl-11-(2,4-dichlorophenyl)-2,3,4,5,10,11-hexahydro-3,3-dimethyl-1H-dibenzo[b,e][1,4]diazepin-1-one (compound no. 38): m/z=465 (M+H)⁺.

### Example 90

### 11-(2,4-dichlorophenyl)-3-methyl-2,3,4,5,10,11-hexahydro-1H-dibenzo[b,e][1,4]diazepin-1-one Compound no. 422

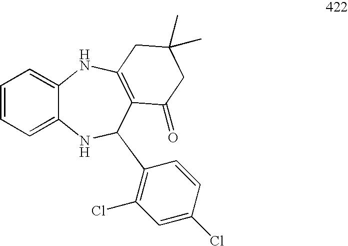

The title compound 422 was prepared from 2,4-dichlorobenzaldehyde following the procedure described for 11-(2,4-dichlorophenyl)-2,3,4,5,10,11-hexahydro-3,3-dimethyl-1H-dibenzo[b,e][1,4]diazepin-1-one 5-3: m/z=373 (M+H)⁺.

### Example 91

### 10-acetyl-11-(2,4-dichlorophenyl)-3-methyl-2,3,4,5,10,11-hexahydro-1H-dibenzo[b,e][1,4]diazepin-1-one Compound no. 294

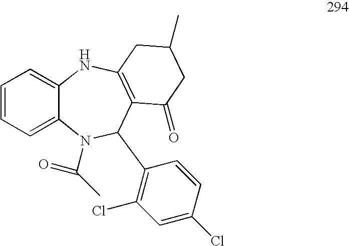

The title compound 294 was prepared from 11-(2,4-dichlorophenyl)-3-methyl-2,3,4,5,10,11-hexahydro-1H-dibenzo[b,e][1,4]diazepin-1-one 442 following the procedure described for 10-acetyl-11-(2,4-dichlorophenyl)-2,3,4,5,10,11-hexahydro-3,3-dimethyl-1H-dibenzo[b,e][1,4]diazepin-1-one (compound no. 38): m/z=415 (M+H)⁺.

### Example 92

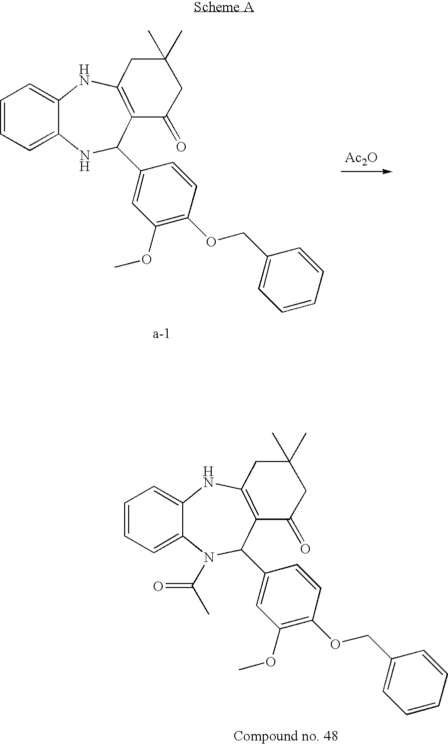

A mixture of a-1 (0.0022 mol) in Ac₂O (10 ml) was stirred and refluxed for 1 hour. A tip spat of DMAP was added. The mixture was stirred for 1 hour. H₂O was added. The mixture was extracted with CH₂Cl₂. The organic layer was separated, dried (over MgSO₄), filtered and the solvent was evaporated until dryness. The residue was purified by column chromatography over silica gel (eluent: CH₂Cl₂/CH₃OH/NH₄OH 99/1/0.05). The pure fractions were collected and the solvent was evaporated. The residue was crystallized from 2-propanone (few)/diethyl ether/EtOH. The precipitate was filtered off and dried, yielding: 0.352 g of Compound no. 48 (melting point: 216° C.).

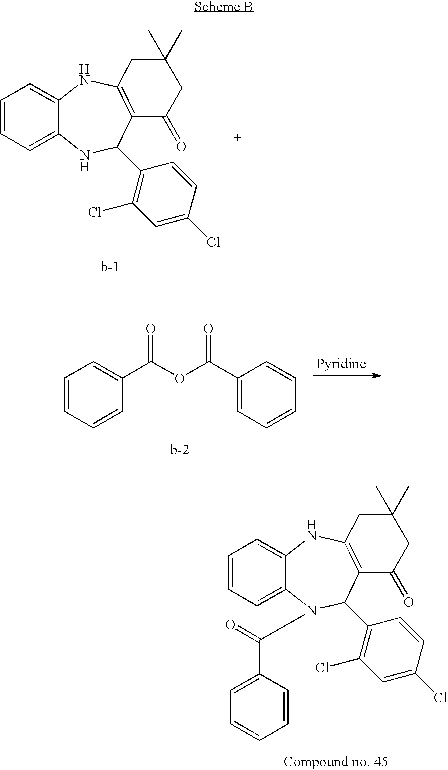

b-2 (0.024 mol, 0.54 g) was added at 0° C. to a solution of b-1 (0.0006 mol) in pyridine (6 ml). The mixture was stirred at room temperature for 12 h. b-2 (0.0024 mol, 0.54 g) was added again at 0° C. The mixture was stirred for 24 h, then evaporated until dryness. The residue was taken up in CH₂Cl₂. The organic layer was washed with H₂O, dried (over MgSO₄), filtered and the solvent was evaporated. The residue was purified by column chromatography over silica gel (eluent: CH₂Cl₂/CH₃OH 98/2). The pure fractions were collected and the solvent was evaporated. The residue was crystallized from 2-propanone/diethyl ether. The precipitate was filtered off and dried, yielding: 0.089 g of compound no. 45 (melting point >250° C.).

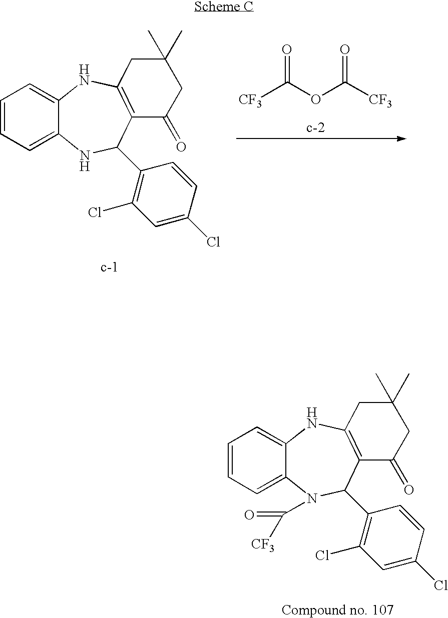

A mixture of c-1 (0.0003 mol) in c-2 (5 ml) was stirred at room temperature for 12 h, then cooled with an ice bath. H₂O was added drop wise. The mixture was taken up in CH₂Cl₂. The organic layer was separated, dried (over MgSO4), filtered and the solvent was evaporated until dryness. The residue (0.165 g) was crystallized from CH₃CN. The precipitate was filtered off and dried, yielding: 0.089 g of Compound no. 107 (74%) (melting point >260° C.).

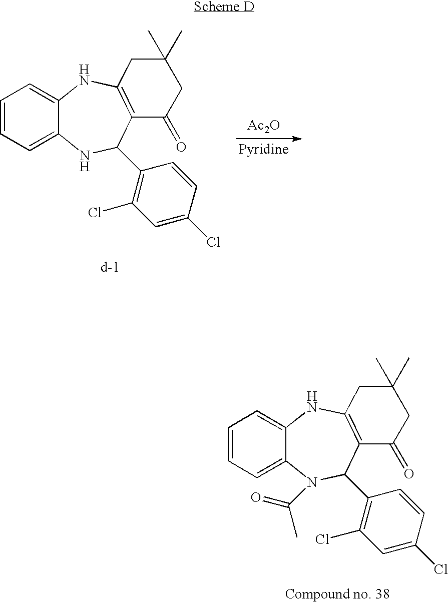

Ac₂O (2 ml) was added drop wise at 0° C. to a solution of d-1 (0.0052 mol) in Pyridine (50 ml). The mixture was stirred at room temperature for 12 h. Ac₂O (2 ml) was added again at 0° C. The mixture was stirred at room temperature for 12 h. The precipitate was filtered, washed with H₂O and dried, yielding: 1.67 g of Compound no. 38 (75%) (melting point >260° C.).

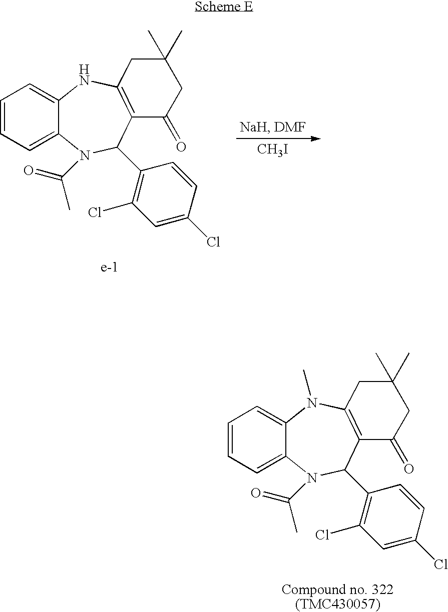

A mixture of e-1 (0.0004 mol) and NaH (0.0004 mol) in DMF (2 ml) was stirred for 10 minutes. CH₃I (0.0004 mol) was then added. The mixture was stirred at room temperature for 12 h and evaporated until dryness. The residue was purified by column chromatography over silica gel (eluent: CH₂Cl₂/CH₃OH 99/1; 10 μm). The pure fractions were collected and the solvent was evaporated. The residue was crystallized from 2-propanone/diethyl ether. The precipitate was filtered off and dried, yielding: 0.037 g of Compound no. 322 (20%) (melting point: 145° C.).

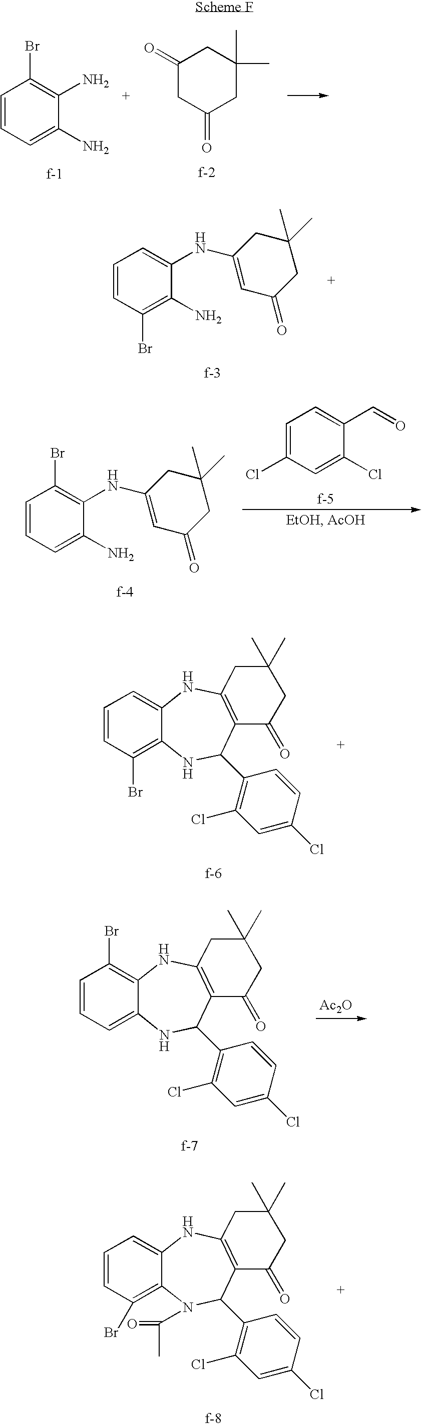

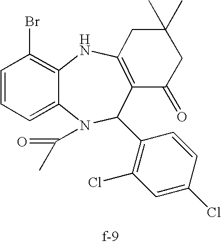

A mixture of f-1 (0.0057 mol) and f-2 (0.0057 mol) in toluene (20 ml) was stirred and refluxed for 12 h, then concentrated under reduced pressure. The residue was purified by column chromatography over silica gel (eluent: CH₂Cl₂/CH₃OH/NH₄OH 96/4/0.2). Two fractions were collected and the solvent was evaporated until dryness, yielding a mixture of f-3 and f-4 (71%).

A mixture of f-3+f-4 (0.004 mol) and f-5 (0.004 mol) in EtOH (10 ml) and AcOH (10 ml) was stirred at 75° C. for 12 h, then evaporated until dryness. The residue was taken up in EtOAc. Saturated NaHCO₃ was added. The mixture was stirred for 1 hour and 30 minutes, then filtered and extracted with EtOAc. The organic layer was separated, dried (over MgSO₄), filtered and the solvent was evaporated until dryness. The residue was purified by column chromatography over silica gel (eluent: CH₂Cl₂/CH₃OH 99.5/0.5). Three fractions were collected and the solvent was evaporated, yielding: 0.2 g of f-7, 1 g of a mixture f-6+f-7 and 0.1 g of f-6 (melting point: 170° C.).

A mixture of f-6+f-7 (0.0004 mol) in Ac₂O (5 ml) was stirred and refluxed for 4 hours, then concentrated under reduced pressure. The residue was purified by column chromatography over silica gel (eluent: CH₂Cl₂/CH₃OH/NH₄OH 97/3/0.1). Two fractions were collected and the solvent was evaporated, yielding: 0.065 g of f-9 and 0.09 g of f-8. A part of f-8 was crystallized from diethyl ether/2-propanone. The precipitate was filtered off and dried, yielding: 0.03 g (melting point >260° C.).

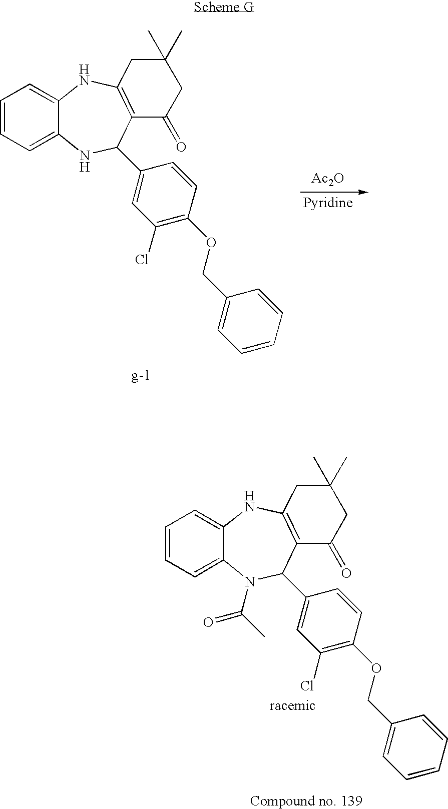

The title compound no. 139 was prepared from Intermediate (5-2) and 4-benzyloxy-3-chlorobenzaldehyde following the procedure described in Example 5: m/z=501 (M+H)⁺.

Separation of the (R)- and (S)-enantiomers of compound no. 139.

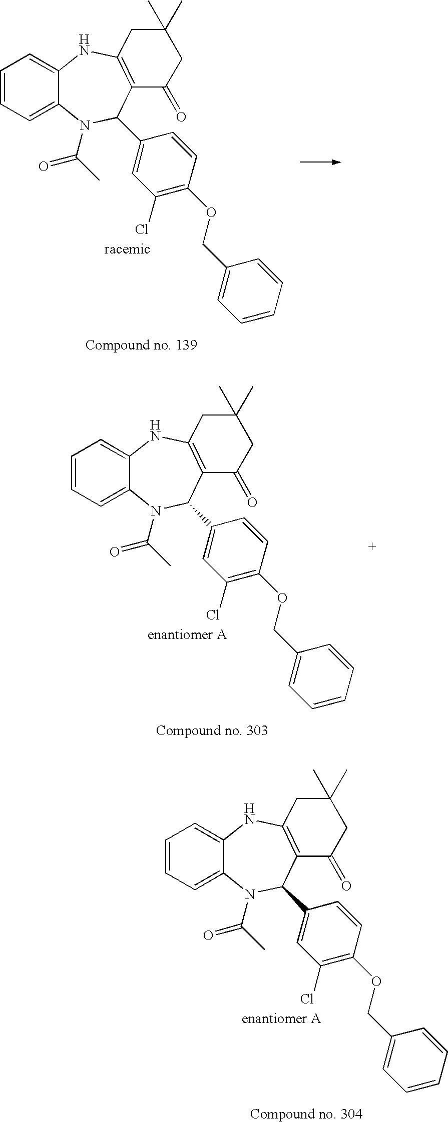

The two enantiomers were separated by SFC with a chiral column (eluent: CO₂/CH₃OH 40/60). Two fractions were collected and the solvent was evaporated, yielding: 0.085 g of enantiomer A and 0.085 g of enantiomer B. Both fractions were crystallized from DIPE/2-propanone. The precipitate was filtered off and dried, yielding: 0.042 g of Compound no. 303 (enantiomer A) (melting point: 130° C.) and 0.055 g of Compound no. 304 (enantiomer B) (melting point: 130° C.).

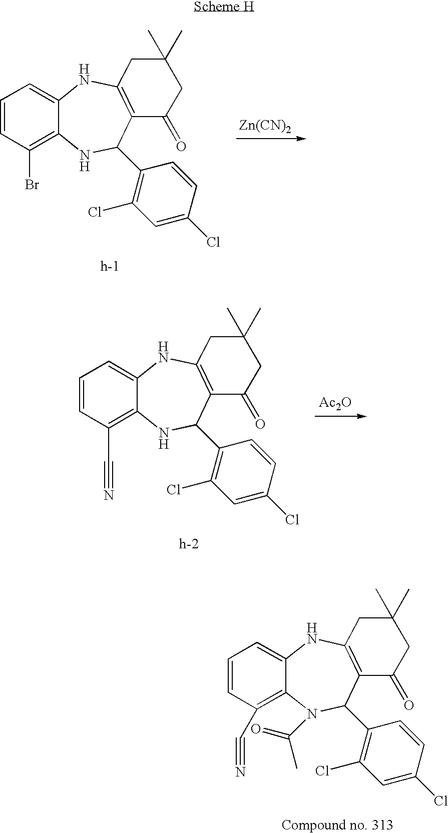

A mixture of h-1 (0.0004 mol), Zn(CN)₂ (0.0007 mol), Pd₂ dba₃ (0.022 g), dppf (0.033 g), Zn (0.0002 mol) and Zn(OAc)₂ (0.0002 mol) in DMA (2 ml) was stirred at 130° C. in a microwave oven for 30 minutes, then poured into H₂O and extracted with EtOAc. The organic layer was washed with H₂O, dried (over MgSO₄), filtered and the solvent was evaporated until dryness. The residue was purified by column chromatography over silica gel (eluent: CH₂Cl₂/EtOAc 95/5). The pure fractions were collected and the solvent was evaporated. The residue (0.14 g) was crystallized from CH₃CN. The precipitate was filtered off and dried, yielding: 0.06 g of h-2 (melting point: 225° C.).

A mixture of h-2 (0.0002 mol) in Ac₂O (4 ml) was stirred and refluxed for 12 hours and then evaporated until dryness. The residue was purified by column chromatography over silica gel (eluent: CH₂Cl₂/CH₃OH 98/2). The pure fractions were collected and the solvent was evaporated. The residue (0.07 g) was crystallized from 2-propanone/diethyl ether. The precipitate was filtered off and dried. Yielding: 0.025 g of Compound no. 313 (melting point: 248° C.).

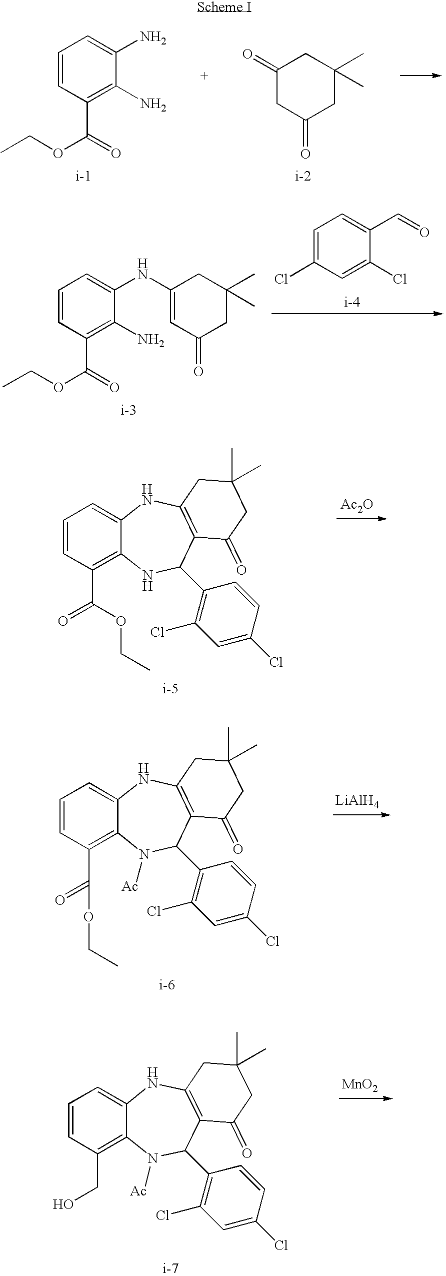

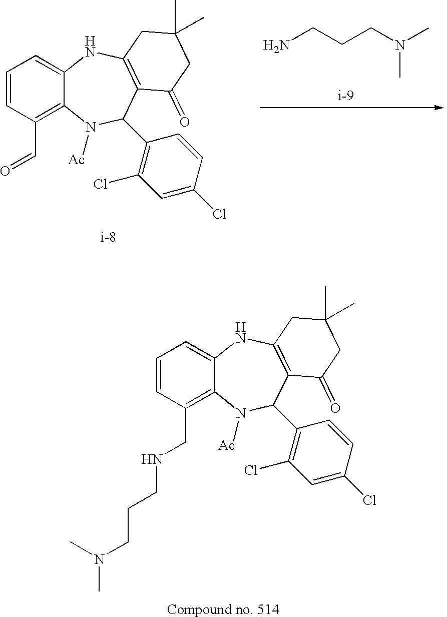

A mixture of i-1 (0.0094 mol) and i-2 (0.0094 mol) in toluene (50 ml) was stirred and refluxed for 12 h in a Dean Stark apparatus, then cooled down to room temperature. The precipitate was filtered off and dried, yielding: 2.3 g of i-3 (76%).

A mixture of i-3 (0.0044 mol) and i-4 (0.0044 mol) in EtOH (12.44 ml) and AcOH (1.23 ml) was stirred at 75° C. for 12 h, then evaporated until dryness. The residue was taken up in EtOAc/NaHCO3 10% aq. The mixture was stirred at room temperature for 1 hour and 30 minutes, then filtered off and dried. The residue (0.4 g) was washed with EtOAc, dried (over MgSO4), filtered and the solvent was evaporated until dryness. The residue (1.77 g) was purified by column chromatography over silica gel (eluent: CH₂Cl₂/CH₃OH/NH₄OH 98.5/1.5/0.1). The pure fractions were collected and the solvent was evaporated, yielding: 1 g of I-5 (52%). A small fraction was crystallized from CH3CN/DIPE (melting point: 208° C.). The remaining fraction of i-5 was used in the next reaction step.

A mixture of i-5 (0.0008 mol) in Ac₂O (60 ml) was stirred and refluxed for 4 hours, then evaporated until dryness, yielding: 0.46 g of i-6 (100%).

i-6 (0.0059 mol) was added at 0° C. to a suspension of LiAlH₄ (0.0018 mol) in THF (4 ml) under N₂ flow. The mixture was stirred at 0° C. for 3 hours. EtOAc then ice were added. The mixture was extracted with EtOAc. The organic layer was separated, dried (over MgSO4), filtered and the solvent was evaporated until dryness. The residue (0.175 g) was purified by column chromatography over silica gel (eluent: CH₂Cl₂/CH₃OH/NH₄OH 95/5/0.5 to 93/7/0.7). The pure fractions were collected and the solvent was evaporated, yielding: 0.032 g of i-7 (12%) (melting point 200° C.).

A mixture of i-7 (0.0005 mol) and MnO₂ (1.5 g) in CH₂Cl₂ (10 ml) was stirred at room temperature for 3 hours, then filtered over celite and washed with CH₂Cl₂. The filtrate was evaporated until dryness. The residue was crystallized from CH₃CN/DIPE. The precipitate was filtered off and dried, yielding: 0.12 g of i-8 (48%).

A mixture of i-8 (0.0001 mol), i-9 (0.0001 mol), BH₃CN— on solid support (0.0001 mol) and AcOH (5 drops) in CH₃OH (5 ml) was stirred at room temperature for 5 hours. The residue was purified by column chromatography over silica gel (eluent: CH₂Cl₂/CH₃OH/NH₄OH 94/6/0.6 to 82/18/1.8). The pure fractions were collected and the solvent was evaporated. The residue (0.035 g) was crystallized from CH₃CN. The precipitate was filtered off and dried. Yielding: 0.022 g of Compound no. 514 (31%) (melting point: 258° C.).

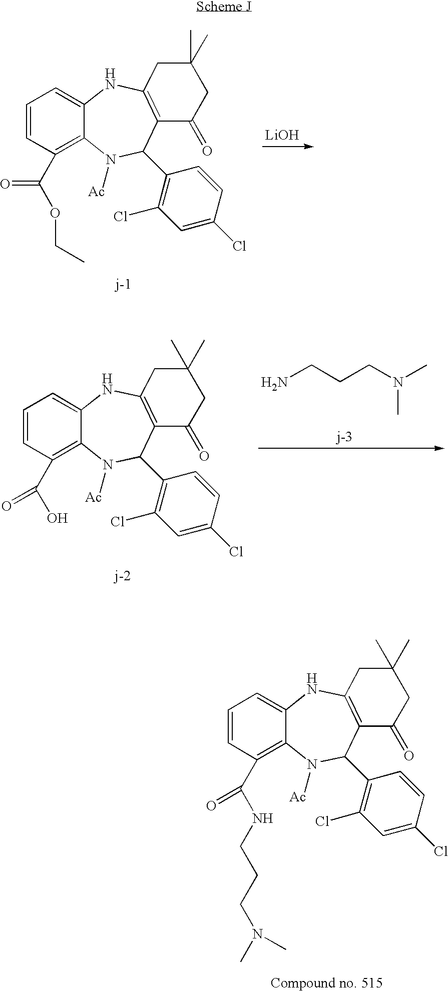

A mixture of j-1 (0.0004 mol) and LiOH (0.0009 mol) in THF (20 ml) and H₂O (20 ml) was stirred at 50° C. for 36 hours. THF was evaporated. The mixture was acidified with HCl 1N until pH was set to 7. The precipitate was filtered. The filtrate was basified with K₂CO₃ 10%. The aqueous layer was acidified with HCl 1N. The precipitate was filtered off and dried, yielding: 0.092 g of j-2 (60%).

A mixture of j-2 (0.0001 mol), j-3 (0.0003 mol), EDCI (0.0003 mol) and HOBT (0.0003 mol) in CH₂Cl₂ (4 ml) and THF (2 ml) was stirred at room temperature for 6 hours, poured into H₂O and extracted with CH₂Cl₂. The organic layer was separated, dried (over MgSO₄), filtered and the solvent was evaporated until dryness. The residue (0.1 g) was purified by column chromatography over silica gel (eluent: CH₂Cl₂/CH₃OH/NH₄OH 92/8/0.8 to 78/20/2). The pure fractions were collected and the solvent was evaporated. The residue (0.054 g) was crystallized from CH₃CN/DIPE. The precipitate was filtered off and dried, yielding: 0.048 g of Compound no. 515 (melting point: 226° C.).

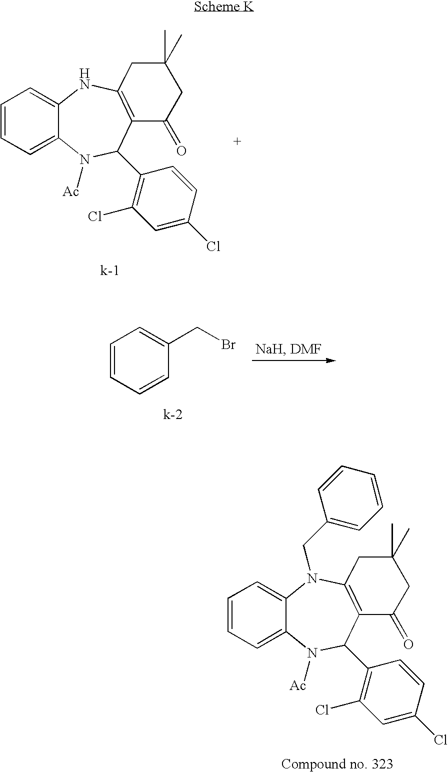

NaH (0.0001 mol) was added to a solution of k−1 (0.0005 mol) in DMF (2.5 ml). The mixture was stirred for 10 minutes. k-2 (0.0001 mol) was added. The mixture was stirred at room temperature for 12 h, then evaporated until dryness. The residue (0.45 g) was purified by column chromatography over silica gel (eluent: CH₂Cl₂/CH₃OH 98/2). The pure fractions were collected and the solvent was evaporated. The residue (0.2 g) was crystallized from 2-propanone. The precipitate was filtered off and dried, yielding: 0.043 g of Compound no. 323 (melting point: 235° C.).

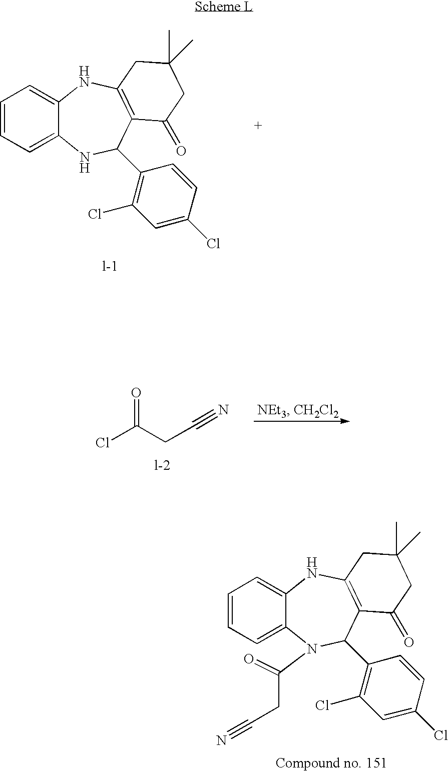

A mixture of l-1 (0.0003 mol), l-2 (0.0004 mol) and NEt₃ (0.065 ml) in CH₂Cl₂ (4 ml) was stirred at room temperature for 48 hours. The mixture was stirred at room temperature for 12 h, then poured into H₂O/CH₂Cl₂. The organic layer was separated, dried (over MgSO₄), filtered and the solvent was evaporated. The residue (0.13 g) was purified by column chromatography over silica gel (eluent: CH₂Cl₂/CH₃OH/NH₄OH 99/1/0.1 to 94/6/0.6). The pure fractions were collected and the solvent was evaporated. The residue (0.05 g) was crystallized from CH₃CN/DIPE. The precipitate was filtered off and dried, yielding: 0.041 g of Compound no. 151 (23%) (melting point >260° C.).

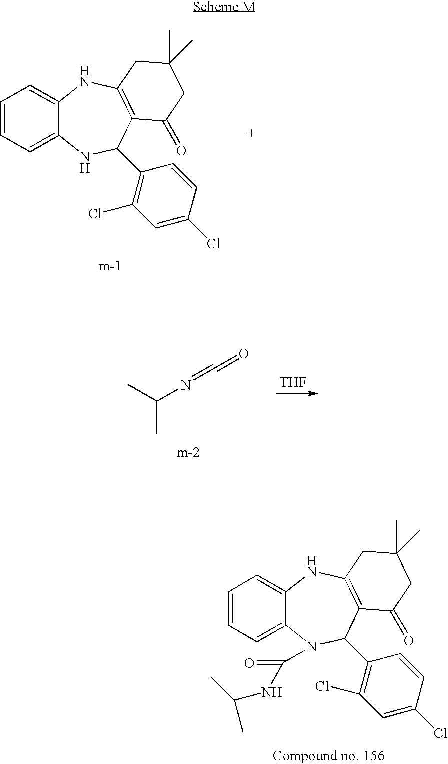

A mixture of m-1 (0.0002 mol) and m-2 (0.0003 mol) in THF (5 ml) was stirred and refluxed for 1 hour and 30 minutes, then taken up in H₂O/CH₂Cl₂ and extracted with CH₂Cl₂. The organic layer was separated, dried (over MgSO₄), filtered and the solvent was evaporated until dryness. The residue was crystallized from CH₃CN/DIPE (few). The precipitate was filtered off and dried, yielding: 0.064 g of Compound no. 156 (53%) (melting point >260° C.).

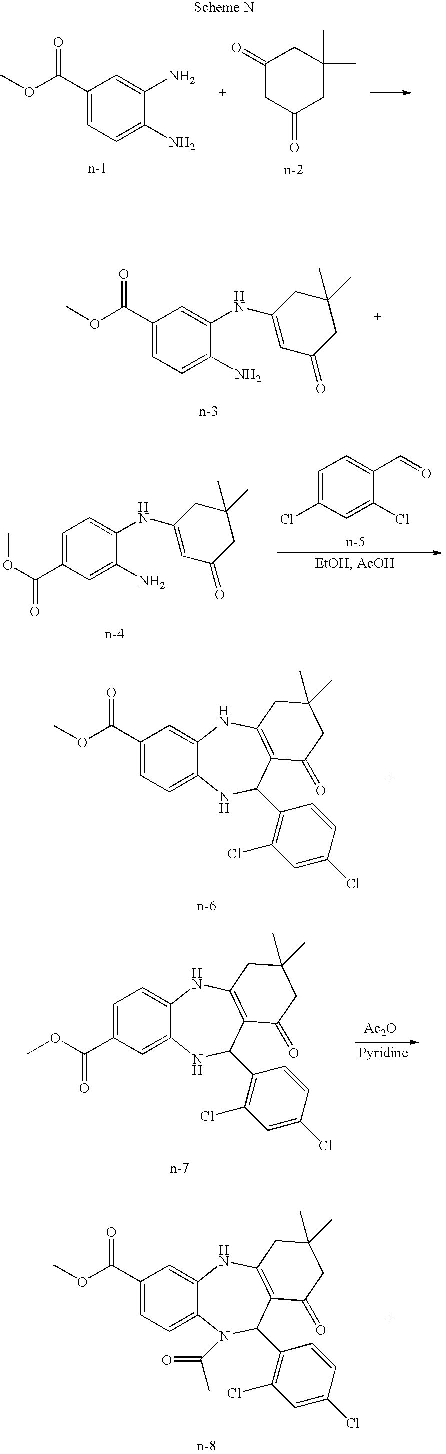

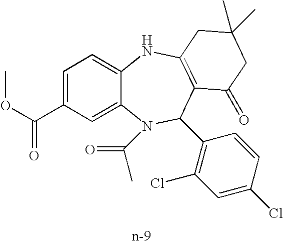

A mixture of n-1 (0.01 mol) and n-2 (0.01 mol) in toluene (20 ml) was stirred and 5 refluxed in a Dean Stark apparatus for 12 h, then evaporated until dryness, yielding: 3 g of n-3+n-4. This mixture of product was used directly in the next reaction step.

A mixture of n-3+n-4 (0.01 mol) and n-5 (0.01 mol) in EtOH (25 ml) and AcOH (25 ml) was stirred at 75° C. for 12 h, then evaporated until dryness. The residue was taken up in EtOAc and saturated solution of NaHCO₃. The mixture was stirred for 1 hour and 30 minutes, and then filtered. The aqueous layer was extracted with EtOAc. The organic layer was separated, dried (over MgSO₄), filtered and the solvent was evaporated. The residue was purified by column chromatography over silica gel (eluent: CH₂Cl₂ 100). The pure fractions were collected and the solvent was evaporated, yielding: 1.25 g of n-6+n-7.

Ac₂O (1 ml) was added to a solution of n-6+n-7 (0.0012 mol) in pyridine (10 ml). The mixture was stirred at room temperature for 12 h, then evaporated until dryness. The residue (0.54 g) was purified by column chromatography over silica gel (eluent: CH₂Cl₂/CH₃OH/NH₄OH 98/2/0.2 to 92/8/0.8). Two fractions were collected and the solvent was evaporated, yielding: 0.14 g F1 (24%) and 0.15 g F2 (25%). Each fraction was crystallized from 2-propanone/diethyl ether. The precipitate was filtered off and dried. Yielding: n-8 (melting point >250° C.) and n-9 (melting point >250° C.).

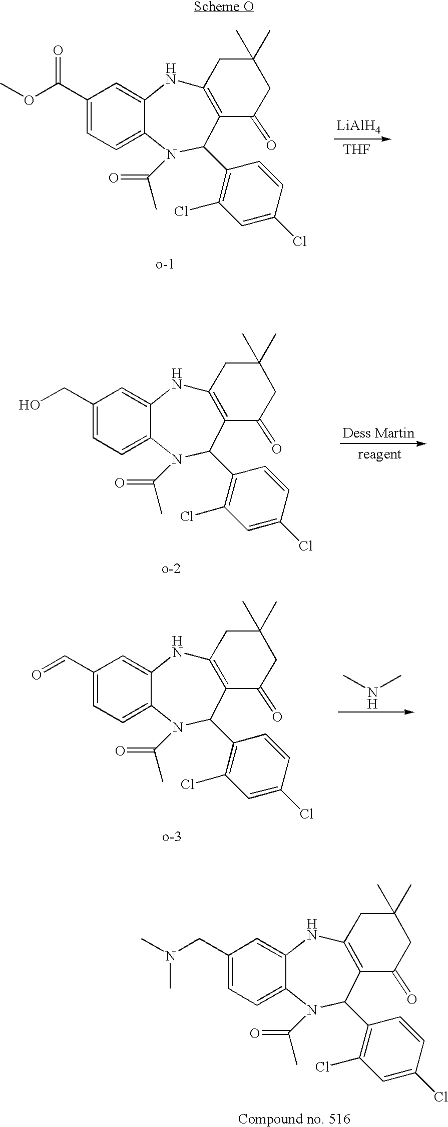

A mixture of o-1 (0.003 mol) and LiAlH₄ (0.012 mol) in THF (60 ml) was stirred at room temperature for 2 hours. H₂O and NaOH 3M were the added carefully. The mixture was stirred for 1 h. The mixture was extracted with CH₂Cl₂/CH₃OH (few). The organic layer was separated, dried (over MgSO₄), filtered and the solvent was evaporated until dryness. The residue was washed with H2O and dried, yielding: 1.54 g o-2 (100%). A small part (0.07 g) was purified by column chromatography over silica gel (eluent: CH₂Cl₂/CH₃OH/NH₄OH 98/2/0.2 to 92/8/0.8). The pure fractions were collected and the solvent was evaporated, yielding: 0.022 g. The remaining product was used in the next reaction step.

Dess Martin reagent (13.34 ml) was added at room temperature of o-2 (0.0031 mol) in CH₂Cl₂ (11.6 ml). The mixture was stirred at room temperature for 1 hour. Saturated NaHCO₃ and Na₂S₂O₄ were added. The mixture was extracted with CH₂Cl₂. The organic layer was separated, dried (over MgSO₄), filtered and the solvent was evaporated until dryness. The residue was crystallized from CH₃CN. The precipitate was filtered off and dried, yielding: 1.3 g of o-3.

A mixture of o-3 (0.0002 mol), dimethylamine (0.0006 mol), BH₃CN— on solid support (0.0006 mol) and AcOH (4 drops) in CH₃OH (5 ml) was stirred at room temperature for 12 h. Dimethylamine (0.5 eq) and BH₃CN— on solid support (0.5 eq) were added again. The mixture was stirred at room temperature for 12 h, then filtered. The filtrate was evaporated. The mixture was taken up in CH₂Cl₂/H₂O. The organic layer was separated, dried (over MgSO4), filtered and the solvent was evaporated. The residue was purified by column chromatography over silica gel (eluent: CH₂Cl₂/CH₃OH/NH₄OH 97/3/0.5). The pure fractions were collected and the solvent was evaporated. The residue (0.085 g) was crystallized from CH₃CN/DIPE. The precipitate was filtered off and dried, yielding: 0.056 g of Compound no. 516 (52%) (melting point >260° C.).

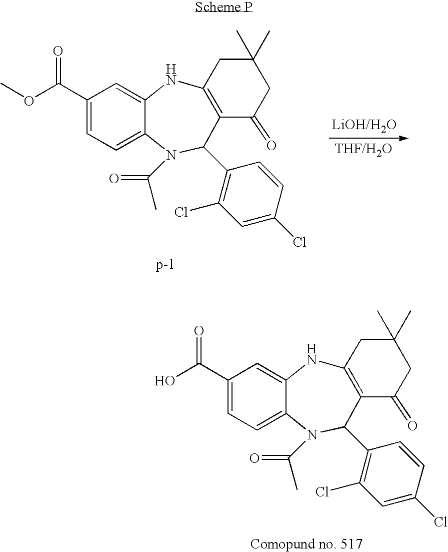

A mixture of p-1 (0.0001 mol) and LiOH/H₂O (0.0004 mol) in THF/H₂O (1/1) (10 ml) was stirred at room temperature for 12 h, and then concentrated under reduced pressure. The aqueous layer was washed with diethyl ether, made acidic with HCl 1N and filtered. The precipitate was dried, yielding: 0.045 g of Compound no. 517 (melting point >250° C.).

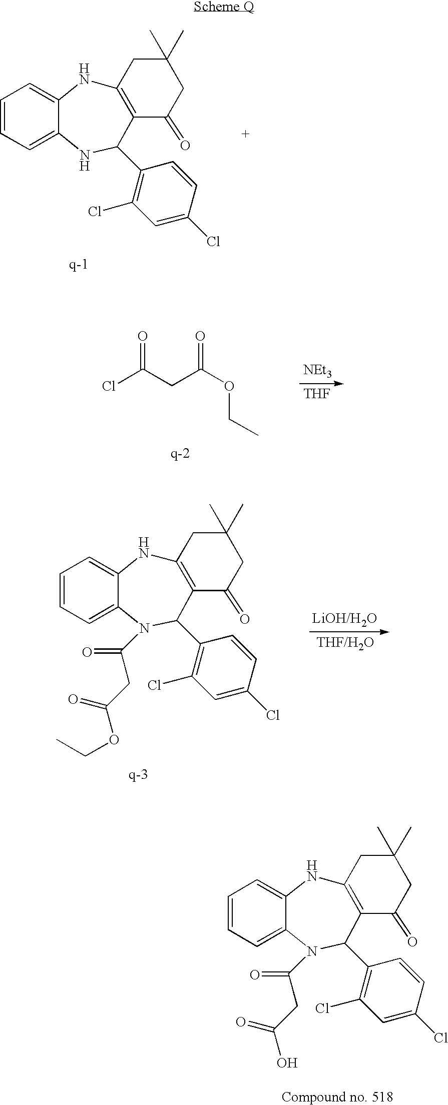

q-2 (0.0012 mol) was added drop wise to a mixture of q-1 (0.001 mol) and NEt₃ (0.0012 mol) in THF (5 ml). The mixture was stirred and refluxed for 12 h, then cooled down to room temperature. The precipitate was filtered, washed with THF. The filtrate was evaporated. The residue was purified by column chromatography over silica gel (eluent: CH₂Cl₂/CH₃OH/NH₄OH 98/2/0.2, 93/7/0.7 then 94/6/0.6). The pure fractions were collected and the solvent was evaporated. The residue (0.09 g, 18%) was crystallized from 2-propanone. The precipitate was filtered off and dried. Yielding: q-3 (melting point: 190° C.).

A mixture of q-3 (0.0001 mol) and LiOH/H₂O (0.0002 mol) in THF (5 ml) and H₂O (5 ml) was stirred at room temperature for 3 hours. THF was evaporated. The residue was extracted with CH₂Cl₂. The aqueous layer was made acidic with HCl 3N. The mixture was filtered off and dried, yielding: 0.055 g of Compound no. 518 (83%) (melting point: 200° C.).

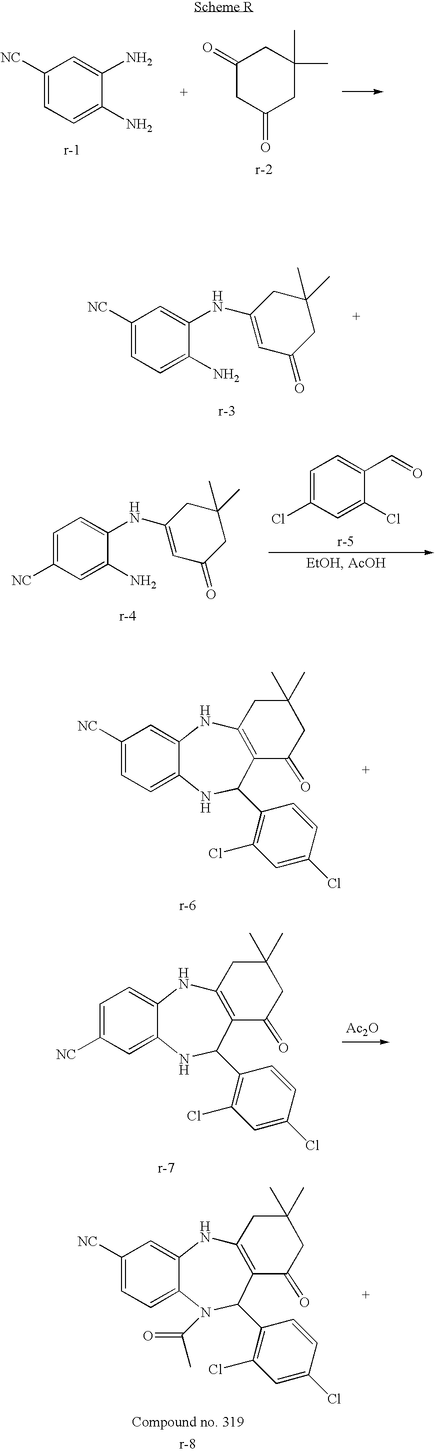

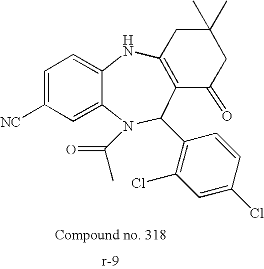

A mixture of r-1 (0.02 mol) and r-2 (0.02 mol) in toluene (100 ml) was stirred and refluxed for 12 h in a Dean Stark apparatus. The solution was concentrated under reduced pressure and the residue was purified by column chromatography over silica gel (eluent: CH₂Cl₂/CH₃OH/NH₄OH 95/5/0.5). The pure fractions were collected and the solvent was evaporated, yielding 2.04 g of the mixture r-3+r-4.

A mixture of r-3+r-4 (0.0063 mol) and r-5 (0.0035 mol) in EtOH (30 ml) and AcOH (3 ml) was stirred at 75° C. for 12 h, then evaporated until dryness. The residue was taken up in EtOAc and saturated solution of NaHCO₃. The mixture was stirred for 1 hour and 30 minutes, filtered and extracted with EtOAc. The organic layer was separated, dried (over MgSO₄), filtered and the solvent was evaporated. The residue was purified by column chromatography over silica gel (eluent: CH₂Cl₂/CH₃OH/NH₄OH 98.5/1.5/0.1). The pure fractions were collected and the solvent was evaporated, yielding 0.072 g of the mixture r-6+r-7.

A mixture of r-6+r-7 (0.0004 mol) in C (5 ml) was stirred and refluxed for 2 hours, then evaporated until dryness. The residue (0.2 g) was purified by column chromatography over silica gel (eluent: toluene/iPrOH/NH₄OH 90/10/0.5). Two fractions were collected and the solvent was evaporated. Yielding: 0.125 g F1 and 0.037 g F2. Each fraction was crystallized from 2-propanone/diethyl ether. The precipitate was filtered off and dried, yielding: 0.034 g of Compound no. 319 (r-8) (melting point >250° C.) and 0.008 g of Compound no. 318 (r-9) (melting point >250° C.).

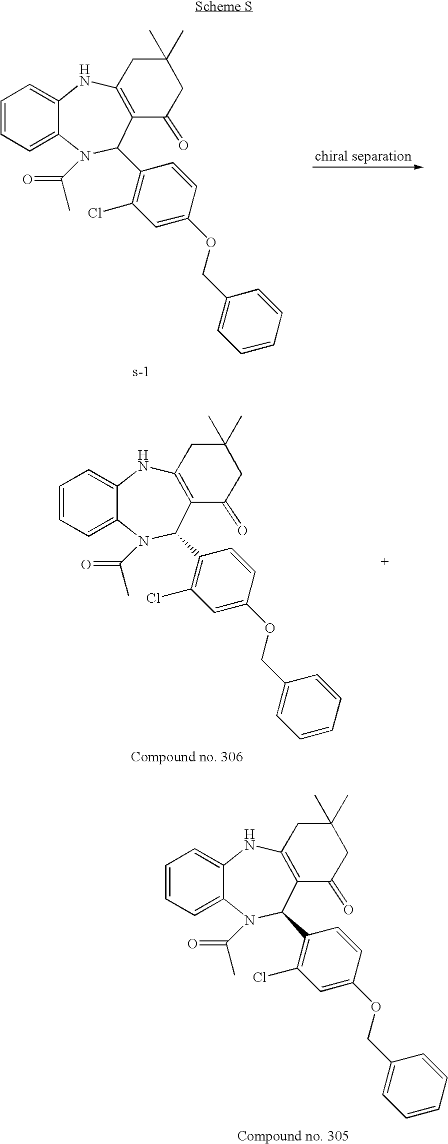

s-1 (0.0001 mol) was purified by SFC with a chiral column (eluent: CO₂/iPrOH 65/35). Two fractions were collected and the solvent was evaporated, yielding: 0.02 g of Compound no. 306 (enantiomer A) and 0.018 g of Compound no. 305 (enantiomer B).

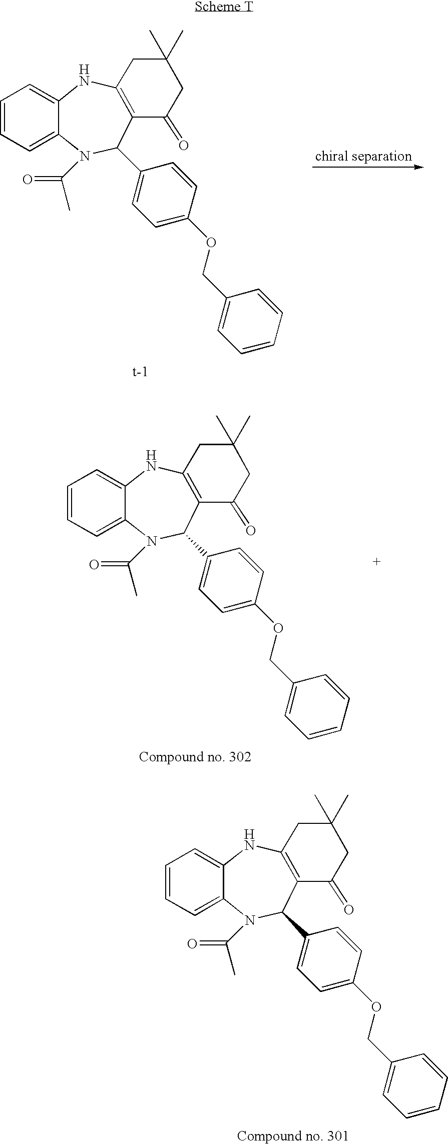

t-1 (0.1 g) was purified by column chromatography over Chiracel pack OD (eluent: EtOH/2-propanol 50/50), yielding 0.054 g of Compound no. 302 (enantiomer A) and 0.044 g of Compound no. 301 (enantiomer B).

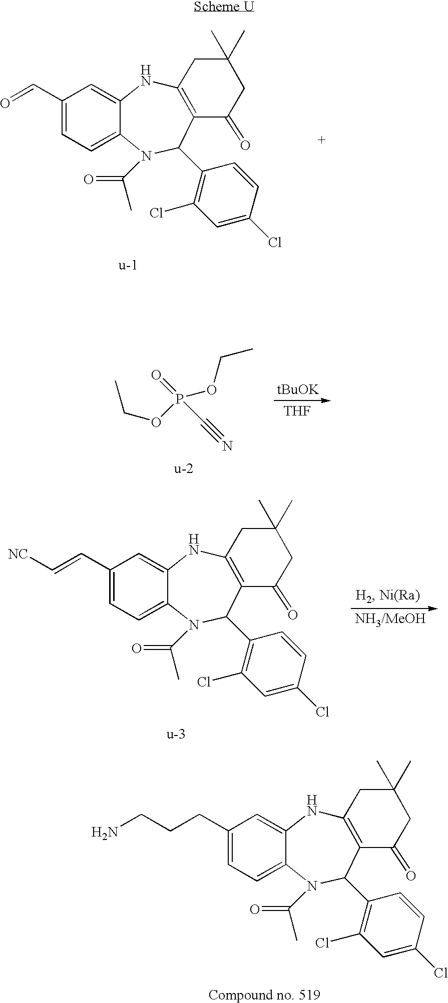

tBuOK (0.00044 mol) was added portion wise to a solution of u-2 (0.00044 mol) in THF (5 ml) at 0° C. The mixture was stirred at this temperature for 15 min and u-1 (0.00022 mol) was then added. The reaction was stirred at room temperature for 2 h and poured into water. The solution was acidified using HCl 3N and extracted with CH₂Cl₂. The organic layer was dried (over MgSO₄), filtered and concentrated under reduced pressure. The residue was purified by column chromatography over silica gel (eluent: CH₂Cl₂/CH₃OH/NH₄OH 95/5/0.5). The pure fractions were collected and the solvent was evaporated, yielding 0.1 g of u-3 (95%).

A mixture of u-3 (0.1 g), Raney Nickel (0.1 g) in a solution of NH₃/MeOH 7 N (10 ml) was hydrogenated under a 3 bars pressure at room temperature for 8 h. The solution was then filtered through a pad of celite using MeOH and concentrated under reduced pressure. The residue was purified by column chromatography over silica gel (eluent: CH₂Cl₂/CH₃OH/NH₄OH 85/15/1). The pure fractions were collected and the solvent was evaporated, yielding 0.024 g. The residue was crystallized from CH₃CN/DIPE, yielding 0.013 g of Compound no. 519 (TMC533774) (13%) (melting point 242° C.).

### Example 93

### 3-(2-Benzyloxyphenyl)-11-(2,4-dichlorophenyl)-2,3,4,5,10,11-hexahydro-dibenzo[b,e][1,4]diazepin-1-one: Compound 417 (diastereomer A) and Compound 419 (diastereomer B)

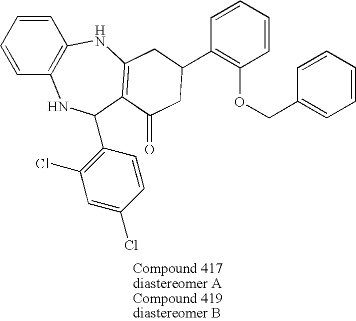

A solution of Intermediate (1-4) (200 mg, 0.520 mmol) and 2,4-dichlorobenzaldehyde (91 mg, 0.520 mmol) in 10 mL dry EtOH and 1 mL AcOH was heated at 75° C. for 5 h. Solvents were evaporated. The residue was dissolved in EtOAc and stirred for 1.5 h with saturated aqueous NaHCO₃, and dried (Na₂SO₄). Two diastereomers were obtained, and purified by silica flash column chromatography (gradient elution from heptane/EtOAc 4:1 to 2:1) to give final Compound No. 417 diastereomer A, (yield: 118 mg, 41.1%): m/z=542 (M+H)⁺, and final Compound No. 419 diastereomer B (yield: 57 mg, 20.2%): m/z=542 (M+H)⁺.

### Example 94

### 3-(2-Benzyloxyphenyl)-11-(3-benzyloxyphenyl)-2,3,4,5,10,11-hexahydro-dibenzo[b,e][1,4]diazepin-1-one Compound No. 418 (diastereomer A) and Compound No. 420 (diastereomer B.

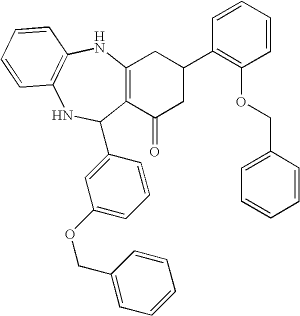

The title compounds were prepared and separated from Intermediate (1-4) and 3-benzyloxybenzaldehyde following the procedure reported for Compounds Nos. 417 and 419: m/z=580 (M+H)⁺.

### Example 95

### 11-(2,4-dichlorophenyl)-2,3,4,5,10,11-hexahydro-3,3-dimethyl-1H-dibenzo[b,e][1,4]diazepin-1-one Compound No. 423.

### Step A.

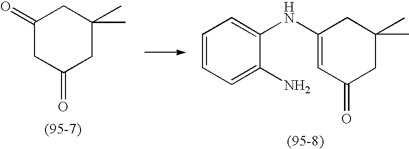

A solution of dimedone (95-7, 5.0 g, 35.67 mmol) and o-phenylenediamine (3.86 g, 35.69 mmol) in 150 mL dry toluene were refluxed overnight in a Dean-Stark trap. After 24 h, the solvent was evaporated to give Intermediate (95-8) as an orange foam which was used without further purification in the next step.

### Step B.

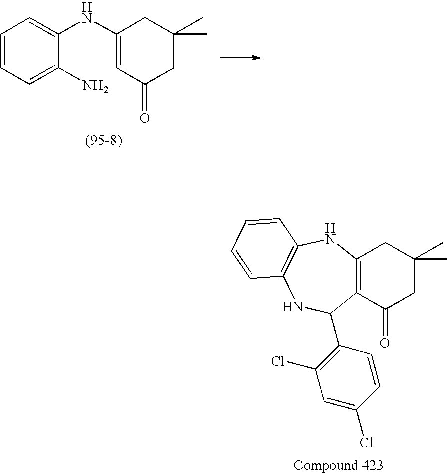

A solution of Intermediate (95-8) (35.7 mmol) and 2,4-dichlorobenzaldehyde (6.24 g, 35.65 mmol) in a mixture of 100 mL dry EtOH and 10 mL AcOH was heated at 75° C. overnight. The reaction mixture was cooled to room temperature and the solvents evaporated. The residue was dissolved in EtOAc and stirred with saturated aqueous NaHCO₃ for 1.5 h. Then, the water layer was removed in a separating funnel and the organic layer was filtered off, the filtrate was washed twice with EtOAc. Organic layers were dried (Na₂SO₄), evaporated and the residue dried under high vacuum, yielding 9.45 g (68.4%) of the final Compound No. 423: m/z=387 (M+H)⁺.

### Example 96

### 11-(2,4-Dichlorophenyl)-2,3,4,5,10,11-hexahydro-3,3,10-trimethyl-1H-dibenzo[b,e][1,4]diazepin-1-one Compound No. 507

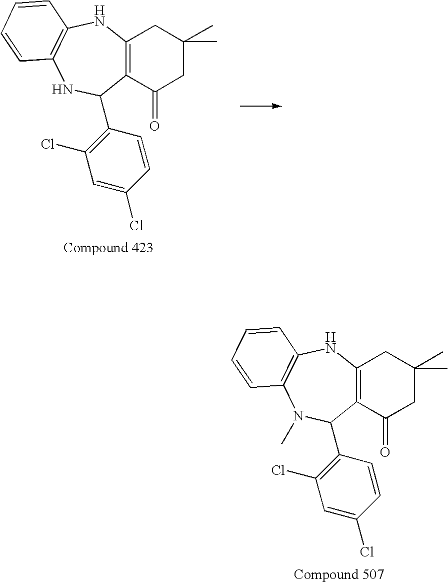

Methyl iodide (97 μL, 1.555 mmol) was added to a solution of Compound No. 423 (0.50 g, 1.291 mmol) and K₂CO₃ (214 mg, 1.55 mmol) in acetone. The tube was sealed and stirred at room temperature overnight. Additional methyl iodide (146 μL, 2.34 mmol) was added and the sealed tube was stirred for 2 days. The reaction mixture was dropped onto water and the solid was filtered off and dried. Purification by preparative TLC (EtOAc/heptane 1:1) followed by sonication in i-Pr₂O and filtration afforded final Compound No. 507: m/z=401 (M+H)⁺.

### Example 97

### 11-(2,4-dichlorophenyl)-10-ethyl-2,3,4,5,10,11-hexahydro-3,3-dimethyl1H-dibenzo[b,e][1,4]diazepin-1-one Compound No. 508.

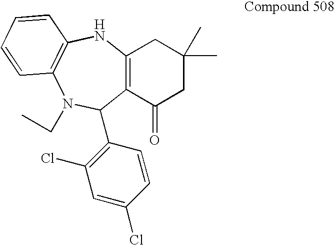

The title compound was prepared from Compound No. 423 and ethyl iodide (1 mL, 12.5 mmol) following the procedure reported for Compound No. 507 m/z=415 (M+H)⁺.

### Example 98

### 11-(2,4-Dichlorophenyl)-2,3,4,5,10,11-hexahydro-3,3-dimethyl-10-propyl-1H-dibenzo[b,e][1,4]diazepin-1-one Compound No. 509

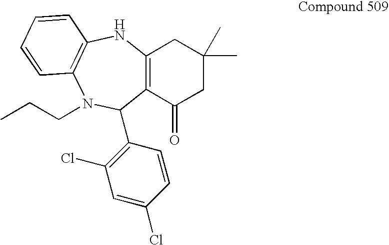

The title compound was prepared from Compound No. 423 and propyl iodide (1.26 mL, 12.9 mmol) following the procedure reported for Compound No. 507 m/z=429 (M+H)⁺.

### Example 99

### 11-(1-Bromo-2-phenylvinyl)-3,3-dimethyl-2,3,4,5,10,11-hexahydro-dibenzo[b,e][1,4]diazepin-1-one Compound No. 500

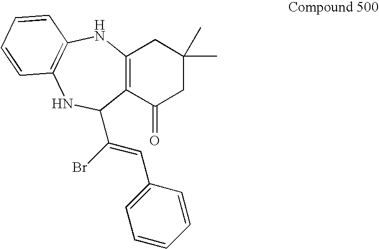

The title compound was prepared from Intermediate (95-8) (11.9 mmol) and 2-bromo-3-phenylacroleine (2.51 g, 11.9 mmol) following the procedure reported for Compound No. 423: m/z=424 (M+H)⁺.

### Example 100

### 11-(1-Chloro-2-phenylvinyl)-3,3-dimethyl-2,3,4,5,10,11-hexahydro-dibenzo[b,e][1,4]diazepin-1-one Compound No. 506

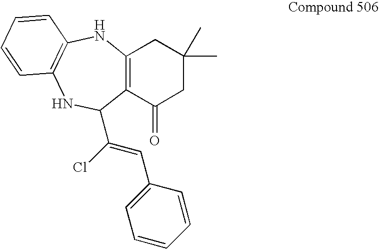

The title compound was prepared from Intermediate (95-8) (11.9 mmol) and 2-chloro-3-phenylacroleine (1.98 g, 11.88 mmol) following the procedure reported for Compound No. 423: m/z=380 (M+H)⁺.

### Example 101

### 11-[3-(4-Chlorobenzoyloxy)phenyl]-3,3-dimethyl-2,3,4,5,10,11-hexahydro-dibenzo[b,e][1,4]diazepin-1-one Compound No. 431

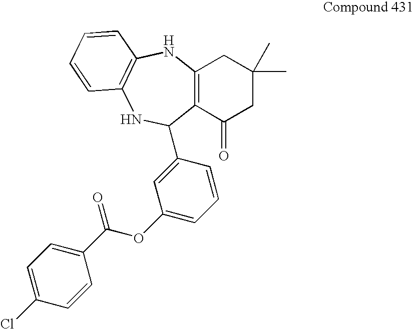

The title compound was prepared from Intermediate (95-8) (3.9 mmol) and 3-[(4-chlorobenzoyl)oxy]benzaldehyde (1.03 g, 3.95 mmol) following the procedure reported for Compound No. 423: m/z=474 (M+H)⁺.

### Example 102

### 11-(2,4-Dichlorophenyl)-2,3,4,5,10,11-hexahydro-3,3,7,8-tetramethyl-1H-dibenzo[b,e][1,4]diazepin-1-one Compound No. 464

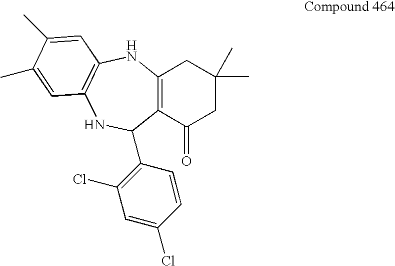

The title compound was prepared from 4,5-dimethyl-o-phenylenediamine (2.48 g, 18.21 mmol), and 2,4-dichlorobenzaldehyde (3.19 g, 18.23 mmol) following the procedure reported for Compound No. 423: m/z=416 (M+H)⁺.

### Example 103

### 11-[3-(4-Chlorobenzoyloxy)phenyl]-3-(2-benzyloxyphenyl)-2,3,4,5,10,11-hexahydro-dibenzo[b,e][1,4]diazepin-1-one Compound No. 512 diastereomer A

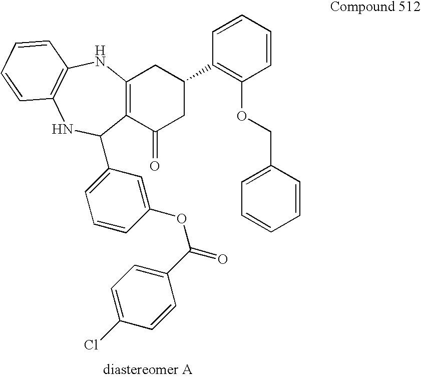

The compound was prepared from Intermediate (93-4) (300 mg, 0.780 mmol) and 3-[(4-chlorobenzoyl)oxy]benzaldehyde (244 mg, 0.936 mmol) following the procedure reported for Compound No. 417: m/z=628 (M+H)⁺.

### Example 104

### 3-(2-benzyloxyphenyl)-11-[3-(4-chlorobenzoyloxy)phenyl]-2,3,4,5,10,11-hexahydro-dibenzo[b,e][1,4]diazepin-1-one—Compound No. 513 diastereomer B

The compound was prepared from Intermediate (93-4) (300 mg, 0.780 mmol) and 3-[(4-chlorobenzoyl)oxy]benzaldehyde (244 mg, 0.936 mmol) following the procedure reported for Compound No. 419: m/z=628 (M+H)⁺.

### Example 105

### 7,8-Dichloro-11-(2,4-dichlorophenyl)-2,3,4,5,10,11-hexahydro-3,3-dimethyl-1H-dibenzo[b,e][1,4]diazepin-1-one Compound No. 465

The title compound was prepared from 4,5-dichloro-o-phenylenediamine, and 2,4-dichlorobenzaldehyde following the procedure reported for Compound No. 423: m/z=455 (M+H)+.

### Example 106

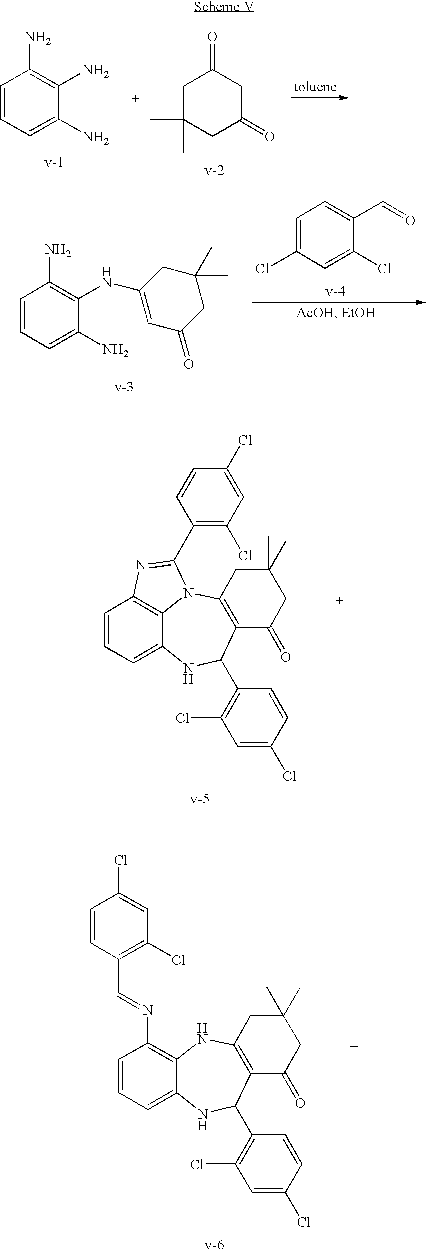

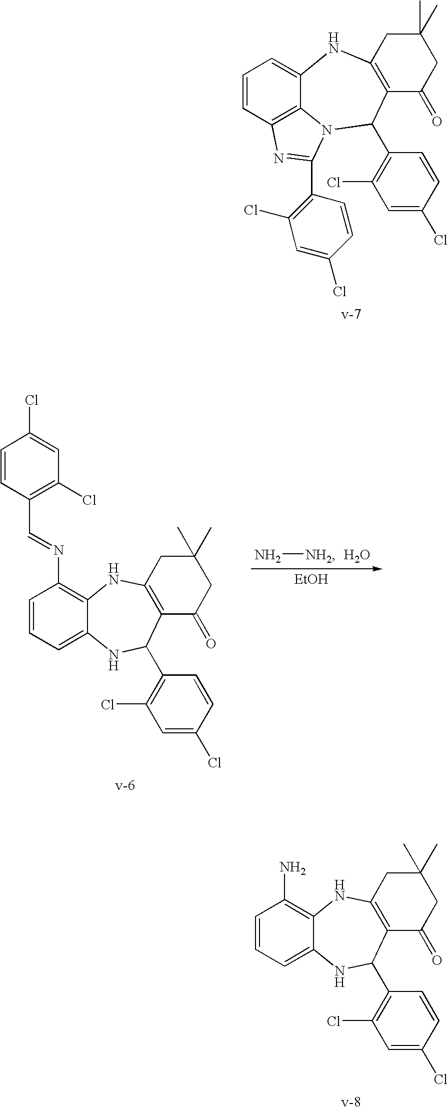

A mixture of v-1 (0.0089 mol) and v-2 (0.0089 mol) in toluene (50 ml) was stirred and refluxed for 12 h in a Dean Starck apparatus, then cooled to room temperature. The precipitate was filtered, washed with diethyl ether and dried, yielding: 2 g of v-3 (100%).

A mixture of v-3 (0.0106 mol) and v-4 (0.0106 mol) in AcOH (2.6 ml) and EtOH (50 ml) was stirred at 75° C. for 24 hours, then cooled to room temperature and concentrated under reduced pressure. The residue was taken up in CH₂Cl₂. The organic layer was washed with K₂CO₃ 10%, dried (over MgSO₄), filtered and the solvent was evaporated. The residue (5.7 g) was purified by column chromatography over silica gel (eluent: CH₂Cl₂/CH₃OH/NH₄OH 100/0/0 to 99/1/0.1). Three fractions were collected and the solvent was evaporated, yielding: 0.27 g of v-5 (4.5%) (melting point >260° C.), 0.4 g of v-6 (6.7%) and 0.34 g of v-7 (5.7%) (melting point >260° C.).

A mixture of v-6 (0.0006 mol) and NH₂—NH₂/H₂O (0.003 mol) in EtOH (20 ml) was stirred and refluxed for 6 hours, then concentrated under reduced pressure. The residue was crystallized from CH₃CN. The precipitate was filtered off and dried, yielding: 0.12 g (50%). Part of this fraction (0.04 g) was crystallized from CH₃CN. The precipitate was filtered off and dried, yielding: 0.03 g of v-8 (melting point: 248° C.).

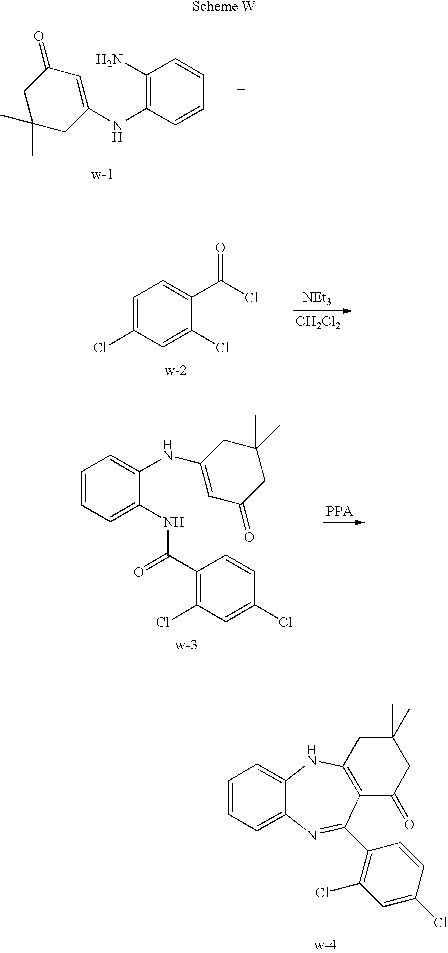

w-2 (0.0024 mol) was added at 5° C. to a solution of w-1 (0.0021 mol) and NEt₃ (0.0032 mol) in CH₂Cl₂ (15 ml). The mixture was stirred at 5° C. for 2 hours, then stirred at room temperature for 2 hours. The precipitate was filtered, washed with CH₂Cl₂ and dried, yielding: 0.15 g of w-3 (17%) (melting point: 240° C.).

A mixture of w-3 (0.0001 mol) and PPA (1.4 g) was stirred at 130° C. for 3 hours, then cooled to room temperature and taken up in K₂CO₃ 10%. The precipitate was filtered, washed with H₂O and taken up in CH₂Cl₂. The organic layer was separated, dried (over MgSO₄), filtered, washed with H₂O and the solvent was evaporated until dryness. The residue was crystallized from CH₃CN/DIPE. The precipitate was filtered off and dried, yielding: 0.032 g of w-4 (44%) (melting point: 252° C.).

Compounds according to the invention are listed in the Tables below including the compounds that were prepared in accordance with Examples 1-106 above. The remaining compounds listed in the Table may be prepared in an analogous manner to that described in the Examples. In these Tables the suffix A against a compound number denotes a diastereomer A, namely the diastereomer which was eluted first from the chromatography system; the suffix B against a compound number denotes a diastereomer B, namely the diastereomer which was eluted second from the chromatography system. Otherwise the compounds are mixtures of stereoisomeric forms.

TABLE 1

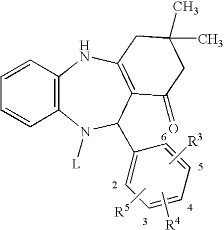

| Comp. | | | | |
|---|---|---|---|---|
| No. | R³ | R⁴ | R⁵ | L |
| 1 | 2-OCH₃ | 4-OCH₃ | H | 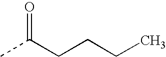 |
| 2 | 4 O—CH₂—CH₃] | H | H | 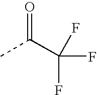 |
| 3 | 2-OCH₃ | 5-OCH₃ | H | 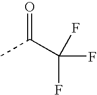 |
| 4 | 4-F | H | H | 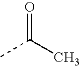 |
| 5 | 4-CF₃ | H | H | 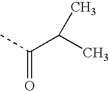 |
| 6 | 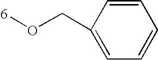 | H | H | 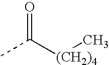 |
| 7 | 4-Cl | H | H | 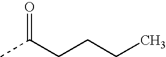 |
| 8 | 4-[N((CH₃)₂] | H | H | 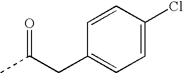 |
| 9 | 4-Cl | H | H | 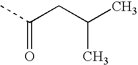 |
| 10 | 4-F | H | H | 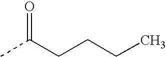 |
| 11 | 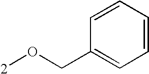 | H | H | 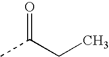 |
| 12 | 4-OCH₃ | H | H | 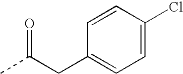 |
| 13 | 4-NO₂ | H | H | 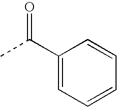 |
| 14 | 4 O—(CH₂)₃—CH₃] | H | H | 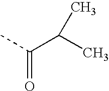 |
| 15 | 2-OCH₃ | 3-Cl | 5-Cl | 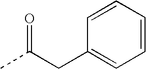 |
| 16 | 2-Cl | 6-Cl | H | 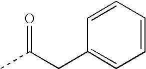 |
| 17 | 2-Cl | 4-Cl | H | 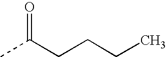 |
| 18 | 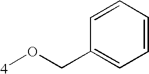 | H | H | 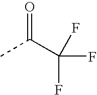 |
| 19 | 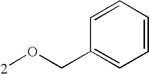 | H | H | 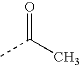 |
| 20 | 4-Cl | H | H | 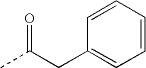 |
| 21 | 4-OCH₃ | H | H | 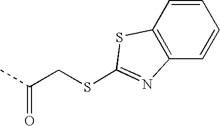 |
| 22 | 2-OCH₃ | 5-OCH₃ | H | 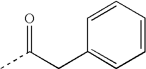 |
| 23 | 2-F | H | H | 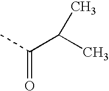 |
| 24 | 2-Cl | 6-F | H | 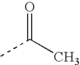 |
| 25 | 2-Cl | 3-Cl | H | 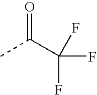 |
| 26 | 2-OCH₃ | 3-Cl | 5-Cl | 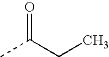 |
| 27 | 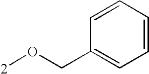 | H | H | 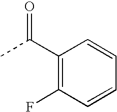 |
| 28 | 2-Cl | 4-Cl | H | 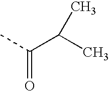 |
| 29 | 2-Cl | 3-Cl | H | 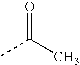 |
| 30 | 2-OCH₃ | 4-OCH₃ | H | 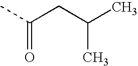 |
| 31 | 3-Cl | H | H | 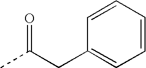 |
| 32 | 4-Cl | H | H | 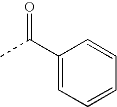 |
| 33 | 4-OCH₃ | H | H | 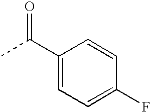 |
| 34 | 3-OCH₃ | H | H | 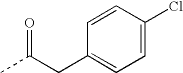 |
| 36 | 2-Cl | 4-Cl | H | 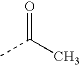 |
| 35 | 2-Cl | 4-Cl | H | 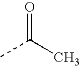 |
| 37 | 2-Cl | 6-F | H | 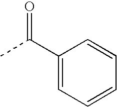 |
| 38 | 2-Cl | 4-Cl | H | 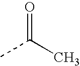 |
| 39 | 3-OH | H | H | 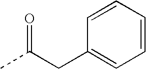 |
| 40 | 2-Cl | 3-Cl | H | 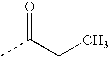 |
| 41 | 4-OCH₃ | H | H | 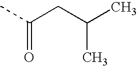 |
| 42 | 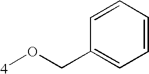 | H | H | 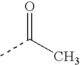 |
| 43 | 2-Cl | 6-F | H | 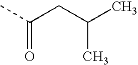 |
| 44 | 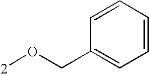 | H | H | 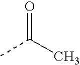 |
| 45 | 2-Cl | 4-Cl | H | 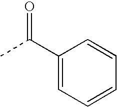 |
| 46 | 2-Cl | 6-Cl | H | 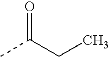 |
| 47 | 3-NO₂ | H | H | 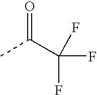 |
| 48 | 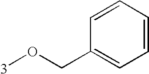 | 4-OCH₃ | H | 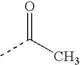 |
| 49 | 4-F | H | H | 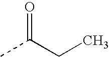 |
| 50 | 2-Cl | 6-Cl | H | 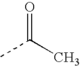 |
| 51 | 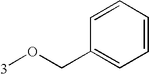 | 4-OCH₃ | H | 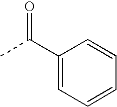 |
| 52 | 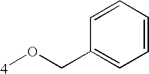 | H | H | 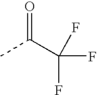 |
| 53 | 2-Cl | 4-Cl | H | 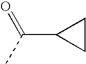 |
| 54 | 4-Cl | H | H | 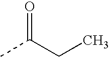 |
| 55 | 4-CF₃ | H | H | 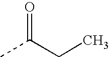 |
| 56 | 4-Cl | H | H | 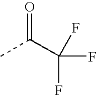 |
| 57 | 4 O—CH₂—CH₃] | H | H | 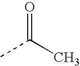 |
| 58 | 2-OCH₃ | 4-OCH₃ | H | 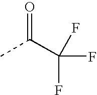 |
| 59 | 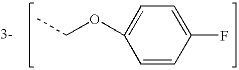 | 4-OCH₃ | H | 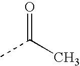 |
| 60 | 2-OCH₃ | 5-OCH₃ | H | 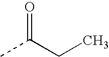 |
| 61 | 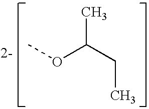 | H | H | 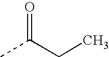 |
| 62 | 2-OCH₃ | 3-Cl | 5-Cl | 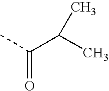 |
| 63 | 2 O—(CH₂)₂—CH₃] | H | H | 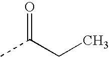 |
| 64 | 3 O—CH₂—CH₃] | 4-OH | H | 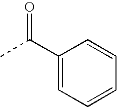 |
| 65 | 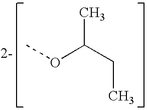 | H | H | 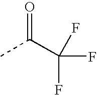 |
| 66 | 2-Cl | H | H | 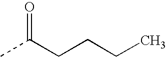 |
| 67 | 4 O—(CH₂)₄—CH₃] | H | H | 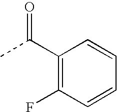 |
| 68 | 3-OCH₃ | 4-OCH₃ | H | 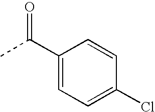 |
| 69 | 2-Cl | 6-F | H | 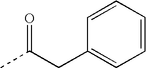 |
| 70 | 2-Cl | H | H | 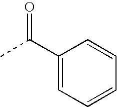 |
| 71 | 2-OCH₃ | 5-OCH₃ | H | 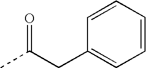 |
| 72 | 2-OCH₃ | 5-OCH₃ | H | 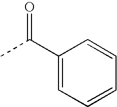 |
| 73 | 3-Cl | 5-Cl | 6-OCH₃ | 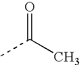 |
| 74 | 4-Br | H | H | 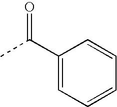 |
| 75 | 2-OCH₃ | 4-OCH₃ | H | 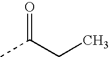 |
| 76 | 4 N(CH₃)₂] | H | H | 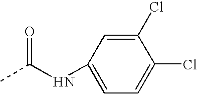 |
| 77 | 4-F | H | H | 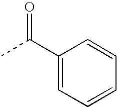 |
| 78 | 3-Cl | 4-Cl | H | 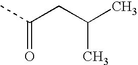 |
| 79 | 3 O—(CH₂)₃—CH₃] | H | H | 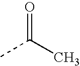 |
| 80 | 3-OCH₃ | 4-OCH₃ | H | 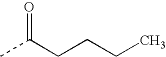 |
| 81 | 2-OCH₃ | H | H | 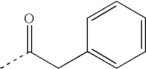 |
| 82 | 3-OCH₃ | H | H | 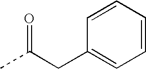 |
| 83 | 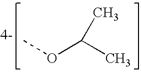 | H | H | 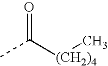 |
| 84 | 3-OCH₃ | 4-OCH₃ | H | 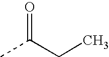 |
| 85 | 4 O—CH₂—CH₃] | H | H | 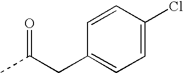 |
| 86 | 4 O—CH₂—CH₃] | H | H | 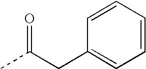 |
| 87 | 2-Cl | H | H | 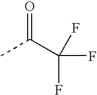 |
| 88 | 4-Cl | H | H | 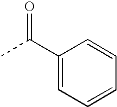 |
| 89 | 4-NO₂ | H | H | 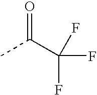 |
| 90 | 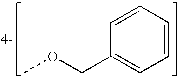 | H | H | 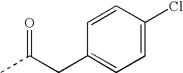 |
| 91 | 4 O—(CH₂)₃—CH₃] | H | H | 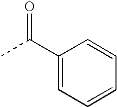 |
| 92 | H | H | H | 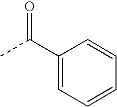 |
| 93 | 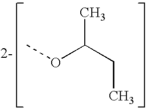 | H | H | 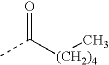 |
| 94 | 2-F | H | H | 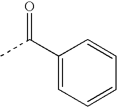 |
| 95 | 4-Br | H | H | 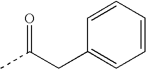 |
| 96 | 2-Br | 3-OCH₃ | 6-OCH₃ | 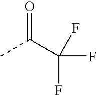 |
| 98 | 4 O—(CH₂)₂—CH₃] | 3-OCH₃ | H | 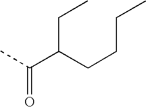 |
| 99 | 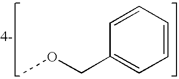 | H | H | 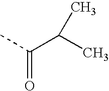 |
| 100 | 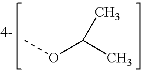 | H | H | 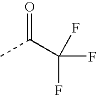 |
| 101 | 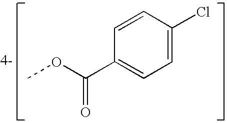 | H | H | 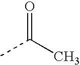 |
| 102 | 4 O—(CH₂)₄—CH₃] | H | H | 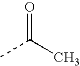 |
| 103 | 3-NO₂ | H | H | 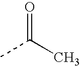 |
| 104 | 3-F | H | H | 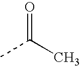 |
| 105 | 3-O—CH₃ | H | H | 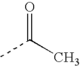 |
| 106 | 3-O—CH₃ | 4-O—CH₃ | H | 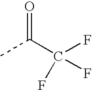 |
| 107 | 2-Cl | 4-Cl | H | 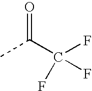 |
| 108 | 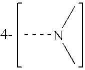 | H | H | 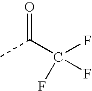 |
| 109 | 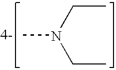 | H | H | 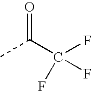 |
| 110 | 4-O—CH₃ | H | H | 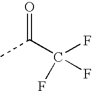 |
| 111 | 4-Cl | H | H | 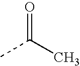 |
| 112 | 2-Cl | H | H | 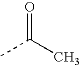 |
| 113 | 4-Br | H | H | 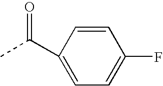 |
| 114 | 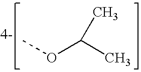 | H | H | 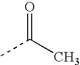 |
| 115 | 4 O—(CH₂)₃—CH₃] | H | H | 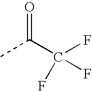 |
| 116 | 2-O—CH₃ | 3-Cl | 5-Cl | 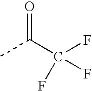 |
| 117 | 3-OH | H | H | 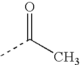 |
| 118 | 2-O—CH₃ | 5-O—CH₃ | H | 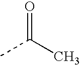 |
| 119 | 3-O—CH₃ | 4-O—CH₃ | H | 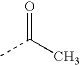 |
| 120 | 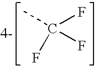 | H | H | 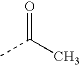 |
| 121 | 3-O—CH₂—CH₃ | 4-OH | H | 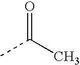 |
| 122 | 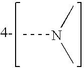 | H | H | 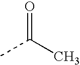 |
| 123 | 3-O—(CH₂)₂—CH₃ | H | H | 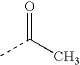 |
| 124 | 3-O—CH₃ | 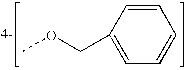 | H | 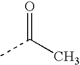 |
| 125 | 2-O—CH₂—CH₃ | H | H | 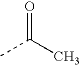 |
| 126 | 3-Cl | H | H | 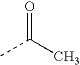 |
| 127 | 4-O—(CH₂)₃—CH₃ | H | H | 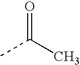 |
| 128 | 2-O—CH₃ | H | H | 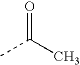 |
| 129 | 3-O—CH₃ | 4-O—(CH₂)₂—CH₃ | H | 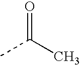 |
| 130 | 2-O—(CH₂)₂—CH₃ | H | H | 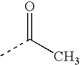 |
| 131 | 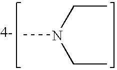 | H | H | 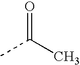 |
| 132 | 2-O—CH₃ | 4-O—CH₃ | H | 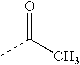 |
| 133 | 3-Cl | 5-Cl | H | 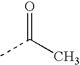 |
| 134 | 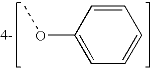 | H | H | 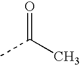 |
| 135 | 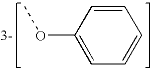 | H | H | 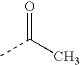 |
| 136 | 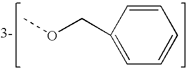 | H | H | 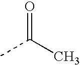 |
| 137 | 4-OH | H | H | 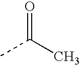 |
| 138 | 2-Cl | 5-Cl | H | 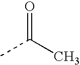 |
| 139 | 3-Cl | 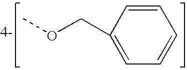 | H | 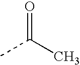 |
| 140 | 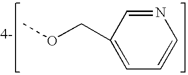 | H | H | 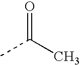 |
| 141 | 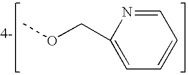 | H | H | 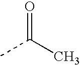 |
| 142 | 2-Cl | 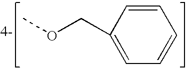 | H | 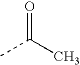 |
| 143 | 3-Cl | 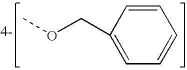 | 5-Cl | 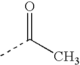 |
| 144 | 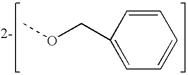 | 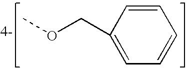 | H | 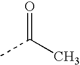 |
| 145 | 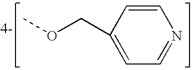 | H | H | 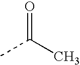 |
| 146 | 2-F | 4-F | H | 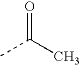 |
| 147 | 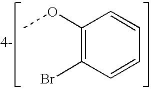 | H | H | 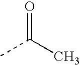 |
| 148 | 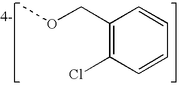 | H | H | 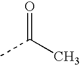 |
| 149 | 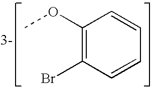 | H | H | 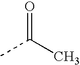 |
| 150 | 2-Cl | 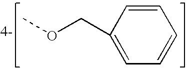 | 6-Cl | 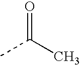 |
| 151 | 2-Cl | 4-Cl | H | 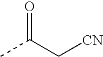 |
| 152 | 2-Cl | 4-Cl | H | 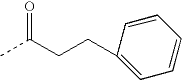 |
| 153 | 2-Cl | 4-Cl | H | 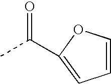 |
| 154 | 2-Cl | 4-Cl | H | 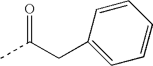 |
| 155 | 2-Cl | 4-Cl | H | 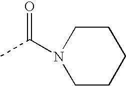 |
| 156 | 2-Cl | 4-Cl | H | 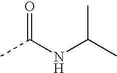 |
| 157 | 2-Cl | 4-Cl | H | 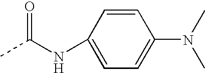 |
| 158 | 2-Cl | 4-Cl | H | 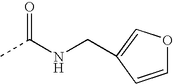 |
| 159 | 2-Cl | 4-Cl | H | 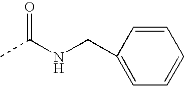 |

TABLE 2

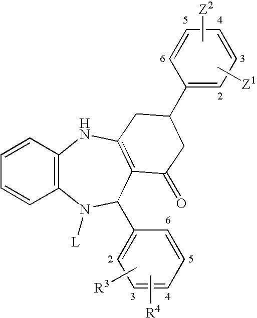

| Comp | | | | | |
|---|---|---|---|---|---|
| .No. | Z¹ | Z² | R³ | R⁴ | L |
| 160 | 4-Cl | H | 4-CH₃ | H | 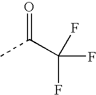 |
| 161 | 4-CH₃ | H | 4-CH₃ | H | 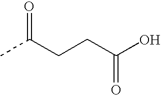 |
| 162 | 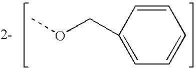 | H | H | H | 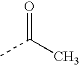 |
| 163 | 2-[—O—(CH₂)₂—CH₃] | | 4-OCH₃ | H | 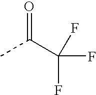 |
| 164 | 4-Cl | H | 4-Cl | H | 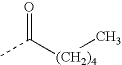 |
| 165 | 4-OCH₃ | H | 4-Cl | H | 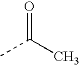 |
| 166 | 2-F | H | 4-CH₃ | H | 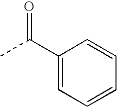 |
| 167 | 4-[—O—(CH₂)₃—CH₃] | H | H | H | 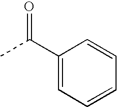 |
| 168 | 3-NO₂ | H | 4-OCH₃ | H | 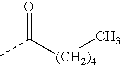 |
| 169 | 4-CH₃ | H | 2-Cl | H | 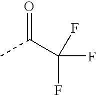 |
| 170 | 3-OCH₃ | 4-OCH₃ | 4-CH₃ | H | 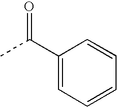 |
| 171 | 4-[—O—(CH₂)₃—CH₃] | H | H | H | 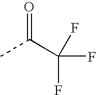 |
| 172 | 3-OCH₃ | 4-OCH₃ | 3-NO₂ | H | 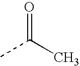 |
| 173 | 2-[—O—CH₂—CH₃] | H | 4-OCH₃ | H | 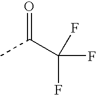 |
| 174 | 3-NO₂ | H | 3-OCH₃ | 4-OCH₃ | 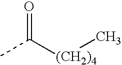 |
| 175 | 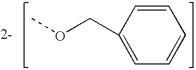 | H | H | H | 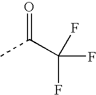 |
| 176 | 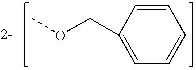 | H | H | H | 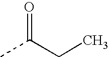 |
| 177 | 3-OCH₃ | 4-OCH₃ | 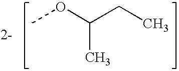 | H | 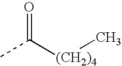 |
| 178 | 3-OCH₃ | 4-OCH₃ | 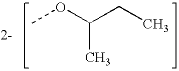 | H | 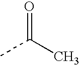 |
| 179 | 4-OCH₃ | H | 2-[—O—CH₂—CH₃] | H | 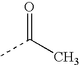 |
| 180 | 3-NO₂ | H | 3-OCH₃ | 4-OCH₃ | 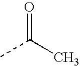 |
| 181 | 3-OCH₃ | 4-OCH₃ | 3-NO₂ | H | 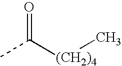 |
| 182 | 4-CH₃ | H | 2-Cl | H | 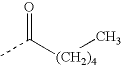 |
| 183 | 3-OCH₃ | 4-OCH₃ | 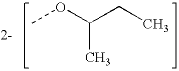 | H | 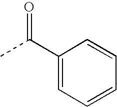 |
| 184 | 4-OCH₃ | 5-OCH₃ | 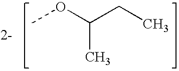 | H | 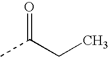 |
| 185 | 3-OCH₃ | 4-OCH₃ | 4-Cl | H | 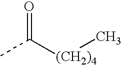 |
| 186 | 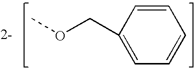 | H | 2-Cl | 4-Cl | 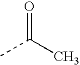 |
| 187 | 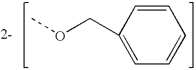 | H | 2-Cl | 4-Cl | 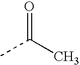 |
| 188 | 4-CH₃ | H | 4-OCH₃ | H | 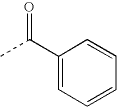 |
| 189 | 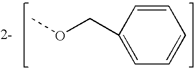 | H | 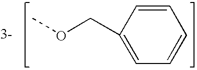 | H | 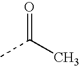 |
| 190 | 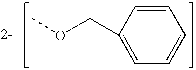 | H | 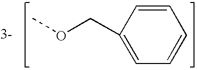 | H | 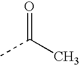 |
| 191 | 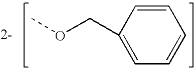 | H | 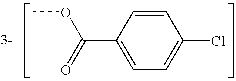 | H | 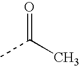 |
| 192 | 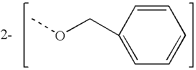 | H | 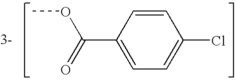 | H | 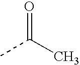 |
| 193 | 4-OCH₃ | 5-OCH₃ | 3-Cl | H | 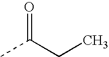 |
| 194 | 4-CH₃ | H | 4-OCH₃ | H | 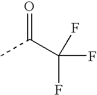 |
| 195 | 4-CH₃ | H | 3-Cl | H | 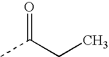 |
| 196 | 4-OCH₃ | H | 4-CH₃ | H | 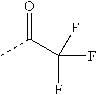 |
| 197 | 4-CH₃ | H | 3-OCH₃ | 4-OCH₃ | 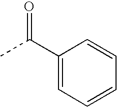 |
| 198 | 3-OCH₃ | 4-OCH₃ | 3-Cl | H | 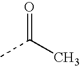 |
| 199 | H | H | 4-CH₃ | H | 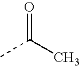 |
| 200 | 4-Cl | H | 4-OCH₃ | H | 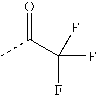 |
| 201 | 4-CH₃ | H | 4-CH₃ | H | 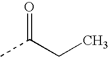 |
| 202 | 4-Cl | H | 3-NO₂ | H | 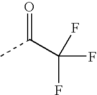 |
| 203 | 4-Cl | H | 3-Cl | H | 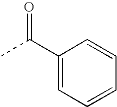 |
| 204 | 4-CH₃ | H | 3-Cl | H | 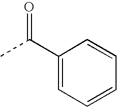 |
| 205 | 3-OCH₃ | 4-OCH₃ | 3-Cl | H | 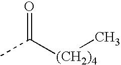 |
| 206 | 4-OCH₃ | H | 4-CH₃ | H | 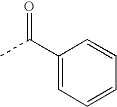 |
| 207 | 3-NO₂ | H | 3-OCH₃ | 4-OCH₃ | 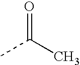 |
| 208 | 4-Cl | H | 3-Cl | H | 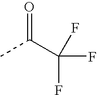 |
| 209 | 4-CH₃ | H | H | H | 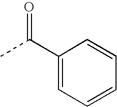 |
| 210 | 4-OCH₃ | H | 4-Cl | H | 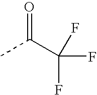 |
| 211 | 3-NO₂ | H | 4-Cl | H | 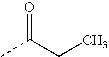 |
| 212 | 3-NO₂ | H | 4-CH₃ | H | 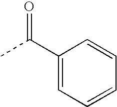 |
| 213 | 4-F | H | 3-OCH₃ | H | 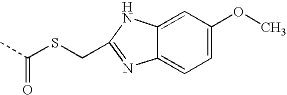 |
| 214 | 3-NO₂ | H | 4-Cl | H | 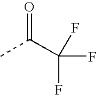 |
| 215 | 4-Cl | H | 4-OCH₃ | H | 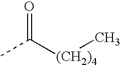 |
| 216 | 4-OCH₃ | H | 2-[—O—CH₂—CH₃] | H | 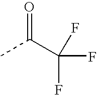 |
| 217 | 4-[—O—(CH₂)₃—CH₃] | H | H | H | 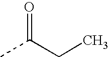 |
| 218 | 3-OCH₃ | 4-OCH₃ | 4-Cl | H | 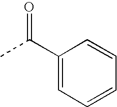 |
| 219 | 2-[—O—CH₂—CH₃] | H | 4-OCH₃ | H | 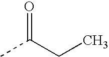 |
| 220 | 2-[—O—(CH₂)₂—CH₃] | H | 4-OCH₃ | H | 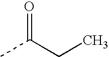 |
| 221 | 3-NO₂ | H | 4-OCH₃ | H | 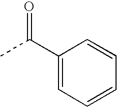 |
| 222 | 2-[—O—(CH₂)₂—CH₃] | H | 4-OCH₃ | H | 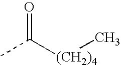 |
| 223 | 4-OCH₃ | H | 4-OCH₃ | H | 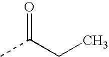 |
| 224 | H | H | 2-F | H | 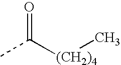 |
| 225 | 3-Cl | H | 4-CH₃ | H | 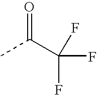 |
| 226 | 3-OCH₃ | 4-OCH₃ | 4-Cl | H | 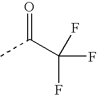 |
| 227 | 4-OCH₃ | H | 4-Cl | H | 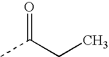 |
| 228 | 3-OCH₃ | 4-OCH₃ | 4-Cl | H | 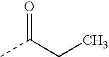 |
| 229 | 2-Cl | H | 4-CH₃ | H | 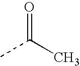 |
| 230 | 4-CH₃ | H | 3-Cl | H | 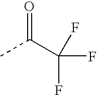 |
| 231 | 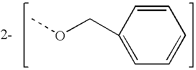 | H | H | H | 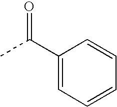 |
| 232 | 4-OCH₃ | H | 4-Cl | H | 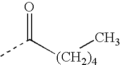 |
| 233 | 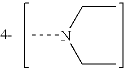 | H | 4-O—CH₃ | H | 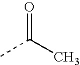 |
| 234 | 3-NO₂ | H | H | H | 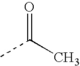 |
| 235 | 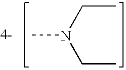 | H | H | H | 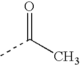 |
| 236 | 2-F | H | H | H | 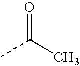 |
| 237 | 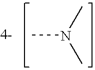 | H | H | H | 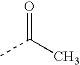 |

TABLE 3

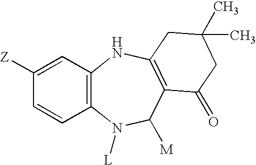

| Comp. | | | |
|---|---|---|---|
| No. | M | L | Z |
| 238 | 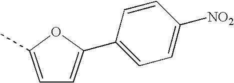 | 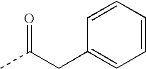 | H |
| 239 | 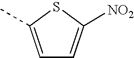 | 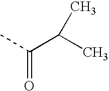 | H |
| 240 | 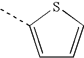 | 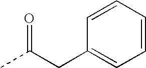 | H |
| 241 | 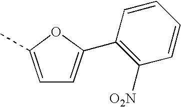 | 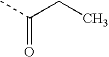 | H |
| 242 | 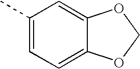 | 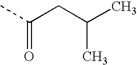 | H |
| 243 | 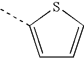 | 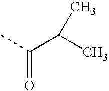 | H |
| 244 | 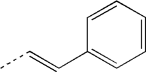 | 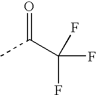 | H |
| 245 | 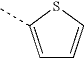 | 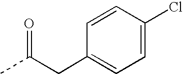 | H |
| 246 | 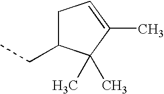 | 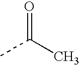 | H |
| 247 | 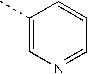 | 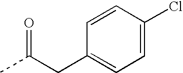 | H |
| 248 | 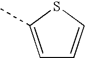 | 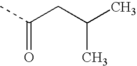 | H |
| 249 | 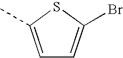 | 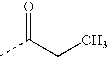 | H |
| 250 | 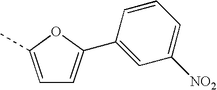 | 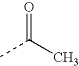 | H |
| 251 | 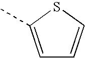 | 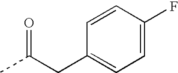 | H |
| 252 | 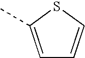 | 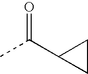 | H |
| 253 | 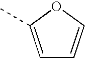 | 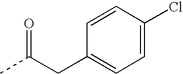 | H |
| 254 | 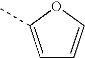 | 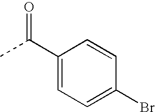 | H |
| 255 | 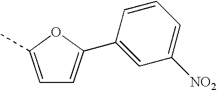 | 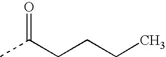 | H |
| 256 | 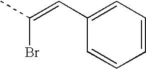 | 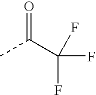 | H |
| 257 | 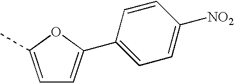 | 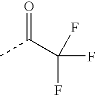 | H |
| 258 | 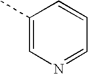 | 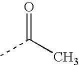 | H |
| 259 | 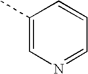 | 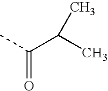 | H |
| 260 | 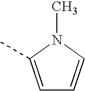 | 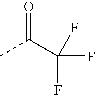 | H |
| 261 | 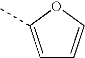 | 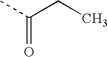 | 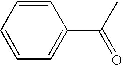 |
| 262 | 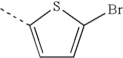 | 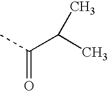 | H |
| 263 | 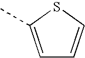 | 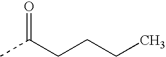 | H |
| 264 | 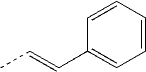 | 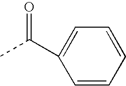 | H |
| 265 | 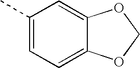 | 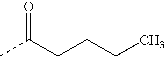 | H |
| 266 | 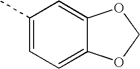 | 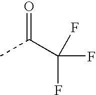 | H |
| 267 | 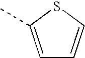 | 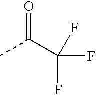 | H |
| 268 | 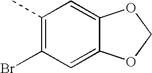 | 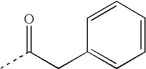 | H |
| 269 | 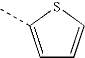 | 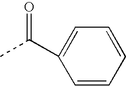 | H |
| 270 | 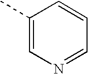 | 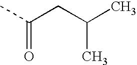 | H |
| 271 | 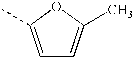 | 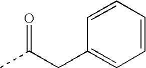 | H |
| 272 | 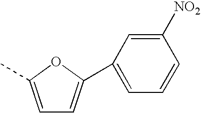 | 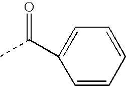 | H |
| 273 | 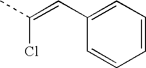 | 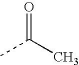 | H |
| 274 | 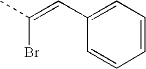 | 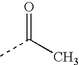 | H |
| 275 | 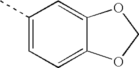 | 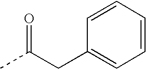 | H |
| 276 | 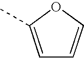 | 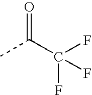 | H |
| 277 | 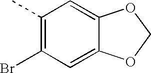 | 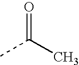 | H |
| 278 | 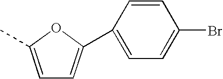 | 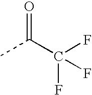 | H |
| 279 | 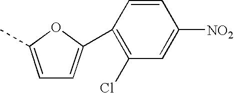 | 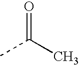 | H |
| 280 | 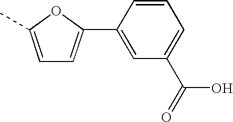 | 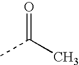 | H |
| 281 | 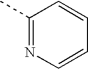 | 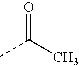 | H |
| 282 | 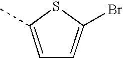 | 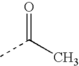 | H |
| 283 | 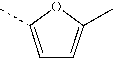 | 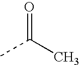 | H |
| 284 | 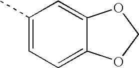 | 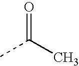 | H |

TABLE 4

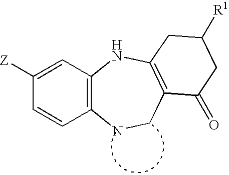

| Comp. No. | R¹ | 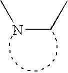 | Z |
|---|---|---|---|
| 285 | phenyl | 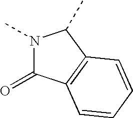 | 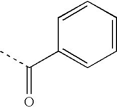 |

TABLE 5

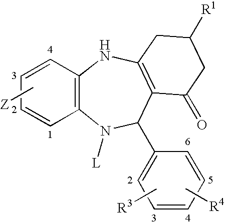

| Comp. | | | | | |
|---|---|---|---|---|---|
| No. | R¹ | R³ | R⁴ | L | Z |
| 286 | 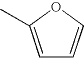 | 3-NO₂ | H | 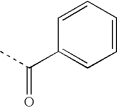 | H |
| 287 | 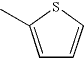 | 4-OCH₃ | H | 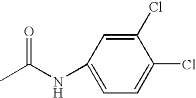 | H |
| 288 | 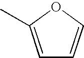 | 3-NO₂ | H | 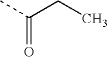 | H |
| 289 | 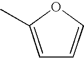 | 4-Cl | H | 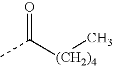 | H |
| 290 | 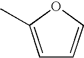 | 3-NO₂ | H | 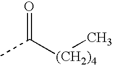 | H |
| 291 | H | 2-Cl | 4-Cl | 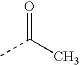 | H |
| 292 | 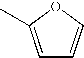 | 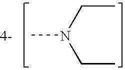 | H | 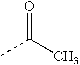 | H |
| 293 | 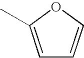 | 3-O—CH₃ | 4-O—CH₃ | 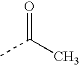 | H |
| 294 | CH₃ | 2-Cl | 4-Cl | 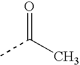 | H |

TABLE 6

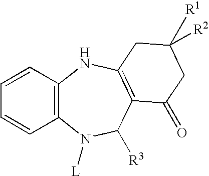

| Comp | | | | |
|---|---|---|---|---|
| .No. | R¹ | R² | R³ | L |
| 295 | 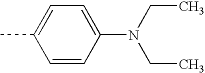 | H | 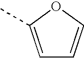 | 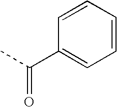 |
| 296 | 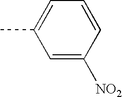 | H | 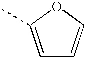 | 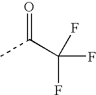 |
| 297 | 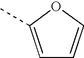 | H | 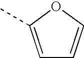 | 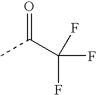 |
| 298 | 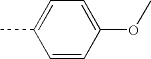 | H | 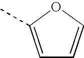 | 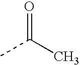 |
| 299 | 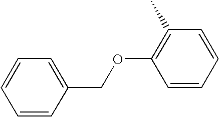 | H | 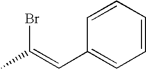 | 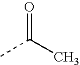 |
| 300 | 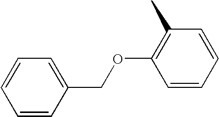 | H | 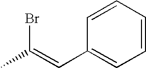 | 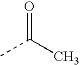 |
| 302 | CH₃ | CH₃ | 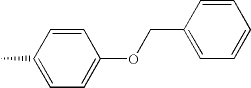 | 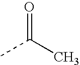 |
| 301 | CH₃ | CH₃ | 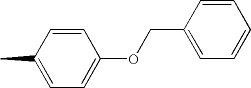 | 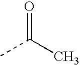 |
| 304 | CH₃ | CH₃ | 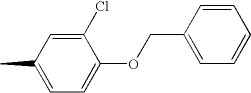 | 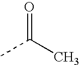 |
| 303 | CH₃ | CH₃ | 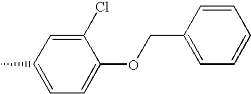 | 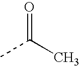 |
| 306 | CH₃ | CH₃ | 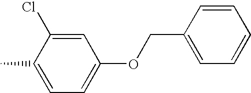 | 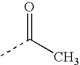 |
| 305 | CH₃ | CH₃ | 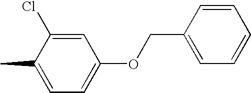 | 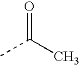 |

TABLE 7

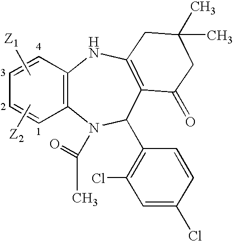

| Comp. | | |
|---|---|---|
| No. | Z¹ | Z² |
| 307 | 2-Cl | 3-Cl |
| 308 | 2-CH₃ | 3-CH₃ |
| 309 | 2-O—CH₃ | 3-O—CH₃ |
| 310 | 2-F | 3-F |
| 311 | 1-Br | H |
| 312 | 4-Br | H |
| 313 | 1-CN | H |
| 314 | 1-CH₃ | H |
| 315 | 4-CH₃ | H |
| 316 | 4-CN | H |
| 317 | 4-O—(CH₂)₃—CH₃ | H |
| 318 | 2-CN | H |
| 319 | 2-N | H |
| 320 | 4-O—CH₃ | H |
| 321 | 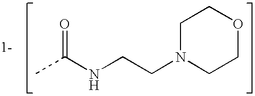 | H |

TABLE 8

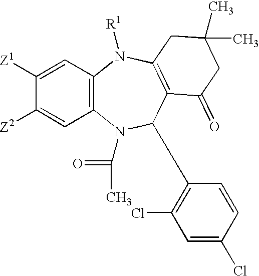

| Comp. | | | |
|---|---|---|---|
| No. | Z¹ | Z² | R¹ |
| 322 | H | H | CH₃ |
| 323 | H | H | 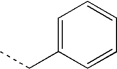 |

TABLE 9

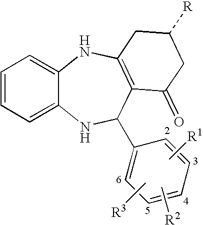

| Comp. | | | | |
|---|---|---|---|---|
| No | R | R¹ | R² | R³ |
| 324 | 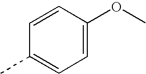 | 2-CF₃ | H | H |
| 325 | 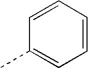 | 4-Cl | H | H |
| 326 | 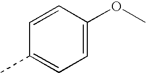 | 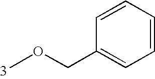 | H | H |
| 327 | 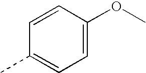 | 3-F | H | H |
| 328 | 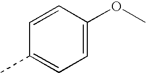 | 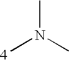 | H | H |
| 329 | 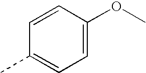 | 4-CF₃ | H | H |
| 330 | 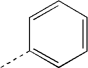 | 3-O—CH₃ | H | H |
| 331 | 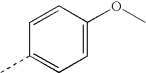 | 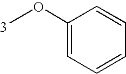 | H | H |
| 332 | 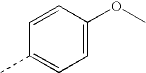 | 4-O—CH₂—CH₃ | H | H |
| 333 | 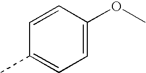 | 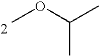 | H | H |
| 334 | 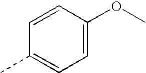 | 2-Cl | 6-F | H |
| 335 | 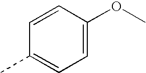 | 2-O—CH₃ | 5-Br | H |
| 336 | 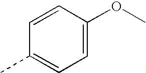 | 2-O—CH₃ | 5-O—CH₃ | H |
| 337 | 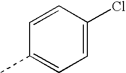 | 3-NO₂ | H | H |
| 338 | 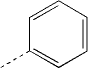 | 2-O—(CH₂)₃—CH₃ | H | H |
| 339 | 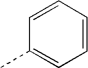 | 3-O—CH₃ | 4-O—CH₃ | 5-O—CH₃ |
| 340 | 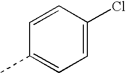 | 2-Cl | H | H |
| 341 | 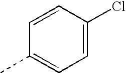 | 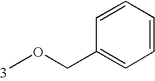 | H | H |
| 342 | 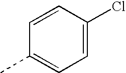 | 3-Br | 4-OH | 5-O—CH₃ |
| 343 | 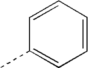 | 4-CF₃ | H | H |
| 344 | 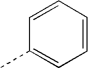 | 4-CH₃ | H | H |
| 345 | 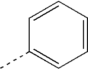 | 2-Br | H | H |
| 346 | 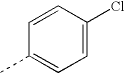 | 2-Cl | 3-Cl | H |
| 347 | 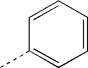 | 2-O—CH₃ | 3-O—CH₃ | H |
| 348 | 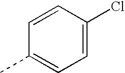 | 2-O—CH₃ | 3-O—CH₃ | H |
| 349 | 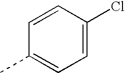 | 2-CF₃ | H | H |
| 350 | 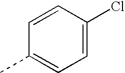 | 3-Cl | H | H |
| 351 | 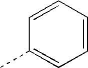 | 2-O—CH₃ | 5-O—CH₃ | H |
| 352 | 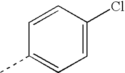 | 3-O—CH₃ | 4-O—CH₃ | H |
| 353 | 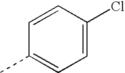 | 3-Br | 4-OH | 5-O—CH₂—CH₃ |
| 354 | 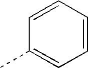 | 3-CH₃ | H | H |
| 355 | 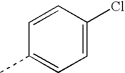 | 3-Cl | 4-Cl | H |
| 356 | 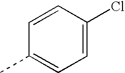 | 2-O—CH₃ | 5-O—CH₃ | H |
| 357 | 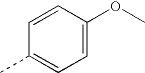 | 2-Cl | 3-Cl | H |
| 358 | 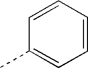 | 3-NO₂ | 4-CH₃ | H |
| 359 | 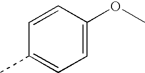 | 3-NO₂ | 4-CH₃ | H |
| 360 | 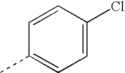 | 3-O—CH₃ | H | H |
| 361 | 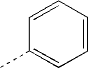 | 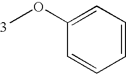 | H | H |
| 362 | 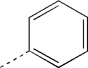 | 4-S—CH₃ | H | H |
| 363 | 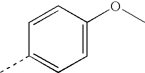 | 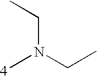 | H | H |
| 364 | 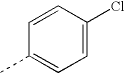 | 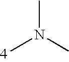 | H | H |
| 365 | 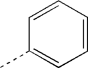 | 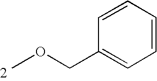 | H | H |
| 366 | 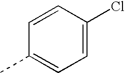 | 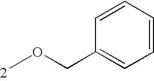 | H | H |
| 367 | 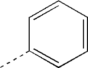 | 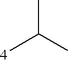 | H | H |
| 368 | 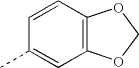 | 4-S—CH₃ | H | H |
| 369 | 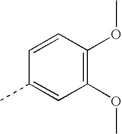 | 2-O—CH₃ | H | H |
| 370 | 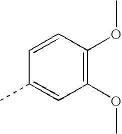 | 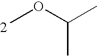 | H | H |
| 371 | 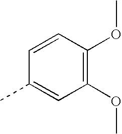 | 2-O—CH₃ | 5-O—CH₃ | H |
| 372 | 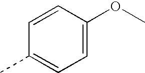 | 2-O—CH₃ | 4-O—CH₃ | H |
| 373 | 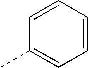 | 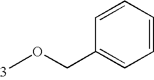 | H | H |
| 374 | 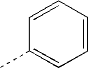 | 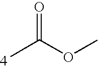 | H | H |
| 375 | 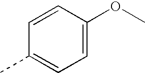 | 2-Cl | 5-NO₂ | H |
| 376 | 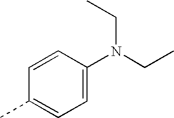 | H | H | H |
| 377 | 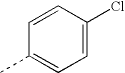 | 4-CF₃ | H | H |
| 378 | 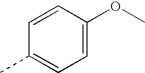 | 3-Br | 4-OH | 5-O—CH₂—CH₃ |
| 379 | 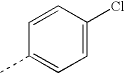 | 3-O—CH₂—CH₃ | H | H |
| 380 | 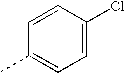 | 3-F | H | H |
| 381 | 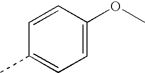 | 2-CH₃ | H | H |
| 382 | 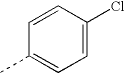 | 4-S—CH₃ | H | H |
| 383 | 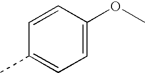 | 2-Cl | H | H |
| 384 | 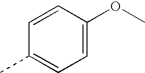 | 4-CH₃ | H | H |
| 385 | 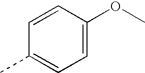 | 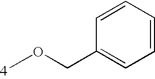 | 3-O—CH₃ | H |
| 386 | 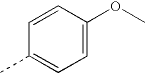 | 3-O—CH₂—CH₃ | 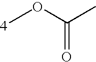 | H |
| 387 | 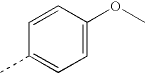 | 4-NO₂ | H | H |
| 388 | 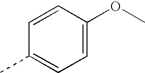 | 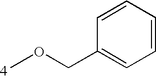 | H | H |
| 389 | 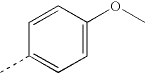 | 3-NO₂ | 4-Cl | H |
| 390 | 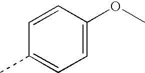 | 2-Br | H | H |
| 391 | 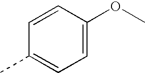 | 3-CH₃ | H | H |
| 392 | 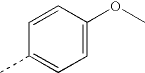 | 3-OH | H | H |
| 393 | 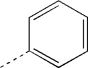 | 2-O—CH₂—CH₃ | H | H |
| 394 | 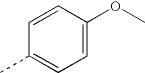 | 3-O—CH₃ | 4-O—(CH₂)₂—CH₃ | H |
| 395 | 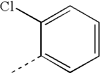 | 4-CH₃ | H | H |
| 396 | 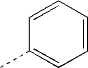 | 2-NO₂ | H | H |
| 397 | 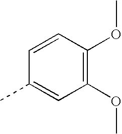 | 3-Br | 4-OH | 5-O—CH₃ |
| 398 | 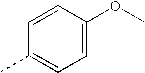 | 2-O—CH₃ | 4-O—CH₃ | 5-O—CH₃ |
| 399 | 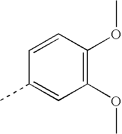 | 3-Br | 4-OH | 5-O—CH₂—CH₃ |
| 400 | 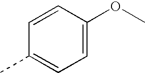 | 2-Br | 4-O—CH₃ | 5-O—CH₃ |
| 401 | 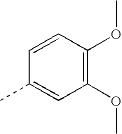 | 2-O—CH₃ | 3-O—CH₃ | H |
| 402 | 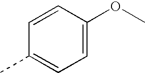 | 3-Br | 4-O—CH₃ | 5-O—CH₃ |
| 403 | 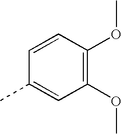 | 2-O—CH₃ | 4-O—CH₃ | 5-O—CH₃ |
| 404 | 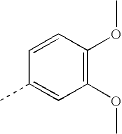 | 2-O—CH₃ | 5-Br | H |
| 405 | 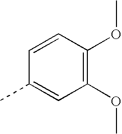 | 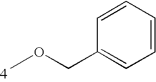 | 3-O—CH₃ | H |
| 406 | 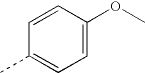 | 3-Br | 4-O—CH₃ | 5-O—CH₂—CH₃ |
| 407 | 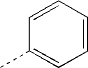 | 2-CF₃ | H | H |
| 408 | 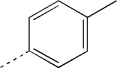 | 4-CH₃ | H | H |
| 409 | 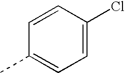 | 4-CH₃ | H | H |
| 410 | 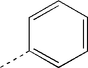 | 3-O—CH₂CH₃ | H | H |
| 411 | 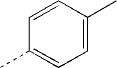 | 2-Cl | H | H |
| 412 | 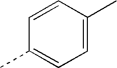 | 2-F | H | H |
| 413 | 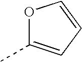 | 4-Cl | H | H |
| 414 | 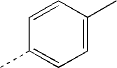 | H | H | H |
| 415 | 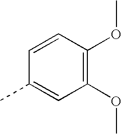 | 4-Cl | H | H |
| 416 | 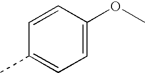 | 4-Cl | H | H |
| 417 | 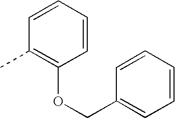 | 2-Cl | 4-Cl | H |
| 418 | 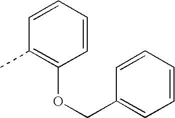 | 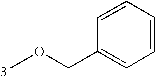 | H | H |
| 419 | 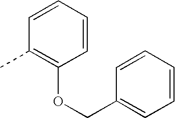 | 2-Cl | 4-Cl | H |
| 420 | 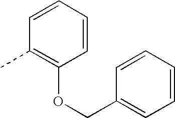 | 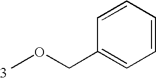 | H | H |
| 421 | H | 2-Cl | 4-Cl | H |
| 422 | CH₃ | 2-Cl | 4-Cl | H |

TABLE 10

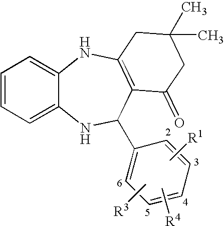

| Comp. | | | |
|---|---|---|---|
| No | R¹ | R² | R³ |
| 423 | 2-Cl | 4-Cl | H |
| 424 | 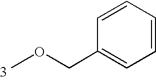 | 4-O—CH₃ | H |
| 425 | 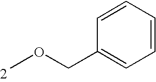 | H | H |
| 426 | 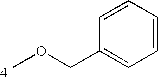 | H | H |
| 427 | 2-Cl | 3-Cl | H |
| 428 | 2-Cl | 6-F | H |
| 429 | 4-CF₃ | H | H |
| 430 | 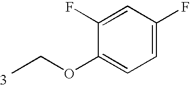 | 4-O—CH₃ | H |
| 431 | 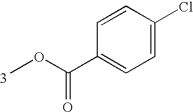 | H | H |
| 432 | 2-COOH | H | H |
| 433 | 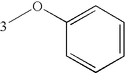 | H | H |
| 434 | 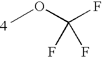 | H | H |
| 435 | 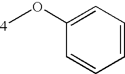 | H | H |
| 436 | 3-Cl | 5-Cl | H |
| 437 | 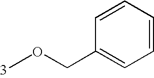 | H | H |
| 438 | 2-Cl | 5-Cl | H |
| 439 | 3-Cl | 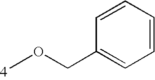 | H |
| 440 | 4-OH | H | H |
| 441 | 2-F | 4-F | H |
| 442 | 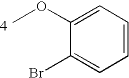 | H | H |
| 443 | 2-Cl | 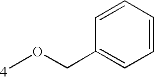 | H |
| 444 | 3-Cl | 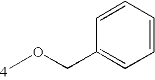 | 5-Cl |
| 445 | 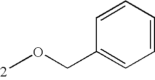 | 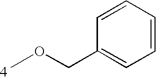 | H |
| 446 | 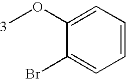 | H | H |
| 447 | 2-Cl | 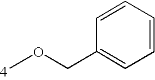 | 6-Cl |
| 448 | 4-NO₂ | H | H |

TABLE 11

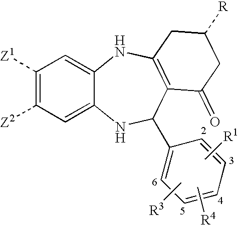

| Comp. | | | | | | |
|---|---|---|---|---|---|---|
| No | R | R¹ | R² | R³ | Z¹ | Z² |
| 449 | H | 3-NO₂ | H | H | H | 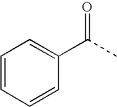 |
| 450 | 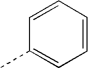 | 4-Cl | H | H | 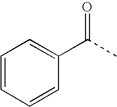 | H |
| 451 | H | 2-O—CH₃ | 5-O—CH₃ | H | 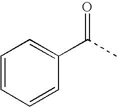 | H |

TABLE 12

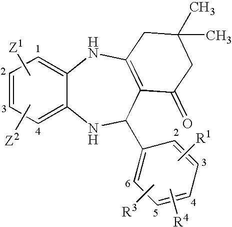

| Comp. | | | | | |
|---|---|---|---|---|---|
| No | R¹ | R² | R³ | Z¹ | Z² |
| 452 | 3-NO₂ | 4-Cl | H | 2-CH₃ | 3-CH₃ |
| 453 | 3-O—CH₃ | 4-O—CH₃ | H | 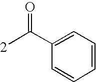 | H |
| 454 | 2-F | 6-F | H | 2-CH₃ | 3-CH₃ |
| 455 | 3-NO₂ | H | H | 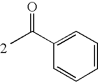 | H |
| 456 | 3-O—CH₃ | 4-O—CH₃ | H | H | 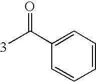 |
| 457 | 2-Cl | H | H | H | 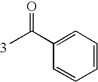 |
| 458 | 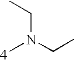 | H | H | H | 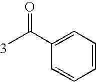 |
| 459 | 4-Cl | H | H | H | 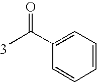 |
| 460 | 3-Cl | H | H | H | 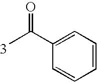 |
| 461 | 4-F | H | H | H | 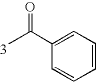 |
| 462 | 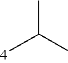 | H | H | H | 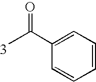 |
| 463 | 2-Cl | H | H | 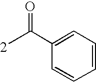 | H |
| 464 | 2-Cl | 4-Cl | H | 2-CH₃ | 3-CH₃ |
| 465 | 2-Cl | 4-Cl | H | 2-Cl | 3-Cl |
| 466 | 2-Cl | 4-Cl | H | 2-F | 3-F |
| 467 | 2-Cl | 4-Cl | H | 2-O—CH₃ | 3-O—CH₃ |
| 468 | 2-Cl | 4-Cl | H | 4-Br | H |
| 469 | 2-Cl | 4-Cl | H | 4-CH₃ | H |
| 470 | 2-Cl | 4-Cl | H | 4-CN | H |
| 471 | 2-Cl | 4-Cl | H | 1-CH₃ | H |
| 472 | 2-Cl | 4-Cl | H | 2-CN | H |
| 473 | 2-Cl | 4-Cl | H | 3-CN | H |
| 474 | 2-Cl | 4-Cl | H | 1-NH₂ | H |

TABLE 13

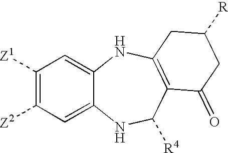

| Comp. | | | | |
|---|---|---|---|---|
| No | R | R⁴ | Z¹ | Z² |
| 475 | 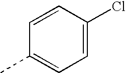 | 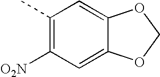 | H | H |
| 476 | 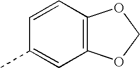 | 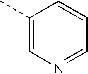 | H | H |
| 477 | 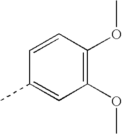 | 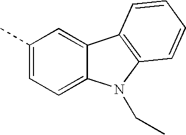 | H | H |
| 478 | 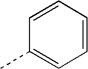 | 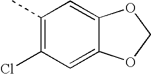 | H | H |
| 479 | 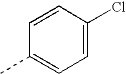 | 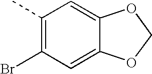 | H | H |
| 480 | 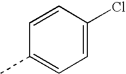 | 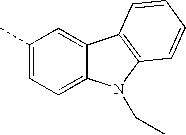 | H | H |
| 481 | 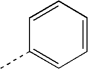 | 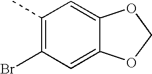 | H | H |
| 482 | 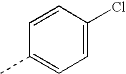 | 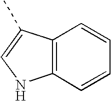 | H | H |
| 483 | 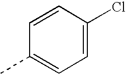 | 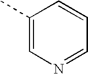 | H | H |
| 484 | 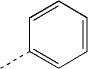 | 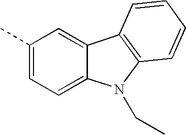 | H | H |
| 485 | 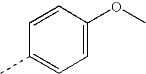 | 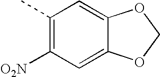 | H | H |
| 486 | 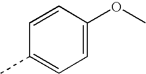 | 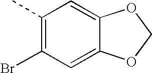 | H | H |
| 487 | 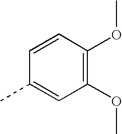 | 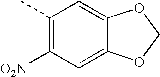 | H | H |
| 488 | 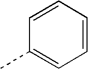 | 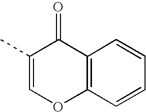 | H | H |
| 489 | 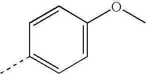 | 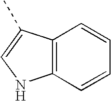 | H | H |
| 490 | 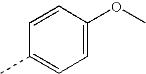 | 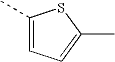 | H | H |
| 491 | 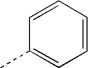 | 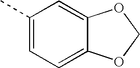 | 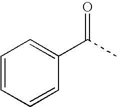 | H |
| 492 | 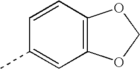 | 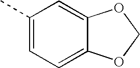 | H | H |
| 493 | 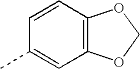 | 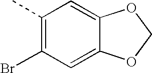 | H | H |
| 494 | 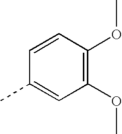 | 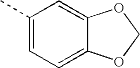 | H | H |
| 495 | 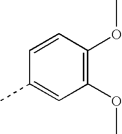 | 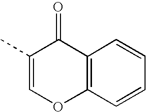 | H | H |
| 496 | 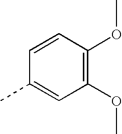 | 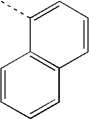 | H | H |
| 497 | 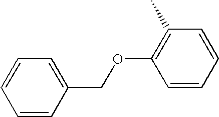 | 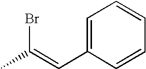 | H | H |
| 498 | 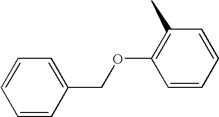 | 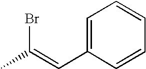 | H | H |

TABLE 14

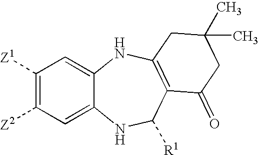

| Comp. | | | |
|---|---|---|---|
| No | R⁴ | Z¹ | Z² |
| 499 | 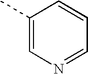 | H | H |
| 500 | 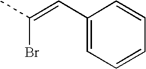 | H | H |
| 501 | 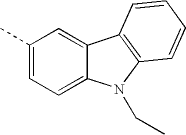 | H | H |
| 502 | 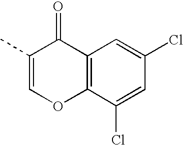 | H | H |
| 503 | 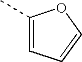 | 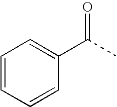 | H |
| 504 | 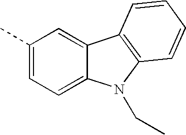 | CH₃ | CH₃ |
| 505 | 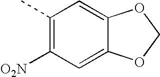 | H | H |
| 506 | 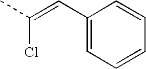 | H | H |

TABLE 15

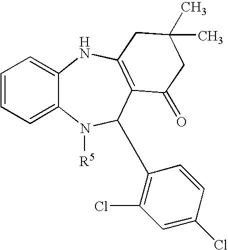

| Comp. | |
|---|---|
| No | R⁵ |
| 507 | CH₃ |
| 508 | CH₂—CH₃ |
| 509 | CH₂—CH₂—CH₃ |
| 510 | 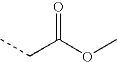 |
| 511 | 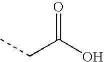 |

TABLE 16

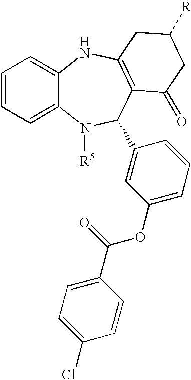

| Comp. | |
|---|---|
| No | R |
| 512 | 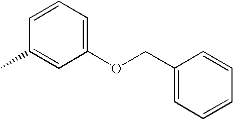 |
| 513 | 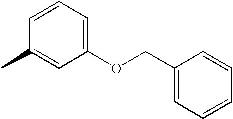 |

TABLE 17

| Comp. | |
|---|---|
| No | Structure |
| 514 | 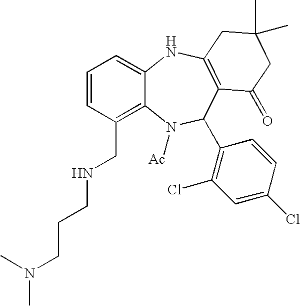 |
| 515 | 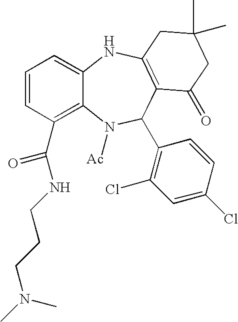 |
| 516 | 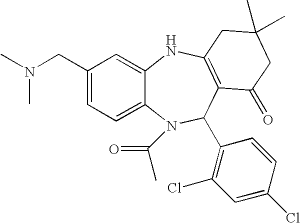 |
| 517 | 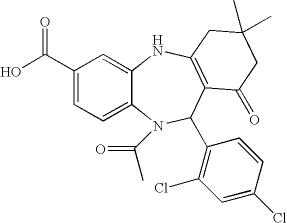 |
| 518 | 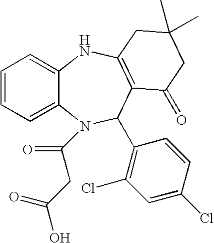 |
| 519 | 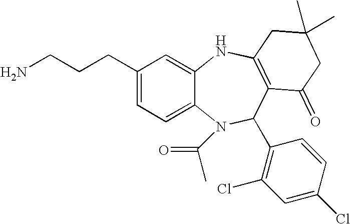 |
| 520 | 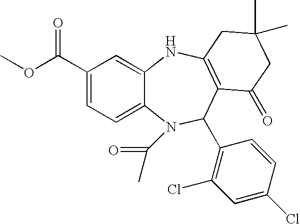 |
| 521 | 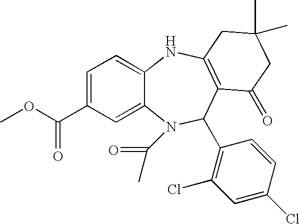 |
| 522 | 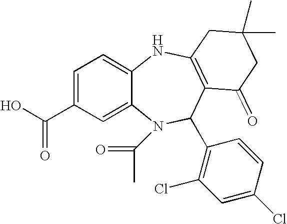 |
| 523 | 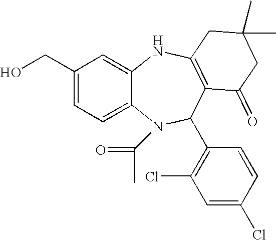 |
| 524 | 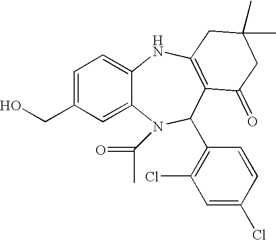 |
| 525 | 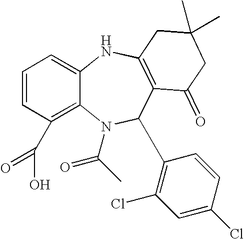 |
| 526 | 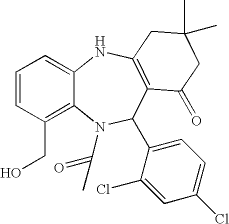 |
| 527 | 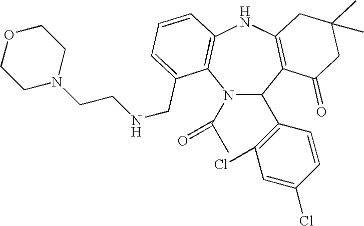 |
| 528 | 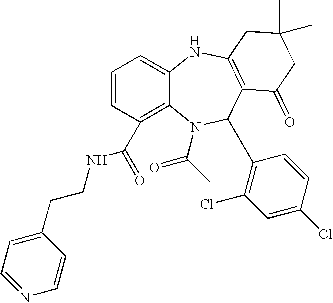 |
| 529 | 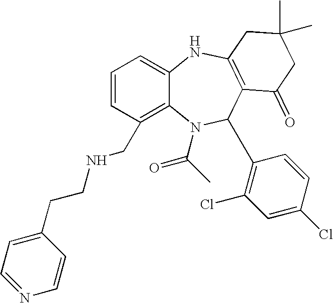 |
| 530 | 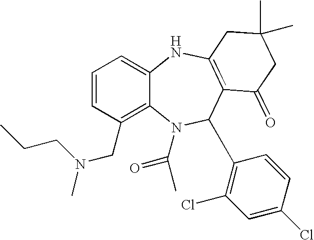 |
| 531 | 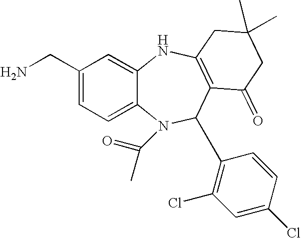 |
| 532 | 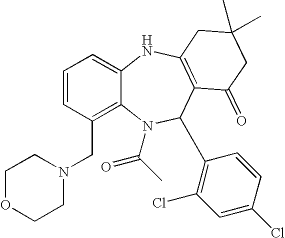 |
| 533 | 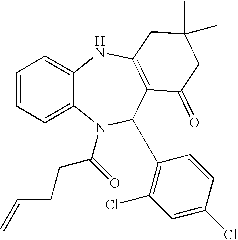 |
| 534 | 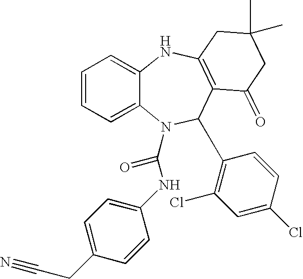 |
| 535 | 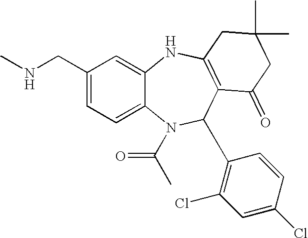 |
| 536 | 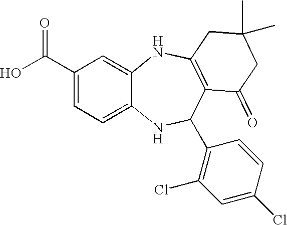 |
| 537 | 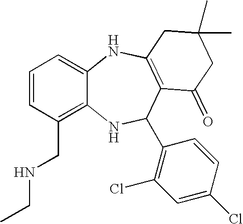 |
| 538 | 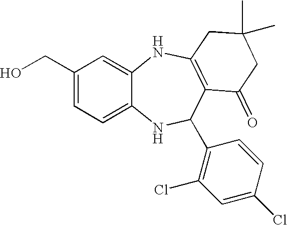 |
| 539 | 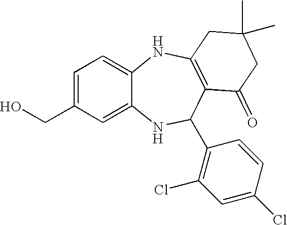 |
| 540 | 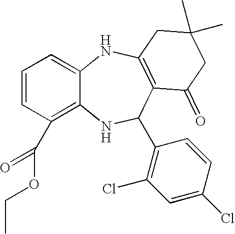 |
| 541 | 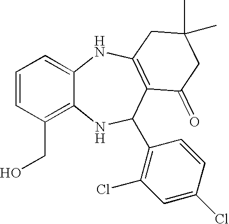 |

### Antiviral Testing

The compounds of formula (I) were tested for anti-HCV activity in an assay determining their activity against NS5b polymerase and in an HCV replicon assay

### A) NS5b Polymerase Assay

### a) Protein Purification

The cDNA encoding NS5B amino acid 1-570 (HC-J4, genotype 1b, pCV-J4L6S, genebank accession number AF054247) was subcloned into the Nhe I and Xho I restriction sites of pET-21b. Expression of the subsequent His-tagged C-terminal 21 amino acid deleted NS5B was performed as follows:

Following transformation in BL21 (DE3) competent cells, bacterial cells were grown in 22 liter LB/Amp media until to reach OD₆₀₀=0.4-0.6. Protein expression was induced by addition of IPTG 0.4 mM, supplemented with 10 μM MgCl₂, and incubated for 14-16 hrs at 20° C. Cells were harvested, resuspended in lysis buffer (20 mM Tris-HCl pH=7.5, 0.3 M NaCl, 10% glycerol, 0.1% NP40, 4 mM MgCl₂, 14 mM beta-mercaptoethanol, with tablet of EDTA-free protease cocktail inhibitors) and lysed by sonification. The cell lysate was cleared by high-speed centrifugation (20K×g for 30 min), captured on Ni-NTA beads for 70 min at 4° C., and eluted with 25 mM Hepes pH 7.5, 0.5 M NaCl, 10% glycerol, 14 mM BME, 500 mM imidazole. The eluent was dialysed against 25 mM Hepes pH 7.5, 10% glycerol, 50 mM NaCl, 14 mM BME, after which the protein was further purified by heparin chromatography using the same buffer with 1 M NaCl for elution. Fractions containing pure protein were collected, dialyzed against storage buffer 25 mM Hepes pH=7.5, 300 mM NaCl, 10% glycerol, 14 mM BME), and flash-freezed in liquid nitrogen. This procedure yielded approximately 40 mg of protein. The protein was judged to be at least 90% pure by SDS PAGE Coomassie staining.

b) Protein Sequence
PDB: 1nb4, Apo form
M A S S M S Y T W T G A L I T P C A A E E S K L P I N P L S N S L L R H H N M
V Y A T T S R S A S L R Q K K V T F D R L Q V L D D H Y R D V L K E M K A K
A S T V K A K L L S I E E A C K L T P P H S A K S K F G Y G A K D V R N L S S
R A V N H I R S V W E D L L E D T E T P I D T T I M A K S E V F C V Q P E K G
G R K P A R L I V F P D L G V R V C E K M A L Y D V V S T L P Q A V M G S S
Y G F Q Y S P K Q R V E F L V N T W K S K K C P M G F S Y D T R C F D S T V
T E S D I R V E E S I Y Q C C D L A P E A R Q A I R S L T E R L Y I G G P L T N
S K G Q N C G Y R R C R A S G V L T T S C G N T L T C Y L K A T A A C R A A
K L Q D C T M L V N G D D L V V I C E S A G T Q E D A A A L R A F T E A M T
R Y S A P P G D P P Q P E Y D L E L I T S C S S N V S V A H D A S G K R V Y Y
L T R D P T T P L A R A A W E T A R H T P I N S W L G N I I M Y A P T L W A R
M I L M T H F F S I L L A Q E Q L E K A L D C Q I Y G A C Y S I E P L D L P Q I
I E R L H G L S A F T L H S Y S P G E I N R V A S C L R K L G V P P L R T W R
H R A R S V R A K L L S Q G G R A A T C G R Y L F N W A V R T K L K L T P I
P A A S Q L D L S G W F V A G Y S G G D I Y H S L S R A R P R A A A L E H H
H H H H
Calc. Mol. Properties 64941.4 g/mol

### c) Biochemical RdRp Assay

Measurement of HCV NS5B polymerization activity was performed by evaluating the amount of radiolabeled GTP incorporated by the enzyme in a newly synthesized RNA using heteropolymeric RNA template/primer. The highthroughput RdRp assay was carried out in 384-well plates using 200 nM enzyme, 0.1 μCi of ³H GTP, 5 mM MgCl₂, 600 nM GTP, 30 nM PolyC, 300 nM 5′-biotinylated oligo(rG13)/poly(rC) in 20 mM Tris pH 7.5, 21 mM KCl, 2.5 mM DTT, 16.7 mM NaCl and 0.17 mM EDTA. Test compounds were dissolved in dimethylsulfoxide. The test compounds were added to the preformed polymerase-template complex, and incubated at room temperature (RT) for 15 min before the addition of NTPs. The 30-μl reaction was terminated after 2 h at 25° C. upon addition of 30-μl PVT-SPA beads (Amersham Biosciences RPNQ0009, 5 mg/ml in 0.5 M EDTA). After incubation at 25° C. for 30 min, the plate was counted using a Packard TopCount microplate reader (30 sec/well, 1 min count delay) and EC50 values were calculated.

### B) Replicon Assay

### a) Stable Replicon Cell Reporter Assays:

The compounds of the present invention were examined for activity in the inhibition of HCV RNA replication in a cellular assay. The assay demonstrated that the present compounds exhibit activity against HCV replicons functional in a cell culture. The cellular assay was based on a bicistronic expression construct, as described by Lohmann et al. (1999) Science vol. 285 pp. 110-113 with modifications described by Krieger et al. (2001) Journal of Virology 75: 4614-4624, in a multi-target screening strategy. In essence, the method was as follows.

The assay utilized the stably transfected cell line Huh-7 luc/neo (hereafter referred to as Huh-Luc). This cell line harbored an RNA encoding a bicistronic expression construct comprising the wild type NS3-NS5B regions of HCV type 1b translated from an Internal Ribosome Entry Site (IRES) from encephalomyocarditis virus (EMCV), preceded by a reporter portion (FfL-luciferase), and a selectable marker portion (neo^{R}, neomycine phosphotransferase). The construct was bordered by 5′ and 3′ NTRs (non-translated regions) from HCV type 1b. Continued culture of the replicon cells in the presence of G418 (neo^{R}) was dependent on the replication of the HCV RNA. The stably transfected replicon cells that expressed HCV RNA, which replicated autonomously and to high levels, encoding inter alia luciferase, were used for screening the antiviral compounds.

### b) Cellular Assay Experimental Method:

The replicon cells were plated in 384 well plates in the presence of the test and control compounds which were added in various concentrations. Following an incubation of three days, HCV replication was measured by assaying luciferase activity (using standard luciferase assay substrates and reagents and a Perkin Elmer ViewLux™ ultraHTS microplate imager). Replicon cells in the control cultures had high luciferase expression in the absence of any inhibitor. The inhibitory activity of the compound on luciferase activity was monitored on the Huh-Luc cells, enabling a dose-response curve for each test compound. IC50 values were then calculated, which value represents the amount of the compound required to decrease by 50% the level of detected luciferase activity, or more specifically, the ability of the genetically linked HCV replicon RNA to replicate.

The activities of the compounds tested in the above assays are given below. A strip, i.e., indicates that no result is available.

TABLE 18

| Compound | NS5B Polymerase Assay | Replicon Assay |
|---|---|---|
| No. | IC₅₀ (μM) | EC₅₀ (μM) |
| 245 | >42.612 | =3.506 |
| 33 | >42.612 | =24.740 |
| 254 | >42.612 | =15.302 |
| 251 | >42.612 | =9.884 |
| 250 | >42.612 | =7.950 |
| 22 | >42.612 | =10.824 |
| 40 | >42.667 | >24.713 |
| 26 | >42.612 | =17.830 |
| 48 | =15.915 | =16.717 |
| 30 | >42.612 | =23.008 |
| 13 | >42.660 | =20.373 |
| 88 | =18.467 | =6.096 |
| 45 | =4.826 | =6.794 |
| 74 | >42.656 | >26.654 |
| 20 | >42.612 | =6.576 |
| 248 | >42.612 | =7.236 |
| 95 | >33.333 | — |
| 275 | >42.658 | =10.085 |
| 84 | >42.677 | =20.852 |
| 259 | =29.507 | >33.046 |
| 8 | >42.666 | =5.652 |
| 263 | >42.670 | =10.392 |
| 1 | >42.659 | =8.527 |
| 85 | >42.671 | =21.418 |
| 5 | >42.661 | =19.866 |
| 262 | >42.667 | =5.538 |
| 29 | >42.662 | =6.795 |
| 50 | >42.662 | >31.996 |
| 82 | >42.675 | =6.804 |
| 52 | >42.667 | =7.444 |
| 25 | >42.674 | =24.634 |
| 100 | >42.663 | =3.595 |
| 56 | >42.663 | =25.337 |
| 266 | >42.663 | =23.975 |
| 58 | >42.661 | =8.783 |
| 87 | >42.663 | >31.997 |
| 89 | >42.662 | =5.323 |
| 47 | >42.662 | =16.405 |
| 268 | >42.660 | =9.041 |
| 243 | >33.333 | =25.540 |
| 257 | >42.659 | =22.403 |
| 55 | =33.253 | >32.007 |
| 75 | >42.677 | =20.485 |
| 54 | >42.659 | =22.348 |
| 10 | >33.333 | =19.511 |
| 265 | >42.674 | =11.344 |
| 83 | >42.676 | =4.161 |
| 19 | >42.669 | >32.001 |
| 11 | >42.672 | =14.661 |
| 6 | >42.664 | =7.321 |
| 267 | >42.678 | =9.391 |
| 2 | >42.659 | =26.500 |
| 66 | >42.667 | =23.012 |
| 86 | >42.678 | =9.693 |
| 255 | >42.663 | =21.784 |
| 73 | >42.667 | =19.936 |
| 81 | >42.675 | =6.720 |
| 64 | >42.664 | =22.852 |
| 3 | >42.661 | >31.996 |
| 99 | >42.658 | =3.690 |
| 90 | >42.667 | >32.000 |
| 98 | >42.674 | =7.333 |
| 264 | >42.673 | =8.865 |
| 9 | >42.667 | =3.614 |
| 15 | >42.667 | =3.094 |
| 31 | >42.667 | =6.364 |
| 247 | >33.333 | =17.296 |
| 28 | >42.667 | =5.524 |
| 23 | >42.667 | =20.314 |
| 240 | >42.667 | =7.729 |
| 94 | >42.667 | =3.395 |
| 49 | >42.667 | =11.194 |
| 21 | >42.667 | =14.234 |
| 38 | =2.844 | =23.797 |
| 253 | >42.667 | =8.698 |
| 68 | >42.667 | =23.134 |
| 260 | >42.667 | =22.501 |
| 72 | >42.667 | =23.610 |
| 91 | >42.667 | =8.139 |
| 46 | >42.667 | =11.148 |
| 16 | >42.667 | =10.570 |
| 24 | >42.667 | =12.961 |
| 60 | >42.667 | =17.884 |
| 14 | >33.333 | =10.584 |
| 62 | >42.667 | =22.629 |
| 34 | >42.667 | =6.810 |
| 246 | >42.667 | =1.876 |
| 242 | >42.667 | =1.766 |
| 41 | >42.667 | =8.444 |
| 92 | >42.667 | =17.569 |
| 17 | >42.667 | =6.595 |
| 39 | >42.667 | =7.420 |
| 7 | >42.667 | =8.310 |
| 37 | >42.667 | =6.928 |
| 12 | >42.667 | =3.110 |
| 170 | >42.667 | =3.748 |
| 252 | >42.667 | =4.759 |
| 271 | >33.333 | =22.883 |
| 258 | >42.667 | >32.000 |
| 239 | >42.667 | — |
| 69 | >42.667 | =4.585 |
| 241 | >42.667 | =17.066 |
| 290 | >42.667 | — |
| 288 | >42.667 | =24.997 |
| 172 | >42.667 | =11.320 |
| 176 | =30.838 | =10.320 |
| 163 | >42.667 | =1.763 |
| 212 | >42.667 | =23.885 |
| 167 | >42.667 | =3.246 |
| 295 | >42.667 | =21.566 |
| 219 | >42.667 | =2.264 |
| 211 | >42.667 | — |
| 214 | >42.667 | — |
| 231 | >42.667 | — |
| 217 | >42.667 | =20.287 |
| 296 | >42.667 | =18.259 |
| 168 | >42.667 | =3.611 |
| 175 | >36.173 | =3.894 |
| 173 | >42.667 | =0.208 |
| 221 | >42.667 | =23.217 |
| 174 | >42.667 | =1.854 |
| 222 | >42.667 | =7.730 |
| 199 | >42.667 | =11.460 |
| 171 | >42.667 | =4.541 |
| 162 | >42.667 | >32.000 |
| 220 | >42.667 | =3.082 |
| 244 | >33.333 | =15.928 |
| 77 | >42.667 | =21.246 |
| 249 | >42.667 | =3.663 |
| 166 | >42.667 | =3.983 |
| 43 | >42.667 | =9.171 |
| 53 | =19.720 | >32.000 |
| 57 | >42.667 | =4.473 |
| 42 | =5.679 | =6.393 |
| 79 | >42.667 | =31.261 |
| 59 | >42.667 | =5.740 |
| 61 | >42.667 | =9.452 |
| 63 | >42.667 | =4.996 |
| 223 | >42.667 | =25.025 |
| 297 | >42.667 | =23.598 |
| 179 | >42.667 | =8.544 |
| 65 | >42.667 | =2.480 |
| 44 | >42.667 | =9.544 |
| 67 | >42.667 | =5.672 |
| 93 | >42.667 | =4.050 |
| 286 | >42.667 | =19.241 |
| 229 | >42.667 | =10.947 |
| 225 | >42.667 | — |
| 261 | >42.667 | =9.304 |
| 70 | >42.667 | =14.626 |
| 96 | >42.667 | =11.418 |
| 238 | >42.667 | — |
| 272 | >42.667 | — |
| 27 | >42.667 | =20.667 |
| 216 | >42.667 | =25.257 |
| 180 | >42.667 | =11.778 |
| 4 | >42.667 | >32.000 |
| 76 | >33.333 | =6.266 |
| 270 | >33.333 | =25.531 |
| 161 | =29.915 | >25.000 |
| 160 | >33.333 | >25.000 |
| 165 | >33.333 | =18.632 |
| 177 | >33.333 | =0.767 |
| 164 | >33.333 | =18.510 |
| 285 | >33.333 | =10.996 |
| 80 | >33.333 | =9.532 |
| 195 | >33.333 | =6.900 |
| 287 | >33.333 | =5.013 |
| 205 | >33.333 | =9.962 |
| 181 | >33.333 | =2.405 |
| 269 | >33.333 | =19.617 |
| 227 | >33.333 | =17.008 |
| 178 | >42.667 | =0.599 |
| 215 | >33.333 | =14.135 |
| 185 | >33.333 | =4.787 |
| 232 | >33.333 | =22.568 |
| 188 | >33.333 | =5.161 |
| 182 | >42.667 | =1.817 |
| 197 | >33.333 | =8.971 |
| 256 | — | =3.982 |
| 202 | >33.333 | =9.684 |
| 218 | >33.333 | =14.826 |
| 200 | >33.333 | =9.573 |
| 169 | >33.333 | =17.687 |
| 224 | >33.333 | =16.077 |
| 213 | >33.333 | =12.842 |
| 184 | >33.333 | =4.780 |
| 210 | >33.333 | =12.148 |
| 201 | >33.333 | =9.618 |
| 183 | >42.667 | =1.319 |
| 289 | >33.333 | =15.375 |
| 193 | >33.333 | =5.917 |
| 203 | >33.333 | =9.939 |
| 196 | >33.333 | =7.322 |
| 208 | >33.333 | =11.148 |
| 206 | >33.333 | =10.917 |
| 228 | >33.333 | =17.242 |
| 198 | >33.333 | =9.223 |
| 209 | >33.333 | =11.802 |
| 204 | >33.333 | =9.959 |
| 230 | >33.333 | =19.583 |
| 194 | >33.333 | =5.222 |
| 51 | — | =7.658 |
| 78 | >33.333 | =22.168 |
| 226 | >33.333 | =16.342 |
| 186 | =40.853 | =2.027 |
| 36 | >41.341 | =17.039 |
| 35 | =1.283 | =20.495 |
| 189 | >42.667 | =3.991 |
| 191 | >42.667 | =2.875 |
| 190 | >42.667 | =1.094 |
| 192 | >42.667 | =4.869 |
| 102 | >42.676 | >32.007 |
| 103 | >42.668 | >32.001 |
| 104 | >42.675 | >32.006 |
| 105 | >42.661 | >31.996 |
| 106 | >42.661 | >31.996 |
| 107 | >133.334 | =11.546 |
| 108 | >42.660 | >31.995 |
| 109 | >42.667 | >32.000 |
| 110 | >42.674 | =8.112 |
| 111 | >42.669 | >32.001 |
| 112 | >42.669 | >32.001 |
| 113 | >42.660 | >31.996 |
| 114 | >42.669 | >32.001 |
| 115 | >42.666 | =14.369 |
| 116 | >42.662 | >31.996 |
| 117 | >42.657 | >31.993 |
| 118 | >42.667 | >32.000 |
| 119 | >42.667 | >32.000 |
| 120 | >133.334 | =31.781 |
| 121 | >42.667 | >32.000 |
| 122 | >42.667 | >32.000 |
| 123 | >42.667 | >32.000 |
| 124 | =8.563 | =28.867 |
| 125 | >42.667 | >32.000 |
| 126 | >42.667 | >32.000 |
| 127 | >42.667 | =11.472 |
| 128 | >42.667 | >32.000 |
| 129 | >42.667 | >32.000 |
| 130 | >42.667 | >32.000 |
| 131 | >42.667 | >32.000 |
| 132 | >42.667 | =12.754 |
| 133 | >133.334 | =10.960 |
| 134 | =47.268 | =4.636 |
| 135 | =54.699 | =2.470 |
| 136 | >133.334 | =9.505 |
| 137 | >133.334 | >100 |
| 138 | >133.334 | =26.739 |
| 139 | =1.013 | =2.902 |
| 140 | =19.094 | =7.229 |
| 141 | =7.906 | =16.310 |
| 142 | =0.187 | =4.681 |
| 143 | =16.033 | =6.300 |
| 144 | =23.014 | =3.095 |
| 145 | =23.123 | =5.921 |
| 146 | =2.021 | =23.938 |
| 147 | =1.971 | =4.219 |
| 148 | =83.670 | =3.226 |
| 149 | =30.388 | =1.777 |
| 150 | >133.334 | =1.686 |
| 151 | =1.247 | =18.369 |
| 152 | =24.121 | =2.505 |
| 153 | =4.054 | =18.647 |
| 154 | =122.580 | =5.461 |
| 155 | >133.334 | — |
| 156 | =5.153 | =6.688 |
| 157 | =29.513 | =1.946 |
| 158 | — | =31.083 |
| 159 | — | =9.593 |
| 233 | >42.667 | >32.000 |
| 234 | >36.173 | =3.894 |
| 235 | >42.667 | >32.000 |
| 236 | >42.667 | =11.765 |
| 237 | >42.667 | =18.189 |
| 276 | >42.657 | >31.993 |
| 277 | >42.670 | =13.631 |
| 278 | >42.666 | =13.300 |
| 279 | >42.675 | =9.266 |
| 280 | >42.667 | >32.000 |
| 281 | >42.667 | >32.000 |
| 282 | >42.667 | =17.042 |
| 283 | >42.667 | >32.000 |
| 284 | >42.667 | =30.898 |
| 291 | >42.667 | >32.000 |
| 293 | >42.667 | >32.000 |
| 294 | =10.424 | =41.974 |
| 298 | >42.667 | >32.000 |
| 299 | >133.334 | =2.362 |
| 300 | >133.334 | =0.706 |
| 302 | =92.472 | =1.870 |
| 301 | =1.517 | =15.257 |
| 304 | =0.460 | =7.589 |
| 303 | =31.530 | — |
| 306 | =83.119 | =3.921 |
| 305 | =0.035 | =7.739 |
| 309 | >133.334 | =39.644 |
| 310 | =14.520 | =16.441 |
| 311 | =13.328 | =4.028 |
| 312 | =6.115 | =7.809 |
| 313 | =6.043 | =27.481 |
| 314 | =4.432 | =3.004 |
| 315 | =4.776 | =14.636 |
| 316 | =6.973 | =15.385 |
| 317 | >133.334 | =5.752 |
| 318 | =67.086 | — |
| 319 | =12.687 | =32.428 |
| 320 | =6.767 | =33.022 |
| 321 | >133.334 | =72.172 |
| 322 | — | =44.085 |
| 323 | =53.800 | =1.650 |

TABLE 19

| Compound | NS5B Polymerase Assay | Replicon Assay |
|---|---|---|
| No. | IC₅₀ (μM) | EC₅₀ (μM) |
| 324 | >42.612 | =18.527 |
| 325 | >42.612 | =16.290 |
| 326 | >33.333 | =4.692 |
| 327 | >33.333 | =12.494 |
| 328 | >42.670 | =22.713 |
| 329 | >33.333 | =11.085 |
| 330 | >42.680 | =23.202 |
| 331 | >42.663 | =7.441 |
| 332 | >42.675 | =9.740 |
| 333 | >42.678 | — |
| 334 | >42.670 | — |
| 335 | >42.672 | =11.929 |
| 336 | >42.664 | =13.762 |
| 337 | >42.660 | =17.547 |
| 338 | >42.664 | =16.895 |
| 339 | >42.664 | =9.154 |
| 340 | >42.673 | =5.094 |
| 341 | >42.667 | =3.926 |
| 342 | >42.663 | =4.193 |
| 343 | >42.658 | =10.962 |
| 344 | >42.664 | =21.600 |
| 345 | >42.662 | =21.008 |
| 346 | >42.666 | =24.584 |
| 347 | >42.672 | =20.700 |
| 348 | =29.574 | =4.476 |
| 349 | >42.677 | =2.863 |
| 350 | =25.136 | =5.213 |
| 351 | >42.672 | — |
| 352 | >42.665 | =9.608 |
| 353 | >42.667 | =5.304 |
| 354 | >42.664 | =20.534 |
| 355 | >42.666 | =5.483 |
| 356 | >42.665 | =16.095 |
| 357 | >42.666 | =7.006 |
| 358 | >42.670 | =20.435 |
| 359 | >42.663 | =7.332 |
| 360 | >42.667 | =11.225 |
| 361 | >42.667 | =6.650 |
| 362 | =30.574 | =19.271 |
| 363 | >42.667 | =25.826 |
| 364 | >42.667 | =20.793 |
| 365 | =27.340 | =5.240 |
| 366 | >42.667 | =5.308 |
| 367 | >42.667 | =24.606 |
| 368 | >42.667 | =14.940 |
| 369 | >42.667 | =10.001 |
| 370 | >42.667 | =3.425 |
| 371 | >42.667 | =3.737 |
| 372 | >42.667 | =1.944 |
| 373 | >42.667 | =9.415 |
| 374 | >42.667 | =10.106 |
| 375 | >42.667 | =19.933 |
| 376 | >42.667 | =9.450 |
| 377 | >42.667 | =4.463 |
| 378 | >42.667 | =5.944 |
| 379 | >42.667 | =6.789 |
| 380 | >42.667 | =8.626 |
| 381 | >42.667 | =4.766 |
| 382 | >42.667 | =16.660 |
| 383 | >42.667 | =17.252 |
| 384 | =4.582 | =15.911 |
| 385 | >42.667 | =3.535 |
| 386 | >42.667 | >29.590 |
| 387 | >42.667 | =5.558 |
| 388 | >42.667 | =6.350 |
| 389 | >42.667 | =5.577 |
| 390 | >42.667 | =9.067 |
| 391 | >42.667 | =18.488 |
| 392 | >42.667 | =22.649 |
| 393 | >33.333 | =6.844 |
| 394 | >42.667 | =14.075 |
| 395 | >42.667 | =9.906 |
| 396 | >42.667 | =11.306 |
| 397 | >42.667 | =6.131 |
| 398 | >42.667 | =10.911 |
| 399 | >42.667 | =19.434 |
| 400 | >42.667 | =3.278 |
| 401 | >42.667 | =2.880 |
| 402 | >42.667 | =8.593 |
| 403 | >42.667 | =7.115 |
| 404 | >42.667 | =3.242 |
| 405 | >42.667 | =5.344 |
| 406 | >42.667 | =7.290 |
| 407 | >33.333 | =15.646 |
| 408 | >33.333 | =14.159 |
| 409 | >33.333 | =14.892 |
| 410 | >33.333 | =22.575 |
| 411 | >33.333 | =17.869 |
| 412 | >33.333 | =16.678 |
| 413 | >33.333 | =12.031 |
| 414 | >33.333 | =13.640 |
| 415 | >33.333 | =14.666 |
| 416 | >33.333 | =11.166 |
| 417 | >42.667 | =8.328 |
| 418 | >42.667 | =2.162 |
| 419 | >42.667 | =4.815 |
| 420 | >42.667 | =3.235 |
| 421 | >133.334 | =51.929 |
| 423 | =37.863 | =26.399 |
| 424 | =11.606 | =12.534 |
| 425 | >42.674 | =13.245 |
| 426 | =30.781 | =7.306 |
| 427 | >42.667 | =20.672 |
| 428 | >42.667 | =15.991 |
| 429 | >42.667 | >30.966 |
| 430 | >33.333 | =10.832 |
| 431 | =82.880 | >100 |
| 449 | >42.667 | =1.211 |
| 450 | >42.667 | =4.517 |
| 451 | >33.333 | =11.324 |
| 452 | >42.680 | =19.688 |
| 453 | >42.664 | =19.174 |
| 454 | >42.667 | =3.435 |
| 455 | >42.667 | =5.070 |
| 456 | >42.667 | =17.759 |
| 457 | >42.667 | =2.636 |
| 458 | >42.667 | =22.916 |
| 459 | >42.667 | — |
| 460 | >42.667 | =15.648 |
| 461 | >42.667 | =24.525 |
| 462 | =30.855 | >25.000 |
| 463 | >33.333 | =7.432 |
| 464 | >133.334 | =82.697 |
| 475 | >42.661 | — |
| 465 | =59.958 | =3.838 |
| 476 | >42.667 | =22.889 |
| 477 | >42.667 | =4.492 |
| 478 | >42.667 | =2.405 |
| 479 | >42.667 | — |
| 480 | >42.667 | =5.695 |
| 481 | >42.667 | =1.609 |
| 482 | >42.667 | =27.029 |
| 483 | >42.667 | =15.517 |
| 484 | >42.667 | =6.647 |
| 485 | >42.667 | =3.388 |
| 486 | >42.667 | =3.305 |
| 487 | >42.667 | =3.149 |
| 488 | >42.667 | =17.821 |
| 489 | >42.667 | =5.905 |
| 490 | >42.667 | =24.862 |
| 491 | >42.667 | =12.070 |
| 492 | >42.667 | =11.024 |
| 493 | >42.667 | =1.302 |
| 494 | >42.667 | =21.678 |
| 495 | >42.667 | =3.692 |
| 496 | >42.667 | =22.875 |
| 499 | >42.667 | >31.959 |
| 500 | >133.334 | =26.452 |
| 501 | >42.678 | =19.781 |
| 502 | >42.678 | =13.195 |
| 503 | >42.668 | =22.390 |
| 504 | =39.062 | =17.106 |
| 505 | >42.667 | =13.457 |
| 506 | >42.667 | =56.654 |
| 507 | >42.667 | =4.632 |
| 508 | >42.667 | =1.366 |
| 509 | >42.667 | =0.894 |
| 512 | =84.493 | =8.418 |
| 513 | =70.082 | =3.672 |
| 422 | >133.334 | >100 |
| 432 | >42.667 | =17.691 |
| 433 | >133.334 | =3.811 |
| 434 | >133.334 | =22.924 |
| 435 | >133.334 | =6.699 |
| 436 | >133.334 | =19.689 |
| 437 | =32.922 | =6.350 |
| 438 | >133.334 | =15.402 |
| 439 | =3.631 | =4.332 |
| 440 | >133.334 | >100 |
| 441 | >133.334 | >100 |
| 442 | >133.334 | =2.706 |
| 443 | =0.312 | =1.778 |
| 444 | =33.230 | =4.449 |
| 445 | >133.334 | =1.984 |
| 446 | >133.334 | =4.125 |
| 447 | >133.334 | =2.518 |
| 448 | >133.334 | >100 |
| 466 | =68.053 | =6.808 |
| 467 | >133.334 | =30.030 |
| 468 | =25.627 | =5.175 |
| 469 | =32.310 | =8.242 |
| 470 | =23.314 | =16.518 |
| 471 | >133.334 | =14.691 |
| 472 | =9.354 | =16.965 |
| 473 | =49.962 | =19.998 |
| 474 | =1.721 | =34.082 |
| 497 | >133.334 | =3.786 |
| 498 | >133.334 | =2.922 |
| 510 | >133.334 | =4.563 |
| 511 | =1.902 | >100 |

In the following Table 20 there is listed the Mass spectroscopy (MH+) and melting point values for some of the compounds of the invention. An indication of the procedure employed for the preparation of these compounds is also provided.

TABLE 20

| Comp. No. | MH+ | Melting point | prepared according to |
|---|---|---|---|
| 48 | 497 | 216 | Scheme A of Example 92 |
| 45 | 491-495 | >250 | Scheme B of Example 92 |
| 107 | 483-487 | >260 | Scheme C of Example 92 |
| 38 | 429-433 | >250 | Scheme D of Example 92 |
| 120 | 429 | >250 | Scheme D of Example 92 |
| 124 | 497 | 215 | Scheme D of Example 92 |
| 42 | 467 | 170 | Scheme D of Example 92 |
| 273 | 421-423 | 258 | — |
| 322 | 443-447 | 145 | Scheme E of Example 92 |
| 302 | 467 | — | Scheme T of Example 92 |
| 301 | 467 | — | Scheme T of Example 92 |
| 311 | 507-513 | >260 | Scheme F of Example 92 |
| 312 | 507-513 | — | Scheme F of Example 92 |
| 139 | 501-503 | 197 | Scheme G of Example 92 |
| 313 | 454-458 | 248 | Scheme H of Example 92 |
| 314 | 443-447 | — | Scheme F of Example 92 |
| 315 | 443-447 | >260 | Scheme F of Example 92 |
| 316 | 454-458 | — | Scheme H of Example 92 |
| 520 | 487-491 | >250 | Scheme N of Example 92 |
| 521 | 487-491 | >250 | Scheme N of Example 92 |
| 517 | 473-477 | >250 | Scheme P of Example 92 |
| 522 | 473-477 | — | Scheme P of Example 92 |
| 523 | 459-463 | — | Scheme O of Example 92 |
| 524 | 459-463 | — | Scheme O of Example 92 |
| 518 | 473-477 | 200 | Scheme Q of Example 92 |
| 525 | 473-477 | >260 | Scheme P of Example 92 |
| 526 | 459-463 | 200 | Scheme O of Example 92 |
| 304 | 501-503 | 130 | Scheme G of Example 92 |
| 303 | 501-503 | 130 | Scheme G of Example 92 |
| 318 | 454-458 | >250 | Scheme R of Example 92 |
| 319 | 454-458 | >250 | Scheme R of Example 92 |
| 527 | 571-575 | 236 | Scheme I of Example 92 |
| 306 | 501-503 | — | Scheme S of Example 92 |
| 305 | 501-503 | — | Scheme S of Example 92 |
| 515 | 557-561 | 226 | Scheme J of Example 92 |
| 321 | 585-589 | >260 | Scheme J of Example 92 |
| 528 | 577-581 | >260 | Scheme J of Example 92 |
| 514 | 543-547 | 258 | Scheme I of Example 92 |
| 529 | 563-567 | 212 | Scheme I of Example 92 |
| 323 | 519-523 | 235 | Scheme K of Example 92 |
| 530 | 514-518 | >260 | Scheme I of Example 92 |
| 531 | 458-462 | >260 | Scheme O of Example 92 |
| 532 | 528-532 | >260 | Scheme I of Example 92 |
| 151 | 454-458 | >260 | Scheme L of Example 92 |
| 152 | 519-523 | 242 | Scheme L of Example 92 |
| 153 | 481-485 | >260 | Scheme L of Example 92 |
| 533 | 469-473 | 200 | Scheme L of Example 92 |
| 154 | 505-509 | >260 | Scheme L of Example 92 |
| 155 | 498-502 | >260 | Scheme L of Example 92 |
| 156 | 472-476 | >260 | Scheme M of Example 92 |
| 516 | 486-490 | >260 | Scheme O of Example 92 |
| 534 | 546-550 | 220 | Scheme M of Example 92 |
| 535 | 472-476 | >260 | Scheme O of Example 92 |
| 157 | 549-553 | 238 | Scheme M of Example 92 |
| 158 | 510-514 | >260 | Scheme M of Example 92 |
| 159 | 520-524 | >260 | Scheme O of Example 92 |
| 519 | 486-490 | 242 | Scheme U of Example 92 |
| 468 | 465-471 | 170 | Scheme F of Example 92 |
| 469 | 401-405 | 235 | Scheme F of Example 92 |
| 470 | 412-416 | 225 | Scheme H of Example 92 |
| 471 | 401-405 | 225 | Scheme F of Example 92 |
| 536 | 431-434 | >250 | Scheme P of Example 92 |
| 537 | 444-448 | — | Scheme I of Example 92 |
| 538 | 417-421 | 160 | Scheme O of Example 92 |
| 539 | 417-421 | 168 | Scheme O of Example 92 |
| 472 | 412-416 | >250 | Scheme R of Example 92 |
| 473 | 412-416 | — | Scheme R of Example 92 |
| 474 | 402-406 | 248 | Scheme V of Example 106 |
| 540 | 459-463 | 208 | Scheme I of Example 92 |
| 541 | 417-421 | >260 | Scheme I of Example 92 |

1. The use of a compound of the formula (I) for the manufacture of a medicament useful for inhibiting HCV activity in a mammal infected with HCV, said compound being acylated benzodiazepines of the formula (I): 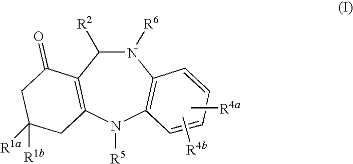
and the salts, stereoisomeric forms, and racemic mixtures thereof in which
R^{1a} and R^{1b} are independently, hydrogen; C₃₋₇cycloalkyl; aryl; Het; or C₁₋₆alkyl optionally substituted independently with one, two or three substituents selected from halo, C₁₋₆alkoxy, aryl and Het; or with a cyano, polyhaloC₁₋₆alkoxy or C₃₋₇cycloalkyl;
R² is hydrogen;
C₁₋₆alkyl optionally substituted independently with one, two or three substituents selected from halo, C₁₋₆alkoxy, aryl and Het; or with a cyano, polyhaloC₁₋₆alkoxy or C₃₋₇cycloalkyl;
C₃₋₇cycloalkyl optionally substituted independently with one, two or three substituents selected from halo, C₁₋₆alkoxy, aryl and Het; or with a cyano, polyhaloC₁₋₆alkoxy or C₃₋₇cycloalkyl,
C₃₋₇cycloalkylC₁₋₆alkyl optionally substituted independently with one, two or three substituents selected from halo, C₁₋₆alkoxy, aryl and Het; or with a cyano, polyhaloC₁₋₆alkoxy or C₃₋₇cycloalkyl;
C₂₋₆alkenyl optionally substituted independently with one, two or three substituents selected from halo, C₁₋₆alkoxy, aryl and Het; or with a cyano, polyhaloC₁₋₆alkoxy or C₃₋₇cycloalkyl;
C₄₋₇cycloalkenyl optionally substituted independently with one, two or three substituents selected from halo, C₁₋₆alkoxy, aryl and Het; or with a cyano, polyhaloC₁₋₆alkoxy or C₃₋₇cycloalkyl;
C₄₋₈cycloalkenylC₁₋₆alkyl optionally substituted independently with one, two or three substituents selected from halo, C₁₋₆alkoxy, aryl and Het; or with a cyano, polyhaloC₁₋₆alkoxy or C₃₋₇cycloalkyl;
aryl²; or
Het²;
R⁶ is hydrogen;
C₁₋₆alkyl optionally substituted with carboxyl, C₁₋₆alkylcarbonyl, C₁₋₆alkoxy-carbonyl, Het-C₁₋₆alkylaminocarbonyl;
—C(═O)—C₁₋₇alkyl, the C₁₋₇alkyl being optionally substituted independently with one, two or three substituents selected from halo, C₁₋₆alkoxy, aryl, Het, cyano, polyhaloC₁₋₆alkoxy, C₃₋₇cycloalkyl, and carboxyl;
—C(═O)—C₂₋₆alkenyl;
—C(═O)—C₃₋₇cycloalkyl, the C₃₋₇cycloalkyl being optionally substituted independently with one, two or three substituents selected from halo, C₁₋₆alkoxy, aryl, Het, cyano, polyhaloC₁₋₆alkoxy, and C₃₋₇cycloalkyl;
—C(═O)-aryl;
—C(═O)-Het;
—C(═O)—NR^{12a}R^{12b},
in which each R^{12a} and R^{12b} is, independently, hydrogen, C₃₋₇cycloalkyl, aryl, Het, or C₁₋₆alkyl optionally substituted independently with one, two or three substituents selected from halo, C₁₋₆alkoxy, aryl, Het, cyano, polyhaloC₁₋₆alkoxy, and C₃₋₇cycloalkyl;
—C(═O)—OR^{13a},
in which R¹³ is hydrogen, C₂₋₆alkenyl, C₃₋₇cycloalkyl, Het, or C₁₋₆alkyl optionally substituted with a C₃₋₇cycloalkyl or Het;
—C(═O)—C₁₋₆alkyloxycarbonylC₁₋₆alkyl;
—C(═O)—Het-thioC₁₋₆alkyl; or
—C(═O)—Het-oxyC₁₋₆alkyl; or
R² and R⁶, together with the intervening grouping in formula (I) of sub-formula: 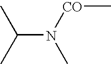
form a ring of formula: 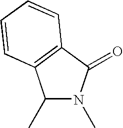
R^{4a} and R^{4b} are independently hydrogen; halo; cyano; C₁₋₆alkyl optionally substituted with halo, hydroxy, Het, —OR^{14a}, or —NR^{14a}R^{14b}; C₁₋₆alkoxy optionally substituted with amino, hydroxy, C₁₋₆alkoxy, hydroxycarbonyl, aryl, or Het; aryloxy; Het-oxy; carboxyl; C₁₋₆alkylcarbonyloxy; C₁₋₆alkoxycarbonyl; arylcarbonyl; —NR^{14a}R^{14b}; or —C(═O)—NR^{14a}R^{14b};
in which each R^{14a} and R^{14b} is, independently, hydrogen; C₃₋₇cycloalkyl; aryl; Het; or C₁₋₆alkyl optionally substituted independently with one, two or three substituents selected from halo, C₁₋₆alkoxy, mono- or diC₁₋₆alkylamino, aryl, Het, cyano, polyhaloC₁₋₆alkoxy, and C₃₋₇cycloalkyl;
R⁵ is hydrogen; C₃₋₇cycloalkyl; or C₁₋₆alkyl optionally substituted with a C₃₋₇cyclo-alkyl, aryl, Het, —C(═O)NR^{15a}R^{15b}, NR^{15a}R^{15b}, —C(═O)R¹⁷, —NR^{15a}C(═O)R¹⁷, —NR^{15a}SOₚR¹⁸, —SOₚR¹⁸, —SOₚNR^{15a}R^{15b}, —C(═O)OR¹⁶, or —NR^{15a}C(═O)OR^{16a} in which
p is 0, 1 or 2;
each R^{15a} and R^{15b} is, independently, hydrogen; C₃₋₇cycloalkyl; aryl; Het; or C₁₋₆alkyl optionally substituted independently with one, two or three substituents selected from halo, C₁₋₆alkoxy, aryl and Het; or with a cyano, polyhaloC₁₋₆alkoxy or C₃₋₇cycloalkyl;
R¹⁶ is hydrogen; C₂₋₆alkenyl; C₃₋₇cycloalkyl; Het; or C₁₋₆alkyl optionally substituted with a C₃₋₇cycloalkyl or Het;
R^{16a} is C₂₋₆alkenyl; C₃₋₇cycloalkyl; Het; or C₁₋₆alkyl optionally substituted with a C₃₋₇cycloalkyl or Het;
R¹⁷ is hydrogen, C₁₋₆alkyl, C₃₋₇cycloalkyl or aryl;
R¹⁸ is hydrogen; polyhaloC₁₋₆alkyl; C₃₋₇cycloalkyl; aryl; Het; or C₁₋₆alkyl optionally substituted with a C₃₋₇cycloalkyl, aryl or Het;
aryl as a group or part of a group is phenyl, naphthyl, indanyl, or 1,2,3,4-tetrahydro-naphthyl, each of which may be optionally independently substituted with
(a) one, two or three substituents selected from halo, C₁₋₆alkyl, polyhaloC₁₋₆alkyl, hydroxy, trifluoromethyl, alkylenedioxy, C₁₋₆alkoxy, C₁₋₆alkylthio, polyhalo-C₁₋₆alkoxy, C₁₋₆alkoxyC₁₋₆alkyl, carboxyl, C₁₋₆alkylcarbonyl, cyano, cyanoC₁₋₆alkyl, nitro, amino, mono- or diC₁₋₆alkylamino, azido, mercapto, C₃₋₇cycloalkyl, pyrrolidinyl, piperidinyl, piperazinyl, 4-C₁₋₆alkylpiperazinyl, 4-C₁₋₆alkylcarbonyl-piperazinyl, and morpholinyl; or
(b) phenyl- or naphthyl-alkoxy optionally substituted with one, two or three substituents defined for (a) above; or
(c) phenyl- or naphthyl-carbonyloxy optionally substituted with one, two or three substituents defined for (a) above; and
Het as a group or part of a group is a 5 or 6 membered saturated, partially unsaturated or completely unsaturated heterocyclic ring containing 1 to 4 heteroatoms each independently selected from nitrogen, oxygen and sulfur, being optionally condensed with one or two benzene rings, and wherein the group Het as a whole may be optionally substituted with one, two or three substituents each independently selected from the group consisting of halo, C₁₋₆alkyl, polyhaloC₁₋₆alkyl, hydroxy, aryl, C₁₋₆alkoxy, polyhaloC₁₋₆alkoxy, C₁₋₆alkoxyC₁₋₆alkyl, carboxyl, C₁₋₆alkylcarbonyl, cyano, nitro, amino, mono- or diC₁₋₆alkylamino, aminocarbonyl, C₃₋₇cycloalkyl, pyrrolidinyl, piperidinyl, piperazinyl, 4-C₁₋₆alkylpiperazinyl, 4-C₁₋₆alkylcarbonyl-piperazinyl, and morpholinyl;
aryl² as a group or part of a group is phenyl, naphthyl, indanyl, or 1,2,3,4-tetrahydro-naphthyl, each of which may be optionally independently substituted with one, two or three substituents selected from
(a) halo, C₁₋₆alkyl, polyhaloC₁₋₆alkyl, hydroxy, trifluoromethyl, alkylenedioxy, C₁₋₆alkoxy, C₁₋₆alkylthio, polyhalo-C₁₋₆alkoxy, C₁₋₆alkylcarbonyloxy, C₁₋₆alkoxyC₁₋₆alkyl, carboxyl, C₁₋₆alkylcarbonyl, cyano, nitro, amino, mono- or diC₁₋₆alkylamino, azido, mercapto, C₃₋₇cycloalkyl, pyrrolidinyl, piperidinyl, piperazinyl, 4-C₁₋₆alkylpiperazinyl, 4-C₁₋₆alkyl-carbonyl-piperazinyl, morpholinyl; phenyl- or napthyl-alkoxy optionally substituted with halogen; phenyl- or naphthyl-carbonyloxy optionally substituted with halogen, polyhaloC₁₋₆alkoxy, C₁₋₆alkoxyC₁₋₆alkyl, carboxyl, C₁₋₆alkylcarbonyl, cyano, nitro, amino, mono- or diC₁₋₆alkylamino, azido, mercapto, C₃₋₇cycloalkyl, pyrrolidinyl, piperidinyl, piperazinyl, 4-C₁₋₆alkylpiperazinyl, 4-C₁₋₆alkyl-carbonyl-piperazinyl, morpholinyl; or
(b) a radical of formula —(X)ₙ-aryl or —(X)ₙ-Het in which n is 0 or 1 and X is —C₁₋₆alkanediyl-, C₁₋₆alkenediyl-, —NR²⁰—, —NR²⁰—C₁₋₆alkanediyl-, —NR²⁰—CO—C₁₋₆alkanediyl-, —CO—NR¹⁰—C₁₋₆alkanediyl-, —O—, —CO—NR¹⁰—, —NR²⁰—CO—, —NR²⁰—SO₂—, —SO₂—NR²⁰—, —O—C₁₋₆alkanediyl-, —O—CO—, —CO—, —O—CO—C₁₋₆alkanediyl-, —S— or —S—C₁₋₆alkanediyl-
in which R²⁰ is hydrogen, C₃₋₇cycloalkyl, aryl, Het, C₁₋₆alkyl optionally substituted independently with one, two or three substituents selected from halo, C₁₋₆alkoxy, aryl, Het, cyano, polyhaloC₁₋₆alkoxy, and C₃₋₇cycloalkyl;
Het² as a group or part of a group is a 5 or 6 membered saturated, partially unsaturated or completely unsaturated heterocyclic ring containing 1 to 4 heteroatoms each independently selected from nitrogen, oxygen and sulfur, being optionally condensed with one or two benzene rings, and wherein the group Het as a whole may be optionally substituted with one, two or three substituents each independently selected from the group consisting of halo, C₁₋₆alkyl, polyhaloC₁₋₆alkyl, hydroxy, oxo, aryl, C₁₋₆alkoxy, polyhaloC₁₋₆alkoxy, C₁₋₆alkoxyC₁₋₆alkyl, carboxyl, C₁₋₆alkylcarbonyl, cyano, nitro, amino, mono- or diC₁₋₆alkylamino, cycloalkyl, pyrrolidinyl, piperidinyl, piperazinyl, 4-C₁₋₆alkylpiperazinyl, 4-C₁₋₆alkylcarbonyl-piperazinyl, morpholinyl; or Het² is substituted with a radical of formula —(X)ₙ-aryl or —(X)ₙ-Het in which n is 0 or 1 and X is —C₁₋₆alkanediyl-, C₁₋₆alkenediyl-, —NR²¹—, —NR²¹—C₁₋₆alkanediyl-, —NR²¹—CO—C₁₋₆alkanediyl-, —CO—NR²¹—C₁₋₆alkanediyl-, —O—, —O—C₁₋₆alkanediyl-, —O—CO—, —O—CO—C₁₋₆alkanediyl-, —S—, or —S—C₁₋₆alkanediyl-
in which R²¹ is hydrogen, C₃₋₇cycloalkyl, aryl, Het, C₁₋₆alkyl optionally substituted independently with one, two or three substituents selected from halo, C₁₋₆alkoxyaryl and Het; or with a cyano, polyhaloC₁₋₆alkoxy or C₃₋₇cycloalkyl.

2. A compound of the formula (I) 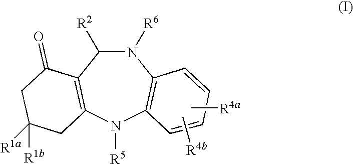
and the salts, stereoisomeric forms, and racemic mixtures thereof in which
R^{1a} and R^{1b} are independently, hydrogen, aryl, Het, or C₁₋₆alkyl;
R² is C₂₋₆alkenyl optionally substituted independently with one or two substituents selected from halo, and aryl;
aryl²; or
Het²;
R⁶ is hydrogen;
C₁₋₆alkyl optionally substituted with carboxyl, C₁₋₆alkylcarbonyl, C₁₋₆alkoxy-carbonyl, Het-C₁₋₆alkylaminocarbonyl;
—C(═O)—C₁₋₇alkyl, the C₁₋₇alkyl being optionally substituted independently with one, two or three substituents selected from halo, aryl, and cyano;
—C(═O)—C₂₋₆alkenyl;
—C(═O)-aryl;
—C(═O)—Het;
—C(═O)—NR^{12a}R^{12b},
in which each R^{12a} and R^{12b} is, independently, hydrogen, aryl, or C₁₋₆alkyl optionally substituted independently with one or two substituents selected from aryl and Het;
R^{4a} and Keb are independently hydrogen; halo; cyano; C₁₋₆alkyl optionally substituted with halo, hydroxy, Het, —OR^{14a}, or —NR^{14a}R^{14b}; C₁₋₆alkoxy optionally substituted with amino, hydroxy, C₁₋₆alkoxy, hydroxycarbonyl, aryl, or Het; aryloxy; Het-oxy; carboxyl; C₁₋₆alkylcarbonyloxy; C₁₋₆alkoxycarbonyl; —NR^{14a}R^{14b}; or —C(═O)—NR^{14a}R^{14b};
in which each R^{14a} and R^{14b} is, independently, hydrogen; or C₁₋₆alkyl optionally substituted independently with one or two substituents selected from mono- or diC₁₋₆alkylamino, and Het;
R⁵ is hydrogen; or C₁₋₆alkyl optionally substituted with aryl;
aryl as a group or part of a group is phenyl or naphthyl, each of which may be optionally independently substituted with
(a) one, two or three substituents selected from halo, C₁₋₆alkyl, trifluoromethyl, C₁₋₆alkoxy, carboxyl, C₁₋₆alkylcarbonyl, cyano, cyanoC₁₋₆alkyl, nitro, mono- or diC₁₋₆alkylamino; or
(b) phenyl-alkoxy optionally substituted with one, two or three substituents defined for (a) above;
Het as a group or part of a group is a 5 or 6 membered saturated, partially unsaturated or completely unsaturated heterocyclic ring containing 1 to 4 heteroatoms each independently selected from nitrogen, oxygen and sulfur, being optionally condensed with one or two benzene rings, and wherein the group Het as a whole may be optionally substituted with one, two or three substituents each independently selected from the group consisting of C₁₋₆alkyl, and aminocarbonyl;
aryl² as a group or part of a group is phenyl or naphthyl, each of which may be optionally independently substituted with one, two or three substituents selected from
(e) halo, C₁₋₆alkyl, hydroxy, trifluoromethyl, C₁₋₆alkoxy, polyhaloC₁₋₆alkoxy, C₁₋₆alkylcarbonyloxy, carboxyl, nitro, mono- or diC₁₋₆alkylamino; or
(f) a radical of formula —(X)ₙ-aryl or —(X)ₙ-Het in which n is 1 and X is —O—, —CO—NH—, —NH—CO—, —NH—SO₂—, —SO₂—NH—, —O—C₁₋₆alkanediyl-, —O—CO—, —CO—;
Het² as a group or part of a group is a 5 or 6 membered saturated, partially unsaturated or completely unsaturated heterocyclic ring containing 1 to 4 heteroatoms each independently selected from nitrogen, oxygen and sulfur, being optionally condensed with one or two benzene rings, and wherein the group Het as a whole may be optionally substituted with one, two or three substituents each independently selected from the group consisting of halo, C₁₋₆alkyl, aryl, and nitro.

3. A compound of the formula (Ia) 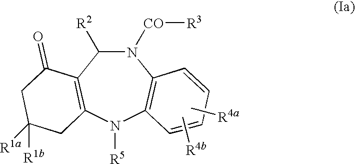
and the salts, stereoisomeric forms, and racemic mixtures thereof in which
R^{1a} and R^{1b} are independently, hydrogen, aryl, Het, or C₁₋₆alkyl;
R² is C₂₋₆alkenyl optionally substituted independently with one or two substituents selected from halo, and aryl;
aryl²; or
Het²;
R³ is C₁₋₇alkyl optionally substituted independently with one, two or three substituents selected from halo, aryl, and cyano;
C₂₋₆alkenyl;
aryl;
Het;
—NR^{12a}R^{12b},
in which each R^{12a} and R^{12b} is, independently, hydrogen, aryl, or C₁₋₆alkyl optionally substituted independently with one or two substituents selected from aryl and Het;
R^{4a} and R^{4b} are independently hydrogen; halo; cyano; C₁₋₆alkyl optionally substituted with halo, hydroxy, Het, —OR^{14a}, or —NR^{14a}R^{14b}; C₁₋₆alkoxy optionally substituted with amino, hydroxy, C₁₋₆alkoxy, hydroxycarbonyl, aryl, or Het; aryloxy; Het-oxy; carboxyl; C₁₋₆alkylcarbonyloxy; C₁₋₆alkoxycarbonyl; —NR^{14a}R^{14b}; or —C(═O)—NR^{14a}R^{14b};
in which each R^{14a} and R^{14b} is, independently, hydrogen; or C₁₋₆alkyl optionally substituted independently with one or two substituents selected from mono- or diC₁₋₆alkylamino, and Het;
R⁵ is hydrogen; or C₁₋₆alkyl optionally substituted with aryl;
aryl as a group or part of a group is phenyl or naphthyl, each of which may be optionally independently substituted with
(a) one, two or three substituents selected from halo, C₁₋₆alkyl, trifluoromethyl, C₁₋₆alkoxy, carboxyl, C₁₋₆alkylcarbonyl, cyano, cyanoC₁₋₆alkyl, nitro, mono- or diC₁₋₆alkylamino; or
(b) phenyl-alkoxy optionally substituted with one, two or three substituents defined for (a) above;
Het as a group or part of a group is a 5 or 6 membered saturated, partially unsaturated or completely unsaturated heterocyclic ring containing 1 to 4 heteroatoms each independently selected from nitrogen, oxygen and sulfur, being optionally condensed with one or two benzene rings, and wherein the group Het as a whole may be optionally substituted with one, two or three substituents each independently selected from the group consisting of C₁₋₆alkyl, and aminocarbonyl;
aryl² as a group or part of a group is phenyl or naphthyl, each of which may be optionally independently substituted with one, two or three substituents selected from
(g) halo, C₁₋₆alkyl, hydroxy, trifluoromethyl, C₁₋₆alkoxy, C₁₋₆alkylcarbonyloxy, polyhaloC₁₋₆alkoxy, nitro, mono- or diC₁₋₆alkylamino; or
(h) a radical of formula —(X)ₙ-aryl or —(X)ₙ-Het in which n is 1 and X is —O—, —CO—NH—, —NH—CO—, —NH—SO₂—, —SO₂—NH—, —O—C₁₋₆alkanediyl-, —O—CO—, —CO—;
Het² as a group or part of a group is a 5 or 6 membered saturated, partially unsaturated or completely unsaturated heterocyclic ring containing 1 to 4 heteroatoms each independently selected from nitrogen, oxygen and sulfur, being optionally condensed with one or two benzene rings, and wherein the group Het as a whole may be optionally substituted with one, two or three substituents each independently selected from the group consisting of halo, C₁₋₆alkyl, aryl, and nitro.

4. A compound of the formula (Ib) 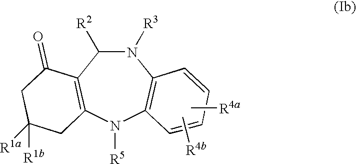
and the salts, stereoisomeric forms, and racemic mixtures thereof in which
R^{1a} and R^{1b} are independently, hydrogen, aryl, or C₁₋₆alkyl;
R² is C₂₋₆alkenyl optionally substituted independently with one or two substituents selected from halo, and aryl;
aryl²; or
Het²;
R³ is hydrogen;
C₁₋₆alkyl optionally substituted with carboxyl, C₁₋₆alkylcarbonyl, C₁₋₆alkoxycarbonyl, Het-C₁₋₆alkylaminocarbonyl;
R^{4a} and R^{4b} are independently hydrogen; halo; cyano; C₁₋₆alkyl optionally substituted with halo, hydroxy, or —NR^{14a}R^{14b}; C₁₋₆alkoxy optionally substituted with C₁₋₆alkoxy; carboxyl; or —NR^{14a}R^{14b};
in which each R^{14a} and R^{14b} is, independently, hydrogen; or C₁₋₆alkyl;
R⁵ is hydrogen;
aryl as a group or part of a group is phenyl or naphthyl, each of which may be optionally independently substituted with
(a) one, two or three substituents selected from halo, and C₁₋₆alkoxy; or
(b) phenyl-alkoxy optionally substituted with one, two or three substituents defined for (a) above;
Het as a group or part of a group is a 5 or 6 membered saturated, partially unsaturated or completely unsaturated heterocyclic ring containing 1 to 4 heteroatoms each independently selected from nitrogen, oxygen and sulfur, being optionally condensed with one or two benzene rings;
aryl² as a group or part of a group is phenyl or naphthyl, each of which may be optionally independently substituted with one, two or three substituents selected from
c) halo, hydroxy, polyhalo-C₁₋₆alkoxy, carboxyl, nitro; or
d) a radical of formula —(X)ₙ-aryl in which n is 1 and X is —O—, —CO—NH—, —SO₂—NH—, —O—C₁₋₆alkanediyl-, —O—CO—, —CO—;
Het² as a group or part of a group is a 5 or 6 membered saturated, partially unsaturated or completely unsaturated heterocyclic ring containing 1 to 4 heteroatoms each independently selected from nitrogen, oxygen and sulfur, being optionally condensed with one or two benzene rings, and wherein the group Het as a whole may be optionally substituted with one, two or three halo.

5. The use as claimed in claim 1 or the compounds according to any one of claims 2-4 wherein
at least one of R^{1a} and R^{1b} is hydrogen, halo, C₁₋₆alkyl, aryl or Het.
R² is hydrogen; C₂₋₆alkenyl optionally substituted with aryl or halo; C₄₋₈cycloalkenyl-C₁₋₆alkyl; aryl²; or Het².
at least one of R^{4a} and R^{4b} is hydrogen or arylcarbonyl.
R⁵ is hydrogen.

6. The use as claimed in claim 1 or the compounds according to any one of claims 2-3 wherein R³ or R⁶ is C₁₋₆alkyl or polyhaloC₁₋₆alkyl.

7. The use as claimed in claim 1 or the compounds according to any one of claims 2 and 4 wherein R³ or R⁶ is hydrogen or C₁₋₆alkyl.

8. A method of treating an HCV infection, comprising administering to a mammal in need thereof an effective amount of a compound of formula (I) as defined in claim 1 or a salt, stereoisomeric form, or racemic mixture thereof.

9. A pharmaceutical composition comprising a therapeutically effective amount of a novel compound of formula (I) according to any one of claims 2-4, and a pharmaceutically acceptable carrier.

10. A process of preparing a pharmaceutical composition as claimed in claim 9, which comprises intimately mixing a pharmaceutically acceptable carrier with a therapeutically effective amount of a compound of formula (I) according to any one of claims 2-4.
